(19) United States
(12) Patent Application Publication
(10) Pub. No.: US 2024/0341176 A1
(43) Pub. Date: Oct. 10, 2024

(54) COMPOUND AND ORGANIC LIGHT-EMITTING DEVICE COMPRISING SAME

(71) Applicant: LG CHEM, LTD., Seoul (KR)

(72) Inventors: Jung Min YOON, Daejeon (KR); Heekyung YUN, Daejeon (KR); Jae Tak LEE, Daejeon (KR); Su Jin HAN, Daejeon (KR); Dong Uk HEO, Daejeon (KR); Sung Kil HONG, Daejeon (KR)

(21) Appl. No.: 18/660,201

(22) Filed: May 9, 2024

Related U.S. Application Data
(63) Continuation-in-part of application No. PCT/KR2022/016048, filed on Oct. 20, 2022.

(30) Foreign Application Priority Data
Nov. 11, 2021 (KR) 10-2021-0154719

(51) Int. Cl. H10K 85/60; C07D 401/14; C09K 11/06; H10K 50/16; H10K 50/17; H10K 50/18

(52) U.S. Cl. CPC H10K 85/654; C07D 401/14; C09K 11/06; H10K 85/615; C09K 2211/1018; H10K 50/16; H10K 50/171; H10K 50/18

(57) ABSTRACT

The present specification relates to a compound of Chemical Formula 1 and an organic light emitting device including the same.

### TECHNICAL FIELD

This application claims priority to and the benefit of Korean Patent Application No. 10-2021-0154719 filed in the Korean Intellectual Property Office on Nov. 11, 2021, the entire contents of which are incorporated herein by reference.

The present specification relates to a compound and an organic light emitting device including the same.

### BACKGROUND ART

An organic light emission phenomenon generally refers to a phenomenon converting electrical energy to light energy using an organic material. An organic light emitting device using an organic light emission phenomenon normally has a structure including a positive electrode, a negative electrode, and an organic material layer therebetween. Here, the organic material layer may have a multi-layered structure composed of different materials in order to improve the efficiency and stability of an organic light emitting device in many cases, and for example, may be composed of a hole injection layer, a hole transport layer, a light emitting layer, an electron transport layer, an electron injection layer, and the like. In such a structure of the organic light emitting device, if a voltage is applied between the two electrodes, holes are injected from the positive electrode into the organic material layer and electrons are injected from the negative electrode into the organic material layer, and when the injected holes and electrons meet each other, an exciton is formed, and light is emitted when the exciton falls down again to a ground state.

There is a continuous need for developing a new material for the aforementioned organic light emitting device.

### DETAILED DESCRIPTION OF THE INVENTION

### Technical Problem

The present specification provides a compound and an organic light emitting device including the same.

### Technical Solution

An exemplary embodiment of the present specification provides a compound of the following Chemical Formula 1.

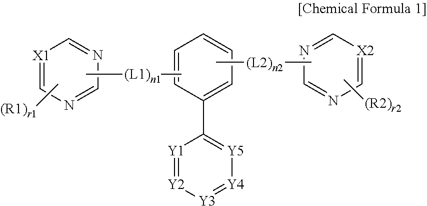

In Chemical Formula 1,
any one or two of Y1 to Y5 is or are N, and the others are CR3,
X1 is N or CR′1,
X2 is N or CR′2,
R′1 is hydrogen; deuterium; a substituted or unsubstituted alkyl group; a substituted or unsubstituted aryl group; a substituted or unsubstituted heteroaryl group; or a moiety bonded to L1, or is bonded to R1 to form a substituted or unsubstituted aromatic hydrocarbon ring,
R′2 is hydrogen; deuterium; a substituted or unsubstituted alkyl group; a substituted or unsubstituted aryl group; a substituted or unsubstituted heteroaryl group; or a moiety bonded to L2, or is boned to R2 to form a substituted or unsubstituted aromatic hydrocarbon ring,
R1 and R2 are the same as or different from each other, and are each independently a substituted or unsubstituted alkyl group; a substituted or unsubstituted aryl group; or a substituted or unsubstituted heteroaryl group, or are bonded to an adjacent group to form a substituted or unsubstituted aromatic hydrocarbon ring,
R3 is hydrogen; deuterium; or a substituted or unsubstituted alkyl group,
L1 and L2 are the same as or different from each other, and are each independently a substituted or unsubstituted arylene group,
n1 and n2 are each an integer from 1 to 5,
r1 and r2 are each an integer from 1 to 3,
when n1 is 2 or higher, two or more L1's are the same as or different from each other,
when n2 is 2 or higher, two or more L2's are the same as or different from each other,
when r1 is 2 or higher, two or more R1's are the same as or different from each other, and
when r2 is 2 or higher, two or more R2's are the same as or different from each other.

Further, an exemplary embodiment of the present specification provides an organic light emitting device including: a first electrode; a second electrode; and an organic material layer having one or more layers provided between the first electrode and the second electrode, in which one or more layers of the organic material layer include the compound.

### Advantageous Effects

The compound according to an exemplary embodiment of the present specification may be used as a material for an organic material layer of an organic light emitting device, and it is possible to improve efficiency, achieve a low driving voltage, and/or improve service life characteristics, in the organic light emitting device by using the compound.

### BRIEF DESCRIPTION OF DRAWINGS

FIGS. 1 and 2 illustrate an example of an organic light emitting device according to an exemplary embodiment of the present specification.

### EXPLANATION OF REFERENCE NUMERALS AND SYMBOLS

101: Substrate
102: First electrode
111: Organic material layer
110: Second electrode
103: Hole injection layer
104: First hole transport layer
105: Second hole transport layer
106: Light Emitting Layer
107: Electron injection and transport layer

### Best Mode

Hereinafter, the present specification will be described in more detail.

An exemplary embodiment of the present specification provides the compound of Chemical Formula 1.

Since Chemical Formula 1 according to an exemplary embodiment of the present specification is a compound which has a benzene core to which a monocyclic hetero ring including one N or two N's is bonded, inevitably includes an arylene group as a linking group on both sides of benzene, and includes a monocyclic or bicyclic heteroaryl group including two or three N's as a substituent, and may increase the polarity (dipole moment) of the molecule by having electron depletion structure, electron mobility is smoothly adjusted during the manufacture of an organic light emitting device including the compound represented by Chemical Formula 1, so that an organic light emitting device including the compound represented by Chemical Formula 1 has an effect of low voltage, high efficiency, and long service life.

Throughout the specification of the present application, the term “combination thereof” included in the Markush type expression means a mixture or combination of one or more selected from the group consisting of constituent elements described in the Markush type expression, and means including one or more selected from the group consisting of the above-described constituent elements.

Examples of the substituents in the present specification will be described below, but are not limited thereto.

In the present specification, [symbol] means a moiety to be linked.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present invention, the term “substituted or unsubstituted” means being substituted with one or more substituents selected from the group consisting of deuterium; a halogen group; a cyano group; an alkyl group; a cycloalkyl group; an alkoxy group; an alkenyl group; a haloalkyl group; a silyl group; a boron group; an amine group; an aryl group; and a heteroaryl group, being substituted with a substituent to which two or more substituents among the exemplified substituents are linked, or having no substituent.

In the present specification, the fact that two or more substituents are linked indicates that hydrogen of any one substituent is linked to another substituent. For example, when two substituents are linked to each other, a phenyl group and a naphthyl group may be linked to each other to become a substituent of

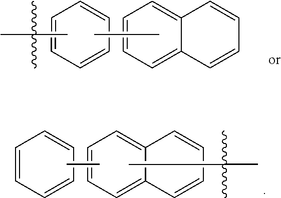

Further, the case where three substituents are linked to one another includes not only a case where (Substituent 1)-(Substituent 2)-(Substituent 3) are consecutively linked to one another, but also a case where (Substituent 2) and (Substituent 3) are linked to (Substituent 1). For example, a phenyl group, a naphthyl group, and an isopropyl group may be linked to one another to become a substituent of

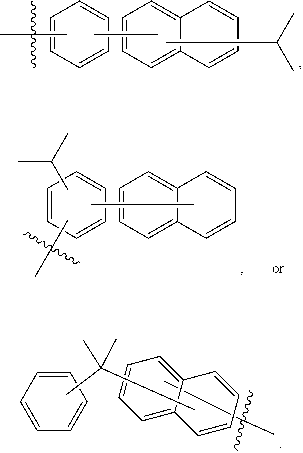

The above-described definition also applies equally to the case where four or more substituents are linked to one another.

In the present specification, examples of a halogen group include a fluoro group, a chloro group, a bromo group or an iodo group.

In the present specification, the alkyl group may be straight-chained or branched, and the number of carbon atoms thereof is not particularly limited, but is preferably 1 to 30. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, a cycloalkyl group is not particularly limited, but has preferably 3 to 30 carbon atoms, and specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, an adamantyl group, and the like, but are not limited thereto.

In the present specification, the alkoxy group may be straight-chained, branched, or cyclic. The number of carbon atoms of the alkoxy group is not particularly limited, but is preferably 1 to 30. Specific examples thereof include a methoxy group, an ethoxy group, an n-propoxy group, an isopropoxy group, an n-butoxy group, an isobutoxy group, a tert-butoxy group, a sec-butoxy group, an n-pentyloxy group, an neopentyloxy group, an isopentyloxy group, an n-hexyloxy group, a 3,3-dimethylbutyloxy group, a 2-ethylbutyloxy group, an n-octyloxy group, an n-nonyloxy group, an n-decyloxy group, a benzyloxy group, a p-methylbenzyloxy group, and the like, but are not limited thereto.

In the present specification, the alkenyl group may be straight-chained or branched, and the number of carbon atoms thereof is not particularly limited, but is preferably 2 to 30. Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl) vinyl-1-yl group, a 2,2-bis(diphenyl-1-yl) vinyl-1-yl group, a stilbenyl group, a styrenyl group, and the like, but are not limited thereto.

In the present specification, the haloalkyl group means that at least one halogen group is substituted instead of hydrogen in an alkyl group in the definition of the alkyl group.

In the present specification, an aryl group is not particularly limited, but has preferably 6 to 30 carbon atoms, and the aryl group may be monocyclic or polycyclic.

When the aryl group is a monocyclic aryl group, the number of carbon atoms is not particularly limited, but is preferably 6 to 30. Specific examples of the monocyclic aryl group include a phenyl group, a biphenyl group, a terphenyl group, and the like, but are not limited thereto.

When the aryl group is a polycyclic aryl group, the number of carbon atoms thereof is not particularly limited, but is preferably 10 to 30. Specific examples of the polycyclic aryl group include a naphthyl group, an anthracene group, a phenanthrene group, a triphenylene group, a pyrene group, a phenalene group, a perylene group, a chrysene group, a fluorene group, and the like, but are not limited thereto.

In the present specification, the fluorene group may be substituted, and adjacent groups may be bonded to each other to form a ring.

Examples of the case where the fluorene group is substituted include

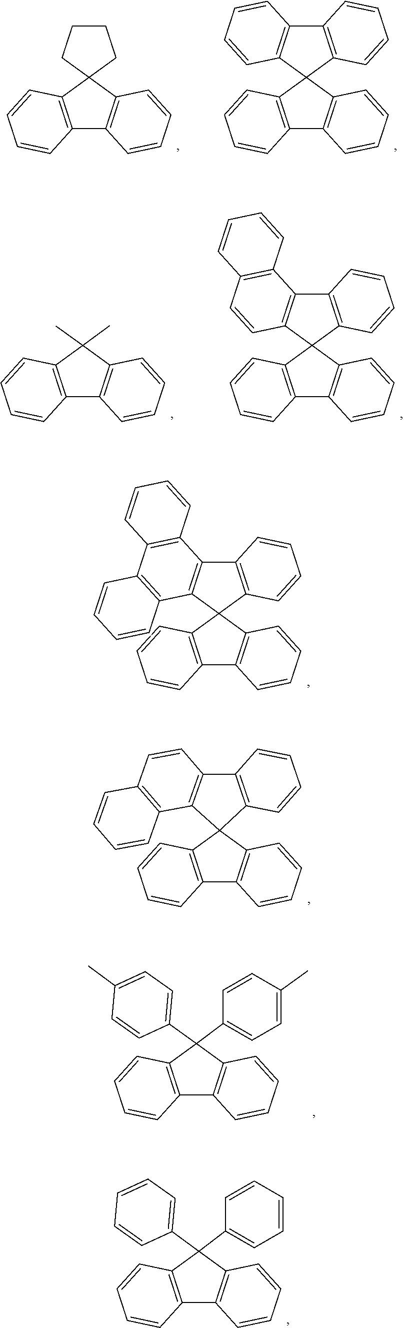

and the like, but are not limited thereto.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting ortho positions in a benzene ring, and two substituents substituting the same carbon in an aliphatic ring may be interpreted as groups “adjacent” to each other.

In the present specification, a heteroaryl group includes one or more atoms other than carbon, that is, one or more heteroatoms, and specifically, the heteroatom may include one or more atoms selected from the group consisting of O, N, Se, P, Si, S, and the like. The number of carbon atoms of the heteroaryl group is not particularly limited, but is preferably 2 to 30, and the heteroaryl group may be monocyclic or polycyclic. Examples of the heteroaryl group include a thiophene group, a furan group, a pyrrole group, an imidazole group, a thiazole group, an oxazole group, an oxadiazole group, a pyridine group, a bipyridine group, a pyrimidine group, a triazine group, a triazole group, an acridine group, a pyridazine group, a pyrazine group, a quinoline group, a quinazoline group, a quinoxaline group, a phthalazine group, a pyridopyrimidine group, a pyridopyrazine group, a pyrazinopyrazine group, an isoquinoline group, an indole group, a carbazole group, a benzoxazole group, a benzimidazole group, a benzothiazole group, a benzocarbazole group, a benzothiophene group, a dibenzothiophene group, a benzofuran group, a phenanthridine group, a phenanthroline group, an isoxazole group, a thiadiazole group, a dibenzofuran group, a dibenzosilole group, a phenoxathiine group, a phenoxazine group, a phenothiazine group, a dihydroindenocarbazole group, a spirofluorenexanthene group, a spirofluorenethioxanthene group, and the like, but are not limited thereto.

In the present specification, the silyl group may be an alkylsilyl group, an arylsilyl group, a heteroarylsilyl group, and the like. The above-described examples of the alkyl group may be applied to the alkyl group in the alkylsilyl group, the above-described examples of the aryl group may be applied to the aryl group in the arylsilyl group, and the examples of the heterocyclic group may be applied to the heteroaryl group in the heteroarylsilyl group.

In the present specification, a boron group may be—BG₁₀₀G₁₀₁, and G₁₀₀ and G₁₀₁ are the same as or different from each other, and may be each independently selected from the group consisting of hydrogen; deuterium; halogen; a nitrile group; a substituted or unsubstituted monocyclic or polycyclic cycloalkyl group having 3 to 30 carbon atoms; a substituted or unsubstituted straight-chained or branched alkyl group having 1 to 30 carbon atoms; a substituted or unsubstituted monocyclic or polycyclic aryl group having 6 to 30 carbon atoms; and a substituted or unsubstituted monocyclic or polycyclic heteroaryl group having 2 to 30 carbon atoms. Specific examples of the boron group include a dimethylboron group, a diethylboron group, a t-butylmethylboron group, a diphenylboron group, and the like, but are not limited thereto.

In the present specification, an amine group may be selected from the group consisting of —NH₂, an alkylamine group, an N-alkylarylamine group, an arylamine group, an N-arylheteroarylamine group, an N-alkylheteroarylamine group, and a heteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, an anthracenylamine group, a 9-methyl-anthracenylamine group, a diphenylamine group, a ditolylamine group, an N-phenyltolylamine group, an N-phenylbiphenylamine group, an N-phenylnaphthylamine group, an N-biphenylnaphthylamine group, an N-naphthylfluorenylamine group, an N-phenylphenanthrenylamine group, an N-biphenylphenanthrenylamine group, an N-phenylfluorenylamine group, an N-phenyl terphenylamine group, an N-phenanthrenylfluorenylamine group, an N-biphenylfluorenylamine group, and the like, but are not limited thereto.

In the present specification, an N-alkylarylamine group means an amine group in which an alkyl group and an aryl group are substituted with N of the amine group. The alkyl group and the aryl group in the N-alkylarylamine group are the same as the above-described examples of the alkyl group and the aryl group.

In the present specification, an N-arylheteroarylamine group means an amine group in which an aryl group and a heteroaryl group are substituted with N of the amine group. The aryl group and heteroaryl group in the N-arylheteroarylamine group are the same as the above-described examples of the aryl group and the heteroaryl group.

In the present specification, an N-alkylheteroarylamine group means an amine group in which an alkyl group and a heteroaryl group are substituted with N of the amine group. The alkyl group and the heteroaryl group in the N-alkylheteroarylamine group are the same as the above-described examples of the alkyl group and the heteroaryl group.

In the present specification, examples of an arylamine group include a substituted or unsubstituted monoarylamine group or a substituted or unsubstituted diarylamine group. The arylamine group including two or more aryl groups may include monocyclic aryl groups, polycyclic aryl groups, or both monocyclic aryl groups and polycyclic aryl groups. For example, the aryl group in the arylamine group may be selected from among the examples of the aryl group described above.

In the present specification, examples of a heteroarylamine group include a substituted or unsubstituted monoheteroarylamine group or a substituted or unsubstituted diheteroarylamine group. The heteroarylamine group including two or more heteroaryl groups may include a monocyclic heteroaryl group, a polycyclic heteroaryl group, or both a monocyclic heteroaryl group and a polycyclic heteroaryl group. For example, the heteroaryl group in the heteroarylamine group may be selected from the above-described examples of the heteroaryl group.

In the present specification, the alkyl group in the alkylthioxy group and the alkylsulfoxy group is the same as the above-described examples of the alkyl group. Specifically, examples of the alkylthioxy group include a methylthioxy group, an ethylthioxy group, a tert-butylthioxy group, a hexylthioxy group, an octylthioxy group, and the like, and examples of the alkylsulfoxy group include a methylsulfoxy group, an ethylsulfoxy group, a propylsulfoxy group, a butylsulfoxy group, and the like, but the examples are not limited thereto.

In the present specification, specific examples of a phosphine oxide group include an alkylphosphine oxide group, an arylphosphine oxide group, and the like, and more specific examples thereof include a diphenylphosphine oxide group, a dinaphthylphosphine oxide group and the like, but are not limited thereto.

In the present specification, the aryl group in the aryloxy group, the arylthioxy group, the arylsulfoxy group and the arylphosphine group is the same as the examples of the aryl group described above. Specifically, examples of the aryloxy group include a phenoxy group, a p-tolyloxy group, an m-tolyloxy group, a 3,5-dimethyl-phenoxy group, a 2,4,6-trimethylphenoxy group, a p-tert-butylphenoxy group, a 3-biphenyloxy group, a 4-biphenyloxy group, a 1-naphthyloxy group, a 2-naphthyloxy group, a 4-methyl-1-naphthyloxy group, a 5-methyl-2-naphthyloxy group, a 1-anthryloxy group, a 2-anthryloxy group, a 9-anthryloxy group, a 1-phenanthryloxy group, a 3-phenanthryloxy group, a 9-phenanthryloxy group, and the like, examples of the arylthioxy group include a phenylthioxy group, a 2-methylphenylthioxy group, a 4-tert-butylphenylthioxy group, and the like, and examples of the arylsulfoxy group include a benzenesulfoxy group, a p-toluenesulfoxy group, and the like, but the examples are not limited thereto.

In the present specification, the “adjacent groups are bonded to each other to form a ring” among the substituents means that a substituent is bonded to an adjacent group to form a substituted or unsubstituted hydrocarbon ring; or a substituted or unsubstituted hetero ring.

In the present specification, in a substituted or unsubstituted ring formed by bonding adjacent groups, the “ring” means a substituted or unsubstituted hydrocarbon ring; or a substituted or unsubstituted hetero ring.

In the present specification, a hydrocarbon ring may be an aromatic hydrocarbon ring, an aliphatic hydrocarbon ring, or a fused ring of an aromatic hydrocarbon and an aliphatic hydrocarbon, and may be selected from the examples of the cycloalkyl group or the aryl group, except for the hydrocarbon ring which is not monovalent.

In the present specification, a hetero ring includes one or more atoms other than carbon, that is, one or more heteroatoms, and specifically, the heteroatom may include one or more atoms selected from the group consisting of O, N, Se, S, and the like. The hetero ring may be monocyclic or polycyclic and may be an aromatic ring, an aliphatic ring, or a fused ring of the aromatic ring and the aliphatic ring, and the aromatic hetero ring may be selected from the examples of the heteroaryl group, except for the aromatic hetero ring which is not monovalent.

In the present specification, an aliphatic hetero ring means an aliphatic ring including one or more of hetero atoms. Examples of the aliphatic hetero ring include oxirane, tetrahydrofuran, 1,4-dioxane, pyrrolidine, piperidine, morpholine, oxepane, azocane, thiocane, and the like, but are not limited thereto.

In the present specification, the arylene group means an aryl group having two bonding sites, that is, a divalent group. Descriptions on the aryl group provided above may be applied thereto except for each being a divalent group.

Unless otherwise defined in the present specification, all technical and scientific terms used in the present specification have the same meaning as commonly understood by one with ordinary skill in the art to which the present invention pertains. Although methods and materials similar to or equivalent to those described in the present specification may be used in the practice or in the test of exemplary embodiments of the present invention, suitable methods and materials will be described below. All publications, patent applications, patents, and other references mentioned in the present specification are hereby incorporated by reference in their entireties, and in the case of conflict, the present specification, including definitions, will control unless a particular passage is mentioned. In addition, the materials, methods, and examples are illustrative only and are not intended to be limiting.

Hereinafter, the compound represented by Chemical Formula 1 will be described in detail.

According to an exemplary embodiment of the present specification, Chemical Formula 1 is any one of the following Chemical Formulae 1-1 to 1-7.

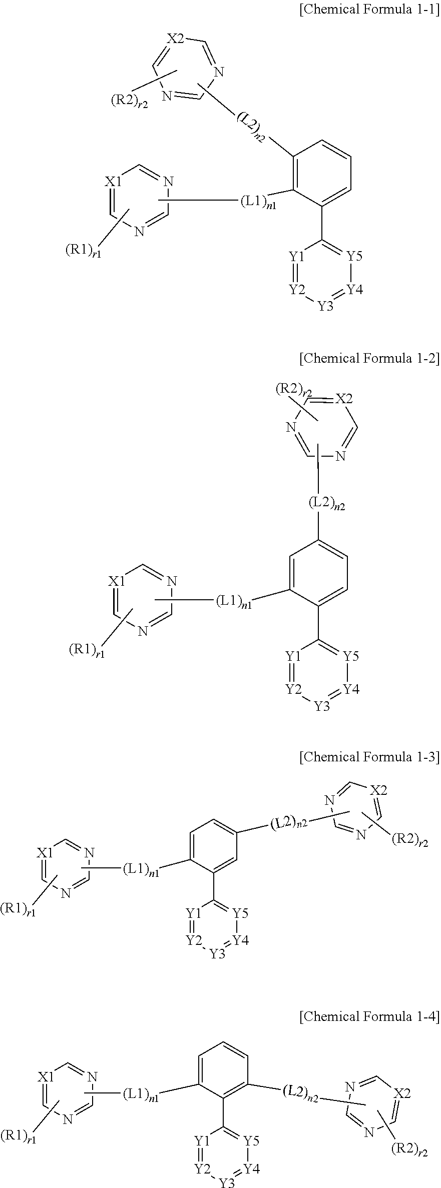

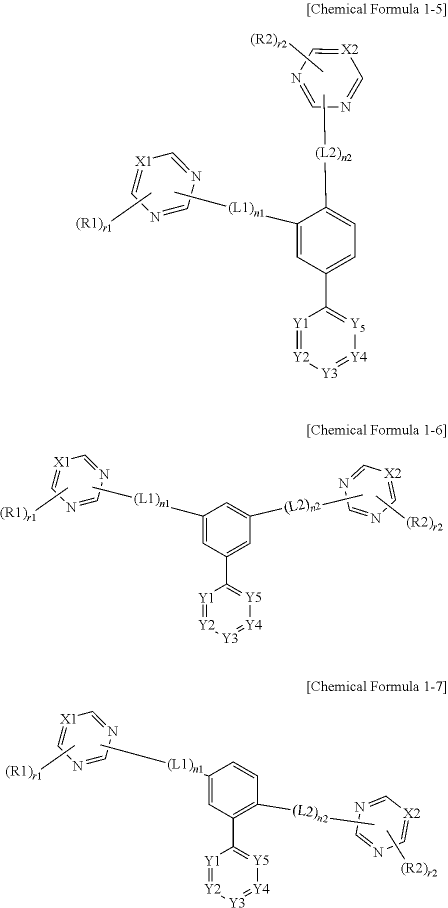

In Chemical Formulae 1-1 to 1-7,
the definitions of Y1 to Y5, X1, X2, R1, R2, r1, r2, L1, L2, n1 and n2 are the same as those defined in Chemical Formula 1.

According to an exemplary embodiment of the present specification, Y1 is N, and Y2 to Y5 are each independently CR3.

According to an exemplary embodiment of the present specification, Y2 is N, and Y1 and Y3 to Y5 are each independently CR3.

According to an exemplary embodiment of the present specification, Y3 is N, and Y1, Y2, Y4 and Y5 are each independently CR3.

According to an exemplary embodiment of the present specification, Y1 and Y2 are N, and Y3 to Y5 are each independently CR3.

According to an exemplary embodiment of the present specification, Y1 and Y3 are N, and Y2, Y4 and Y5 are each independently CR3.

According to an exemplary embodiment of the present specification, Y1 and Y4 are N, and Y2, Y3 and Y5 are each independently CR3.

According to an exemplary embodiment of the present specification, Y1 and Y5 are N, and Y2, Y3 and Y4 are each independently CR3.

According to an exemplary embodiment of the present specification, Y2 and Y3 are N, and Y1, Y4 and Y5 are each independently CR3.

According to an exemplary embodiment of the present specification, Y2 and Y4 are N, and Y1, Y3 and Y5 are each independently CR3.

According to an exemplary embodiment of the present specification, Chemical Formula 1 is any one of the following Chemical Formulae 2 to 10.

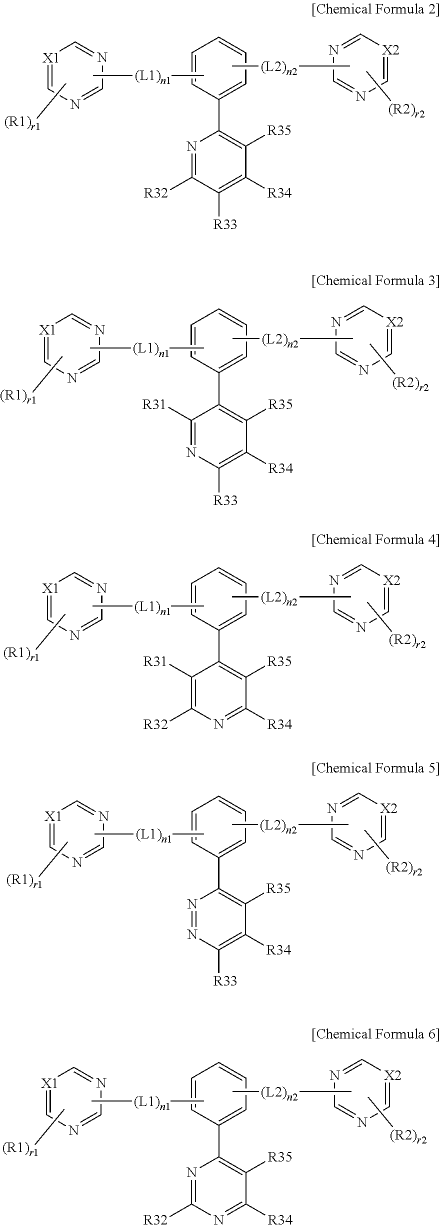

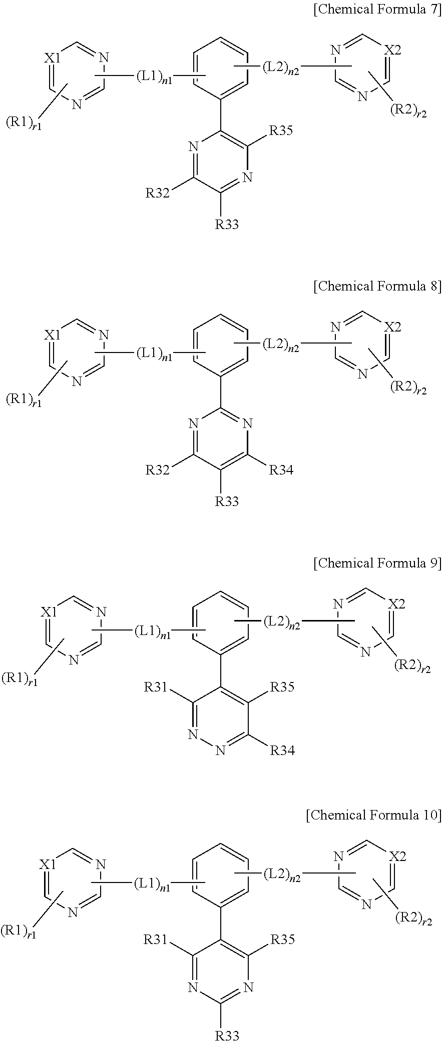

In Chemical Formulae 2 to 10,
the definitions of X1, X2, R1, R2, r1, r2, L1, L2, n1 and n2 are the same as those defined in Chemical Formula 1, and
R31 to R35 are the same as or different from each other, and are each independently hydrogen; deuterium; or a substituted or unsubstituted alkyl group.

According to an exemplary embodiment of the present specification, X1 is N or CR′1.

According to another exemplary embodiment of the present specification, R′1 is hydrogen; deuterium; a substituted or unsubstituted alkyl group; a substituted or unsubstituted aryl group; or a substituted or unsubstituted heteroaryl group.

According to still another exemplary embodiment of the present specification, R′1 is a moiety bonded to L1.

According to yet another exemplary embodiment of the present specification, R′1 is bonded to R1 to form a substituted or unsubstituted aromatic hydrocarbon ring.

According to still yet another exemplary embodiment of the present specification, R′1 is bonded to R1 to form a substituted or unsubstituted monocyclic or polycyclic aromatic hydrocarbon ring having 2 to 30 carbon atoms.

According to a further exemplary embodiment of the present specification, R′1 is bonded to R1 to form a substituted or unsubstituted benzene.

According to an exemplary embodiment of the present specification, X2 is N or CR′1.

According to another exemplary embodiment of the present specification, R′2 is hydrogen; deuterium; a substituted or unsubstituted alkyl group; a substituted or unsubstituted aryl group; or a substituted or unsubstituted heteroaryl group.

According to still another exemplary embodiment of the present specification, R′2 is a moiety bonded to L2.

According to yet another exemplary embodiment of the present specification, R′2 is bonded to R2 to form a substituted or unsubstituted aromatic hydrocarbon ring.

According to still yet another exemplary embodiment of the present specification, R′2 is bonded to R2 to form a substituted or unsubstituted monocyclic or polycyclic aromatic hydrocarbon ring having 2 to 30 carbon atoms.

According to a further exemplary embodiment of the present specification, R′2 is bonded to R2 to form a substituted or unsubstituted benzene.

According to an exemplary embodiment of the present specification, n1 is 1.

According to an exemplary embodiment of the present specification, n1 is 2.

According to an exemplary embodiment of the present specification, n1 is 3.

In the present specification, in Chemical Formula 1, when n1 is 2 or more, it means that the two or more L1's are the same or different from each other, and each L1 is linked in series. For example, when n1 is 3, and L1 is each a phenylene group, a naphthylene group, and a phenylene group, they may be linked as described below, but are not limited thereto, and the order or linking position of each L1 may be different.

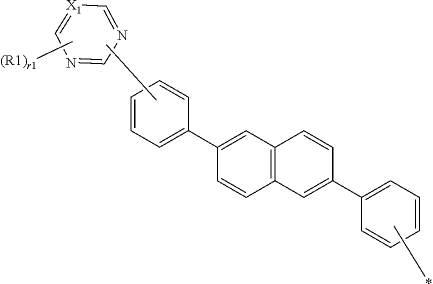

Further, the

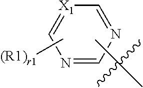

means being bonded to a substituent at the third end located in the structure exemplified above, that is, a phenylene group, for example, when n1 is 3, * means a moiety bonded to Chemical Formula 1, and [symbol] means a moiety bonded to the terminus of L1.

The description on n1 also applies equally to the description on n2.

According to an exemplary embodiment of the present specification, n2 is 1.

According to an exemplary embodiment of the present specification, n2 is 2.

According to an exemplary embodiment of the present specification, n2 is 3.

According to an exemplary embodiment of the present specification, the

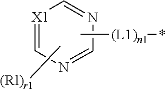

of Chemical Formula 1 is any one of the following structures.

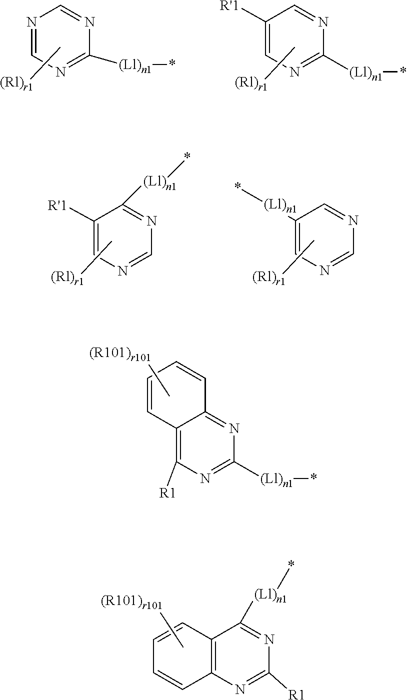

In the structures,
* is a moiety bonded to Chemical Formula 1, the definitions of L1, n1, R1, r1 and R′1 are the same as those defined in Chemical Formula 1,
R101 is hydrogen; deuterium; a substituted or unsubstituted alkyl group; a substituted or unsubstituted aryl group; or a substituted or unsubstituted heteroaryl group,
r101 is an integer from 1 to 4, and
when r101 is 2 or higher, two or more R101's are the same as or different from each other.

According to an exemplary embodiment of the present specification, the

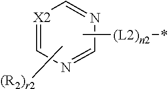

of Chemical Formula 1 is any one of the following structures.

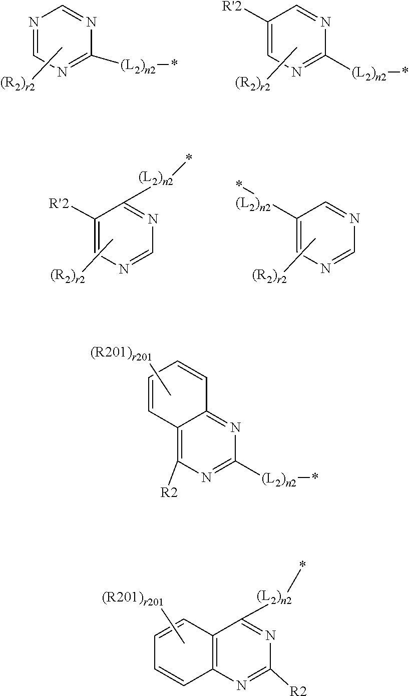

In the structures,
* is a moiety bonded to Chemical Formula 1,
the definitions of L2, n2, R2, r2 and R′2 are the same as those defined in Chemical Formula 1,
R201 is hydrogen; deuterium; a substituted or unsubstituted alkyl group; a substituted or unsubstituted aryl group; or a substituted or unsubstituted heteroaryl group,
R201 is an integer from 1 to 4, and
when r201 is 2 or higher, two or more R201's are the same as or different from each other.

According to an exemplary embodiment of the present specification, the

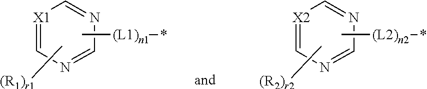

of Chemical Formula 1 are the same as each other.

According to an exemplary embodiment of the present specification, the

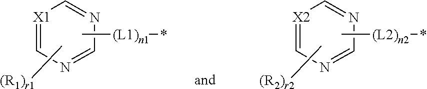

of Chemical Formula 1 are different from each other.

According to an exemplary embodiment of the present specification, R1 and R2 are the same as or different from each other, and are each independently a straight-chained or branched alkyl group having 1 to 30 carbon atoms; a monocyclic or polycyclic aryl group having 6 to 30 carbon atoms, unsubstituted or substituted with a monocyclic or polycyclic heteroaryl group having 2 to 30 carbon atoms, which is unsubstituted or substituted with a straight-chained or branched alkyl group having 1 to 30 carbon atoms, a monocyclic or polycyclic cycloalkyl group having 3 to 30 carbon atoms, or a straight-chained or branched alkyl group having 1 to 30 carbon atoms; or a monocyclic or polycyclic heteroaryl group having 2 to 30 carbon atoms, which is unsubstituted or substituted with a straight-chained or branched alkyl group having 1 to 30 carbon atoms, or are bonded to an adjacent group to form a monocyclic or polycyclic aromatic hydrocarbon ring having 6 to 30 carbon atoms, R′1 is hydrogen; deuterium; a straight-chained or branched alkyl group having 1 to 30 carbon atoms; a monocyclic or polycyclic aryl group having 6 to 30 carbon atoms; or a moiety bonded to L1, or is bonded to R1 to form a monocyclic or polycyclic aromatic hydrocarbon ring having 6 to 30 carbon atoms, R′2 is hydrogen; deuterium; a straight-chained or branched alkyl group having 1 to 30 carbon atoms; a monocyclic or polycyclic aryl group having 6 to 30 carbon atoms; or a moiety bonded to L2, or is bonded to R2 to form a monocyclic or polycyclic aromatic hydrocarbon ring having 6 to 30 carbon atoms, L1 and L2 are the same as or different from each other, and are each independently a monocyclic or polycyclic arylene group having 6 to 30 carbon atoms, and R3 is hydrogen; deuterium; or a straight-chained or branched alkyl group having 1 to 30 carbon atoms.

According to an exemplary embodiment of the present specification, R1 and R2 are the same as or different from each other, and are each independently a substituted or unsubstituted straight-chained or branched alkyl group having 1 to 30 carbon atoms; a substituted or unsubstituted monocyclic or polycyclic aryl group 6 to 30 carbon atoms; or a substituted or unsubstituted monocyclic or polycyclic heteroaryl group having 2 to 30 carbon atoms, or are bonded to an adjacent group to form a substituted or unsubstituted monocyclic or polycyclic aromatic hydrocarbon ring having 6 to 30 carbon atoms.

According to an exemplary embodiment of the present specification, R1 and R2 are the same as or different from each other, and are each independently a substituted or unsubstituted straight-chained or branched alkyl group having 1 to 20 carbon atoms; a substituted or unsubstituted monocyclic or polycyclic aryl group 6 to 20 carbon atoms; or a substituted or unsubstituted monocyclic or polycyclic heteroaryl group having 2 to 20 carbon atoms, or are bonded to an adjacent group to form a substituted or unsubstituted monocyclic or polycyclic aromatic hydrocarbon ring having 6 to 20 carbon atoms.

According to an exemplary embodiment of the present specification, R1 and R2 are the same as or different from each other, and are each independently a substituted or unsubstituted straight-chained or branched alkyl group having 1 to 10 carbon atoms; a substituted or unsubstituted monocyclic or polycyclic aryl group 6 to 10 carbon atoms; or a substituted or unsubstituted monocyclic or polycyclic heteroaryl group having 2 to 10 carbon atoms, or are bonded to an adjacent group to form a substituted or unsubstituted monocyclic or polycyclic aromatic hydrocarbon ring having 6 to 10 carbon atoms.

According to an exemplary embodiment of the present specification, R1 and R2 are the same as or different from each other, and are each independently a straight-chained or branched alkyl group having 1 to 30 carbon atoms; a monocyclic or polycyclic aryl group having 6 to 30 carbon atoms, which is unsubstituted or substituted with a monocyclic or polycyclic heteroaryl group having 2 to 30 carbon atoms, which is unsubstituted or substituted with a straight-chained or branched alkyl group having 1 to 30 carbon atoms, a monocyclic or polycyclic cycloalkyl group having 3 to 30 carbon atoms, or a straight-chained or branched alkyl group having 1 to 30 carbon atoms; or a monocyclic or polycyclic heteroaryl group having 2 to 30 carbon atoms, which is unsubstituted or substituted with a straight-chained or branched alkyl group having 1 to 30 carbon atoms, or are bonded to an adjacent group to form a monocyclic or polycyclic aromatic hydrocarbon ring having 6 to 30 carbon atoms.

According to an exemplary embodiment of the present specification, R1 and R2 are the same as or different from each other, and are each independently a straight-chained or branched alkyl group having 1 to 20 carbon atoms; a monocyclic or polycyclic aryl group having 6 to 20 carbon atoms, which is unsubstituted or substituted with a monocyclic or polycyclic heteroaryl group having 2 to 20 carbon atoms, which is unsubstituted or substituted with a straight-chained or branched alkyl group having 1 to 20 carbon atoms, a monocyclic or polycyclic cycloalkyl group having 3 to 20 carbon atoms, or a straight-chained or branched alkyl group having 1 to 20 carbon atoms; or a monocyclic or polycyclic heteroaryl group having 2 to 20 carbon atoms, which is unsubstituted or substituted with a straight-chained or branched alkyl group having 1 to 20 carbon atoms, or are bonded to an adjacent group to form a monocyclic or polycyclic aromatic hydrocarbon ring having 6 to 20 carbon atoms.

According to an exemplary embodiment of the present specification, R1 and R2 are the same as or different from each other, and are each independently a straight-chained or branched alkyl group having 1 to 10 carbon atoms; a monocyclic or polycyclic aryl group having 6 to 10 carbon atoms, which is unsubstituted or substituted with a monocyclic or polycyclic heteroaryl group having 2 to 10 carbon atoms, which is unsubstituted or substituted with a straight-chained or branched alkyl group having 1 to 10 carbon atoms, a monocyclic or polycyclic cycloalkyl group having 3 to 10 carbon atoms, or a straight-chained or branched alkyl group having 1 to 10 carbon atoms; or a monocyclic or polycyclic heteroaryl group having 2 to 10 carbon atoms, which is unsubstituted or substituted with a straight-chained or branched alkyl group having 1 to 10 carbon atoms, or are bonded to an adjacent group to form a monocyclic or polycyclic aromatic hydrocarbon ring having 6 to 10 carbon atoms.

According to an exemplary embodiment of the present specification, R1 and R2 are the same as or different from each other, and are each independently a methyl group; a phenyl group unsubstituted or substituted with a methyl group, a tert-butyl group, a cyclohexyl group, or a pyridine group unsubstituted or substituted with a methyl group; a biphenyl group; a naphthyl group; or a pyridine group unsubstituted or substituted with a methyl group or a phenyl group, and are bonded to an adjacent group to form benzene.

According to an exemplary embodiment of the present specification, R′1 is hydrogen; deuterium; a substituted or unsubstituted straight-chained or branched alkyl group having 1 to 30 carbon atoms; a substituted or unsubstituted monocyclic or polycyclic aryl group having 6 to 30 carbon atoms; or a moiety bonded to L1, or is bonded to R1 to form a substituted or unsubstituted monocyclic or polycyclic aromatic hydrocarbon ring having 6 to 30 carbon atoms.

According to an exemplary embodiment of the present specification, R′1 is hydrogen; deuterium; a substituted or unsubstituted straight-chained or branched alkyl group having 1 to 20 carbon atoms; a substituted or unsubstituted monocyclic or polycyclic aryl group having 6 to 20 carbon atoms; or a moiety bonded to L1, or is bonded to R1 to form a substituted or unsubstituted monocyclic or polycyclic aromatic hydrocarbon ring having 6 to 20 carbon atoms.

According to an exemplary embodiment of the present specification, R′1 is hydrogen; deuterium; a substituted or unsubstituted straight-chained or branched alkyl group having 1 to 10 carbon atoms; a substituted or unsubstituted monocyclic or polycyclic aryl group having 6 to 10 carbon atoms; or a moiety bonded to L1, or is bonded to R1 to form a substituted or unsubstituted monocyclic or polycyclic aromatic hydrocarbon ring having 6 to 10 carbon atoms.

According to an exemplary embodiment of the present specification, R′1 is hydrogen; deuterium; a straight-chained or branched alkyl group having 1 to 30 carbon atoms; a monocyclic or polycyclic aryl group having 6 to 30 carbon atoms; or a moiety bonded to L1, or is bonded to R1 to form a monocyclic or polycyclic aromatic hydrocarbon ring having 6 to 30 carbon atoms.

According to an exemplary embodiment of the present specification, R′1 is hydrogen; deuterium; a straight-chained or branched alkyl group having 1 to 20 carbon atoms; a monocyclic or polycyclic aryl group having 6 to 20 carbon atoms; or a moiety bonded to L1, or is bonded to R1 to form a monocyclic or polycyclic aromatic hydrocarbon ring having 6 to 20 carbon atoms.

According to an exemplary embodiment of the present specification, R′1 is hydrogen; deuterium; a straight-chained or branched alkyl group having 1 to 10 carbon atoms; a monocyclic or polycyclic aryl group having 6 to 10 carbon atoms; or a moiety bonded to L1, or is bonded to R1 to form a monocyclic or polycyclic aromatic hydrocarbon ring having 6 to 10 carbon atoms.

According to an exemplary embodiment of the present specification, R′1 is hydrogen; deuterium; a methyl group; an isopropyl group; a phenyl group; or a moiety bonded to L1, or is bonded to R1 to form benzene.

According to an exemplary embodiment of the present specification, R′2 is hydrogen; deuterium; a substituted or unsubstituted straight-chained or branched alkyl group having 1 to 30 carbon atoms; a substituted or unsubstituted monocyclic or polycyclic aryl group having 6 to 30 carbon atoms; or a moiety bonded to L2, or is bonded to R2 to form a substituted or unsubstituted monocyclic or polycyclic aromatic hydrocarbon ring having 6 to 30 carbon atoms.

According to an exemplary embodiment of the present specification, R′2 is hydrogen; deuterium; a substituted or unsubstituted straight-chained or branched alkyl group having 1 to 20 carbon atoms; a substituted or unsubstituted monocyclic or polycyclic aryl group having 6 to 20 carbon atoms; or a moiety bonded to L2, or is bonded to R2 to form a substituted or unsubstituted monocyclic or polycyclic aromatic hydrocarbon ring having 6 to 20 carbon atoms.

According to an exemplary embodiment of the present specification, R′2 is hydrogen; deuterium; a substituted or unsubstituted straight-chained or branched alkyl group having 1 to 10 carbon atoms; a substituted or unsubstituted monocyclic or polycyclic aryl group having 6 to 10 carbon atoms; or a moiety bonded to L2, or is bonded to R2 to form a substituted or unsubstituted monocyclic or polycyclic aromatic hydrocarbon ring having 6 to 10 carbon atoms.

According to an exemplary embodiment of the present specification, R′2 is hydrogen; deuterium; a straight-chained or branched alkyl group having 1 to 30 carbon atoms; a monocyclic or polycyclic aryl group having 6 to 30 carbon atoms; or a moiety bonded to L2, or is bonded to R2 to form a monocyclic or polycyclic aromatic hydrocarbon ring having 6 to 30 carbon atoms.

According to an exemplary embodiment of the present specification, R′2 is hydrogen; deuterium; a straight-chained or branched alkyl group having 1 to 20 carbon atoms; a monocyclic or polycyclic aryl group having 6 to 20 carbon atoms; or a moiety bonded to L2, or is bonded to R2 to form a monocyclic or polycyclic aromatic hydrocarbon ring having 6 to 20 carbon atoms.

According to an exemplary embodiment of the present specification, R′2 is hydrogen; deuterium; a straight-chained or branched alkyl group having 1 to 10 carbon atoms; a monocyclic or polycyclic aryl group having 6 to 10 carbon atoms; or a moiety bonded to L2, or is bonded to R2 to form a monocyclic or polycyclic aromatic hydrocarbon ring having 6 to 10 carbon atoms.

According to an exemplary embodiment of the present specification, R′2 is hydrogen; deuterium; a methyl group; an isopropyl group; a phenyl group; or a moiety bonded to L2, or is bonded to R2 to form benzene.

According to an exemplary embodiment of the present specification, L1 and L2 are the same as or different from each other, and are each independently a substituted or unsubstituted monocyclic or polycyclic arylene group having 6 to 30 carbon atoms.

According to an exemplary embodiment of the present specification, L1 and L2 are the same as or different from each other, and are each independently a substituted or unsubstituted monocyclic or polycyclic arylene group having 6 to 20 carbon atoms.

According to an exemplary embodiment of the present specification, L1 and L2 are the same as or different from each other, and are each independently a substituted or unsubstituted monocyclic or polycyclic arylene group having 6 to 10 carbon atoms.

According to an exemplary embodiment of the present specification, L1 and L2 are the same as or different from each other, and are each independently a monocyclic or polycyclic arylene group having 6 to 30 carbon atoms.

According to an exemplary embodiment of the present specification, L1 and L2 are the same as or different from each other, and are each independently a monocyclic or polycyclic arylene group having 6 to 20 carbon atoms.

According to an exemplary embodiment of the present specification, L1 and L2 are the same as or different from each other, and are each independently a monocyclic or polycyclic arylene group having 6 to 10 carbon atoms.

According to an exemplary embodiment of the present specification, L1 and L2 are the same as or different from each other, and are each independently a phenylene group; or a naphthylene group.

According to an exemplary embodiment of the present specification, R3 is hydrogen; deuterium; or a substituted or unsubstituted straight-chained or branched alkyl group having 1 to 30 carbon atoms.

According to an exemplary embodiment of the present specification, R3 is hydrogen; deuterium; or a substituted or unsubstituted straight-chained or branched alkyl group having 1 to 20 carbon atoms.

According to an exemplary embodiment of the present specification, R3 is hydrogen; deuterium; or a substituted or unsubstituted straight-chained or branched alkyl group having 1 to 10 carbon atoms.

According to an exemplary embodiment of the present specification, R3 is hydrogen; deuterium; or a straight-chained or branched alkyl group having 1 to 30 carbon atoms.

According to an exemplary embodiment of the present specification, R3 is hydrogen; deuterium; or a straight-chained or branched alkyl group having 1 to 20 carbon atoms.

According to an exemplary embodiment of the present specification, R3 is hydrogen; deuterium; or a straight-chained or branched alkyl group having 1 to 10 carbon atoms.

According to an exemplary embodiment of the present specification, R3 is hydrogen; deuterium; or a methyl group.

According to an exemplary embodiment of the present specification, R101 is hydrogen.

According to an exemplary embodiment of the present specification, R201 is hydrogen.

According to an exemplary embodiment of the present specification, R31 to R35 are the same as or different from each other, and are each independently hydrogen; deuterium; or a substituted or unsubstituted straight-chained or branched alkyl group having 1 to 30 carbon atoms.

According to an exemplary embodiment of the present specification, R31 to R35 are the same as or different from each other, and are each independently hydrogen; deuterium; or a substituted or unsubstituted straight-chained or branched alkyl group having 1 to 20 carbon atoms.

According to an exemplary embodiment of the present specification, R31 to R35 are the same as or different from each other, and are each independently hydrogen; deuterium; or a substituted or unsubstituted straight-chained or branched alkyl group having 1 to 10 carbon atoms.

According to an exemplary embodiment of the present specification, R31 to R35 are the same as or different from each other, and are each independently hydrogen; deuterium; or a straight-chained or branched alkyl group having 1 to 30 carbon atoms.

According to an exemplary embodiment of the present specification, R31 to R35 are the same as or different from each other, and are each independently hydrogen; deuterium; or a straight-chained or branched alkyl group having 1 to 20 carbon atoms.

According to an exemplary embodiment of the present specification, R31 to R35 are the same as or different from each other, and are each independently hydrogen; deuterium; or a straight-chained or branched alkyl group having 1 to 10 carbon atoms.

According to an exemplary embodiment of the present specification, R31 to R35 are the same as or different from each other, and are each independently hydrogen; deuterium; or a methyl group.

According to an exemplary embodiment of the present specification, Chemical Formula 1 is any one selected from the following compounds.

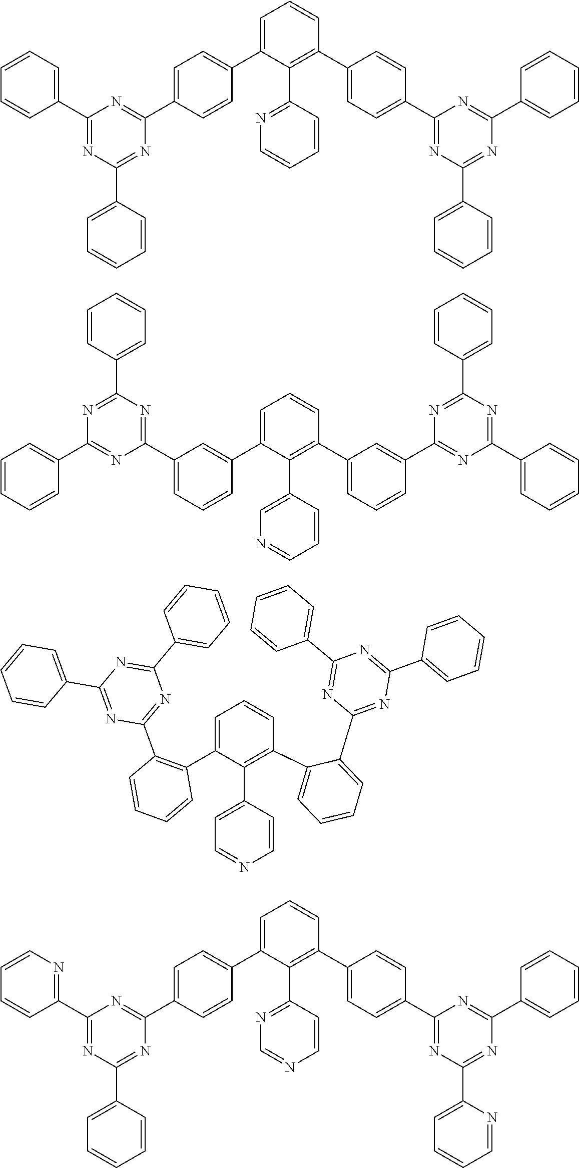

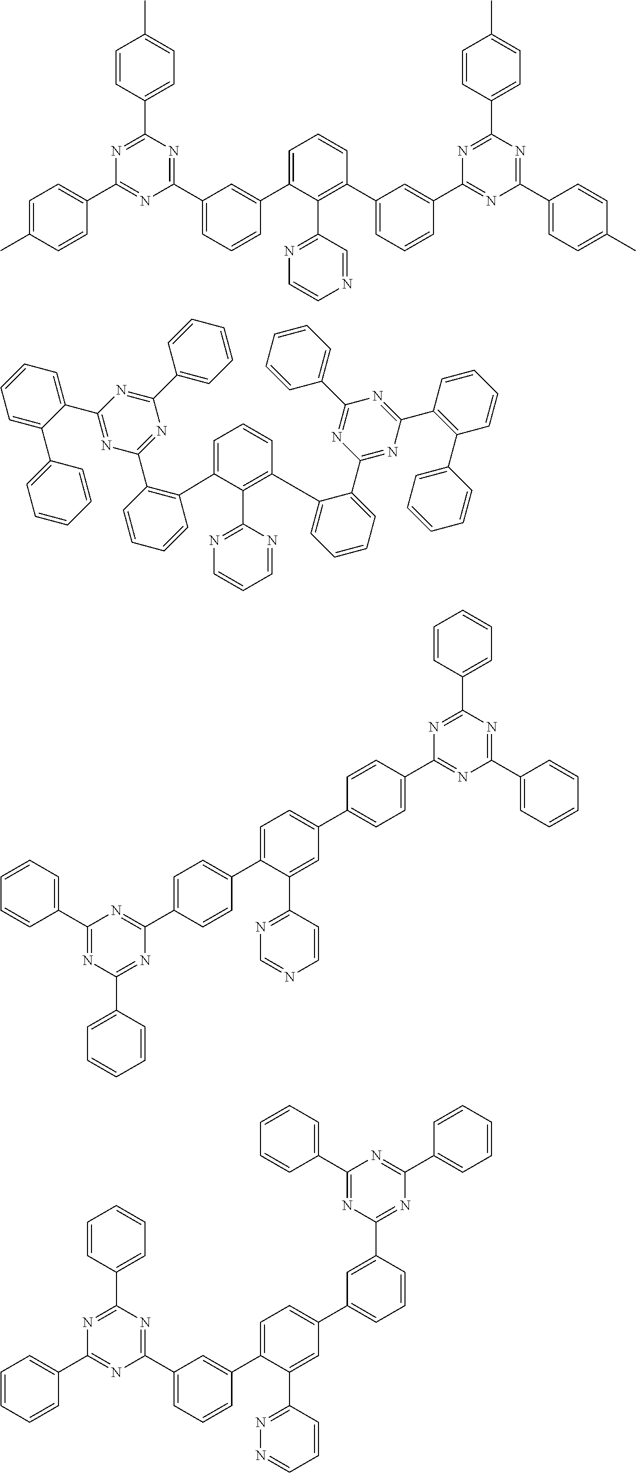

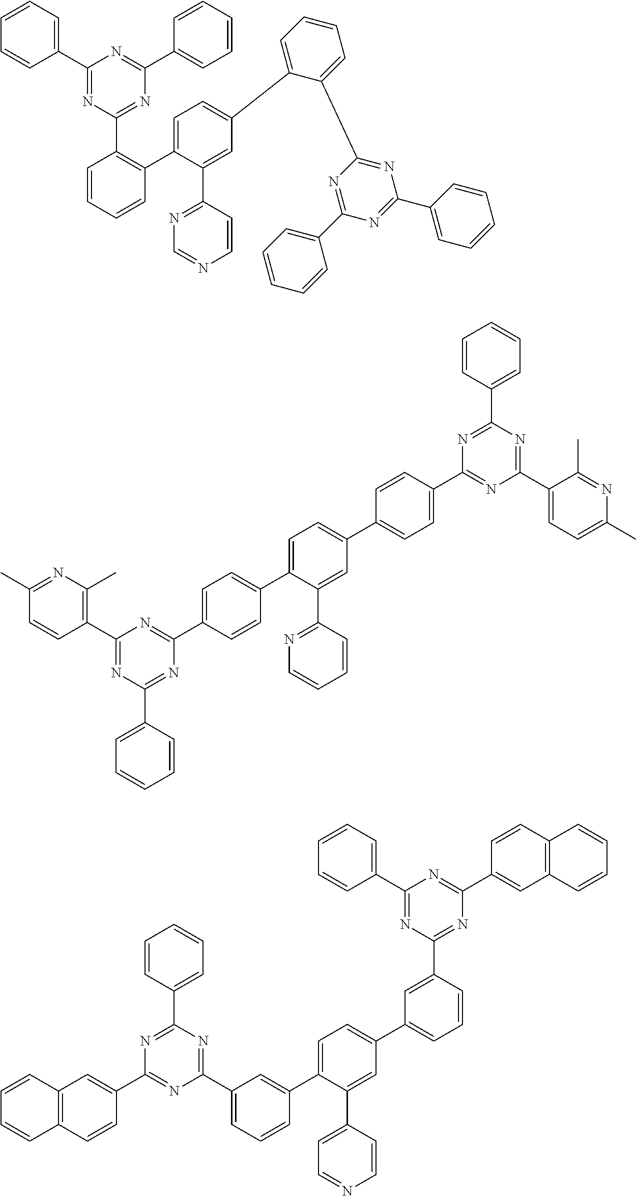

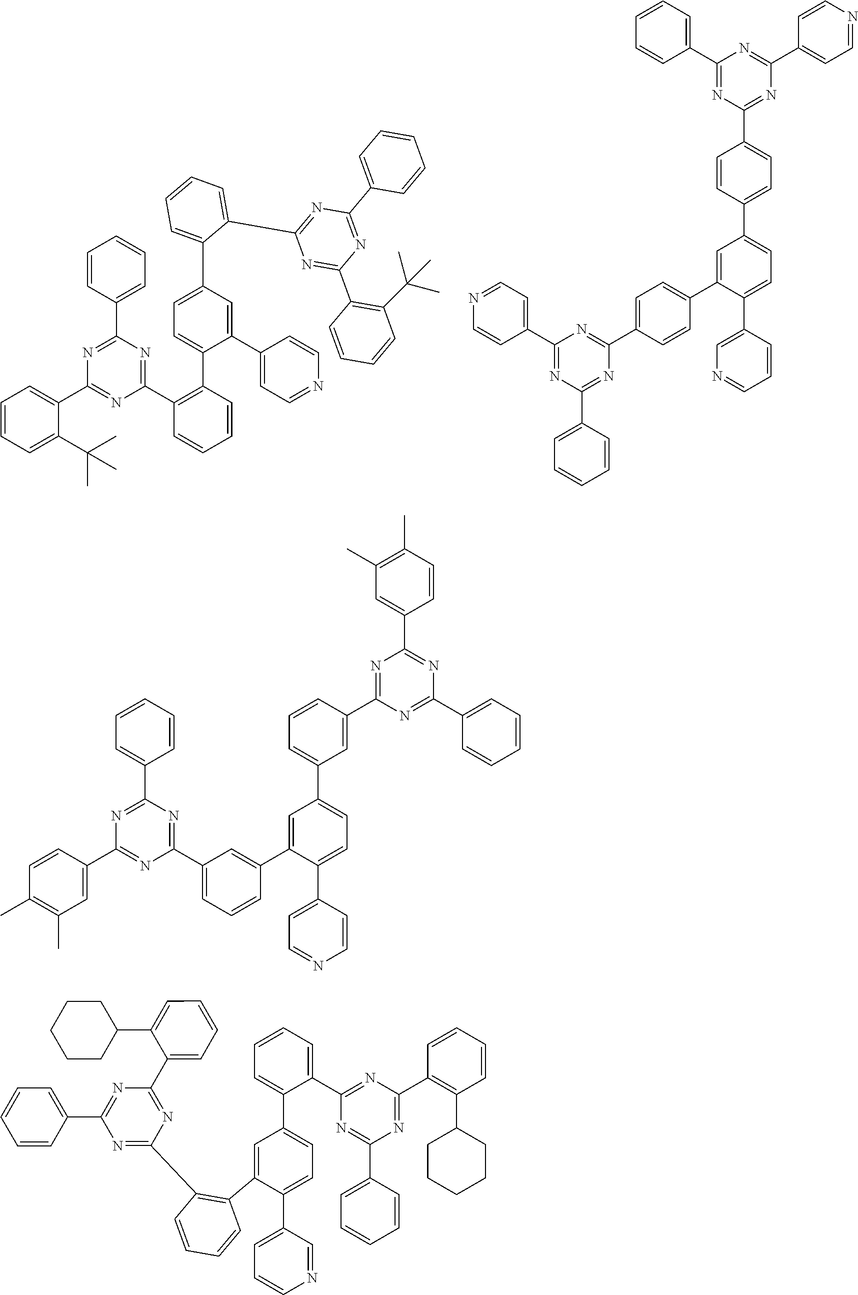

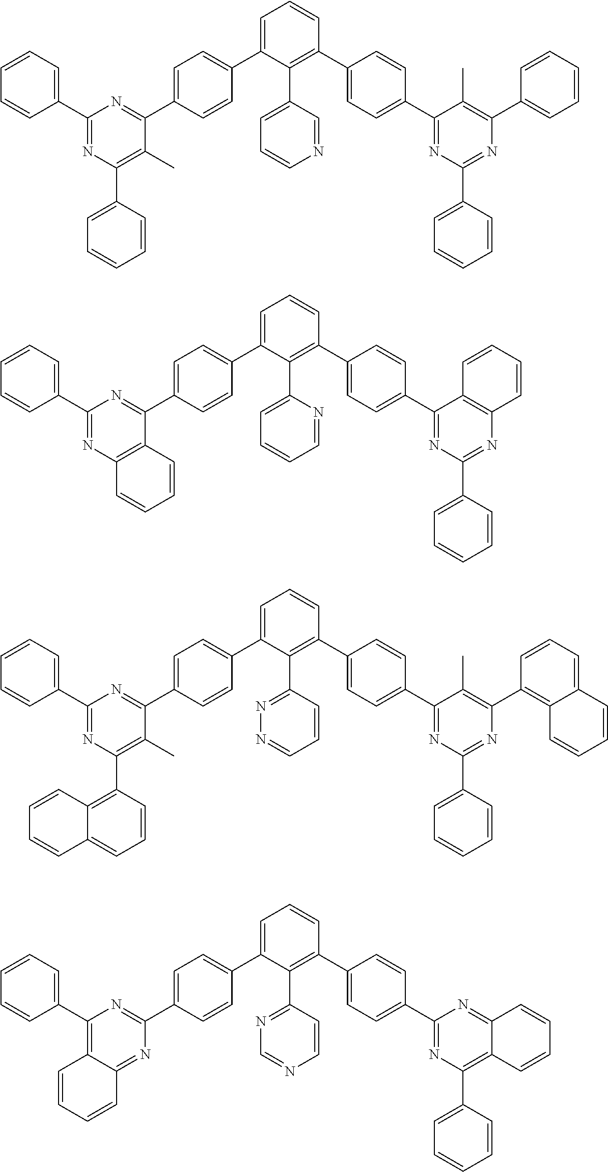 [SMILES: Cc1c(-c2ccccc2)nc(-c2ccccc2)nc1-c1ccc(-c2cccc(-c3ccc(-c4nc(-c5ccccc5)nc(-c5ccccc5)c4C)cc3)c2-c2cccnc2)cc1 ; c1ccc(-c2nc(-c3ccc(-c4cccc(-c5ccc(-c6nc(-c7ccccc7)nc7ccccc67)cc5)c4-c4ccccn4)cc3)c3ccccc3n2)cc1 ; Cc1c(-c2ccc(-c3cccc(-c4ccc(-c5nc(-c6ccccc6)nc(-c6cccc7ccccc67)c5C)cc4)c3-c3cccnn3)cc2)nc(-c2ccccc2)nc1-c1cccc2ccccc12 ; c1ccc(-c2nc(-c3ccc(-c4cccc(-c5ccc(-c6nc(-c7ccccc7)c7ccccc7n6)cc5)c4-c4ccncn4)cc3)nc3ccccc23)cc1]

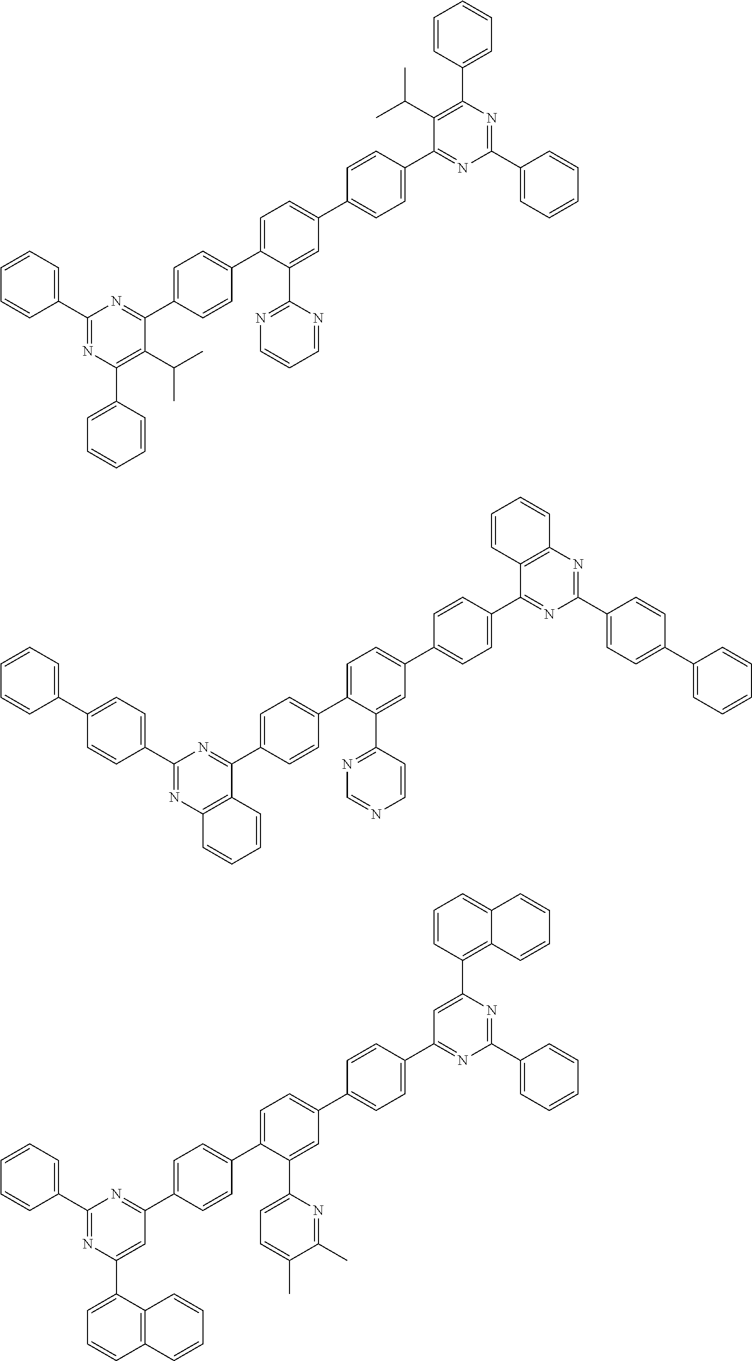 [SMILES: CC(C)c1c(-c2ccccc2)nc(-c2ccccc2)nc1-c1ccc(-c2ccc(-c3ccc(-c4nc(-c5ccccc5)nc(-c5ccccc5)c4C(C)C)cc3)c(-c3ncccn3)c2)cc1 ; c1ccc(-c2ccc(-c3nc(-c4ccc(-c5ccc(-c6ccc(-c7nc(-c8ccc(-c9ccccc9)cc8)nc8ccccc78)cc6)c(-c6ccncn6)c5)cc4)c4ccccc4n3)cc2)cc1 ; Cc1ccc(-c2cc(-c3ccc(-c4cc(-c5cccc6ccccc56)nc(-c5ccccc5)n4)cc3)ccc2-c2ccc(-c3cc(-c4cccc5ccccc45)nc(-c4ccccc4)n3)cc2)nc1C]

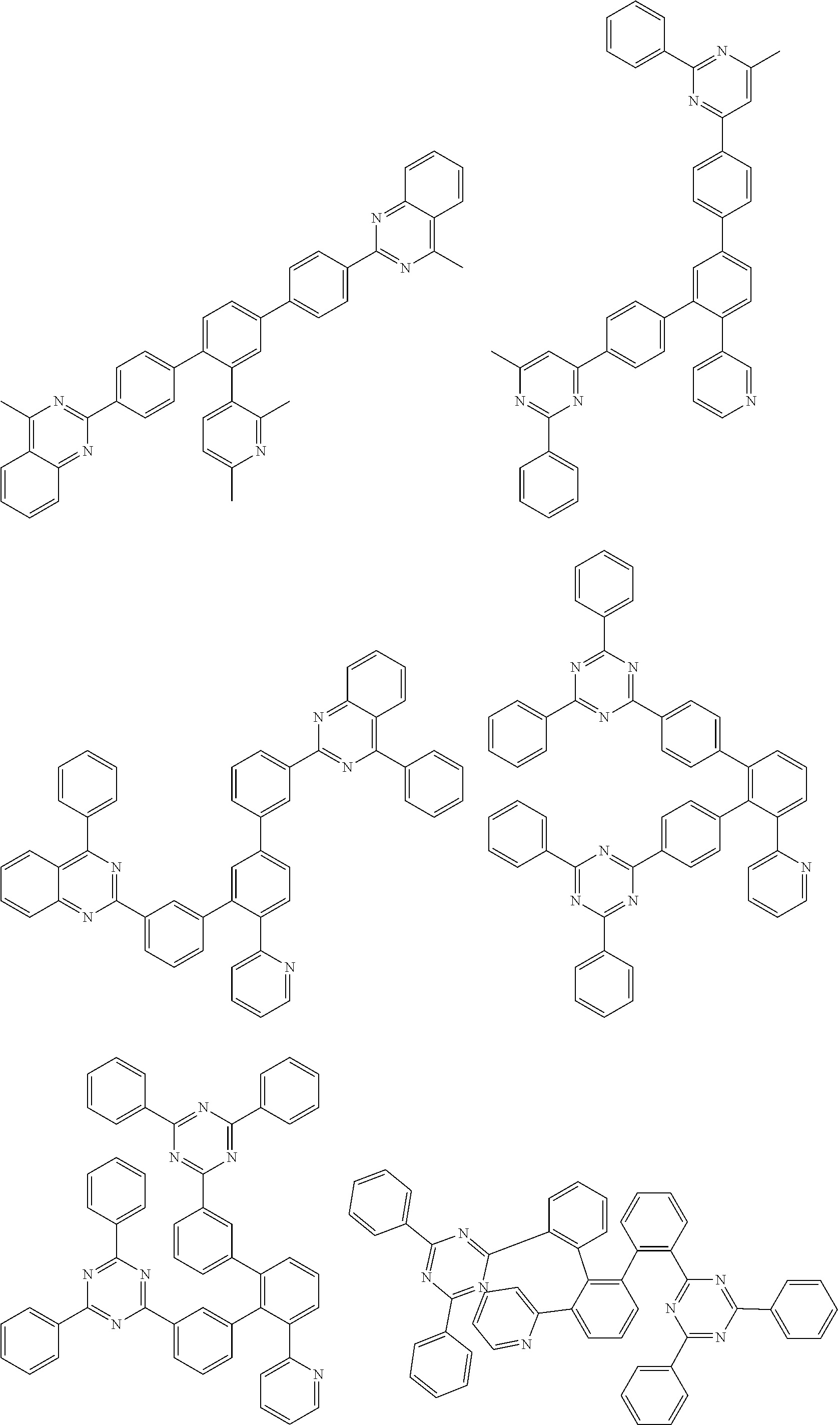

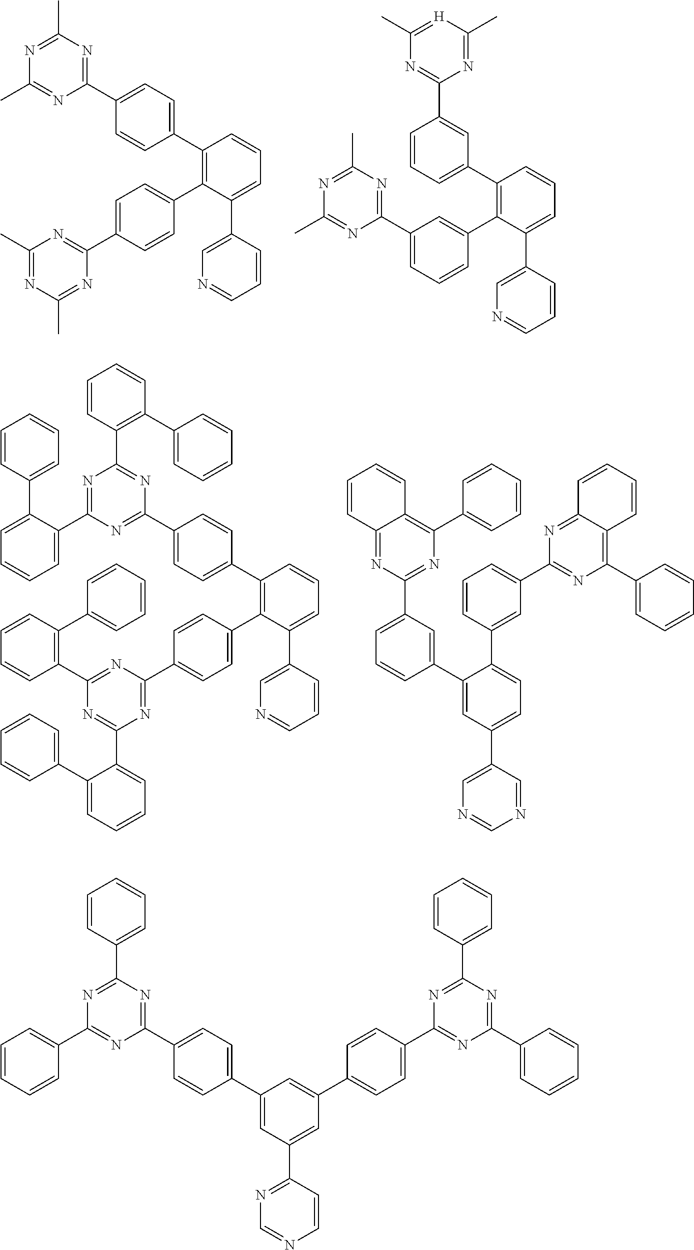

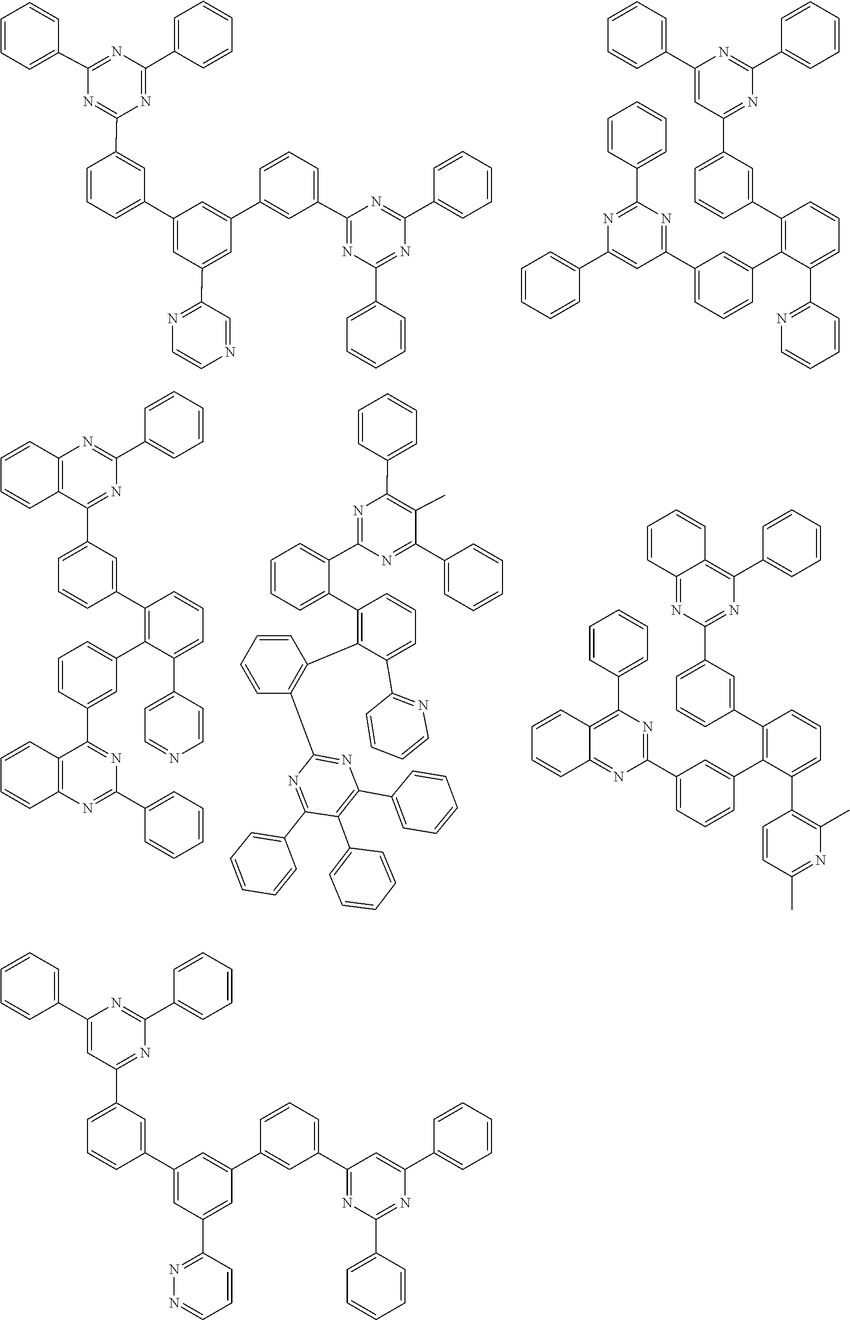

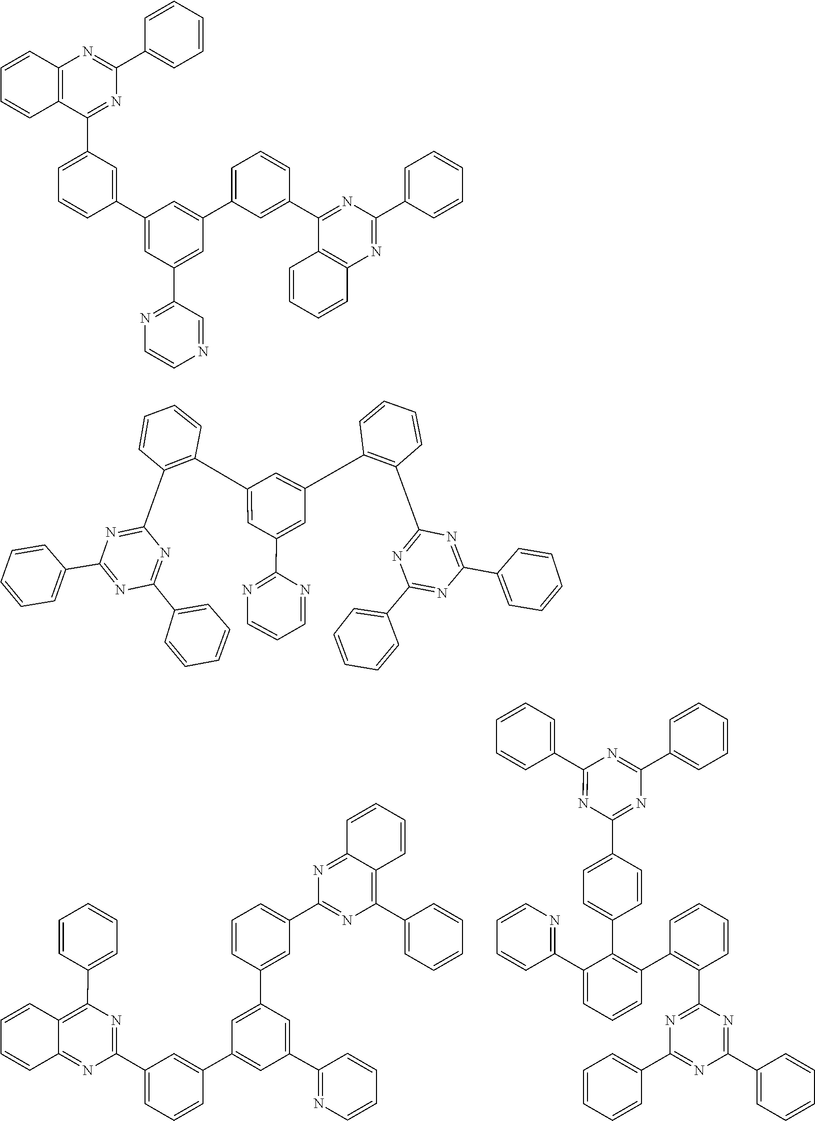

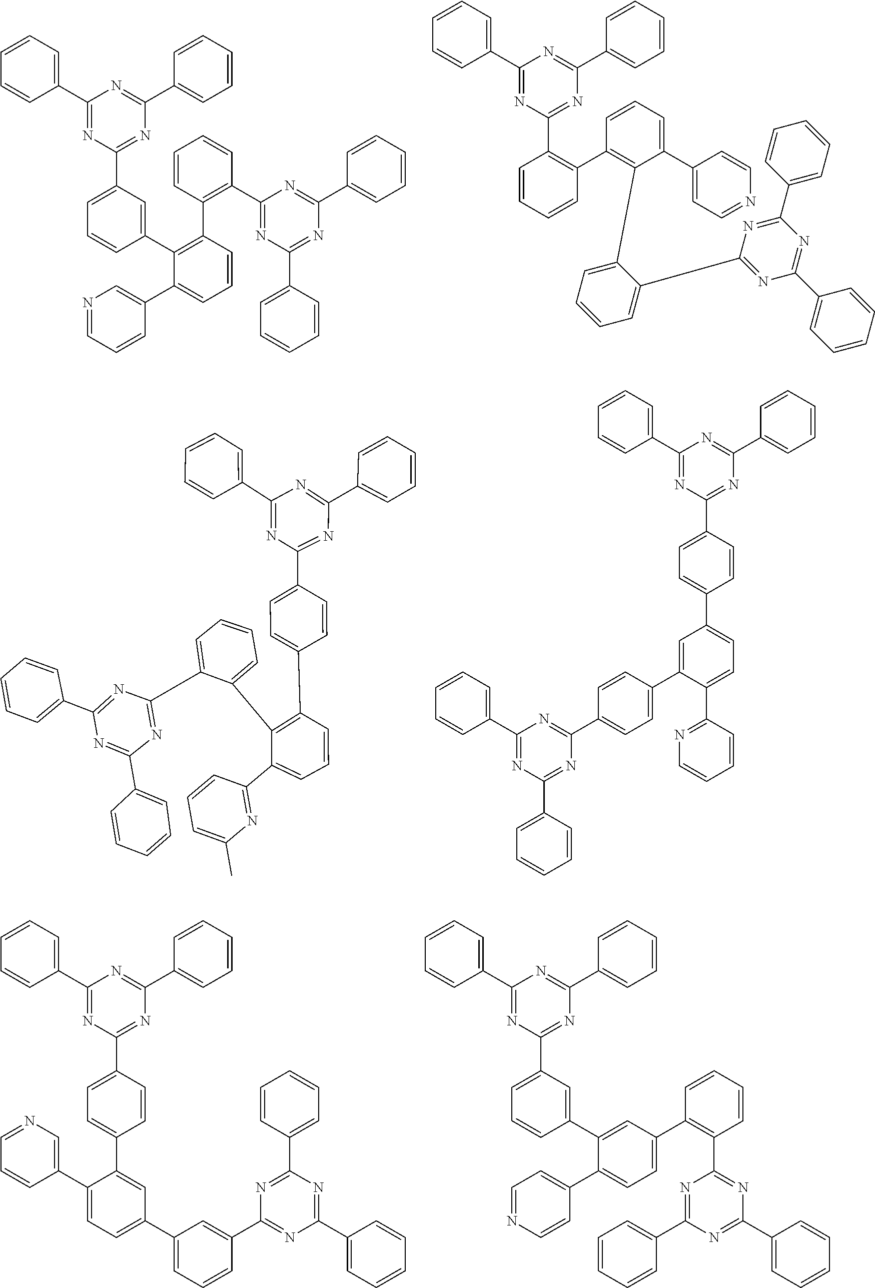

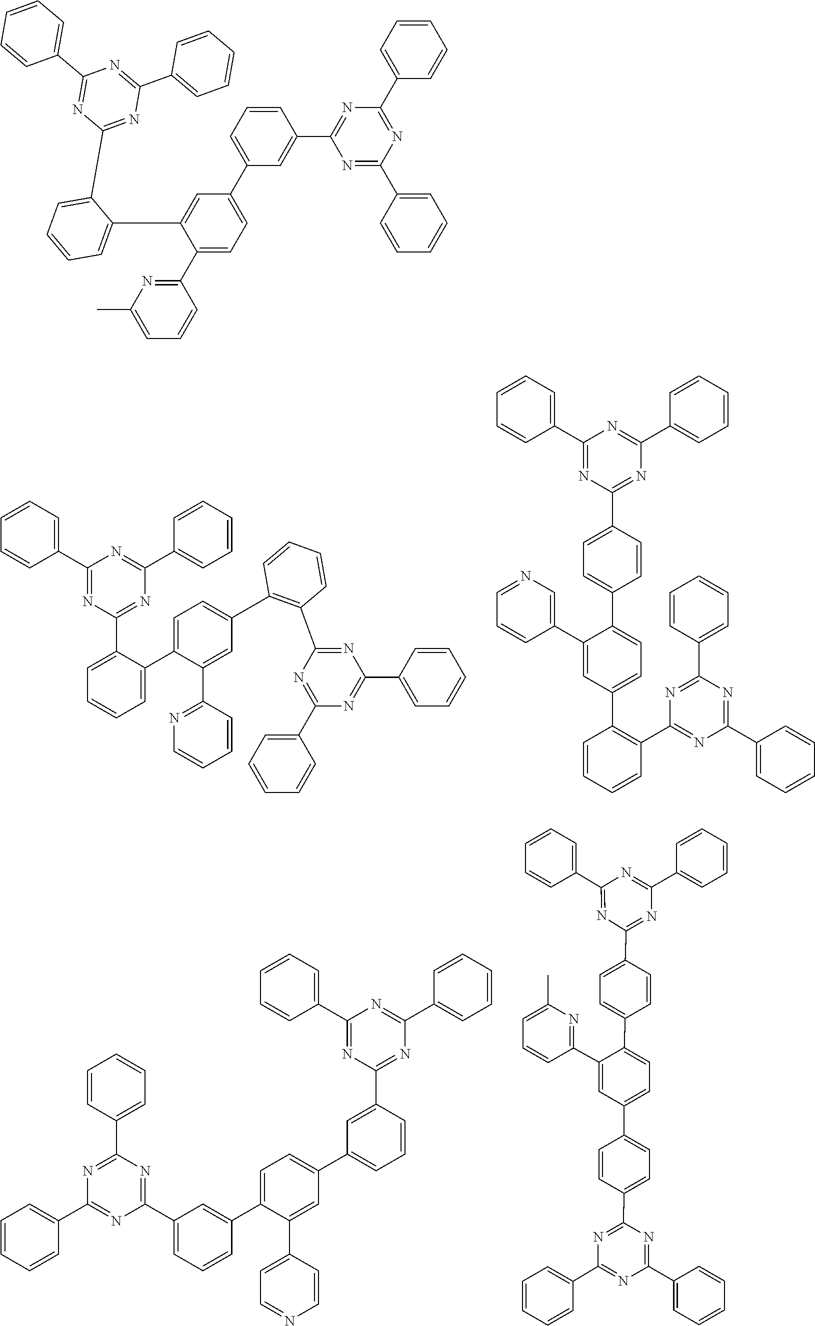

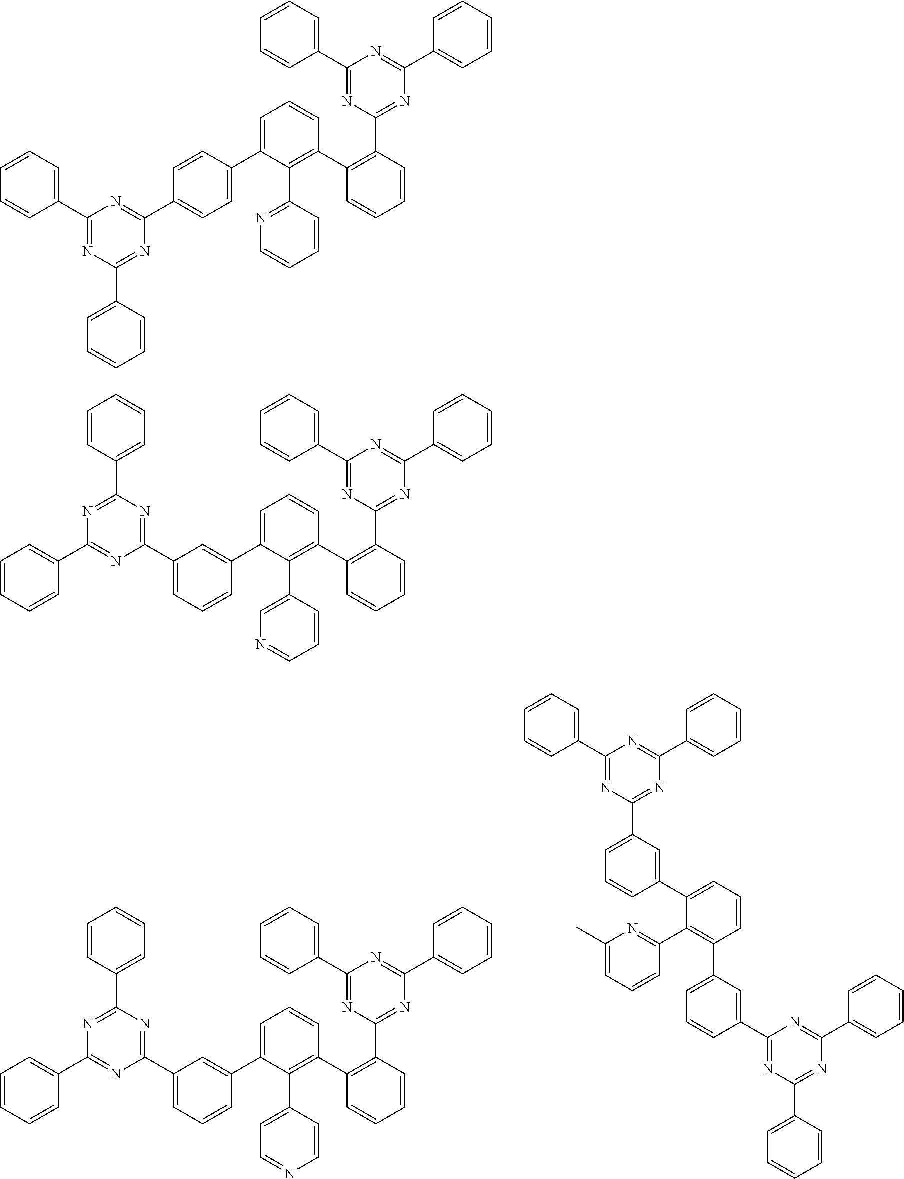

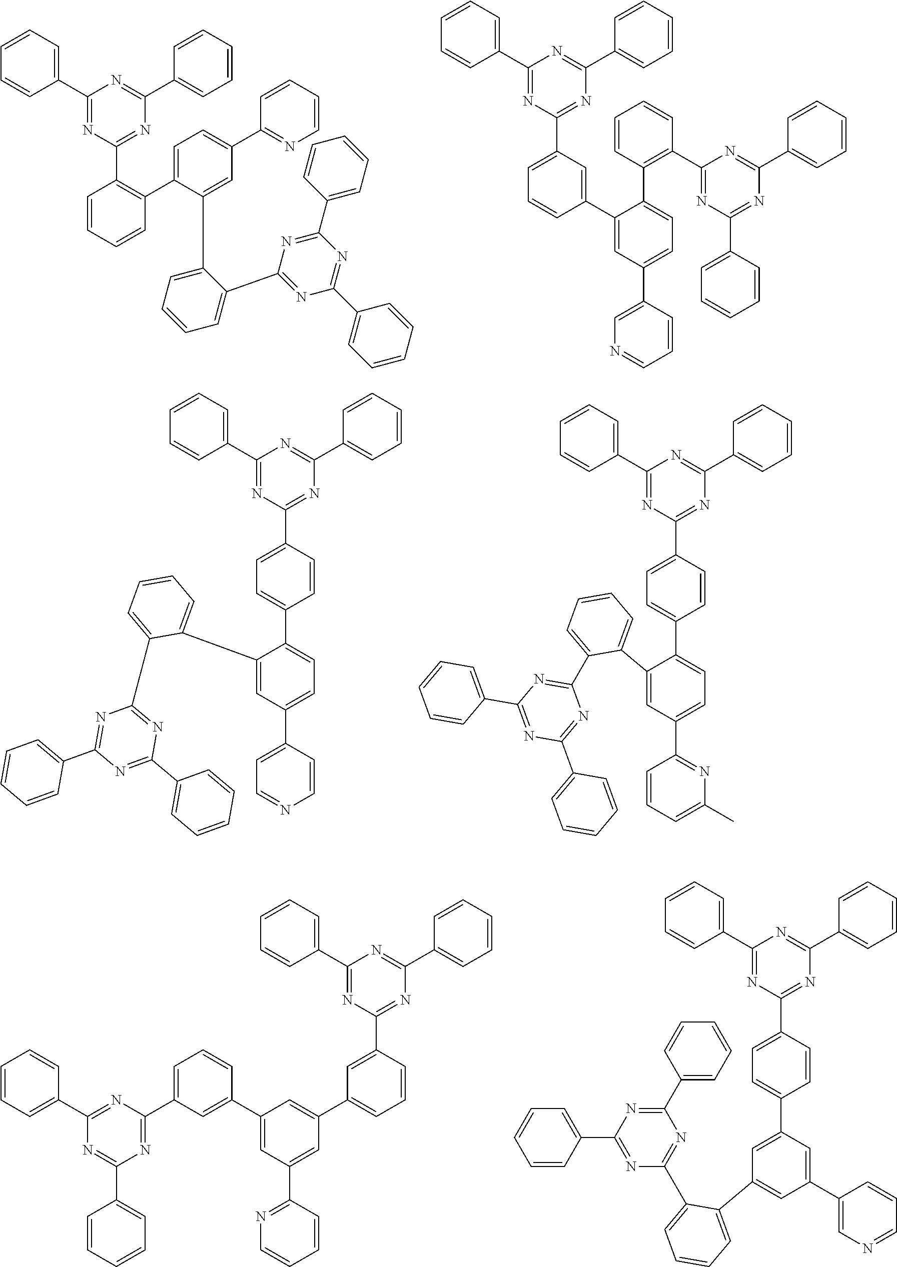

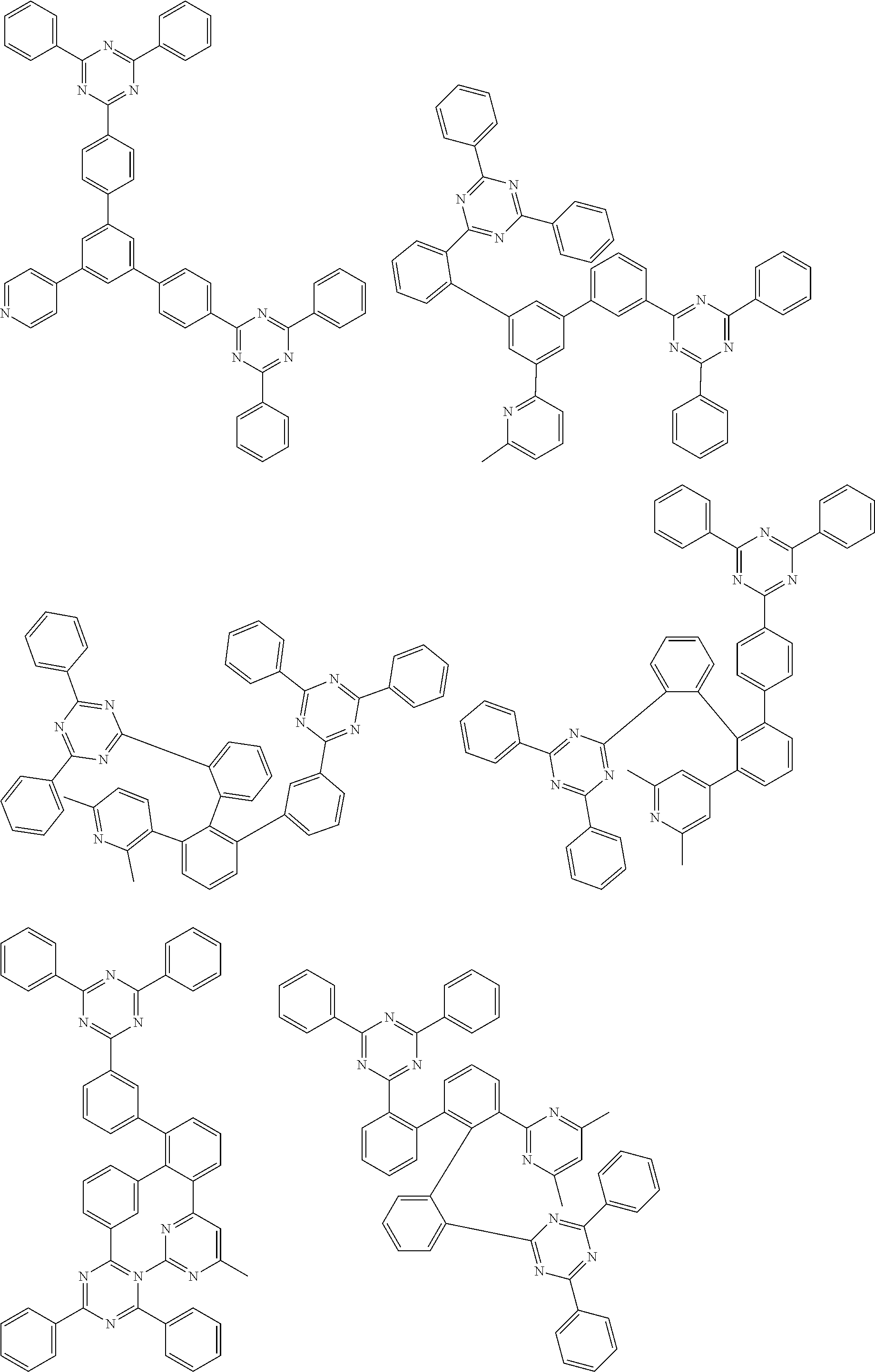

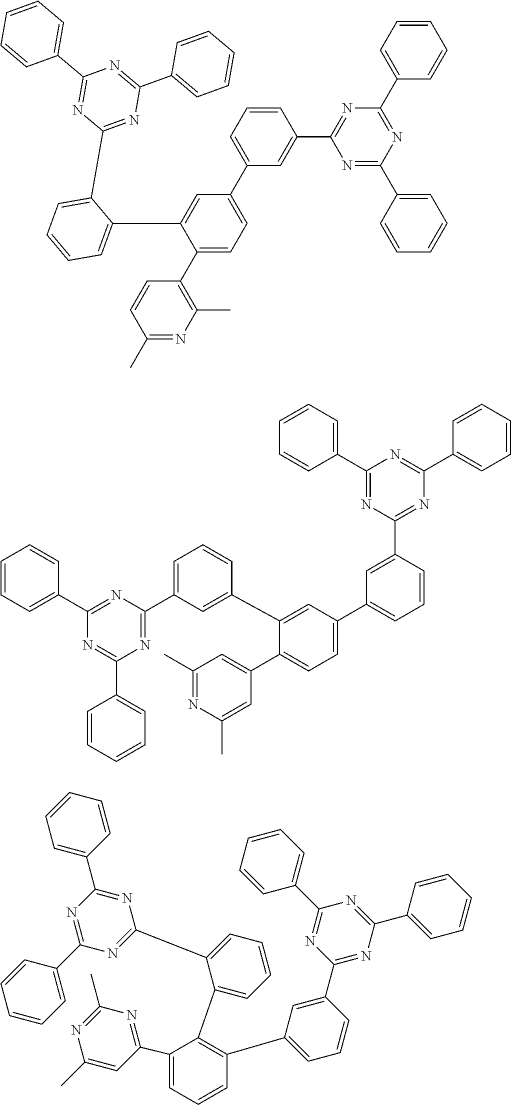

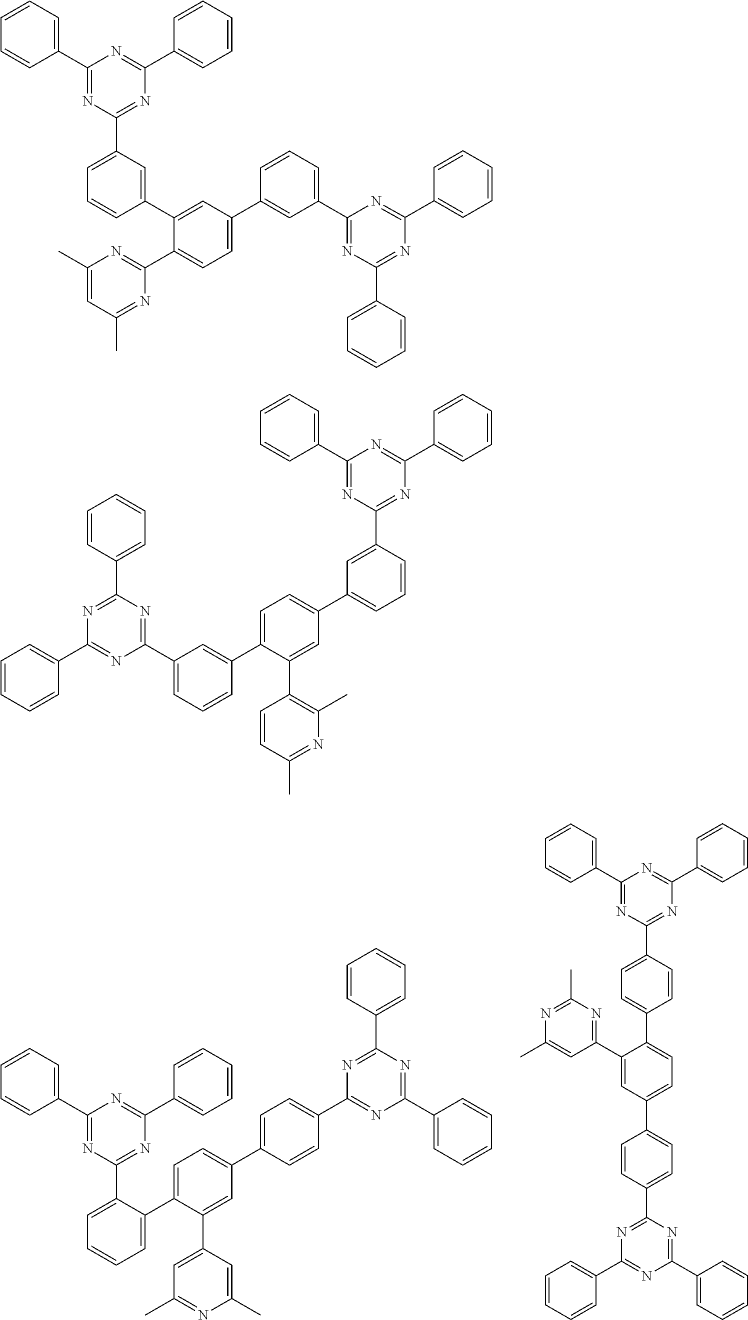 [SMILES: Cc1cc(C)nc(-c2ccc(-c3cccc(-c4nc(-c5ccccc5)nc(-c5ccccc5)n4)c3)cc2-c2cccc(-c3nc(-c4ccccc4)nc(-c4ccccc4)n3)c2)n1 ; Cc1ccc(-c2cc(-c3cccc(-c4nc(-c5ccccc5)nc(-c5ccccc5)n4)c3)ccc2-c2cccc(-c3nc(-c4ccccc4)nc(-c4ccccc4)n3)c2)c(C)n1 ; Cc1cc(-c2cc(-c3ccc(-c4nc(-c5ccccc5)nc(-c5ccccc5)n4)cc3)ccc2-c2ccccc2-c2nc(-c3ccccc3)nc(-c3ccccc3)n2)cc(C)n1 ; Cc1cc(-c2cc(-c3ccc(-c4nc(-c5ccccc5)nc(-c5ccccc5)n4)cc3)ccc2-c2ccc(-c3nc(-c4ccccc4)nc(-c4ccccc4)n3)cc2)nc(C)n1]

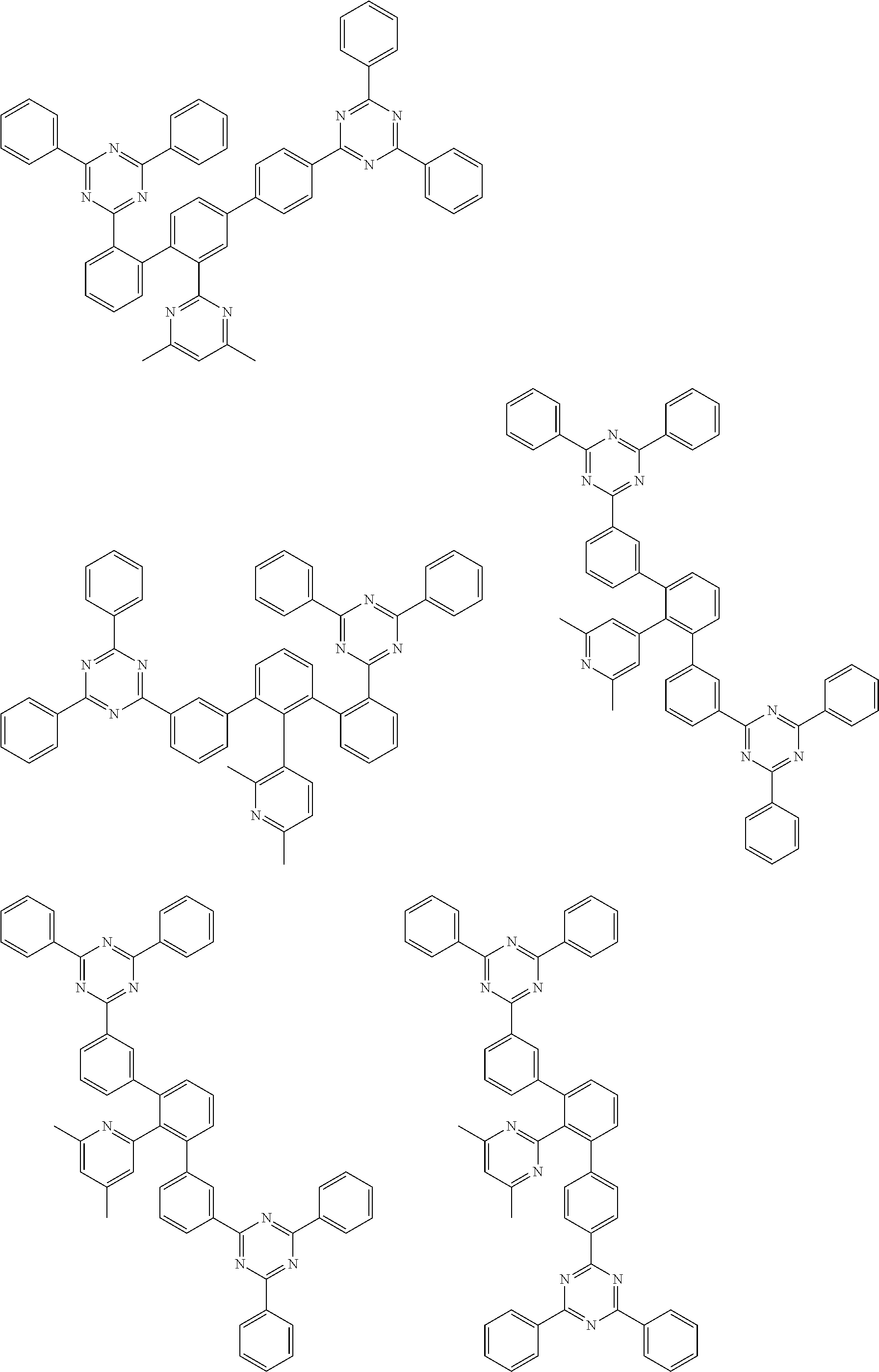

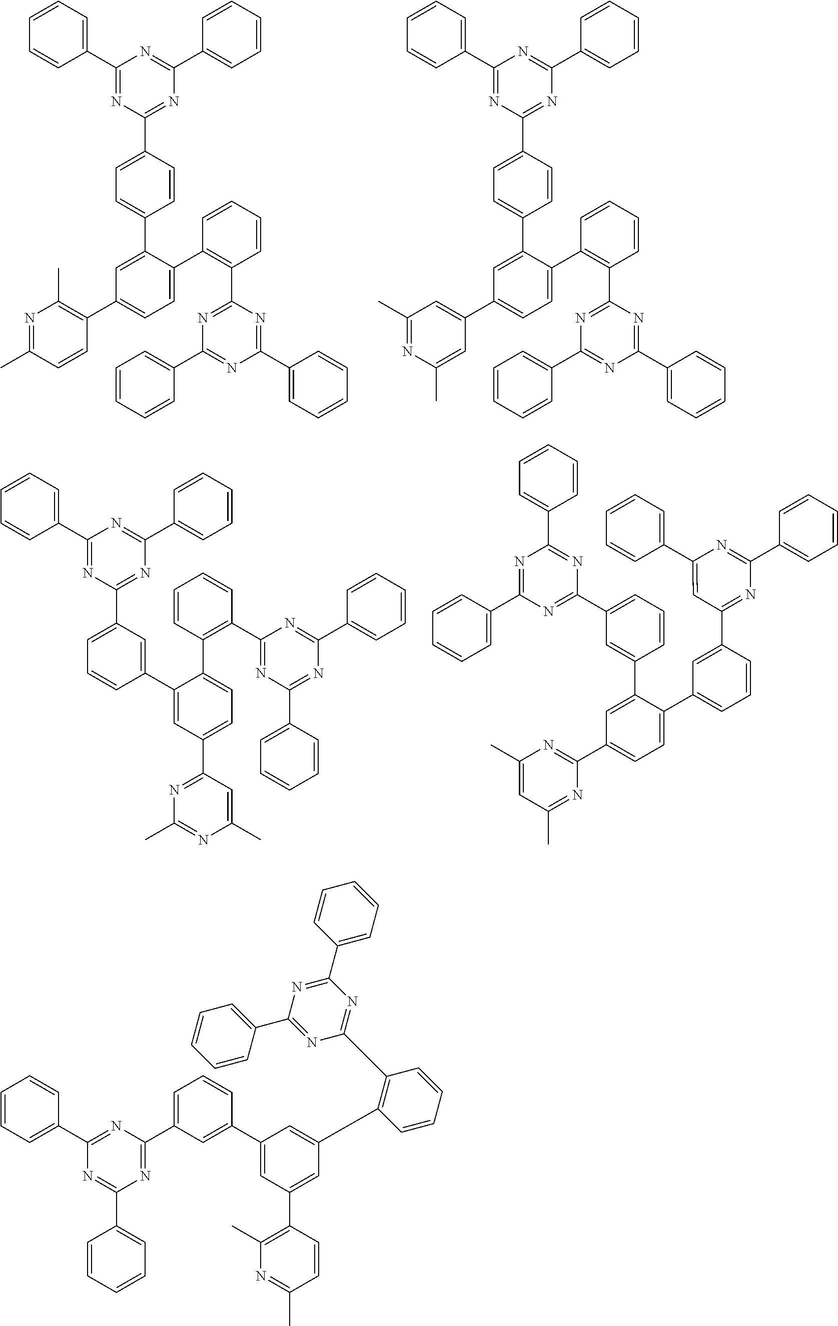

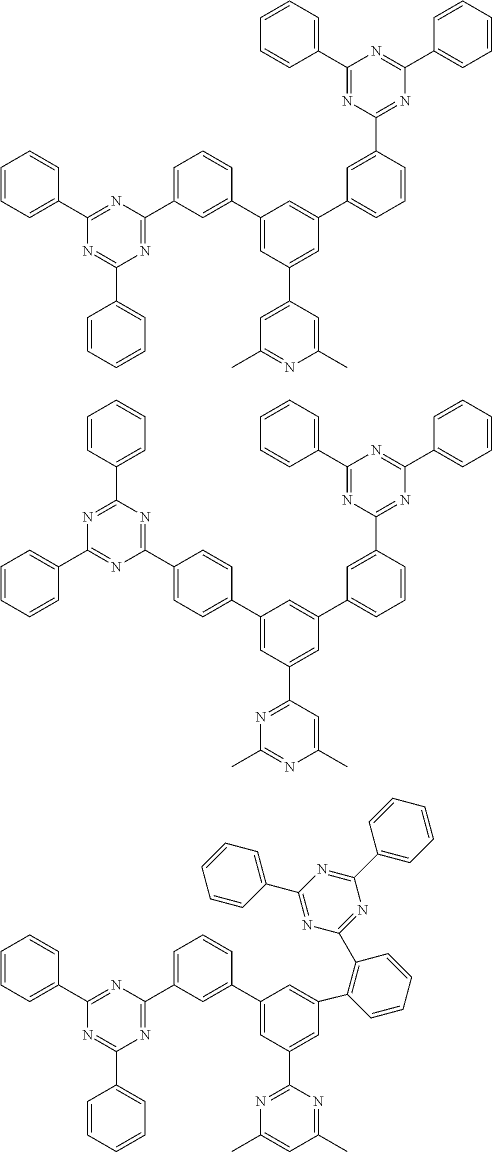

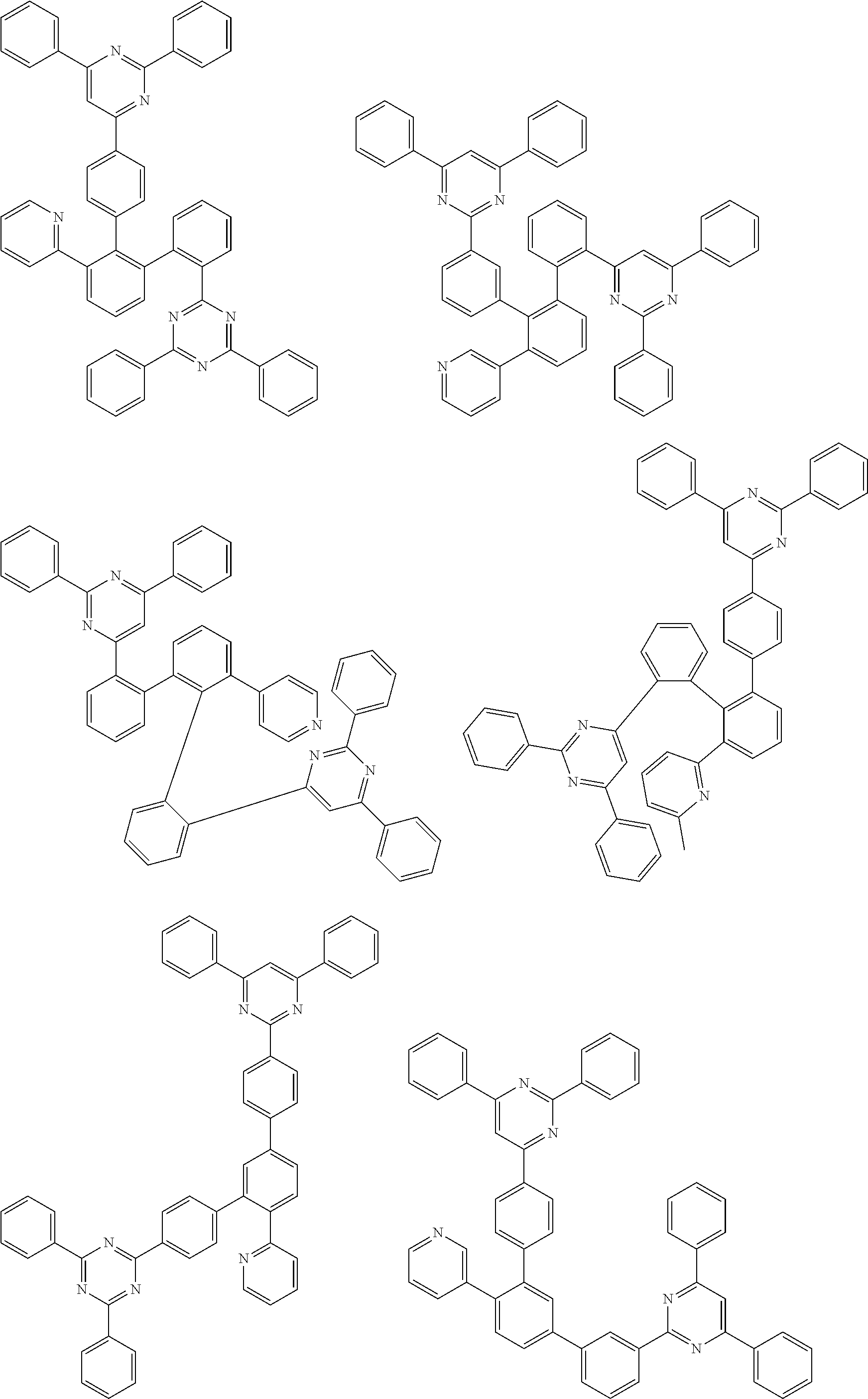

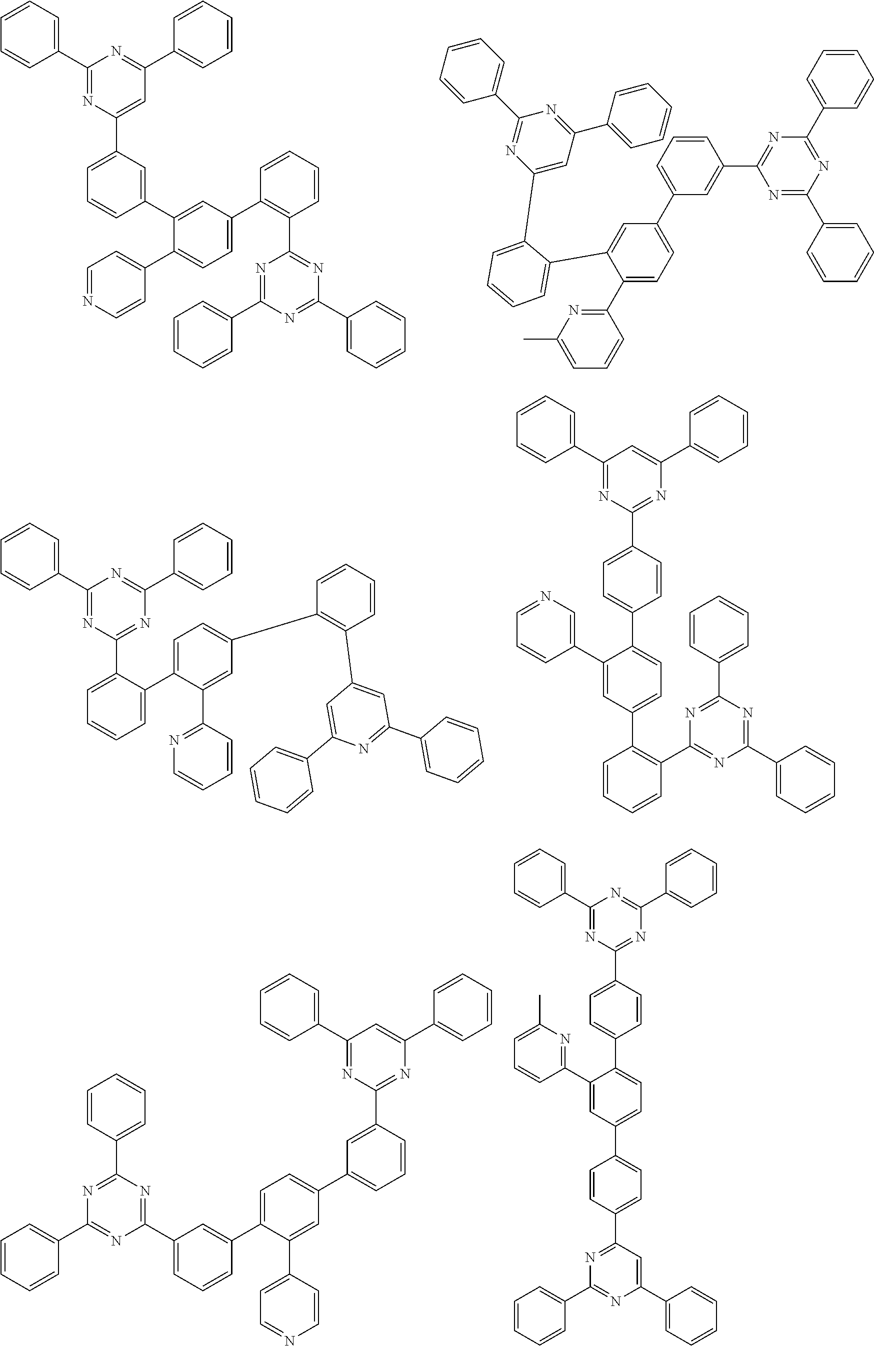

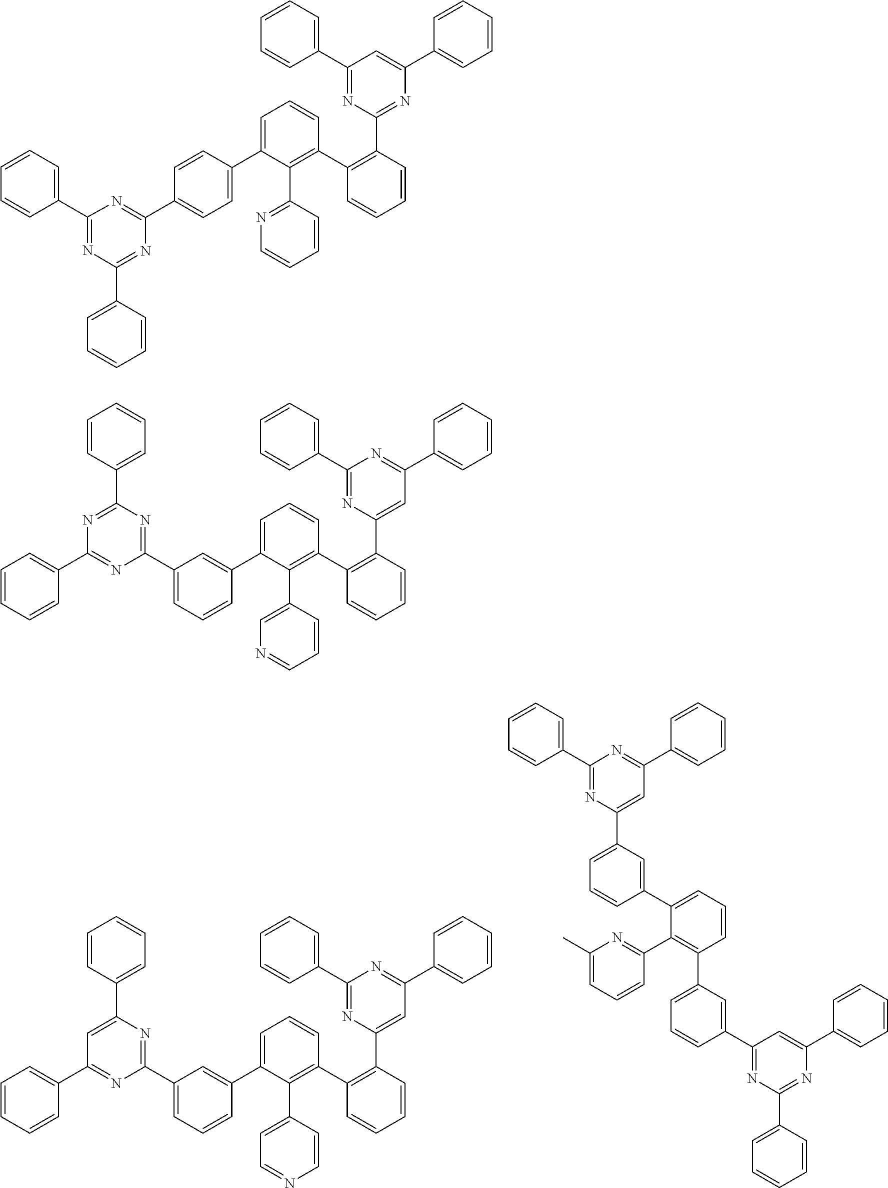

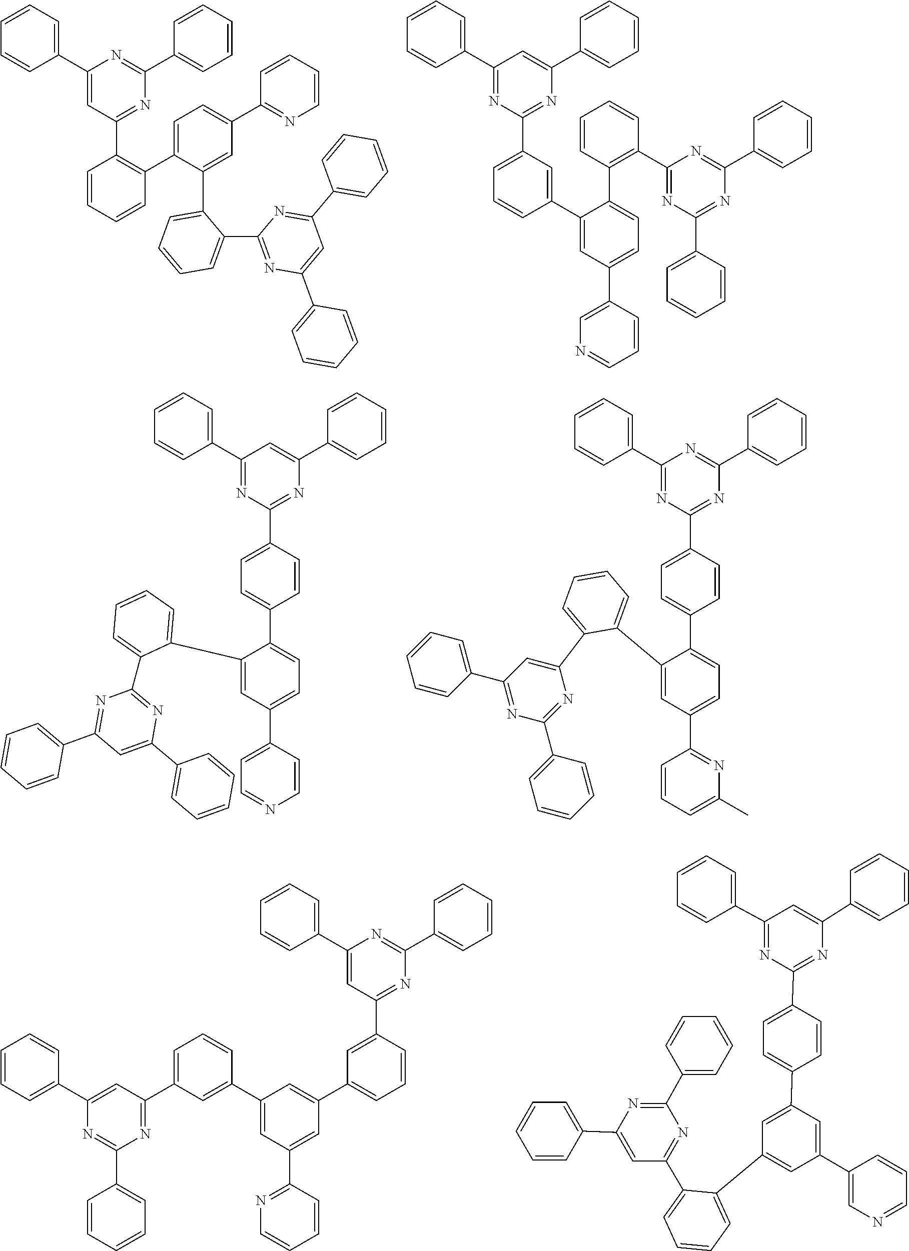

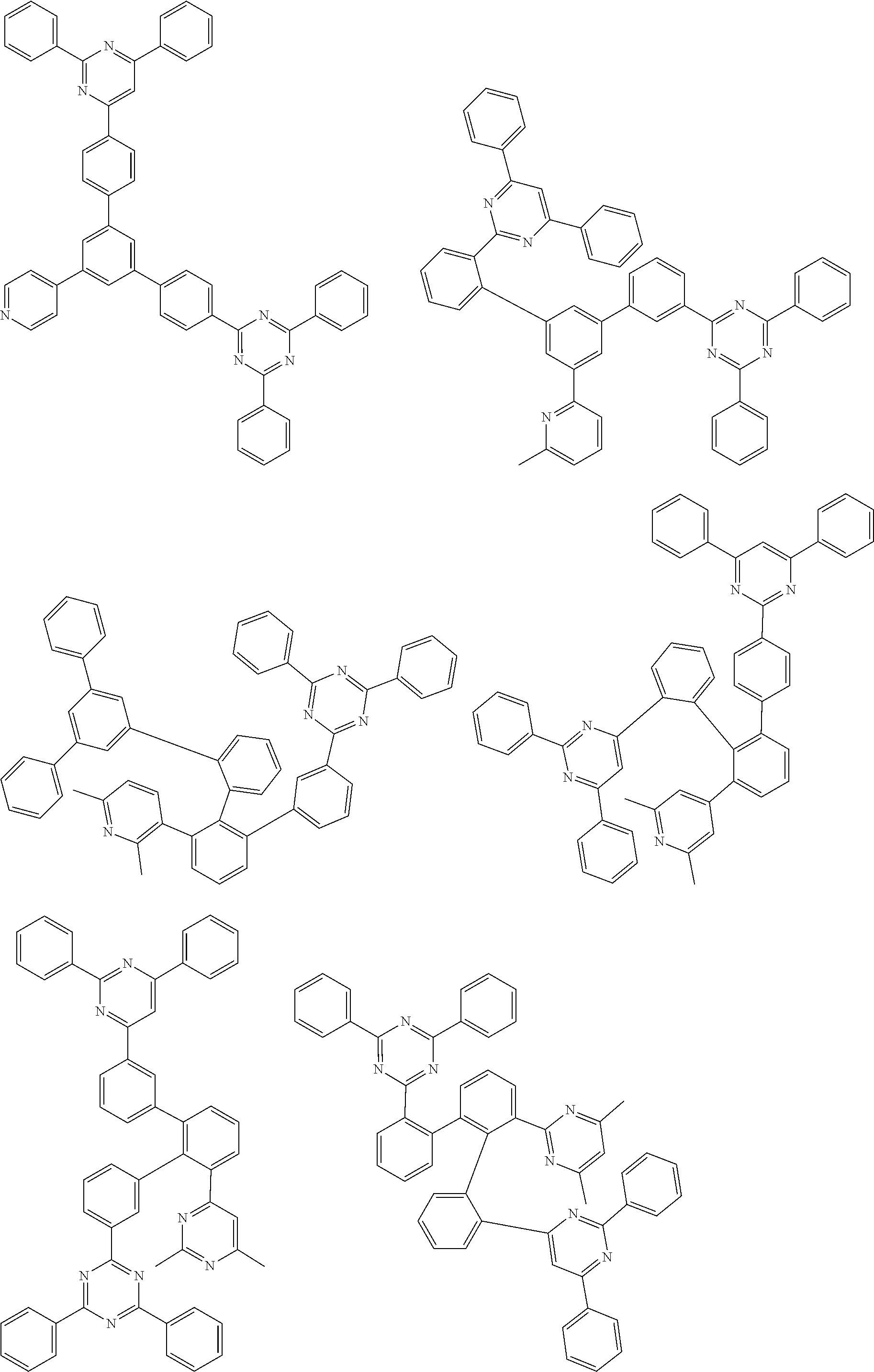

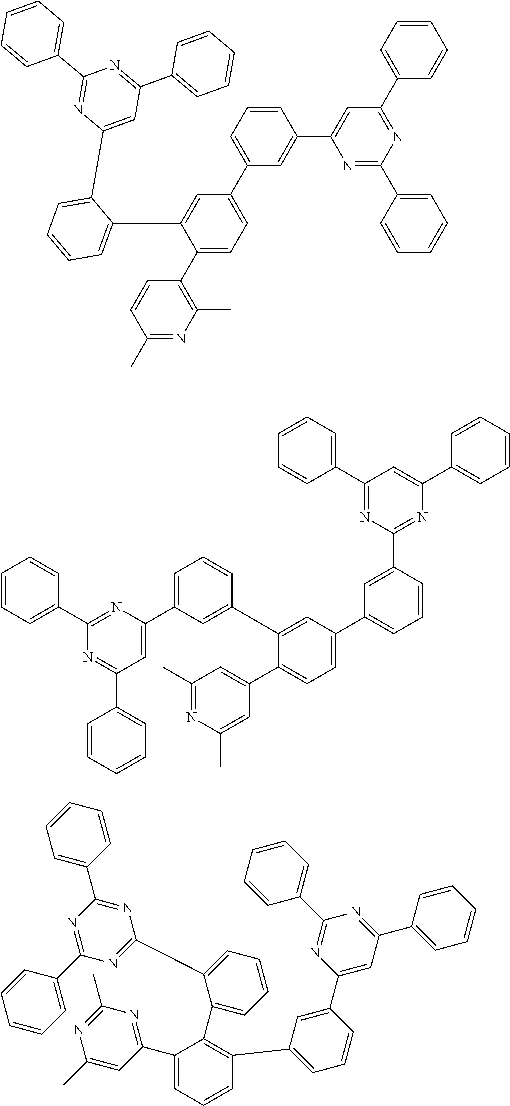

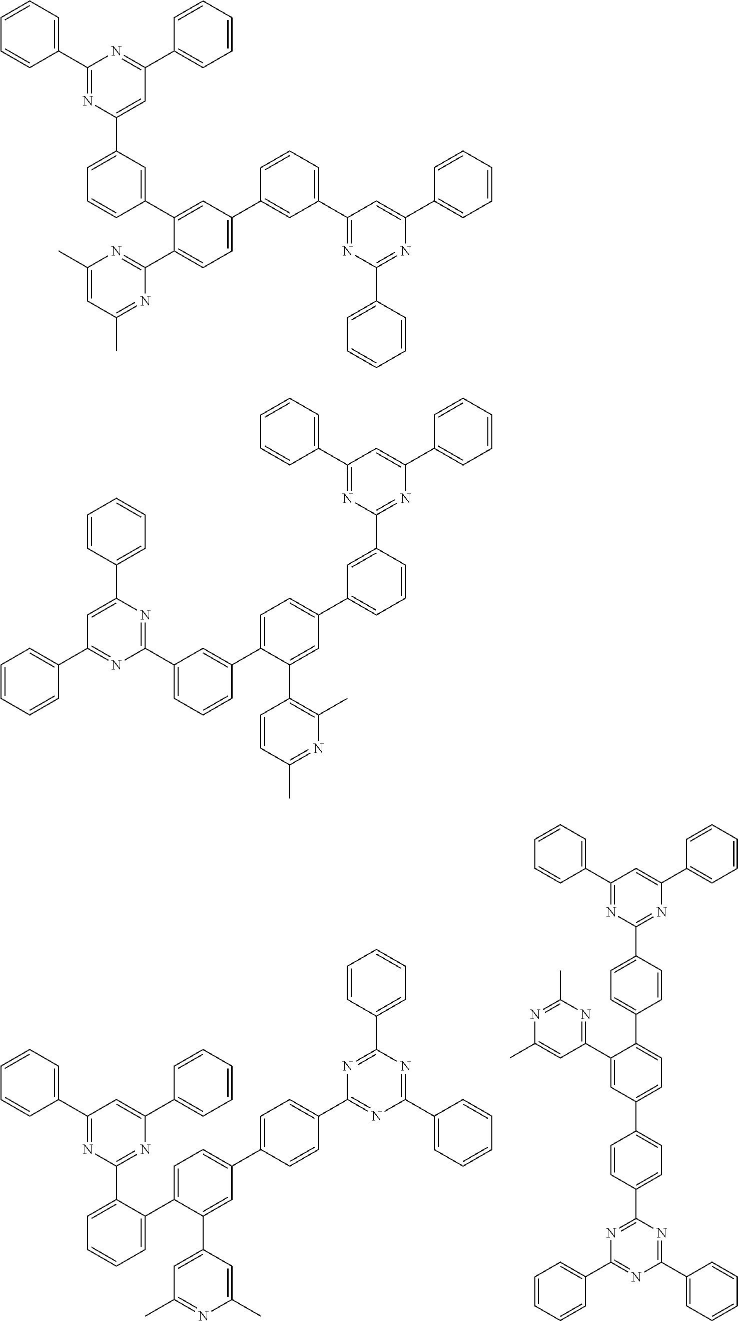 [SMILES: Cc1cc(C)nc(-c2ccc(-c3cccc(-c4cc(-c5ccccc5)nc(-c5ccccc5)n4)c3)cc2-c2cccc(-c3cc(-c4ccccc4)nc(-c4ccccc4)n3)c2)n1 ; Cc1ccc(-c2cc(-c3cccc(-c4nc(-c5ccccc5)cc(-c5ccccc5)n4)c3)ccc2-c2cccc(-c3nc(-c4ccccc4)cc(-c4ccccc4)n3)c2)c(C)n1 ; Cc1cc(-c2cc(-c3ccc(-c4nc(-c5ccccc5)nc(-c5ccccc5)n4)cc3)ccc2-c2ccccc2-c2nc(-c3ccccc3)cc(-c3ccccc3)n2)cc(C)n1 ; Cc1cc(-c2cc(-c3ccc(-c4nc(-c5ccccc5)nc(-c5ccccc5)n4)cc3)ccc2-c2ccc(-c3nc(-c4ccccc4)cc(-c4ccccc4)n3)cc2)nc(C)n1]

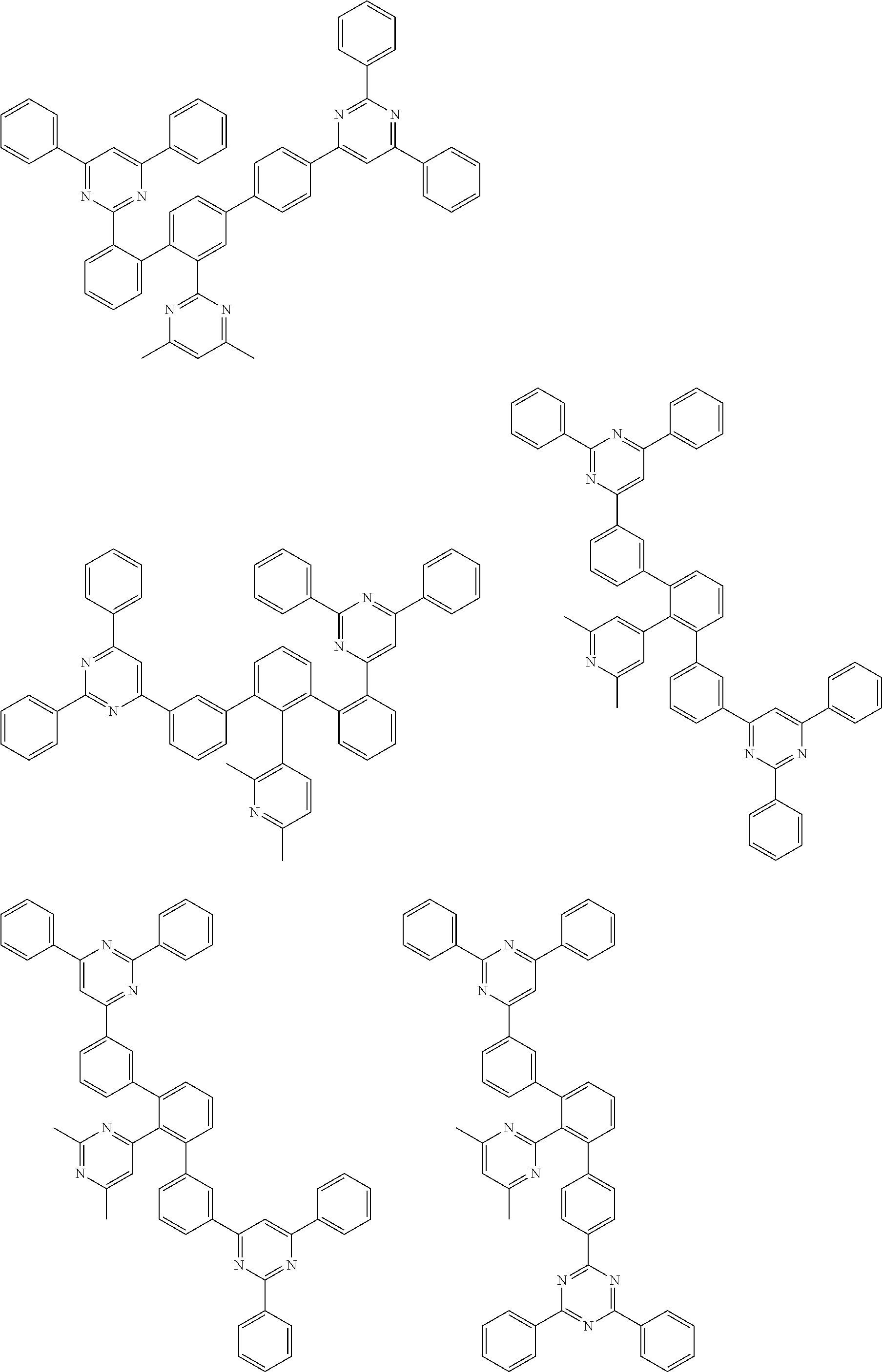

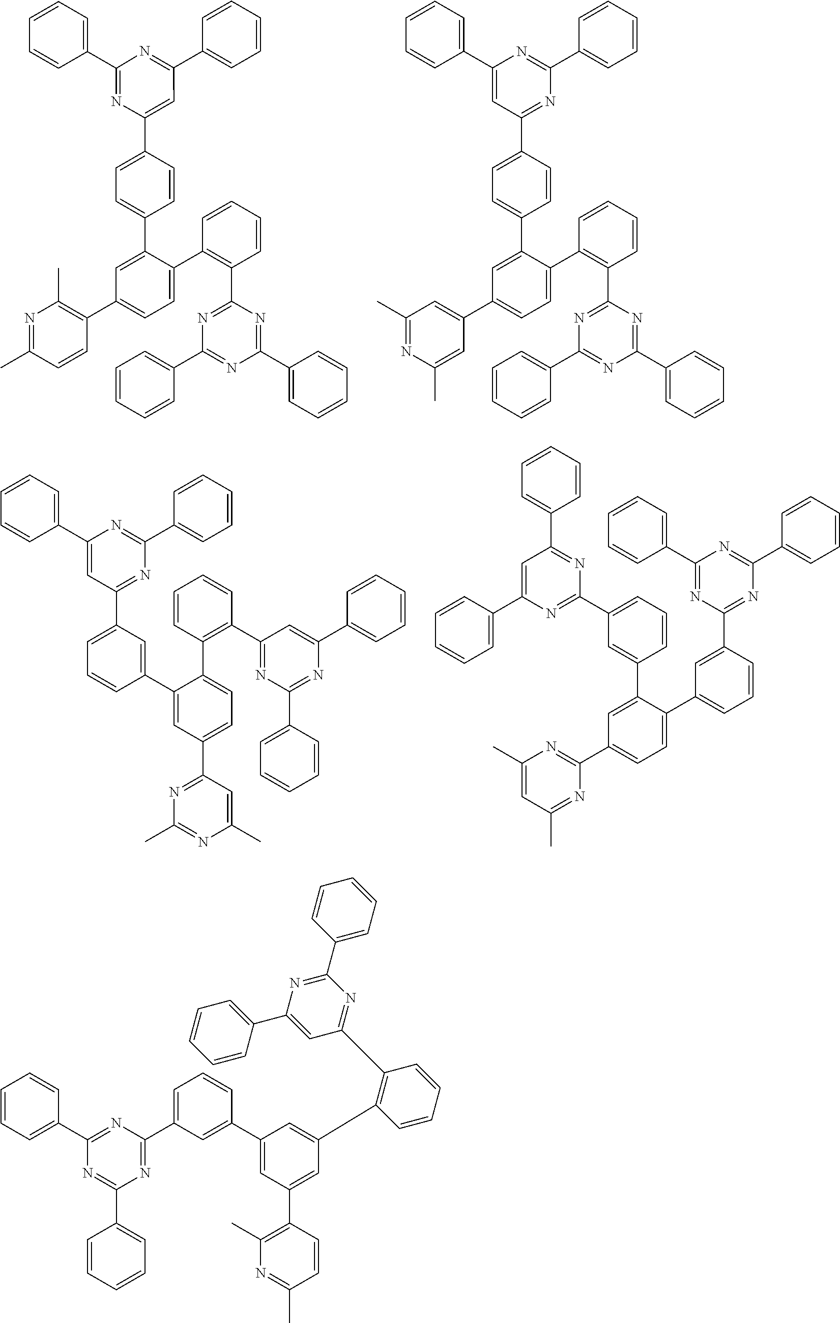

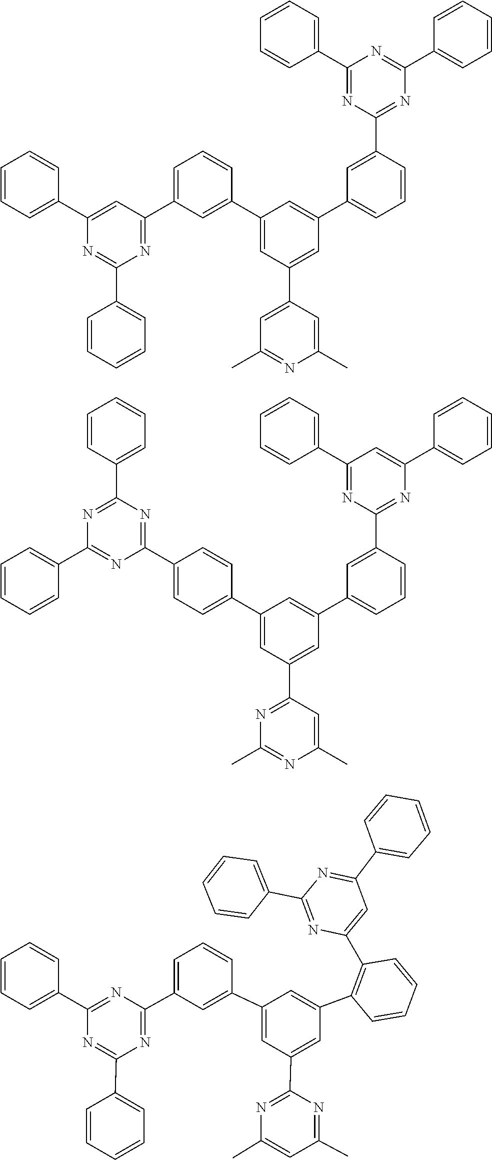

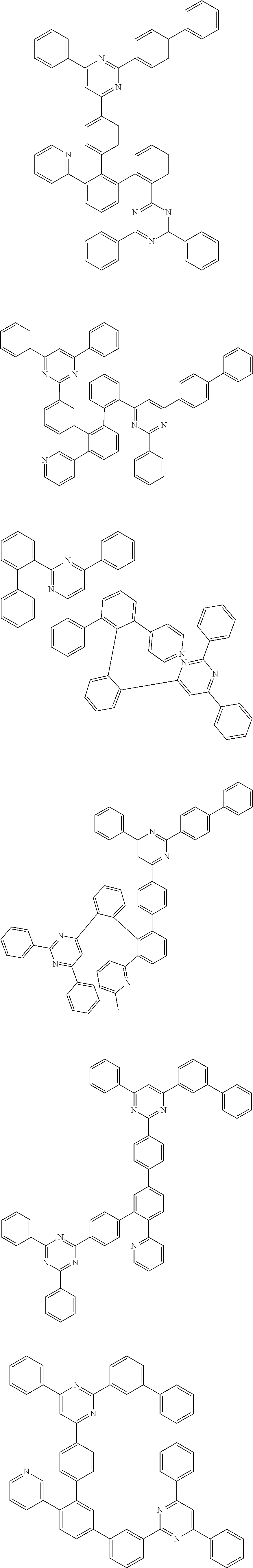

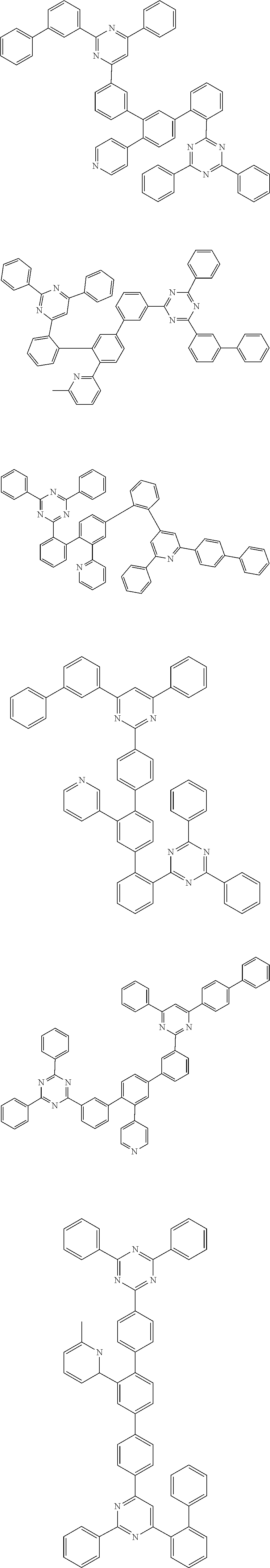

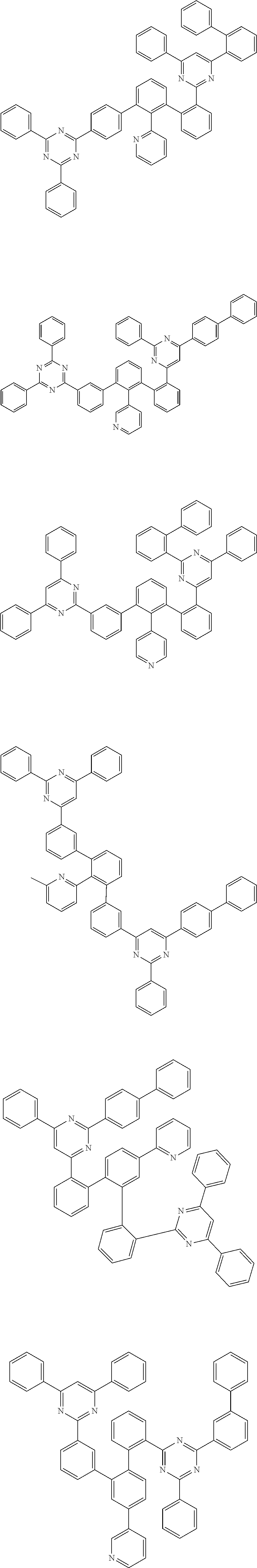

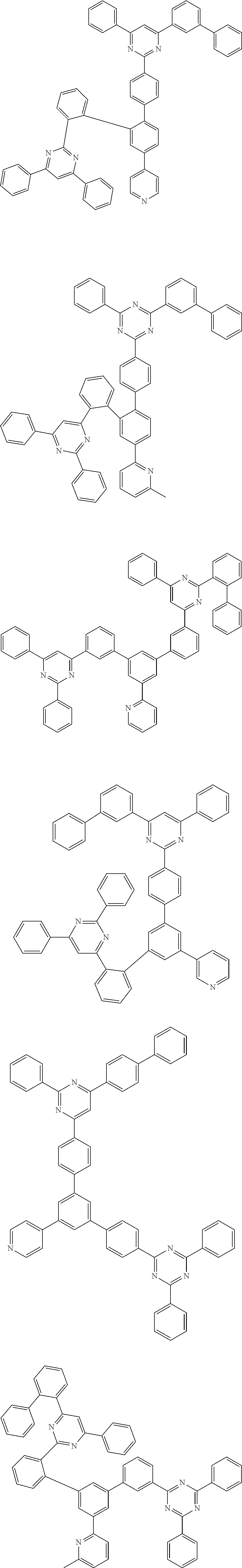

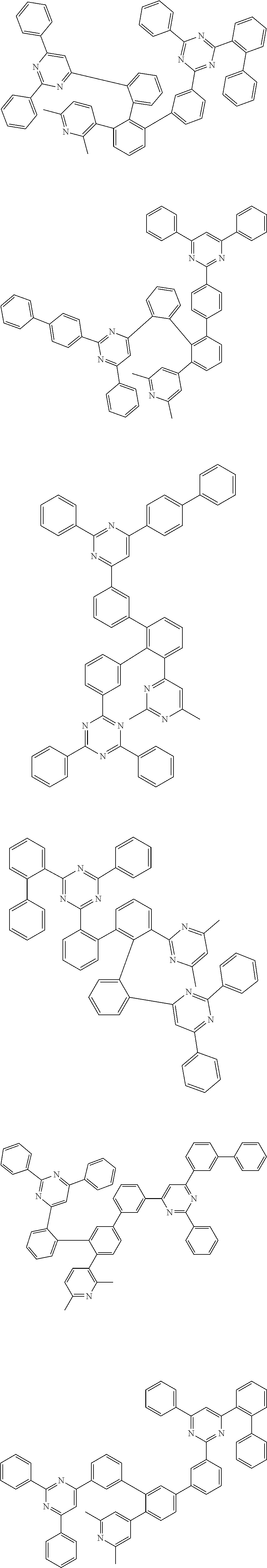

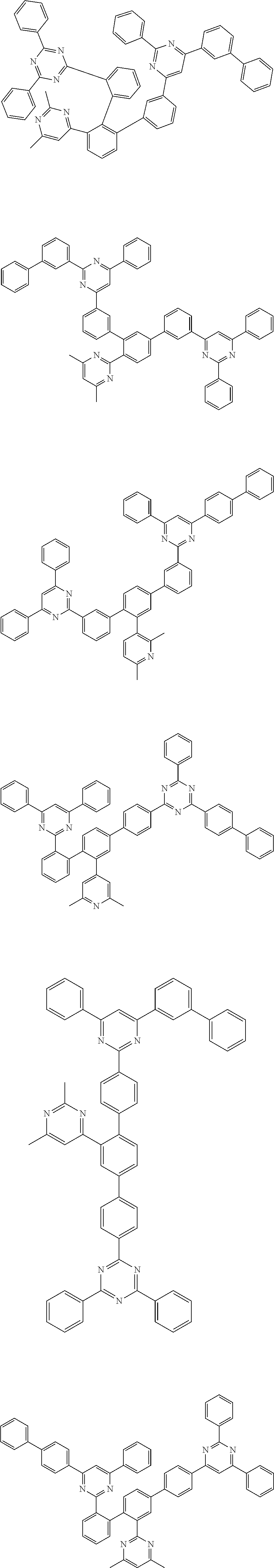

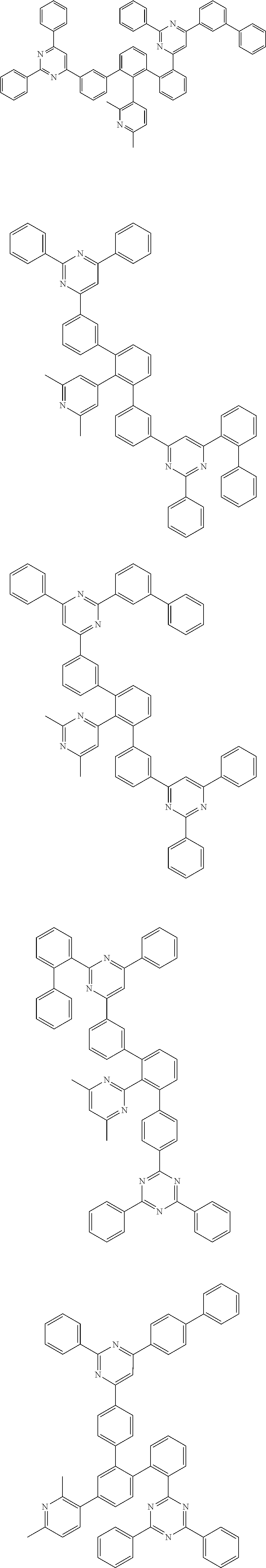

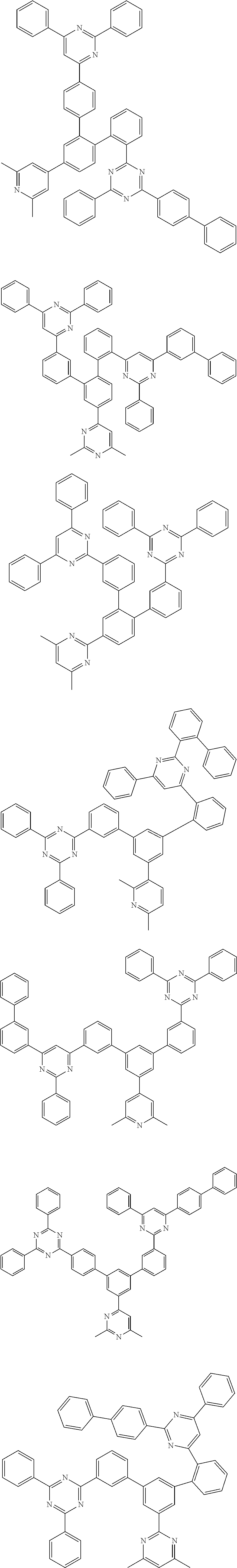

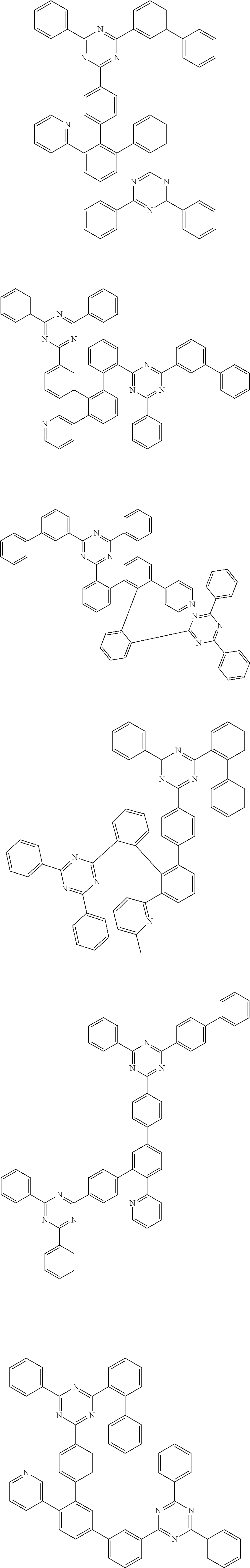

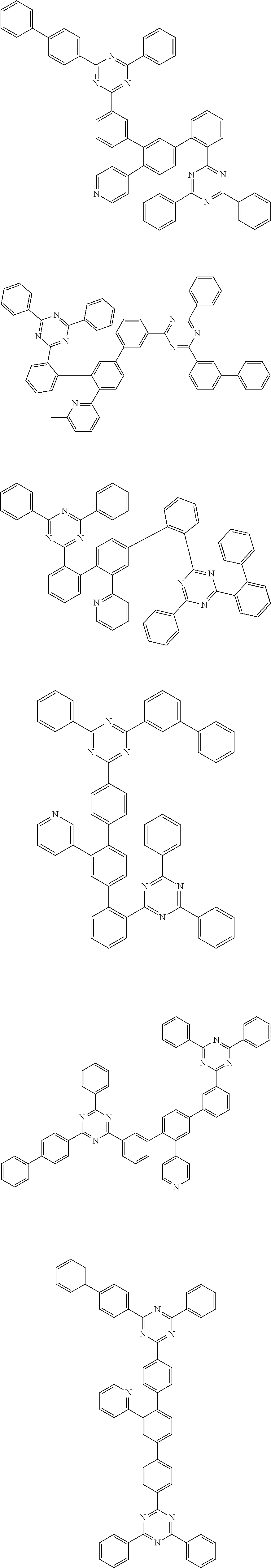

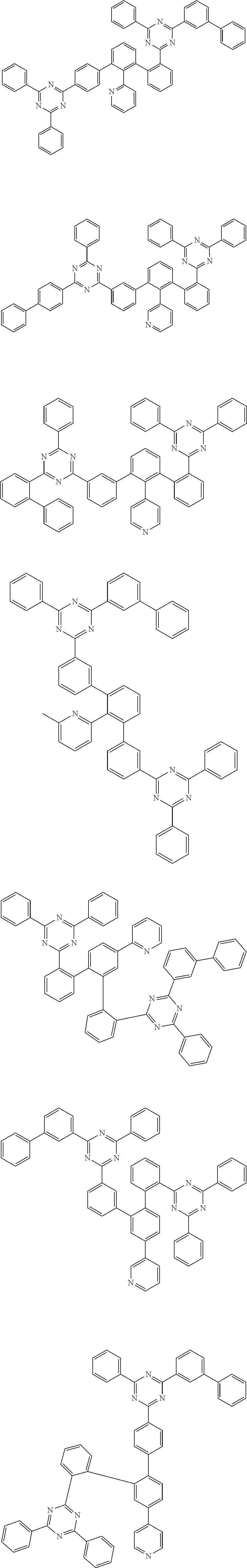

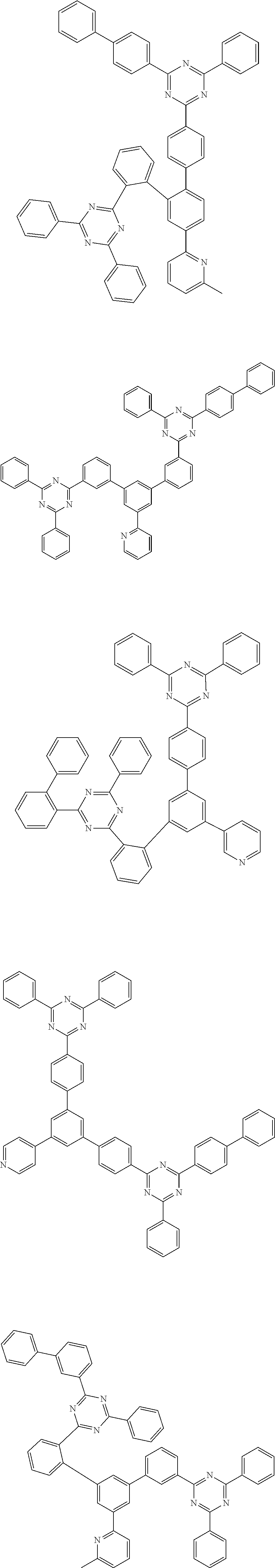

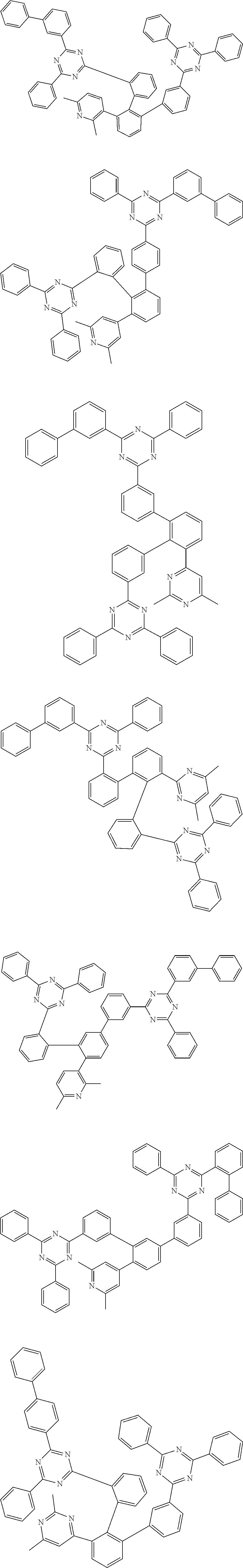

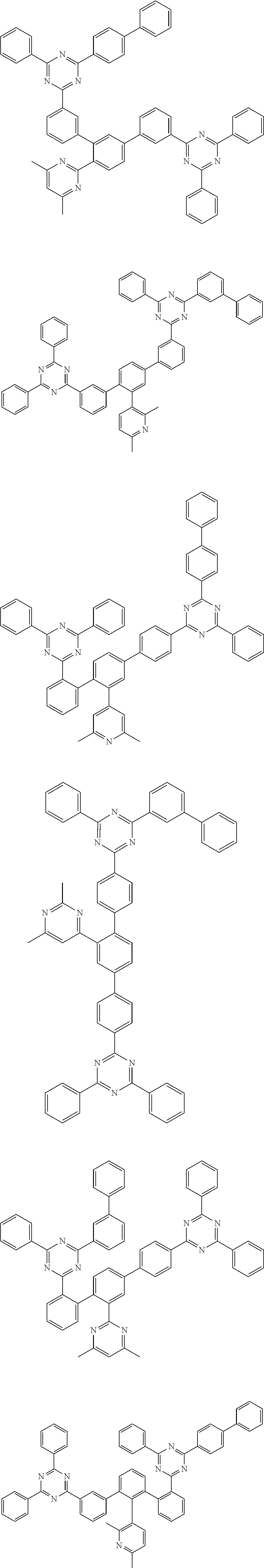

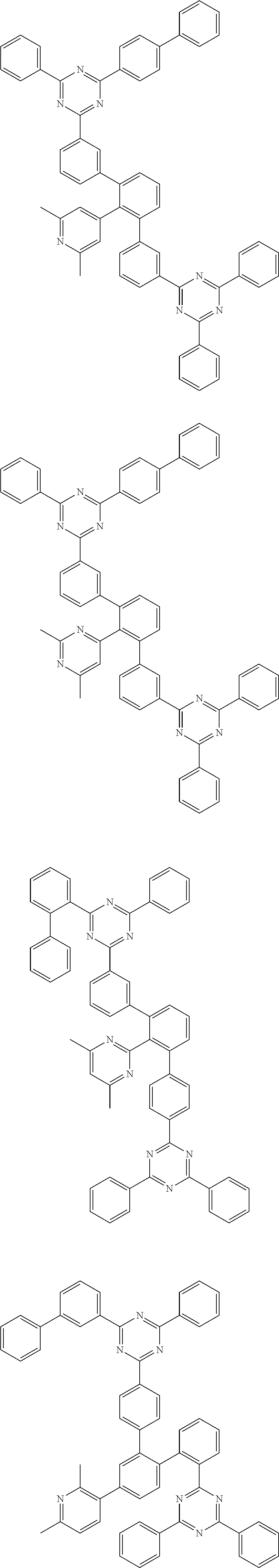 [SMILES: Cc1cc(-c2c(-c3cccc(-c4nc(-c5ccccc5)nc(-c5ccccc5)n4)c3)cccc2-c2cccc(-c3nc(-c4ccccc4)nc(-c4ccc(-c5ccccc5)cc4)n3)c2)cc(C)n1 ; Cc1cc(-c2c(-c3cccc(-c4nc(-c5ccccc5)nc(-c5ccccc5)n4)c3)cccc2-c2cccc(-c3nc(-c4ccccc4)nc(-c4ccc(-c5ccccc5)cc4)n3)c2)nc(C)n1 ; Cc1cc(C)nc(-c2c(-c3ccc(-c4nc(-c5ccccc5)nc(-c5ccccc5)n4)cc3)cccc2-c2cccc(-c3nc(-c4ccccc4)nc(-c4ccccc4-c4ccccc4)n3)c2)n1 ; Cc1ccc(-c2ccc(-c3ccccc3-c3nc(-c4ccccc4)nc(-c4ccccc4)n3)c(-c3ccc(-c4nc(-c5ccccc5)nc(-c5cccc(-c6ccccc6)c5)n4)cc3)c2)c(C)n1]

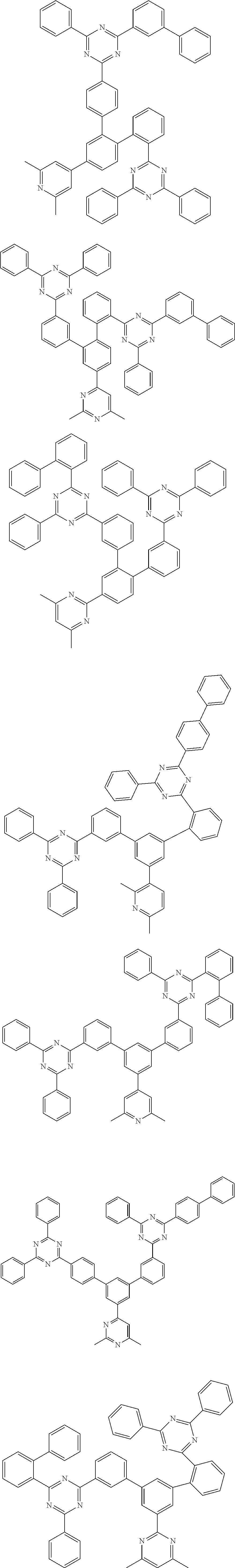

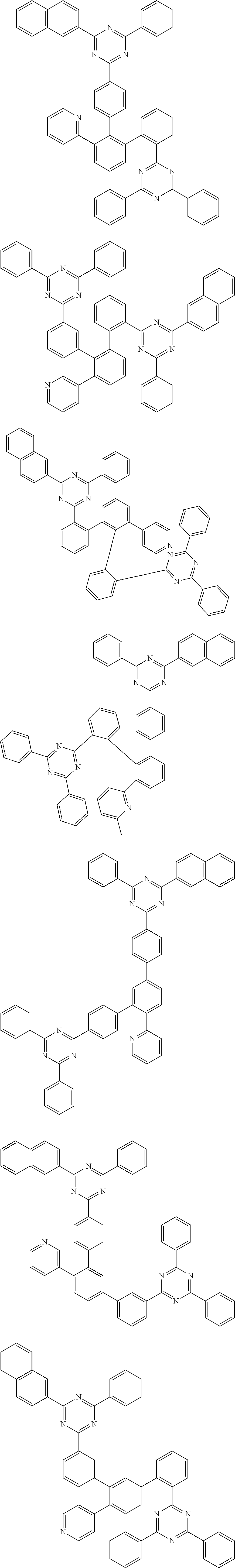

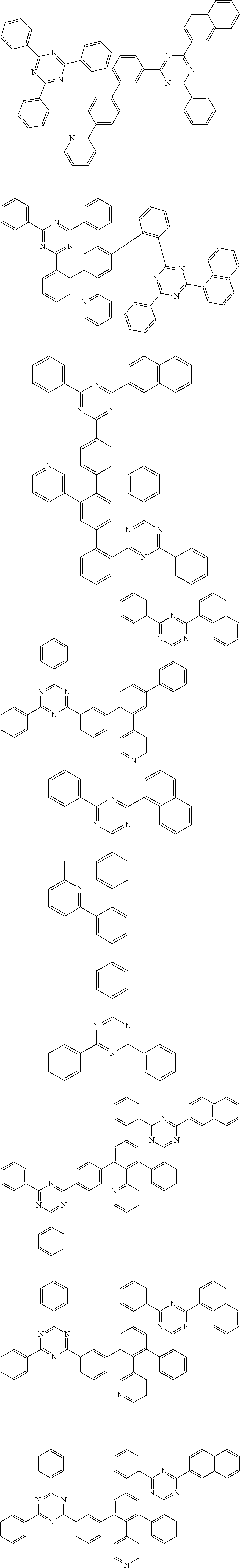

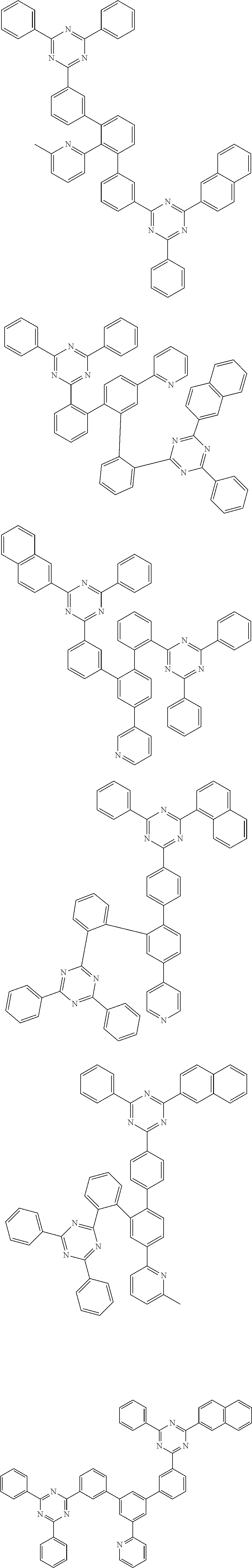

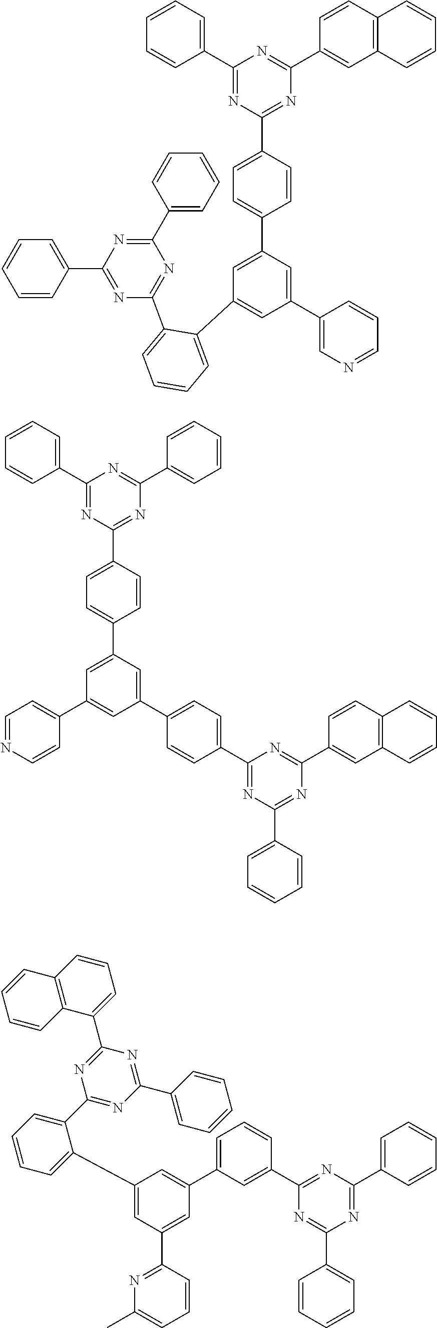

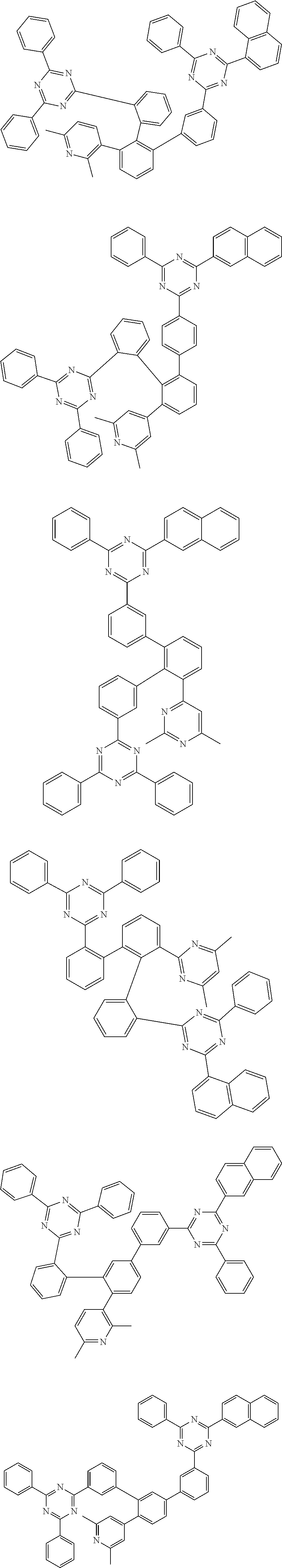

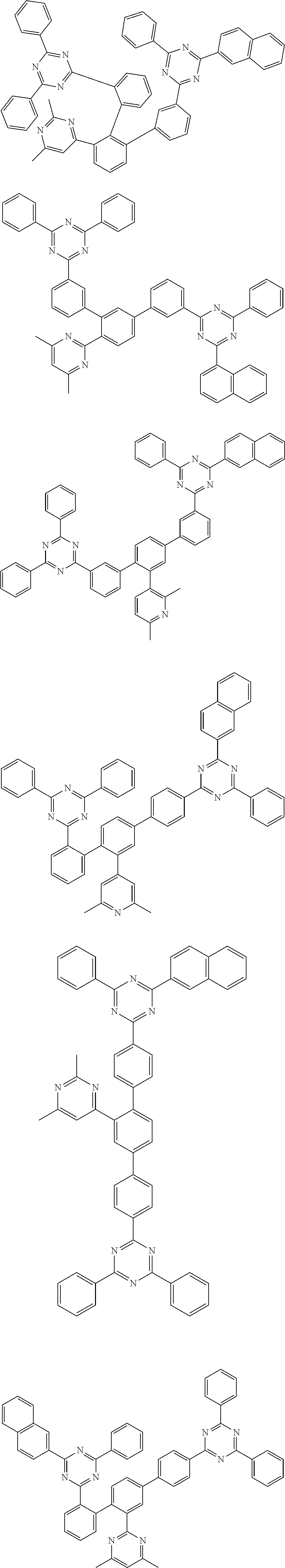

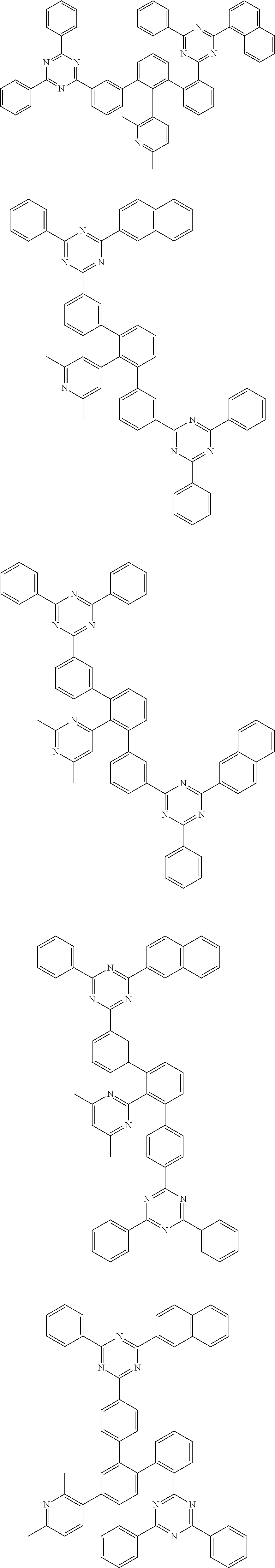

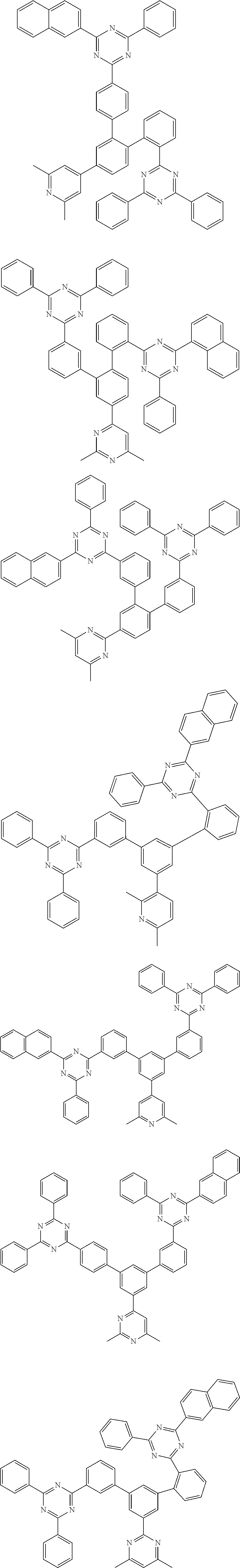

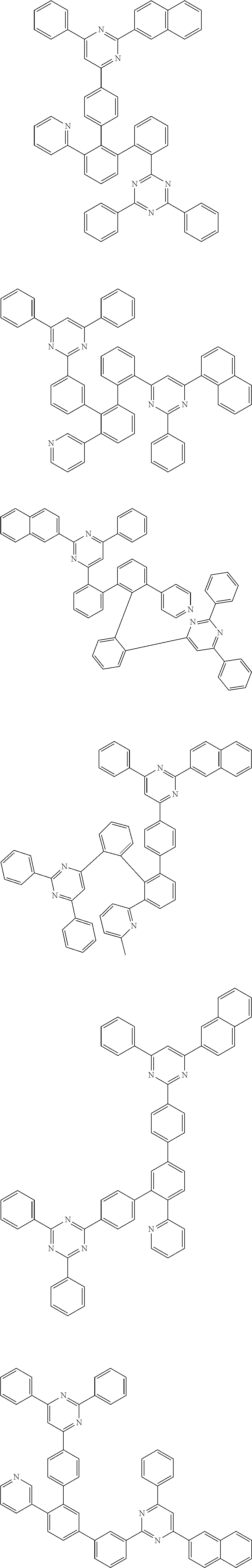

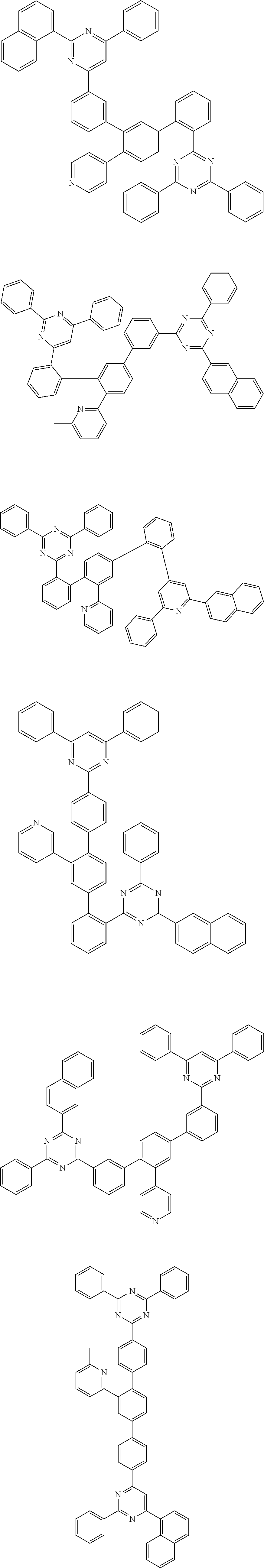

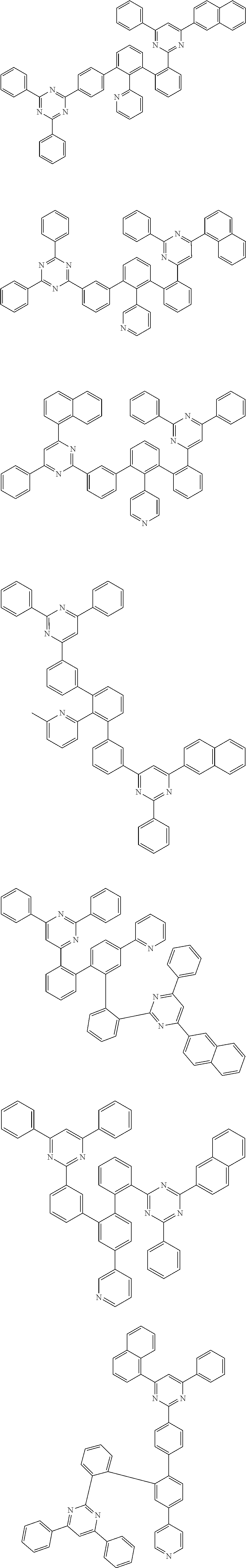

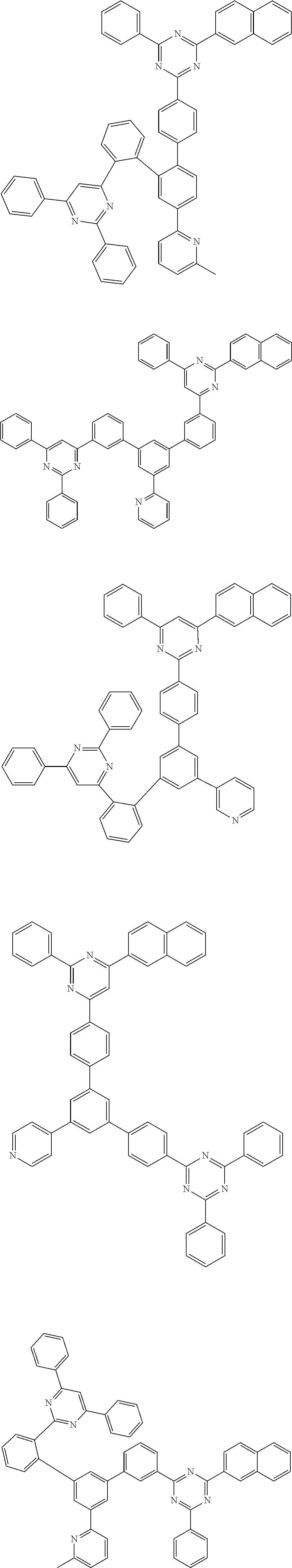

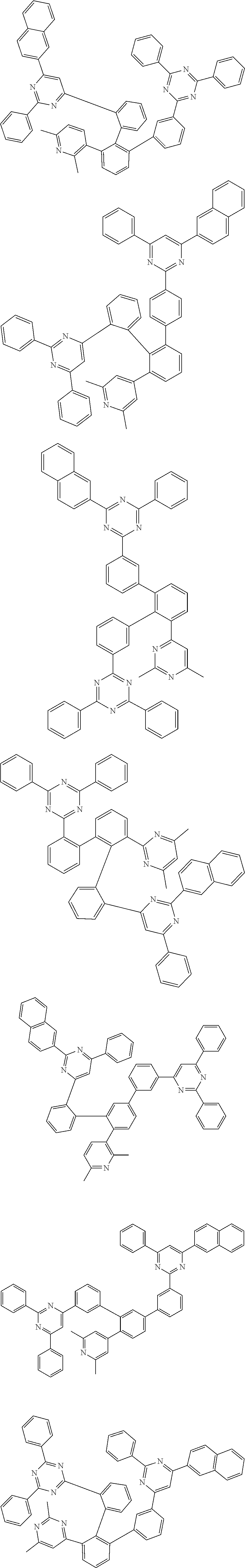

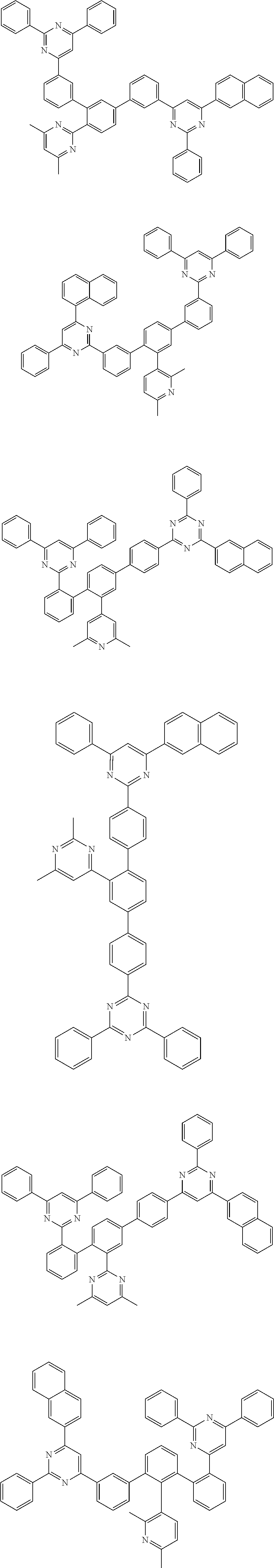

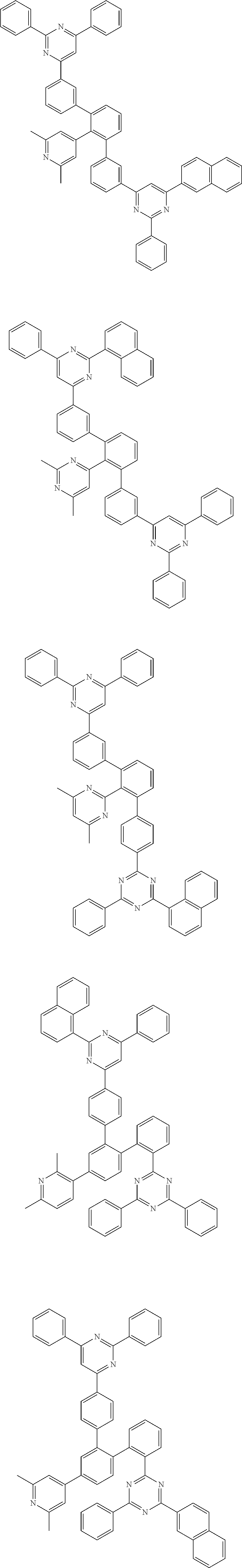

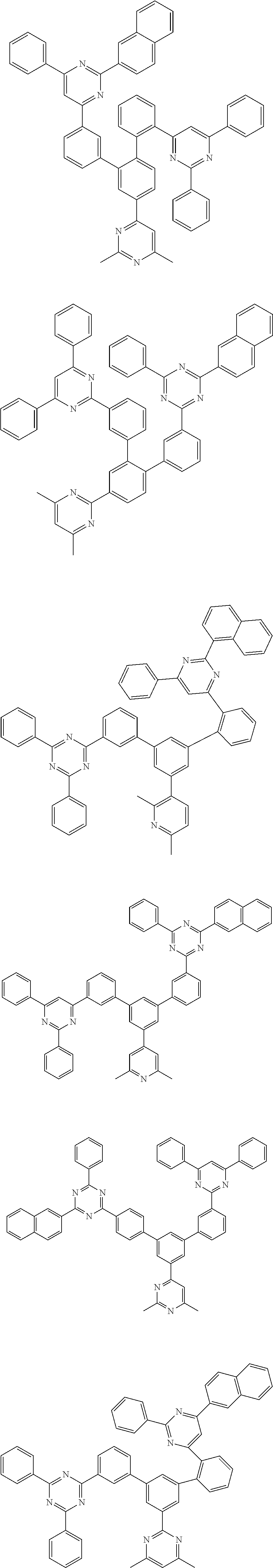

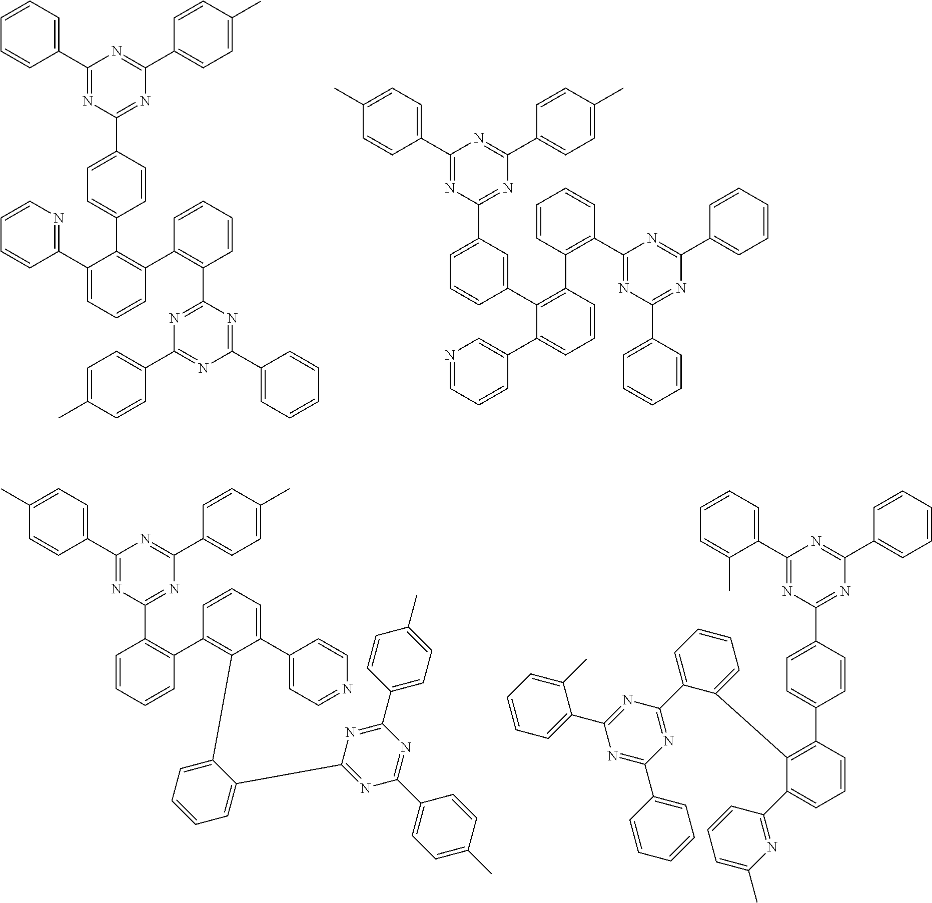

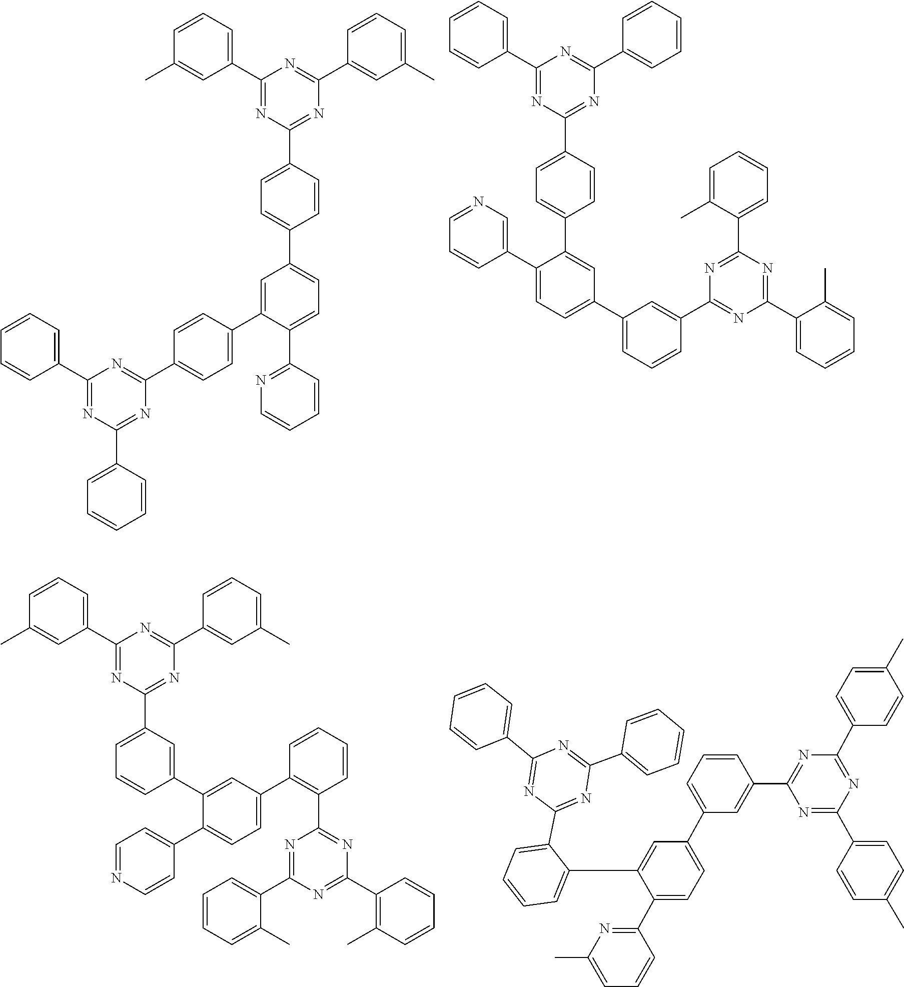

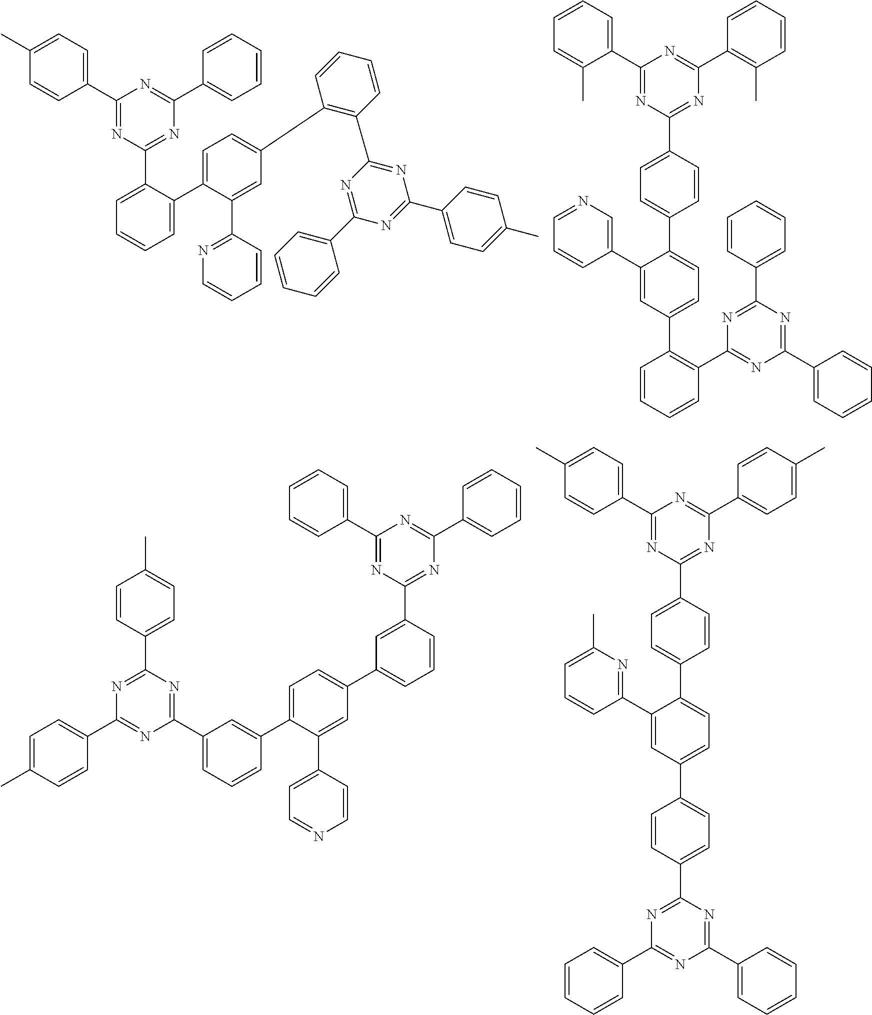

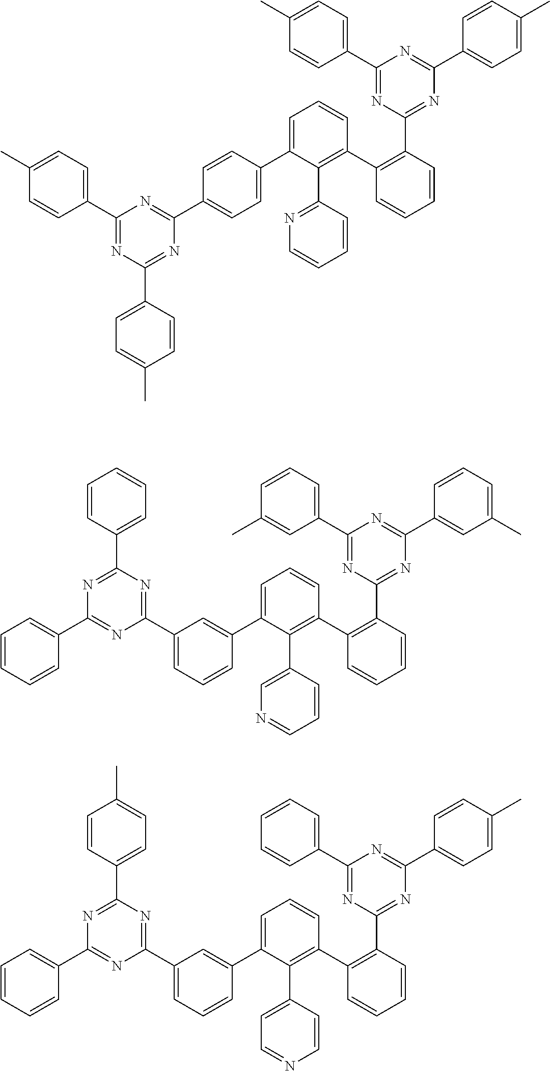

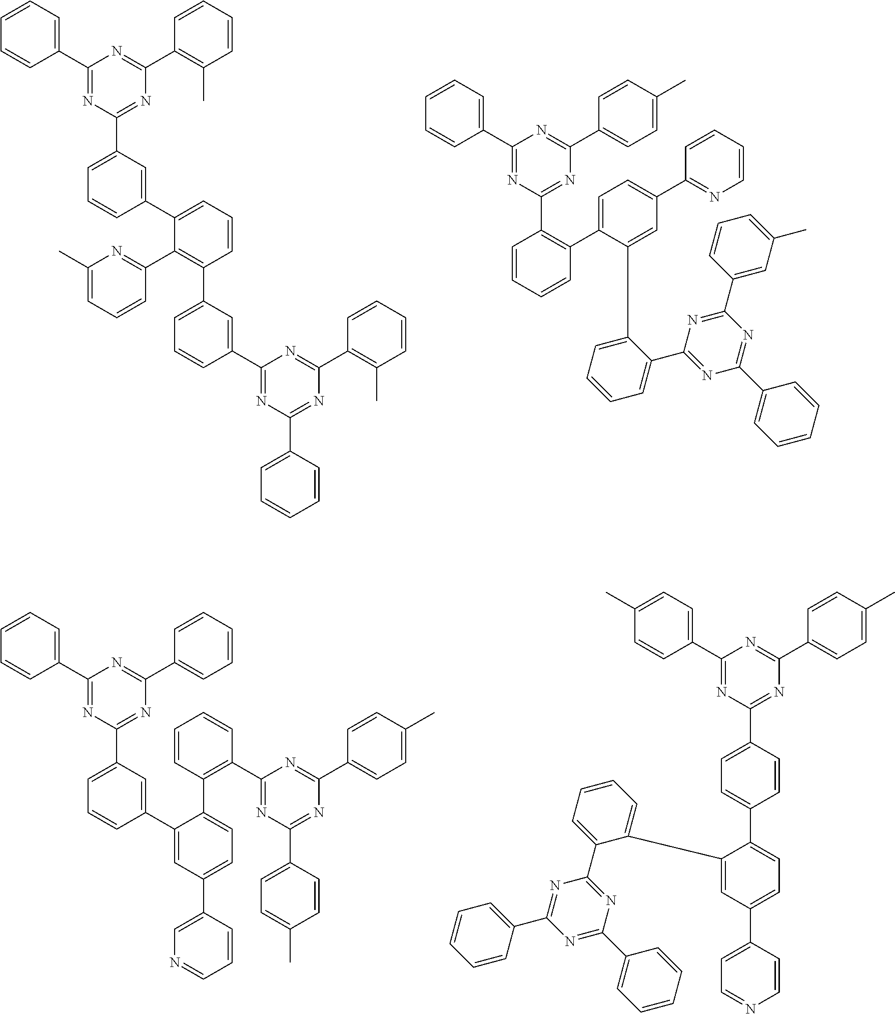 [SMILES: Cc1cccc(-c2c(-c3cccc(-c4nc(-c5ccccc5)nc(-c5ccccc5C)n4)c3)cccc2-c2cccc(-c3nc(-c4ccccc4)nc(-c4ccccc4C)n3)c2)n1 ; Cc1ccc(-c2nc(-c3ccccc3)nc(-c3ccccc3-c3ccc(-c4ccccn4)cc3-c3ccccc3-c3nc(-c4ccccc4)nc(-c4cccc(C)c4)n3)n2)cc1 ; Cc1ccc(-c2nc(-c3ccc(C)cc3)nc(-c3ccccc3-c3ccc(-c4cccnc4)cc3-c3cccc(-c4nc(-c5ccccc5)nc(-c5ccccc5)n4)c3)n2)cc1 ; Cc1ccc(-c2nc(-c3ccc(C)cc3)nc(-c3ccc(-c4ccc(-c5ccncc5)cc4-c4ccccc4-c4nc(-c5ccccc5)nc(-c5ccccc5)n4)cc3)n2)cc1]

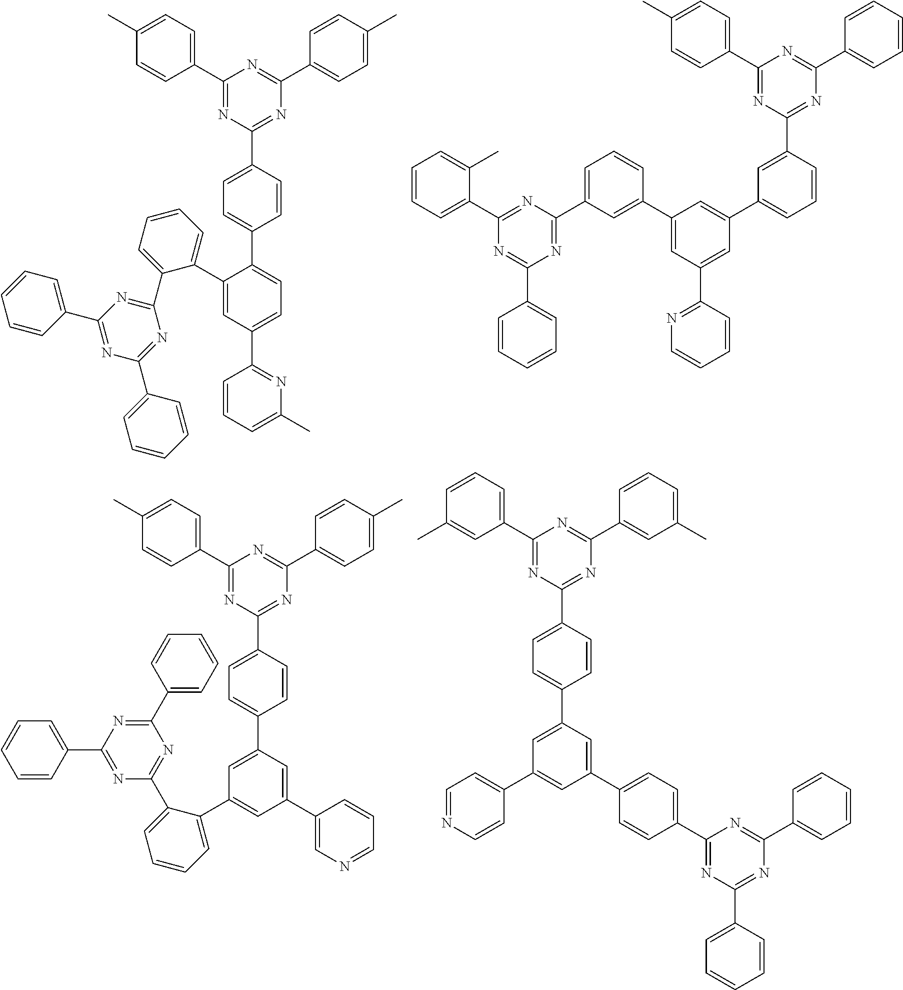

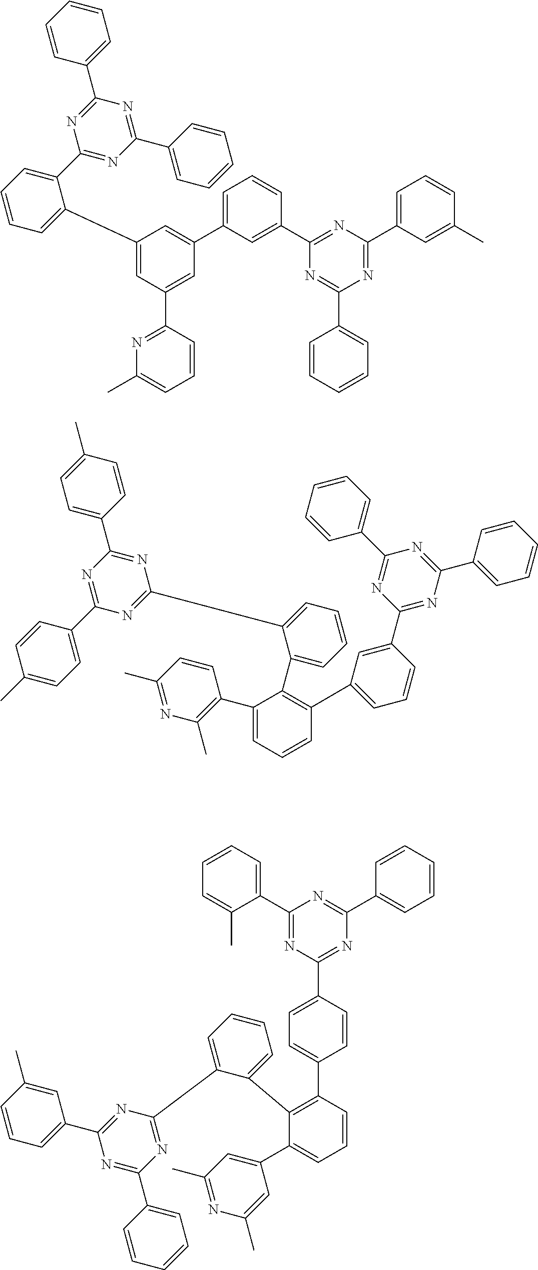

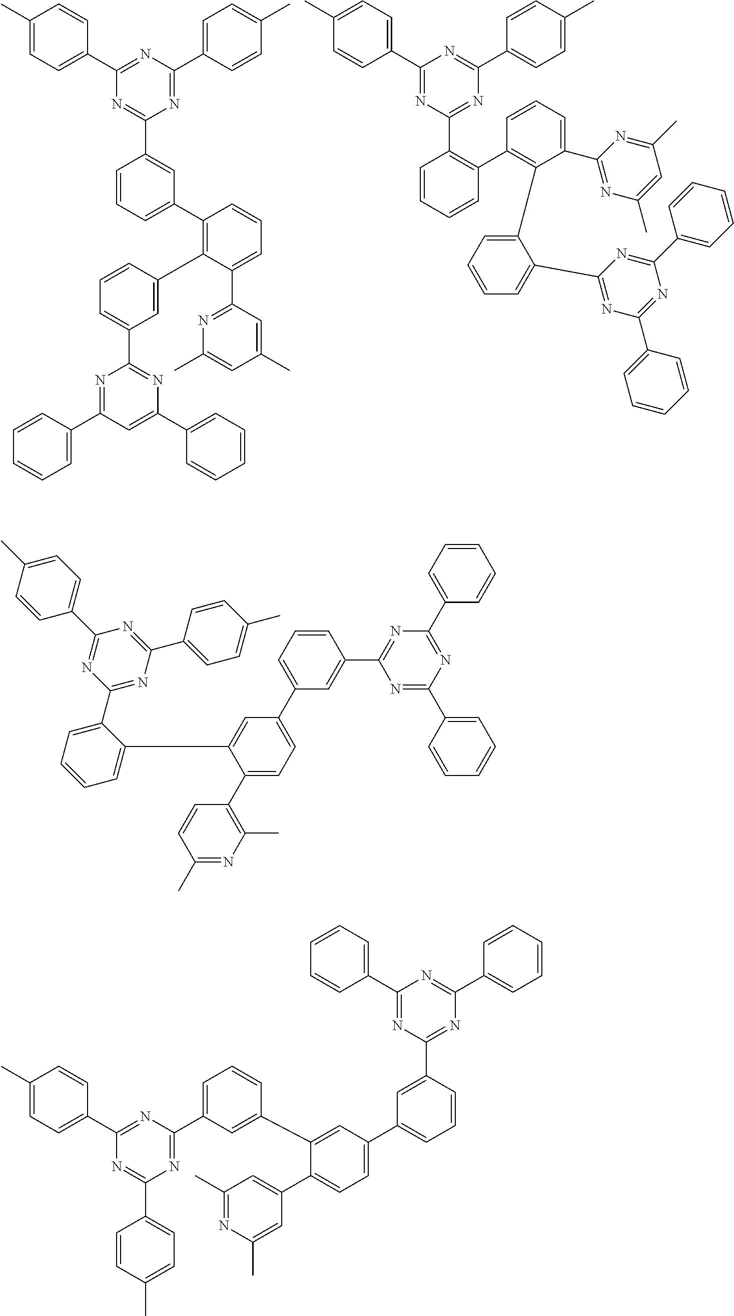

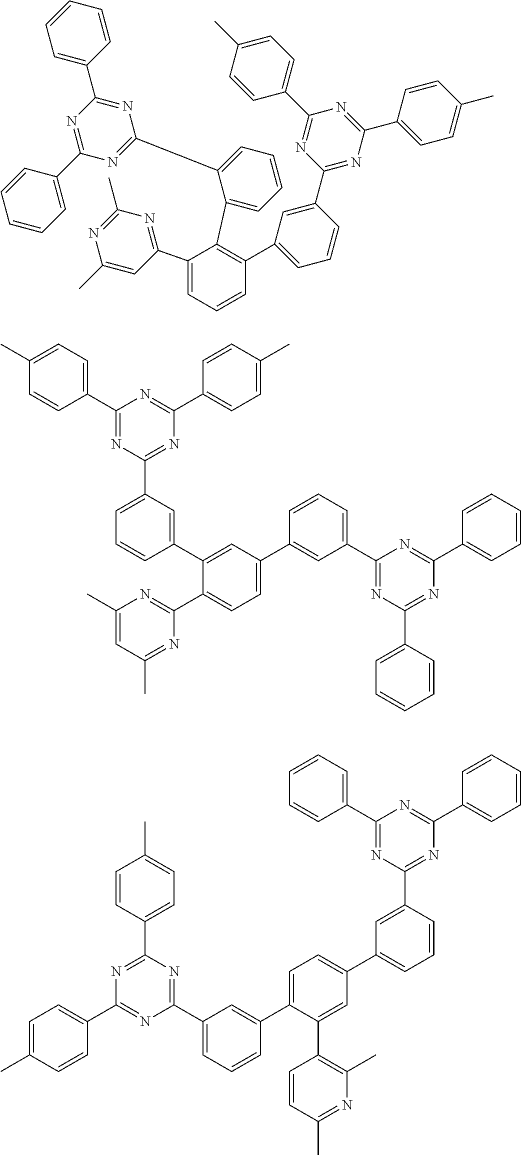

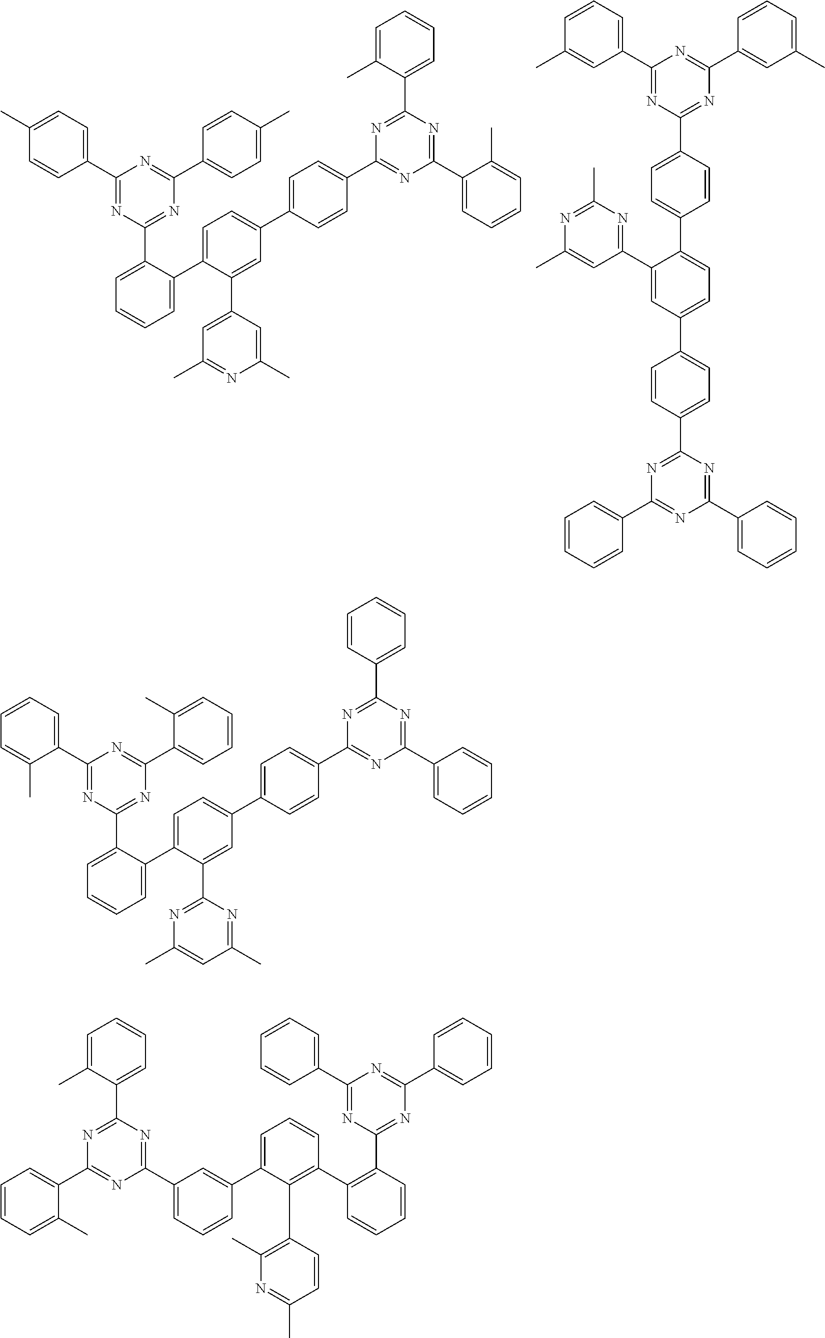

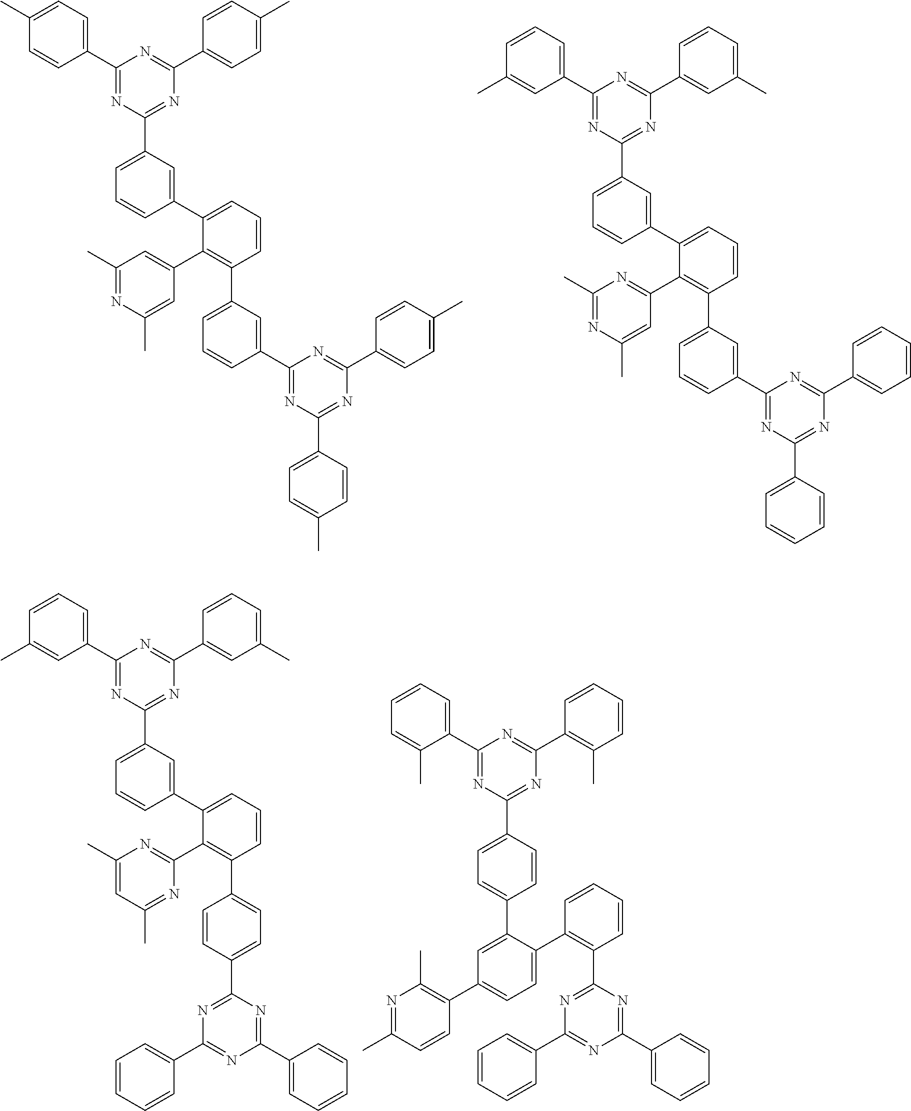

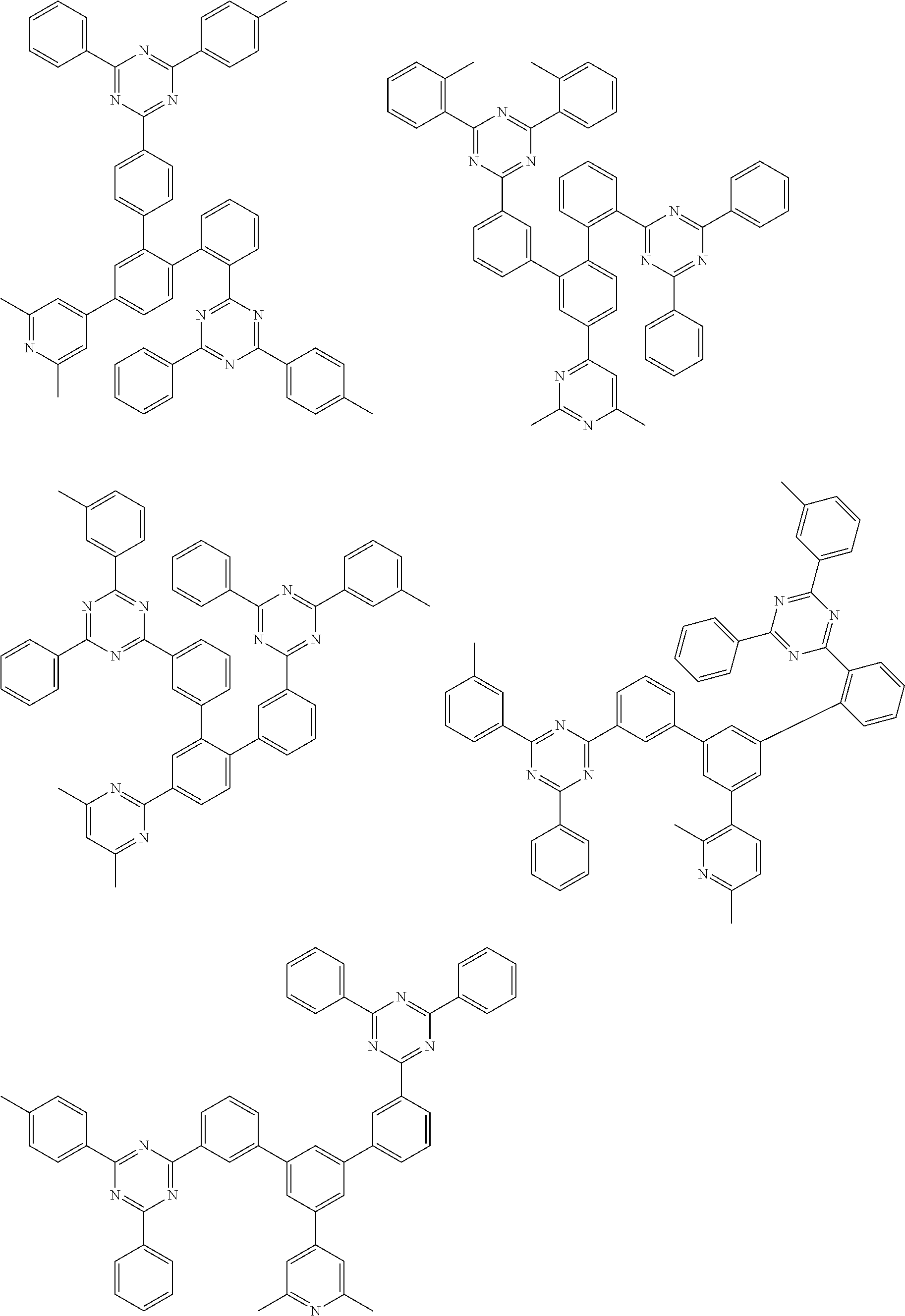

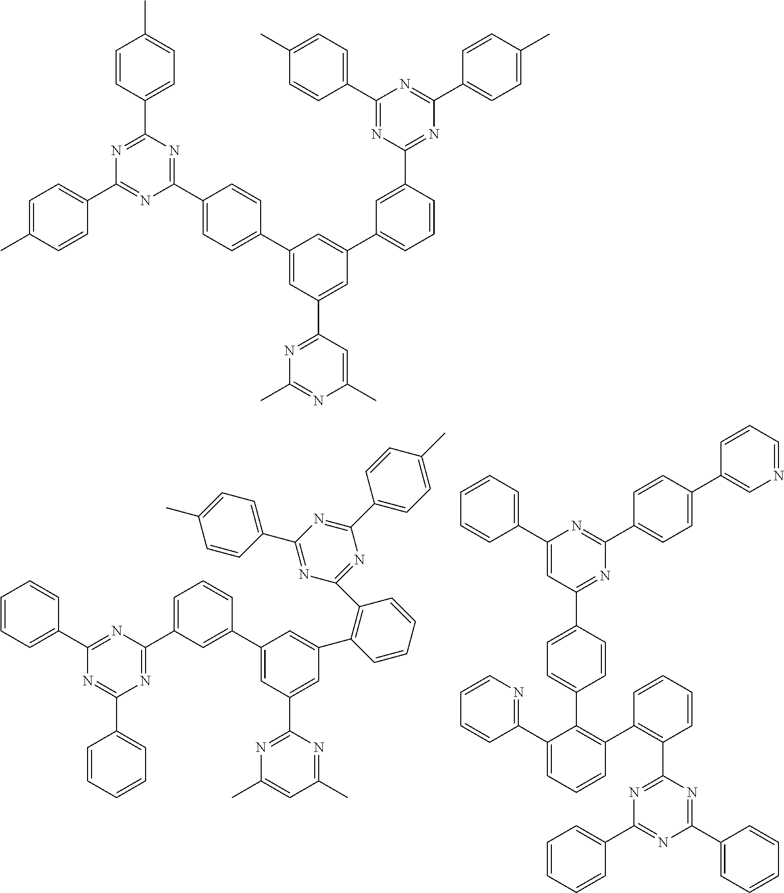

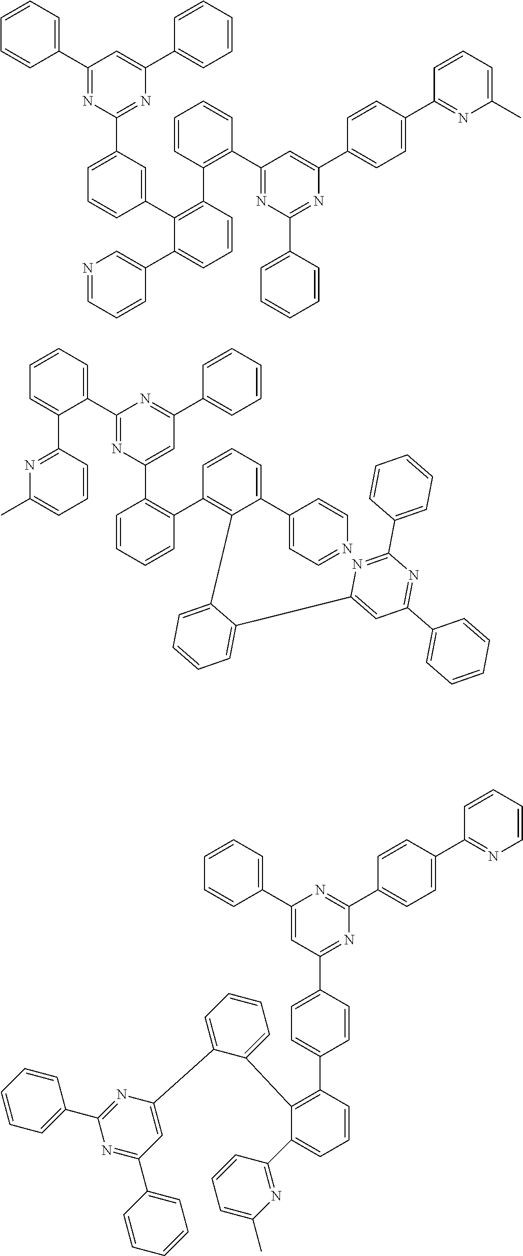

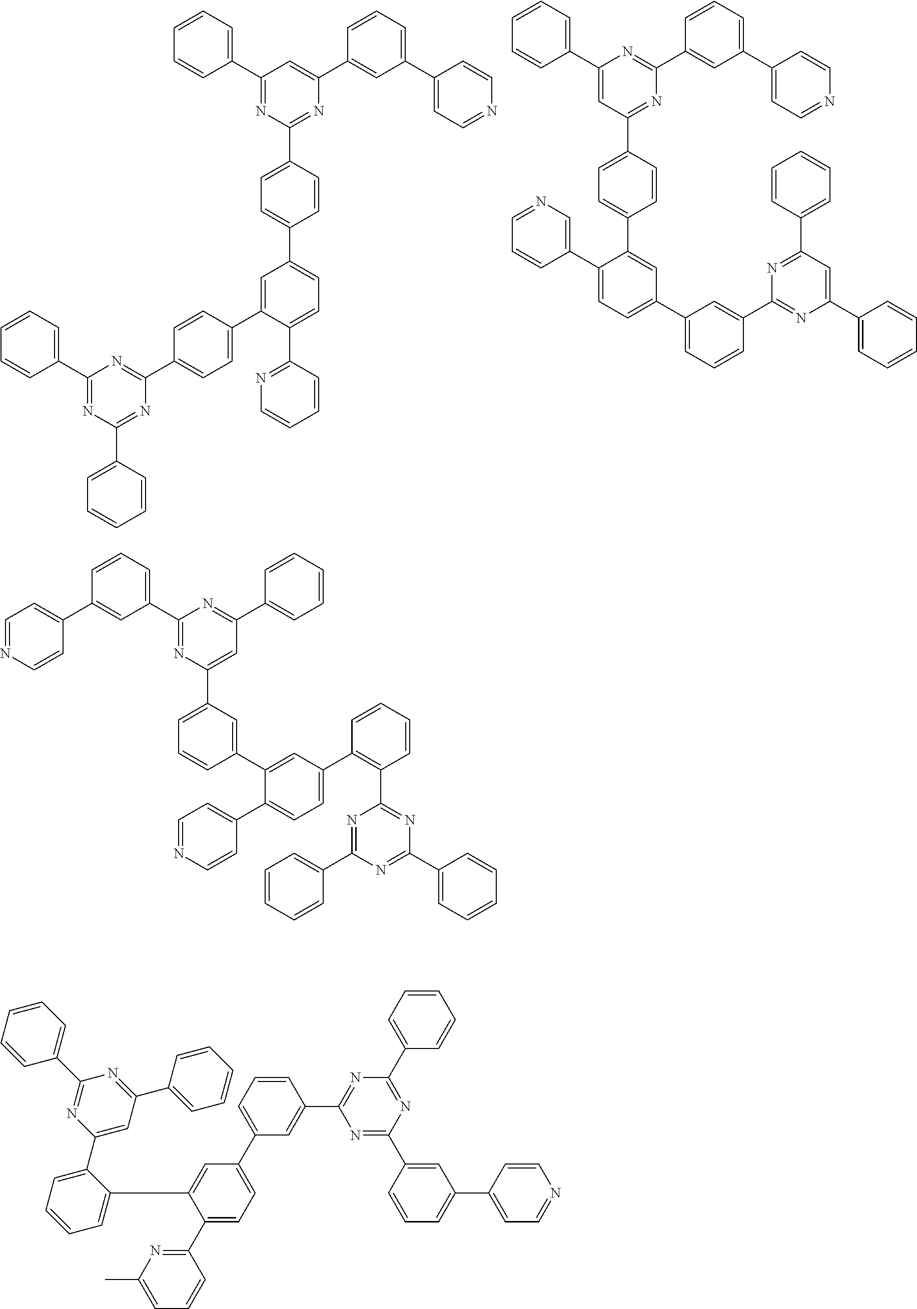

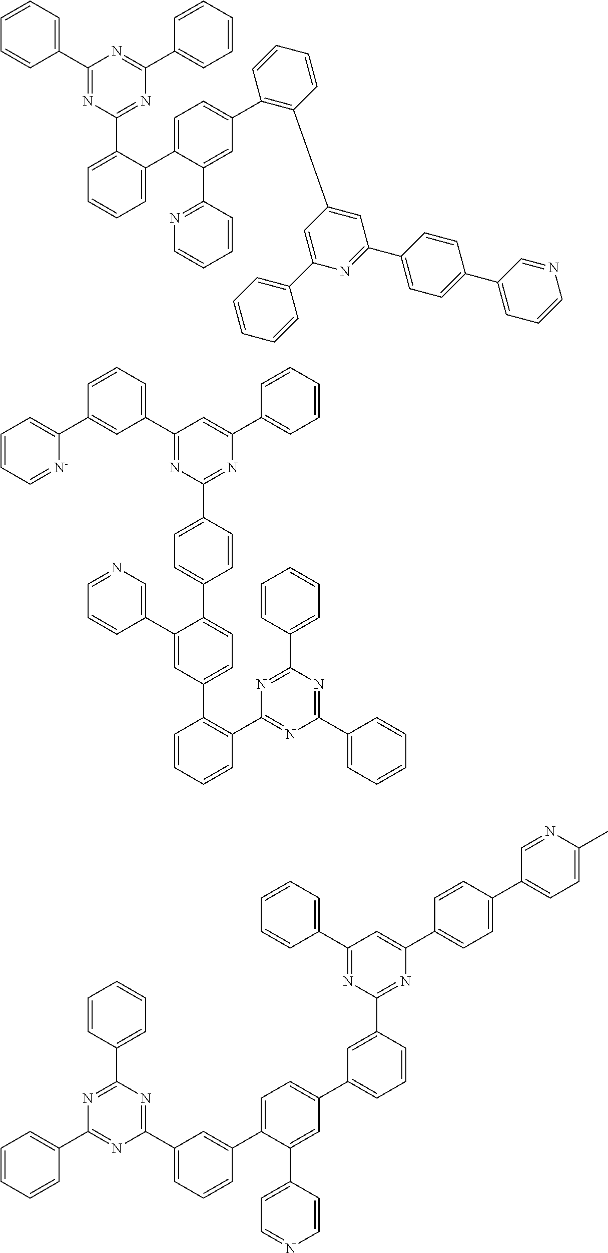

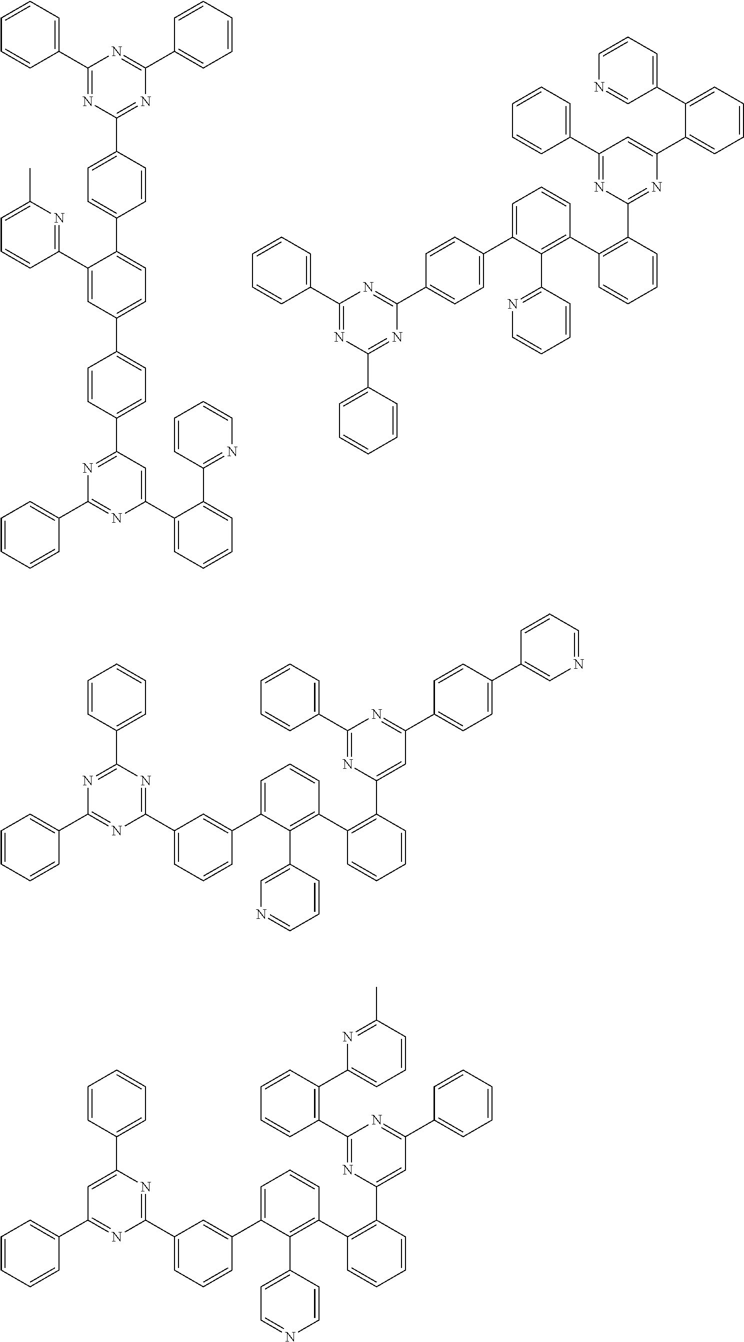 [SMILES: Cc1cccc(-c2cc(-c3ccc(-c4cc(-c5ccccc5-c5ccccn5)nc(-c5ccccc5)n4)cc3)ccc2-c2ccc(-c3nc(-c4ccccc4)nc(-c4ccccc4)n3)cc2)n1 ; c1ccc(-c2cc(-c3ccccc3-c3cccnc3)nc(-c3ccccc3-c3cccc(-c4ccc(-c5nc(-c6ccccc6)nc(-c6ccccc6)n5)cc4)c3-c3ccccn3)n2)cc1 ; c1ccc(-c2nc(-c3ccc(-c4cccnc4)cc3)cc(-c3ccccc3-c3cccc(-c4cccc(-c5nc(-c6ccccc6)nc(-c6ccccc6)n5)c4)c3-c3cccnc3)n2)cc1 ; Cc1cccc(-c2ccccc2-c2nc(-c3ccccc3)cc(-c3ccccc3-c3cccc(-c4cccc(-c5nc(-c6ccccc6)cc(-c6ccccc6)n5)c4)c3-c3ccncc3)n2)n1]

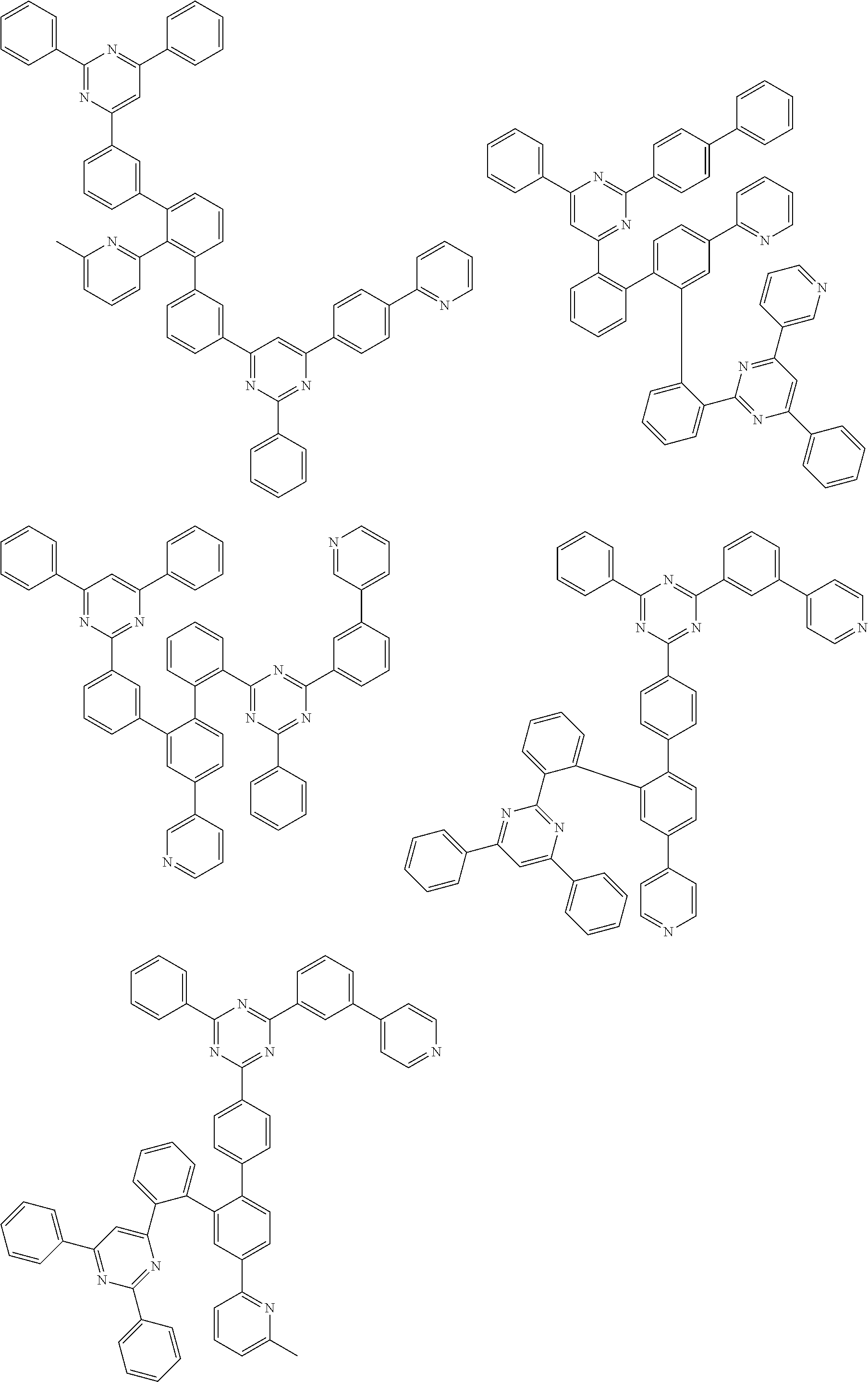

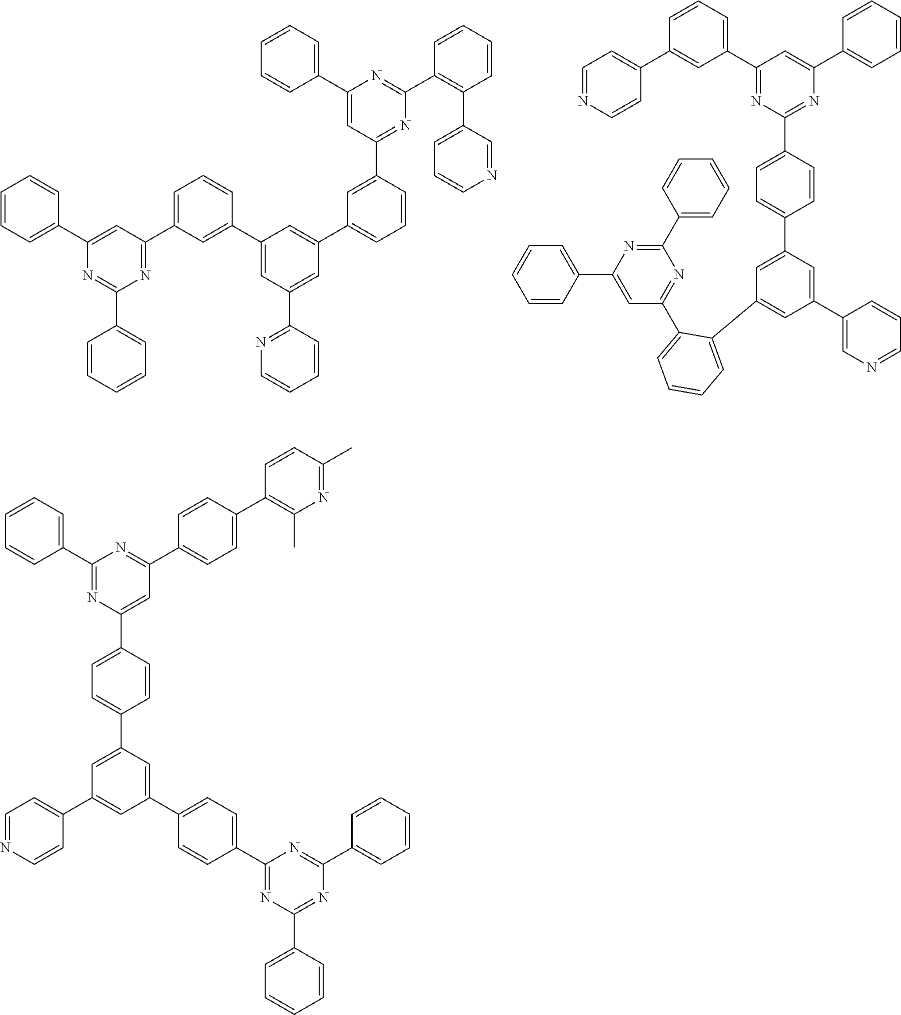 [SMILES: c1ccc(-c2cc(-c3cccc(-c4cc(-c5cccc(-c6cc(-c7ccccc7)nc(-c7ccccc7-c7cccnc7)n6)c5)cc(-c5ccccn5)c4)c3)nc(-c3ccccc3)n2)cc1 ; c1ccc(-c2cc(-c3cccc(-c4ccncc4)c3)nc(-c3ccc(-c4cc(-c5cccnc5)cc(-c5ccccc5-c5cc(-c6ccccc6)nc(-c6ccccc6)n5)c4)cc3)n2)cc1 ; Cc1ccc(-c2ccc(-c3cc(-c4ccc(-c5cc(-c6ccncc6)cc(-c6ccc(-c7nc(-c8ccccc8)nc(-c8ccccc8)n7)cc6)c5)cc4)nc(-c4ccccc4)n3)cc2)c(C)n1]

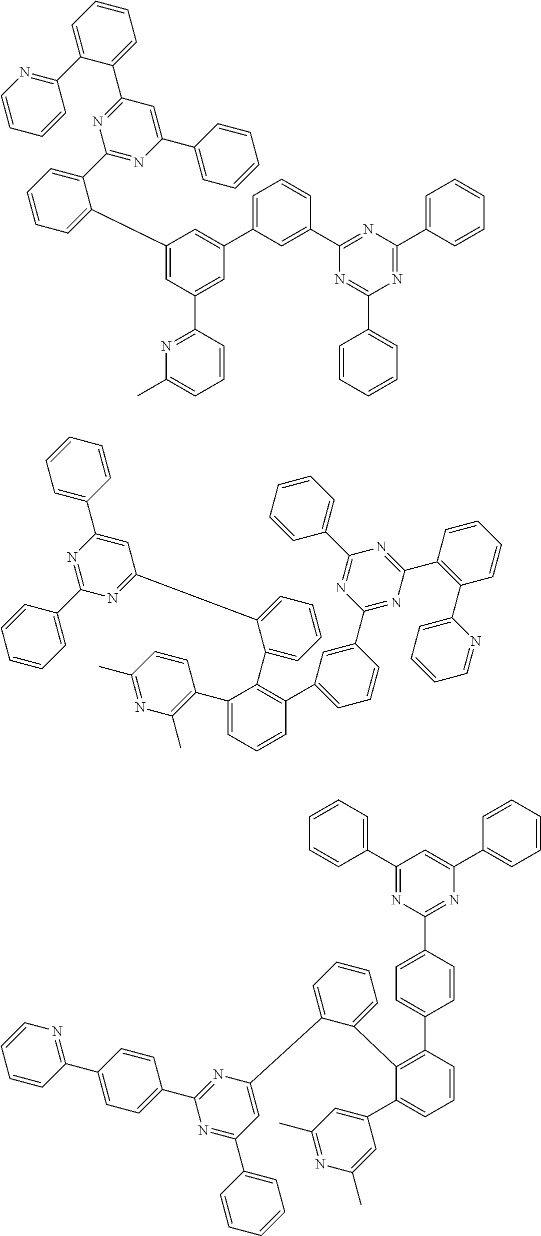 [SMILES: Cc1cccc(-c2cc(-c3cccc(-c4nc(-c5ccccc5)nc(-c5ccccc5)n4)c3)cc(-c3ccccc3-c3nc(-c4ccccc4)cc(-c4ccccc4-c4ccccn4)n3)c2)n1 ; Cc1ccc(-c2cccc(-c3cccc(-c4nc(-c5ccccc5)nc(-c5ccccc5-c5ccccn5)n4)c3)c2-c2ccccc2-c2cc(-c3ccccc3)nc(-c3ccccc3)n2)c(C)n1 ; Cc1cc(-c2cccc(-c3ccc(-c4nc(-c5ccccc5)cc(-c5ccccc5)n4)cc3)c2-c2ccccc2-c2cc(-c3ccccc3)nc(-c3ccc(-c4ccccn4)cc3)n2)cc(C)n1]

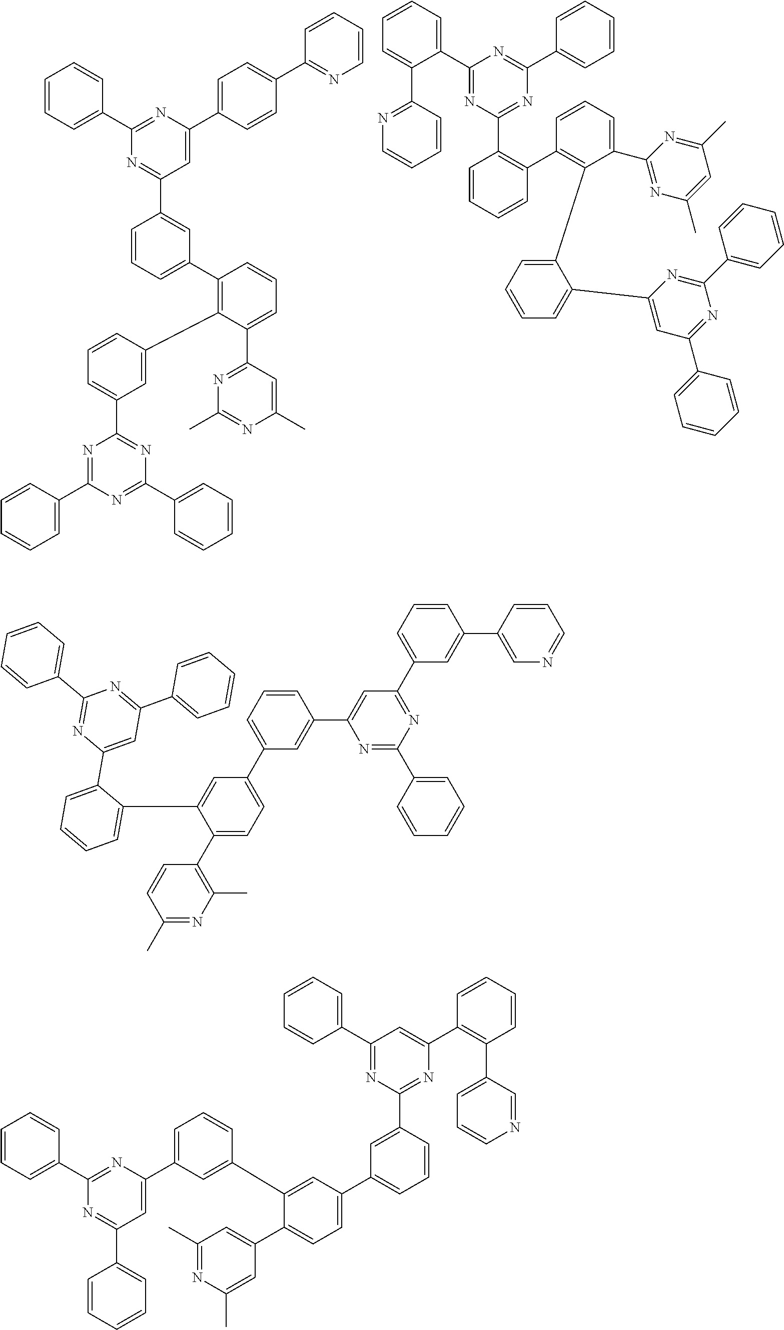

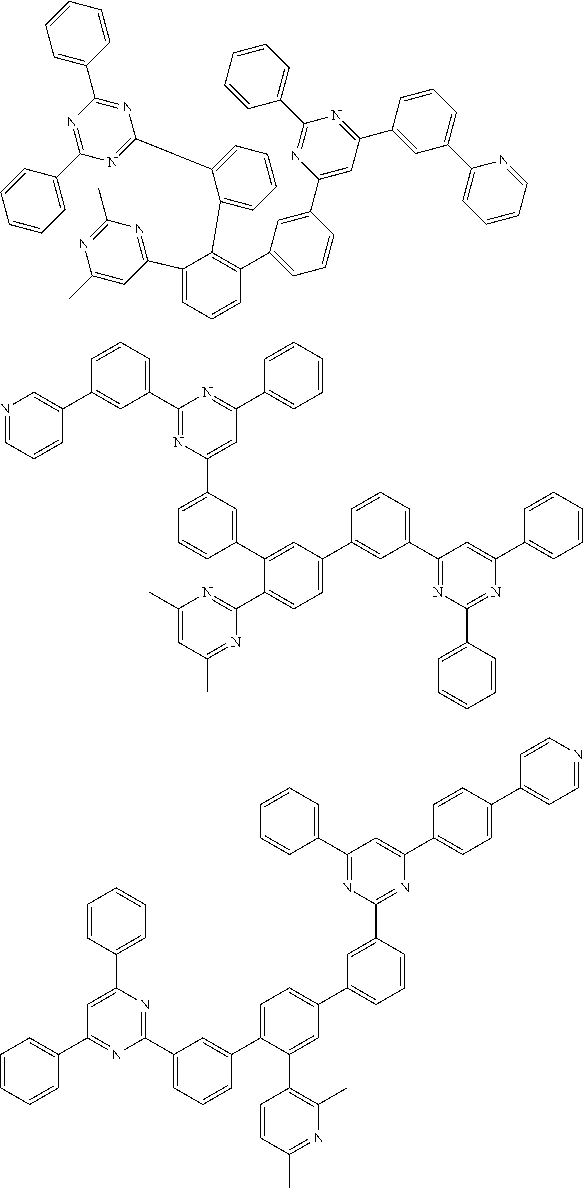 [SMILES: Cc1cc(-c2cccc(-c3cccc(-c4cc(-c5cccc(-c6ccccn6)c5)nc(-c5ccccc5)n4)c3)c2-c2ccccc2-c2nc(-c3ccccc3)nc(-c3ccccc3)n2)nc(C)n1 ; Cc1cc(C)nc(-c2ccc(-c3cccc(-c4cc(-c5ccccc5)nc(-c5ccccc5)n4)c3)cc2-c2cccc(-c3cc(-c4ccccc4)nc(-c4cccc(-c5cccnc5)c4)n3)c2)n1 ; Cc1ccc(-c2cc(-c3cccc(-c4nc(-c5ccccc5)cc(-c5ccc(-c6ccncc6)cc5)n4)c3)ccc2-c2cccc(-c3nc(-c4ccccc4)cc(-c4ccccc4)n3)c2)c(C)n1]

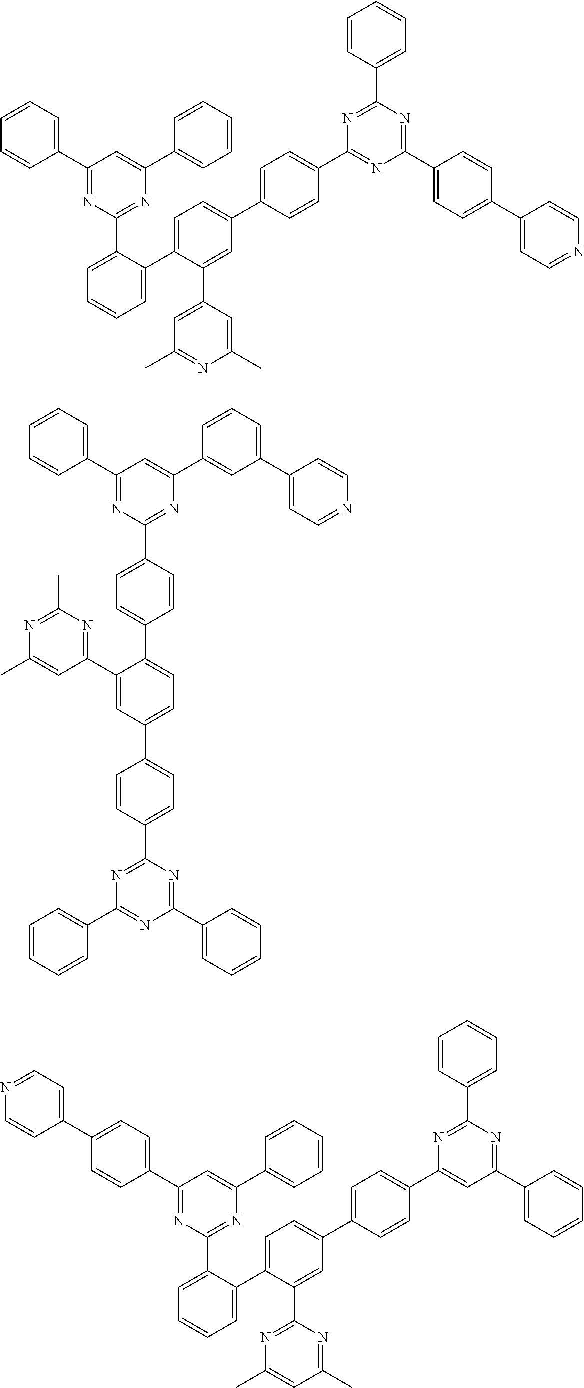 [SMILES: Cc1cc(-c2cc(-c3ccc(-c4nc(-c5ccccc5)nc(-c5ccc(-c6ccncc6)cc5)n4)cc3)ccc2-c2ccccc2-c2nc(-c3ccccc3)cc(-c3ccccc3)n2)cc(C)n1 ; Cc1cc(-c2cc(-c3ccc(-c4nc(-c5ccccc5)nc(-c5ccccc5)n4)cc3)ccc2-c2ccc(-c3nc(-c4ccccc4)cc(-c4cccc(-c5ccncc5)c4)n3)cc2)nc(C)n1 ; Cc1cc(C)nc(-c2cc(-c3ccc(-c4cc(-c5ccccc5)nc(-c5ccccc5)n4)cc3)ccc2-c2ccccc2-c2nc(-c3ccccc3)cc(-c3ccc(-c4ccncc4)cc3)n2)n1]

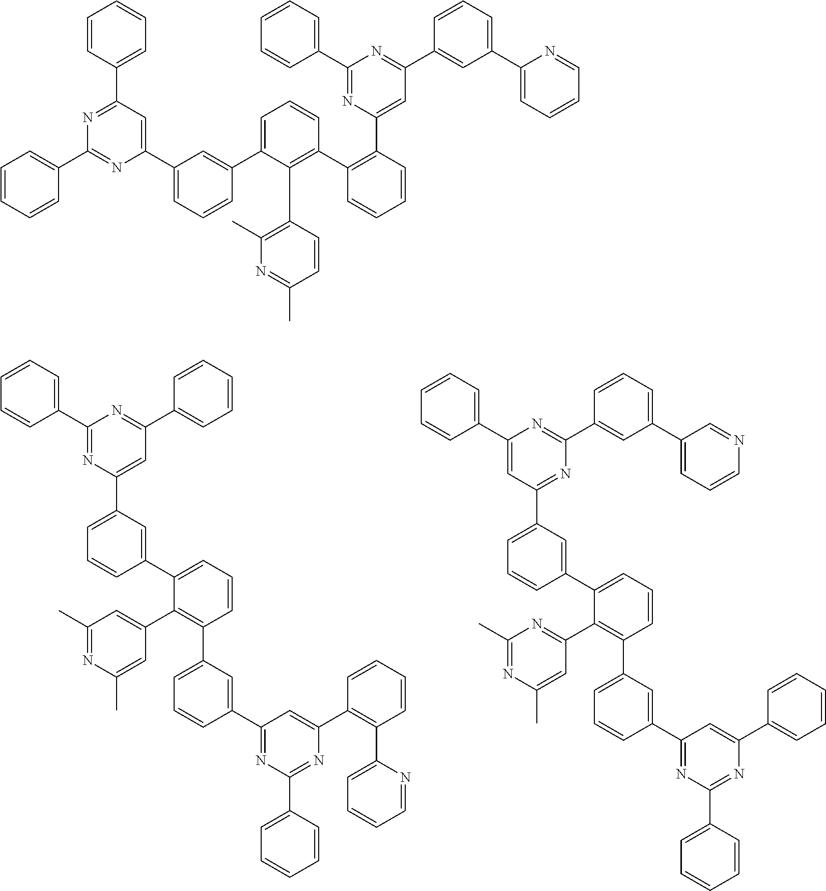 [SMILES: Cc1ccc(-c2c(-c3cccc(-c4cc(-c5ccccc5)nc(-c5ccccc5)n4)c3)cccc2-c2ccccc2-c2cc(-c3cccc(-c4ccccn4)c3)nc(-c3ccccc3)n2)c(C)n1 ; Cc1cc(-c2c(-c3cccc(-c4cc(-c5ccccc5)nc(-c5ccccc5)n4)c3)cccc2-c2cccc(-c3cc(-c4ccccc4-c4ccccn4)nc(-c4ccccc4)n3)c2)cc(C)n1 ; Cc1cc(-c2c(-c3cccc(-c4cc(-c5ccccc5)nc(-c5ccccc5)n4)c3)cccc2-c2cccc(-c3cc(-c4ccccc4)nc(-c4cccc(-c5cccnc5)c4)n3)c2)nc(C)n1]

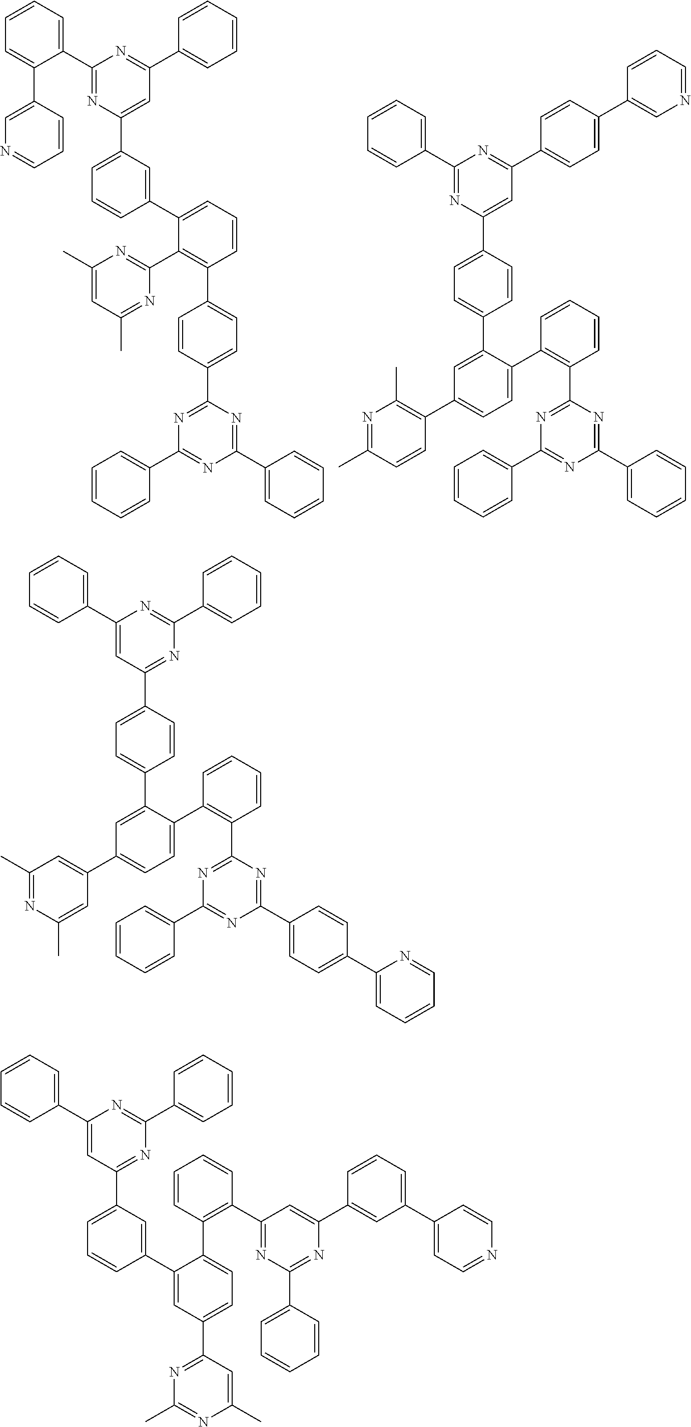

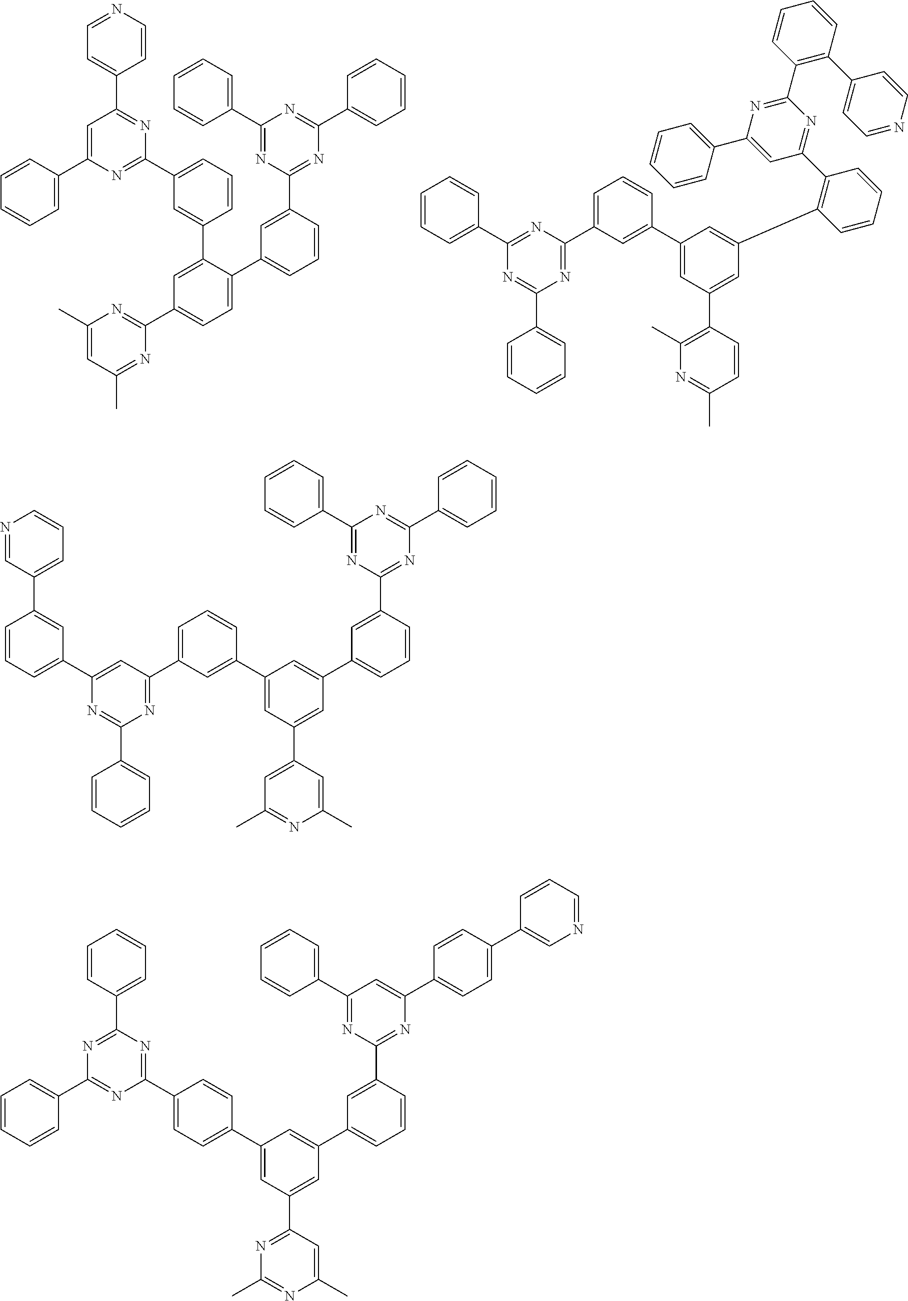 [SMILES: Cc1cc(C)nc(-c2ccc(-c3cccc(-c4nc(-c5ccccc5)nc(-c5ccccc5)n4)c3)c(-c3cccc(-c4nc(-c5ccccc5)cc(-c5ccncc5)n4)c3)c2)n1 ; Cc1ccc(-c2cc(-c3cccc(-c4nc(-c5ccccc5)nc(-c5ccccc5)n4)c3)cc(-c3ccccc3-c3cc(-c4ccccc4)nc(-c4ccccc4-c4ccncc4)n3)c2)c(C)n1 ; Cc1cc(-c2cc(-c3cccc(-c4cc(-c5cccc(-c6cccnc6)c5)nc(-c5ccccc5)n4)c3)cc(-c3cccc(-c4nc(-c5ccccc5)nc(-c5ccccc5)n4)c3)c2)cc(C)n1 ; Cc1cc(-c2cc(-c3ccc(-c4nc(-c5ccccc5)nc(-c5ccccc5)n4)cc3)cc(-c3cccc(-c4nc(-c5ccccc5)cc(-c5ccc(-c6cccnc6)cc5)n4)c3)c2)nc(C)n1]

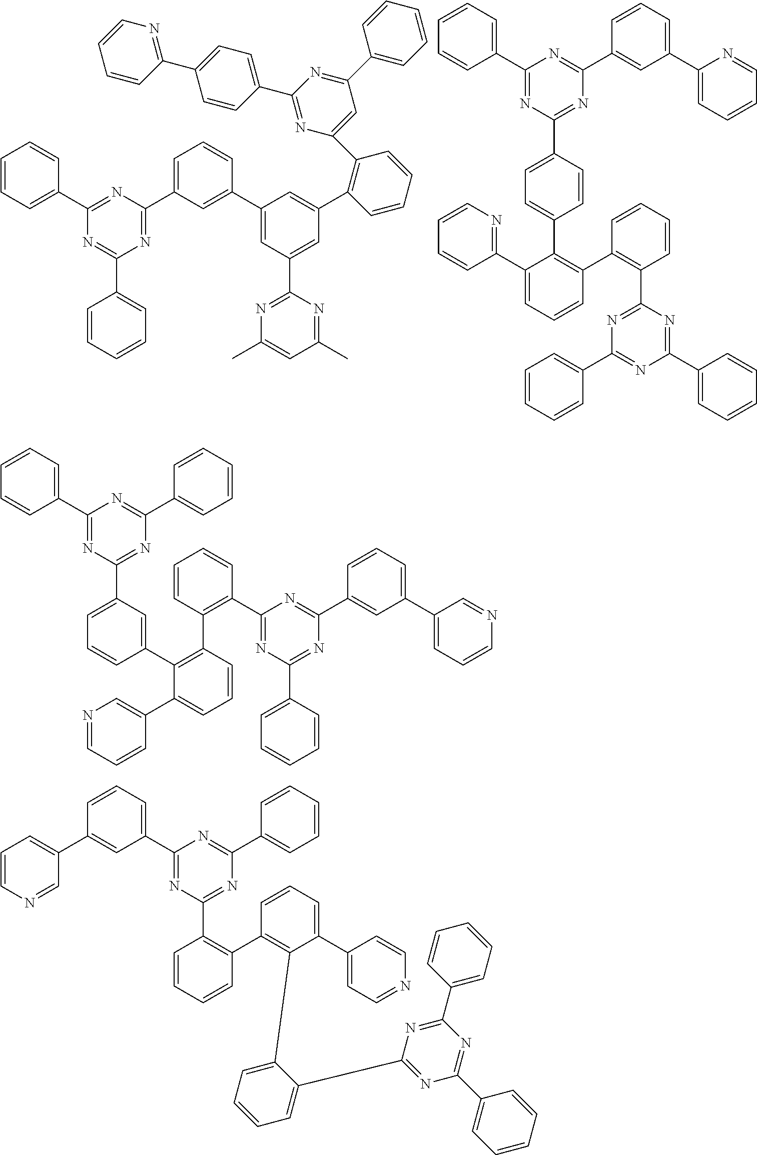

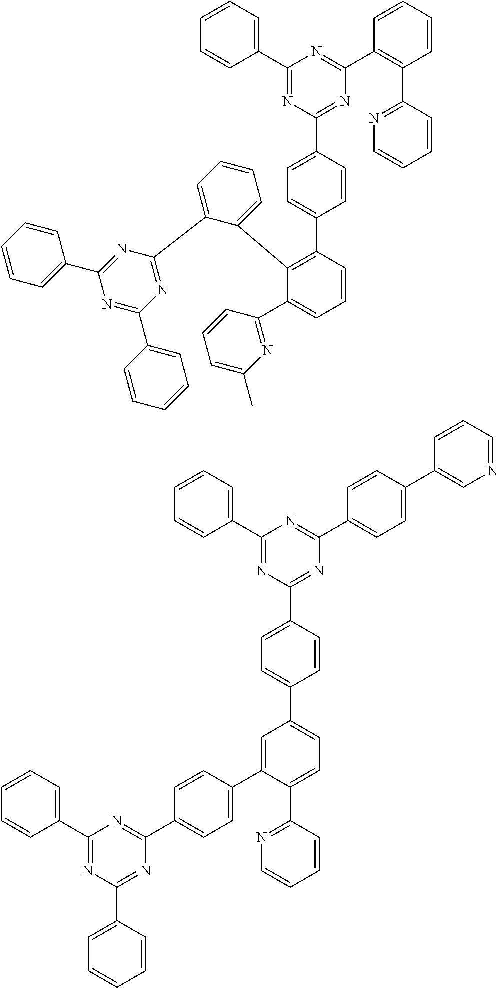 [SMILES: Cc1cccc(-c2cccc(-c3ccc(-c4nc(-c5ccccc5)nc(-c5ccccc5-c5ccccn5)n4)cc3)c2-c2ccccc2-c2nc(-c3ccccc3)nc(-c3ccccc3)n2)n1 ; c1ccc(-c2nc(-c3ccc(-c4cccnc4)cc3)nc(-c3ccc(-c4ccc(-c5ccccn5)c(-c5ccc(-c6nc(-c7ccccc7)nc(-c7ccccc7)n6)cc5)c4)cc3)n2)cc1]

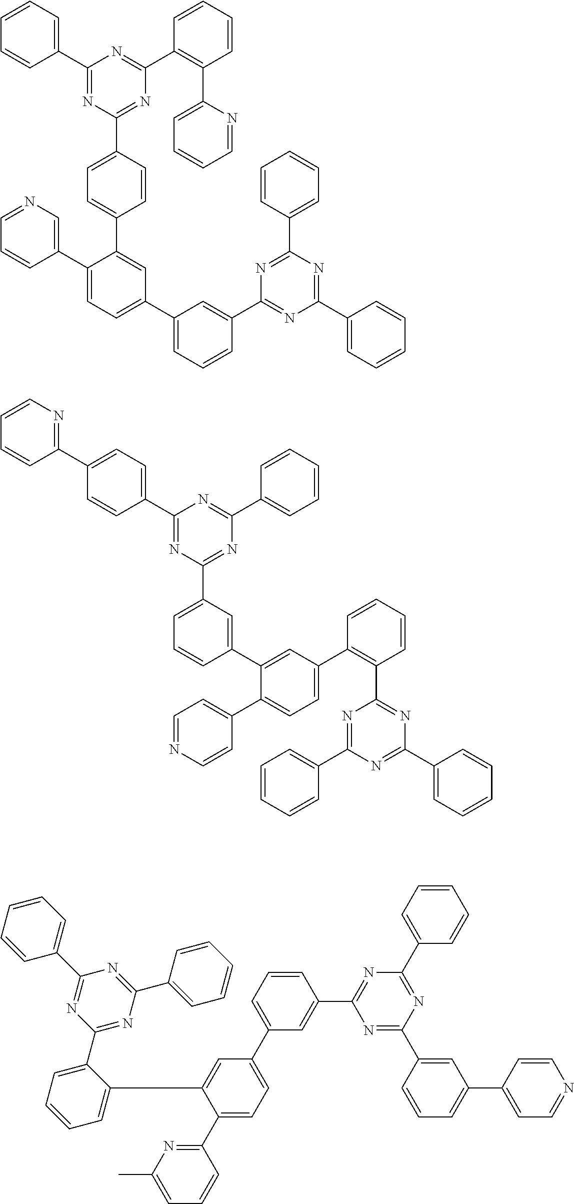 [SMILES: c1ccc(-c2nc(-c3ccccc3)nc(-c3cccc(-c4ccc(-c5cccnc5)c(-c5ccc(-c6nc(-c7ccccc7)nc(-c7ccccc7-c7ccccn7)n6)cc5)c4)c3)n2)cc1 ; c1ccc(-c2nc(-c3ccc(-c4ccccn4)cc3)nc(-c3cccc(-c4cc(-c5ccccc5-c5nc(-c6ccccc6)nc(-c6ccccc6)n5)ccc4-c4ccncc4)c3)n2)cc1 ; Cc1cccc(-c2ccc(-c3cccc(-c4nc(-c5ccccc5)nc(-c5cccc(-c6ccncc6)c5)n4)c3)cc2-c2ccccc2-c2nc(-c3ccccc3)nc(-c3ccccc3)n2)n1]

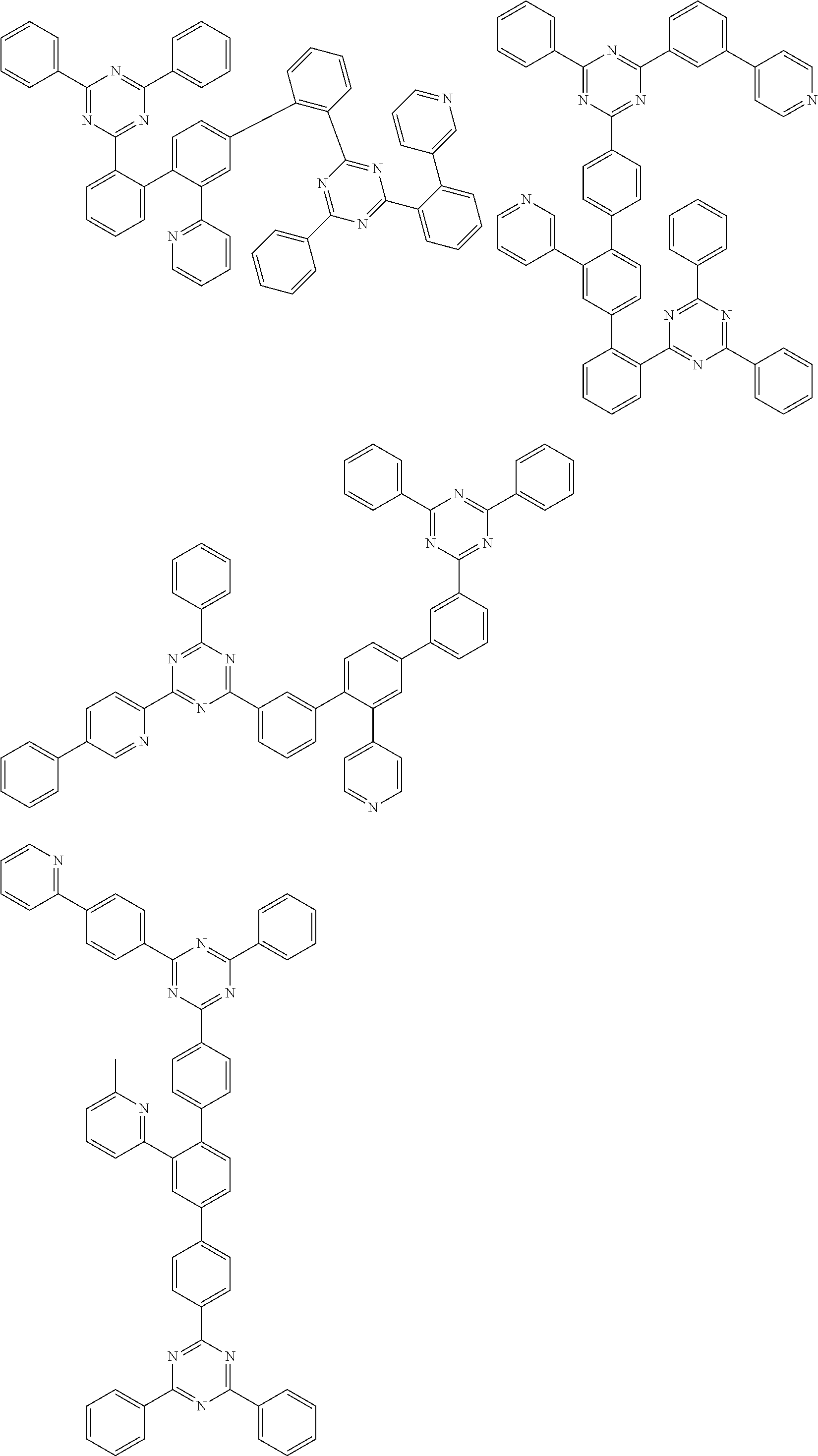

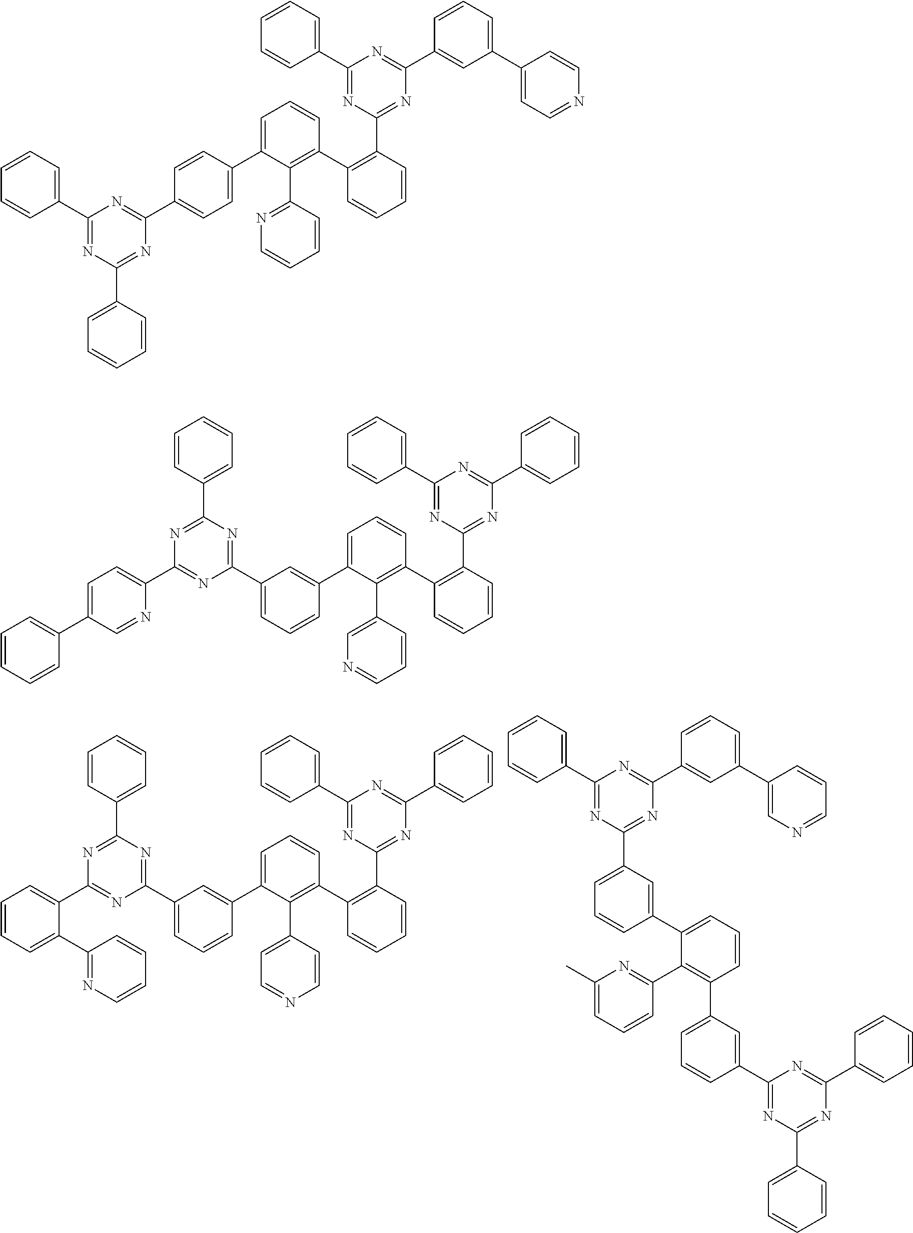

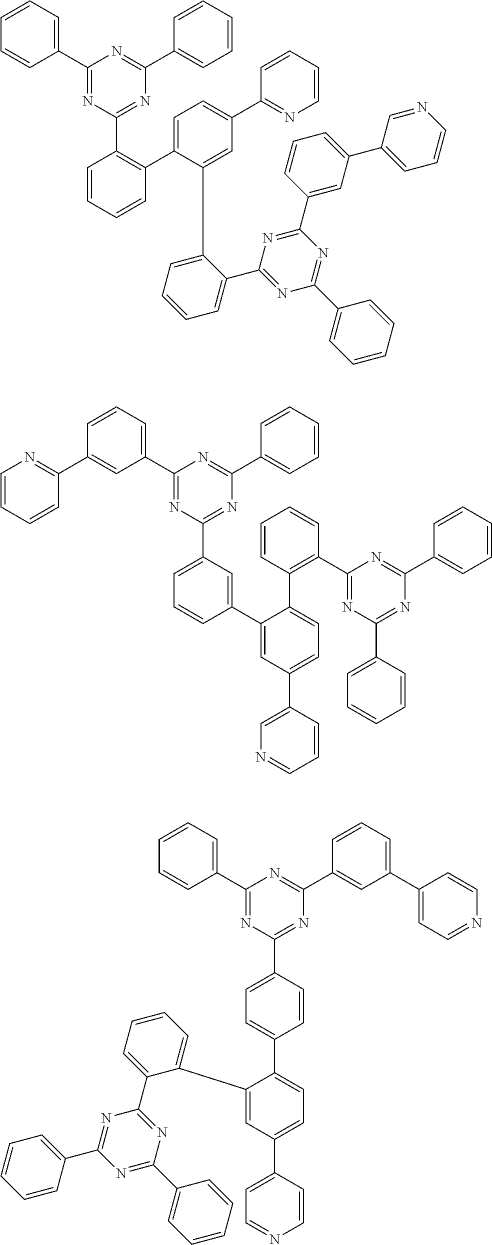 [SMILES: c1ccc(-c2nc(-c3ccccc3)nc(-c3ccccc3-c3ccc(-c4ccccn4)cc3-c3ccccc3-c3nc(-c4ccccc4)nc(-c4cccc(-c5cccnc5)c4)n3)n2)cc1 ; c1ccc(-c2nc(-c3cccc(-c4ccccn4)c3)nc(-c3cccc(-c4cc(-c5cccnc5)ccc4-c4ccccc4-c4nc(-c5ccccc5)nc(-c5ccccc5)n4)c3)n2)cc1 ; c1ccc(-c2nc(-c3ccc(-c4ccc(-c5ccncc5)cc4-c4ccccc4-c4nc(-c5ccccc5)nc(-c5ccccc5)n4)cc3)nc(-c3cccc(-c4ccncc4)c3)n2)cc1]

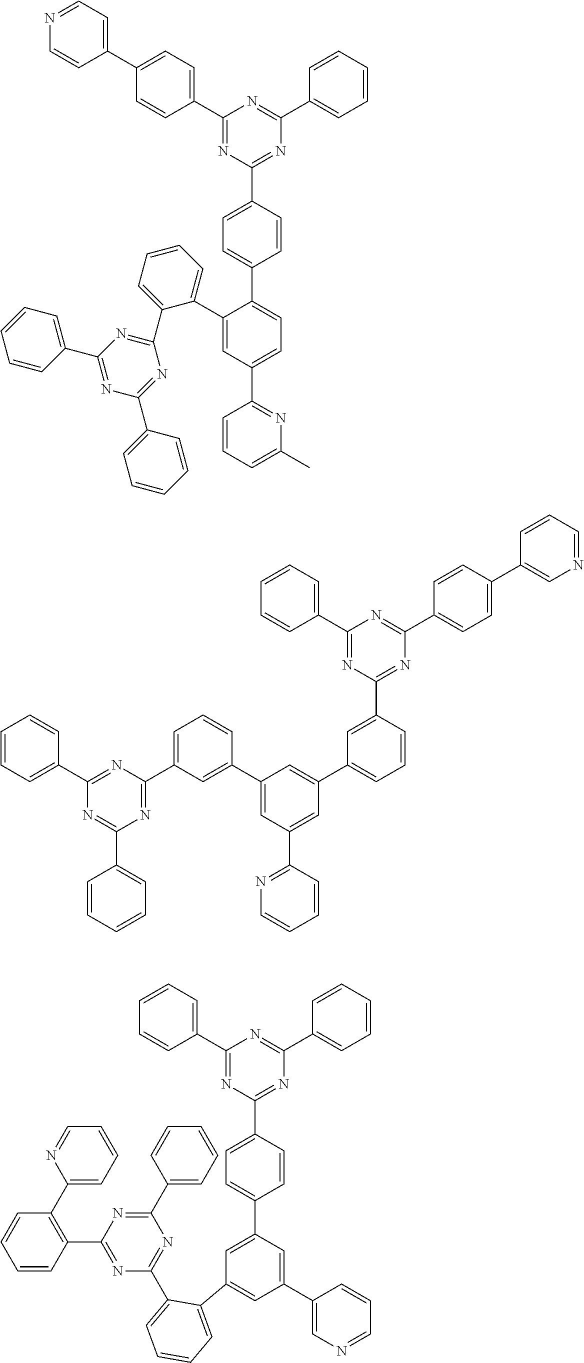 [SMILES: Cc1cccc(-c2ccc(-c3ccc(-c4nc(-c5ccccc5)nc(-c5ccc(-c6ccncc6)cc5)n4)cc3)c(-c3ccccc3-c3nc(-c4ccccc4)nc(-c4ccccc4)n3)c2)n1 ; c1ccc(-c2nc(-c3ccccc3)nc(-c3cccc(-c4cc(-c5cccc(-c6nc(-c7ccccc7)nc(-c7ccc(-c8cccnc8)cc7)n6)c5)cc(-c5ccccn5)c4)c3)n2)cc1 ; c1ccc(-c2nc(-c3ccccc3)nc(-c3ccc(-c4cc(-c5cccnc5)cc(-c5ccccc5-c5nc(-c6ccccc6)nc(-c6ccccc6-c6ccccn6)n5)c4)cc3)n2)cc1]

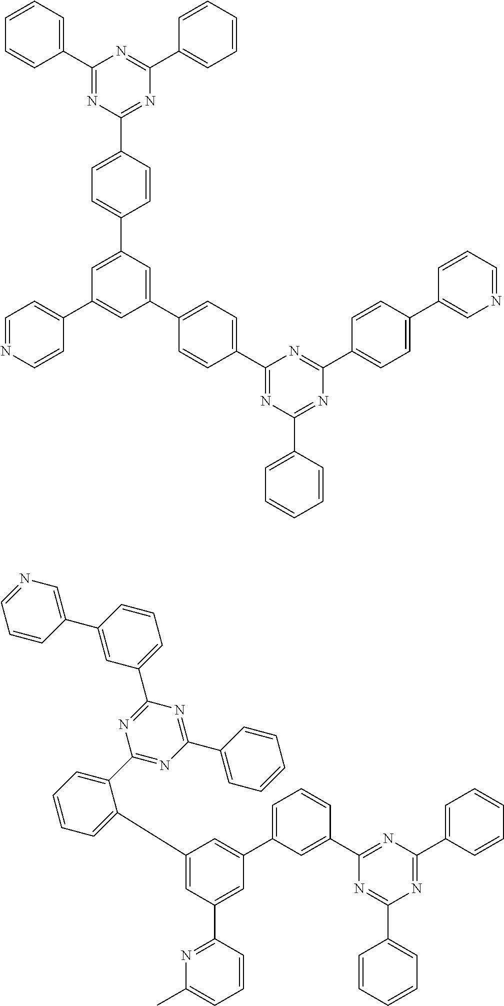 [SMILES: c1ccc(-c2nc(-c3ccccc3)nc(-c3ccc(-c4cc(-c5ccncc5)cc(-c5ccc(-c6nc(-c7ccccc7)nc(-c7ccc(-c8cccnc8)cc7)n6)cc5)c4)cc3)n2)cc1 ; Cc1cccc(-c2cc(-c3cccc(-c4nc(-c5ccccc5)nc(-c5ccccc5)n4)c3)cc(-c3ccccc3-c3nc(-c4ccccc4)nc(-c4cccc(-c5cccnc5)c4)n3)c2)n1]

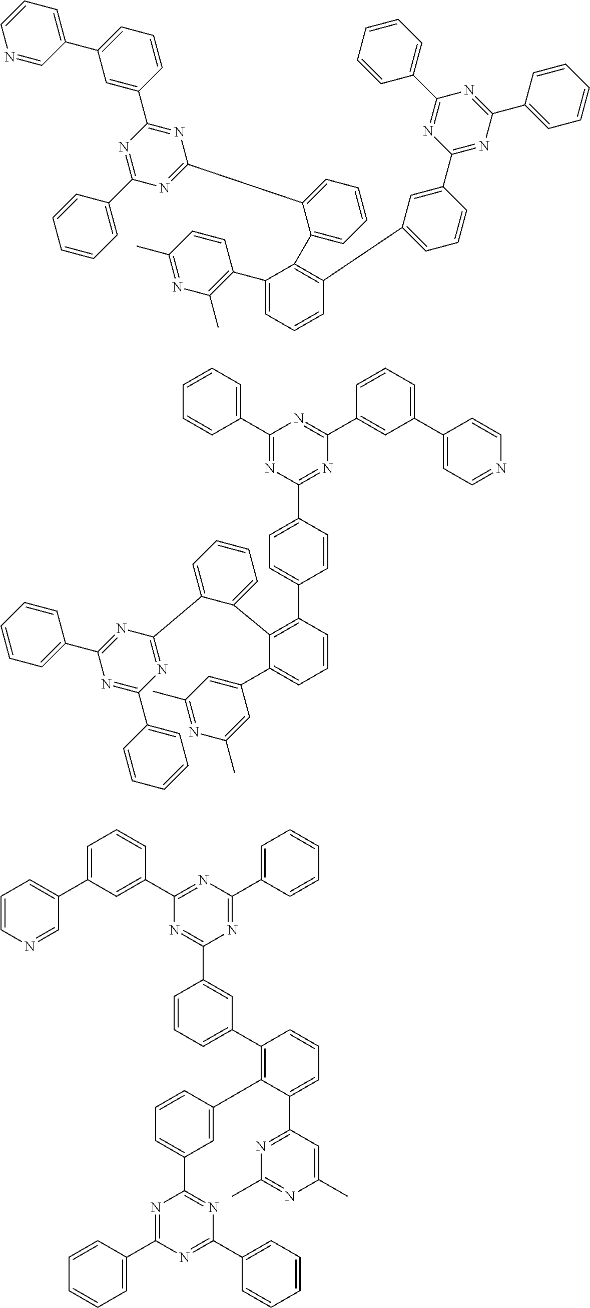

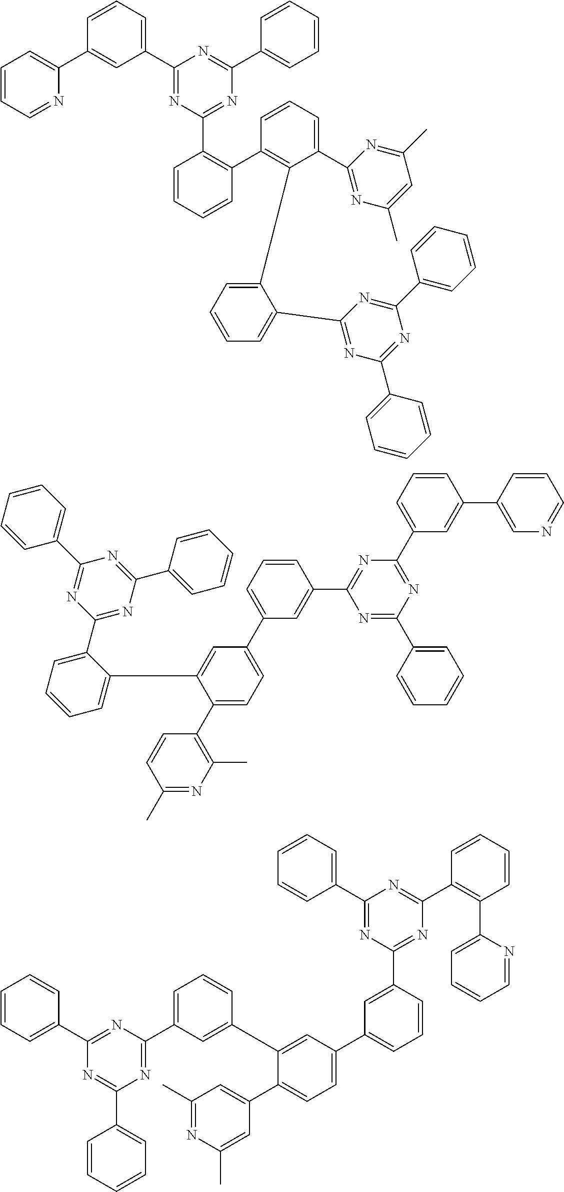

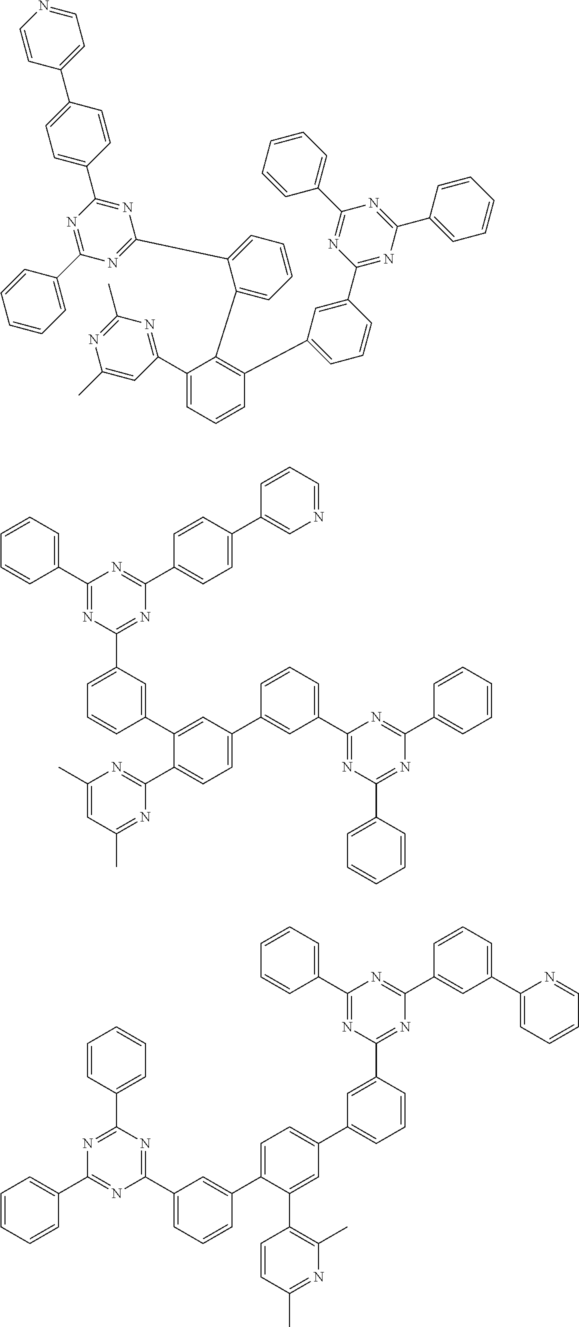 [SMILES: Cc1cc(-c2cccc(-c3cccc(-c4nc(-c5ccccc5)nc(-c5ccccc5)n4)c3)c2-c2ccccc2-c2nc(-c3ccccc3)nc(-c3ccc(-c4ccncc4)cc3)n2)nc(C)n1 ; Cc1cc(C)nc(-c2ccc(-c3cccc(-c4nc(-c5ccccc5)nc(-c5ccccc5)n4)c3)cc2-c2cccc(-c3nc(-c4ccccc4)nc(-c4ccc(-c5cccnc5)cc4)n3)c2)n1 ; Cc1ccc(-c2cc(-c3cccc(-c4nc(-c5ccccc5)nc(-c5cccc(-c6ccccn6)c5)n4)c3)ccc2-c2cccc(-c3nc(-c4ccccc4)nc(-c4ccccc4)n3)c2)c(C)n1]

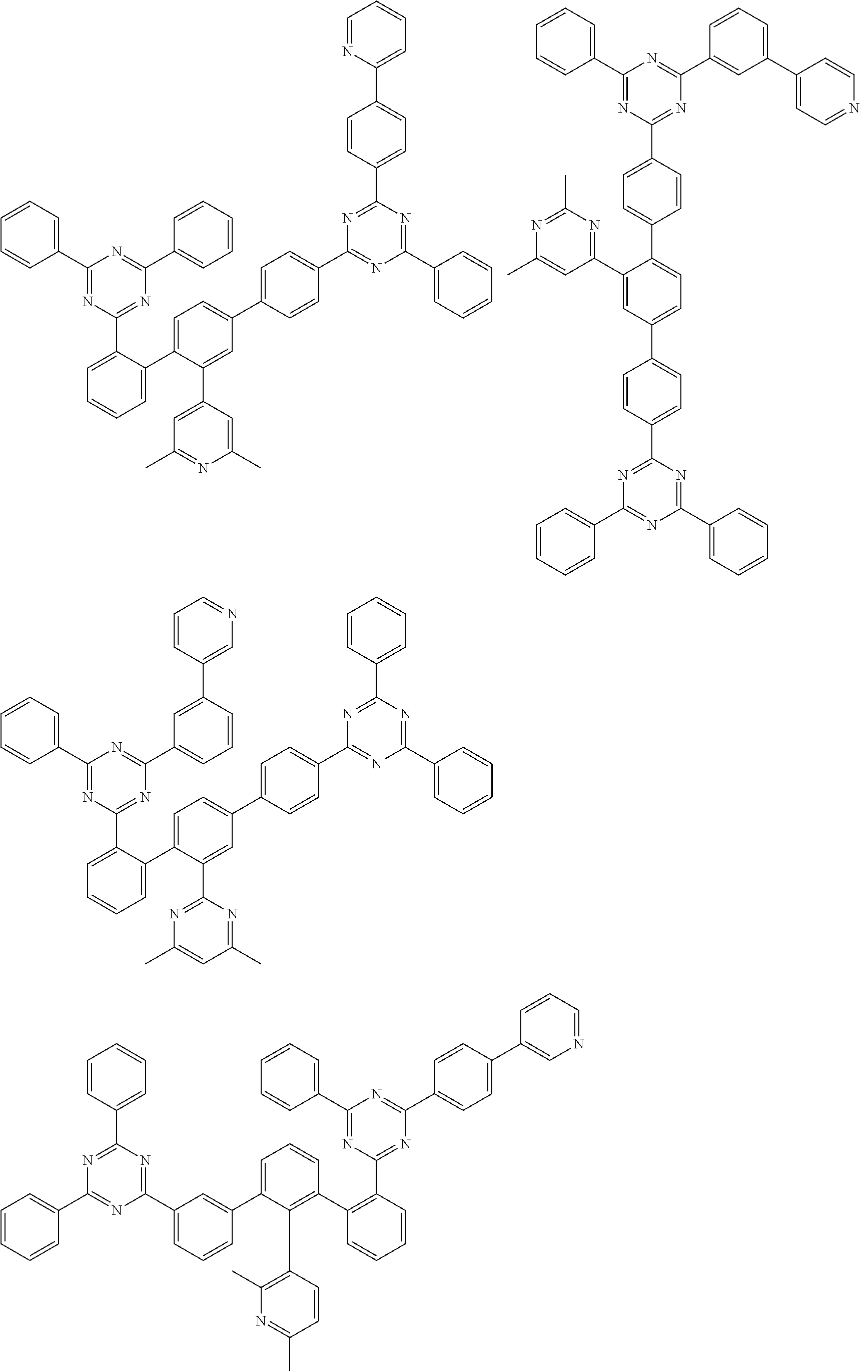

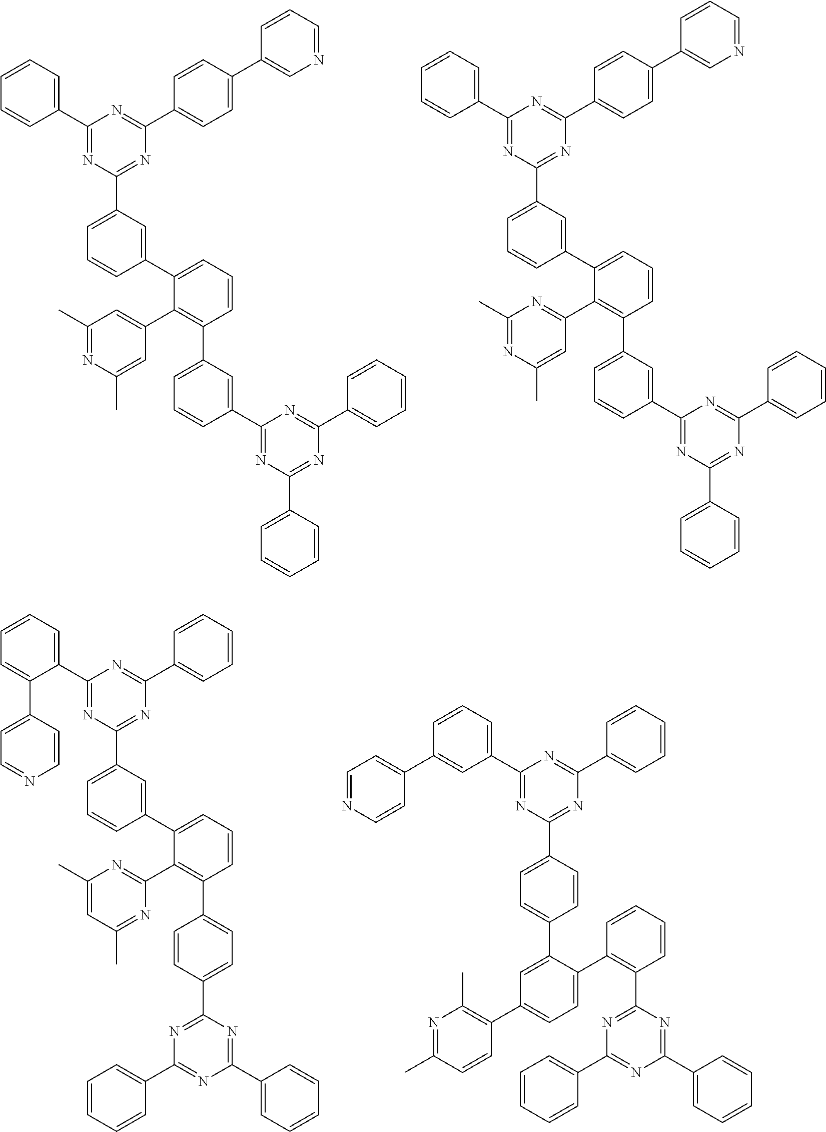 [SMILES: Cc1cc(-c2c(-c3cccc(-c4nc(-c5ccccc5)nc(-c5ccccc5)n4)c3)cccc2-c2cccc(-c3nc(-c4ccccc4)nc(-c4ccc(-c5cccnc5)cc4)n3)c2)cc(C)n1 ; Cc1cc(-c2c(-c3cccc(-c4nc(-c5ccccc5)nc(-c5ccccc5)n4)c3)cccc2-c2cccc(-c3nc(-c4ccccc4)nc(-c4ccc(-c5cccnc5)cc4)n3)c2)nc(C)n1 ; Cc1cc(C)nc(-c2c(-c3ccc(-c4nc(-c5ccccc5)nc(-c5ccccc5)n4)cc3)cccc2-c2cccc(-c3nc(-c4ccccc4)nc(-c4ccccc4-c4ccncc4)n3)c2)n1 ; Cc1ccc(-c2ccc(-c3ccccc3-c3nc(-c4ccccc4)nc(-c4ccccc4)n3)c(-c3ccc(-c4nc(-c5ccccc5)nc(-c5cccc(-c6ccncc6)c5)n4)cc3)c2)c(C)n1]

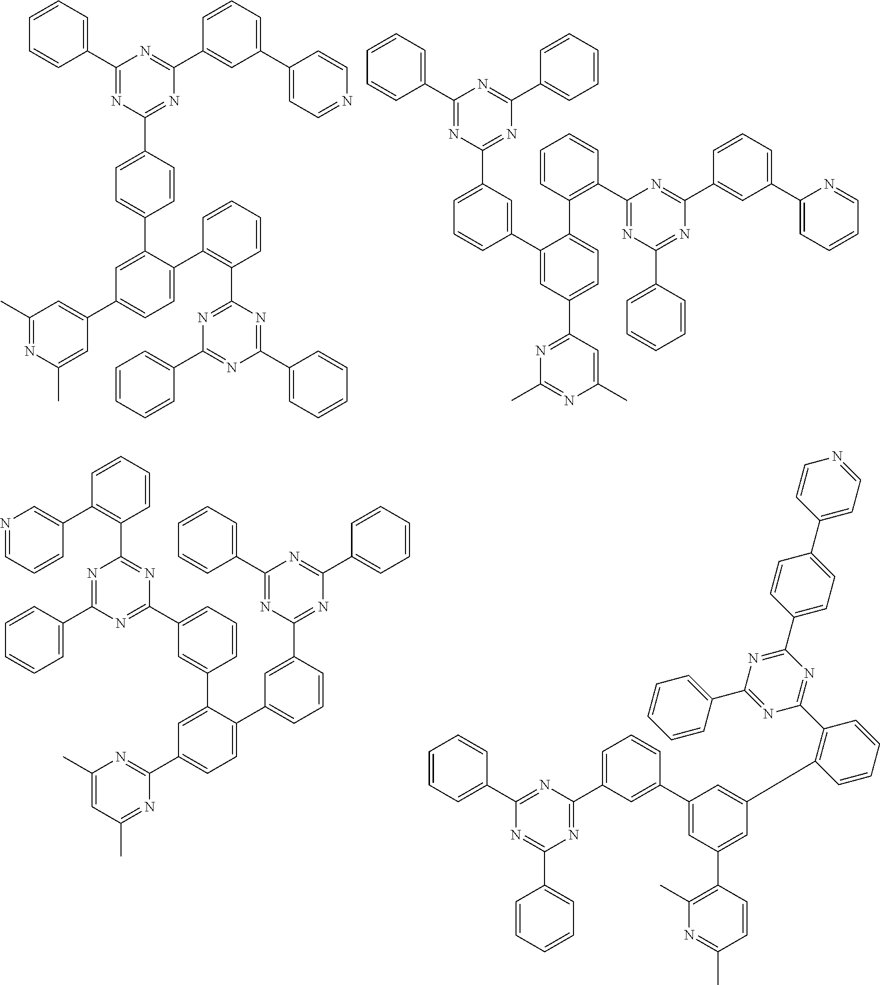

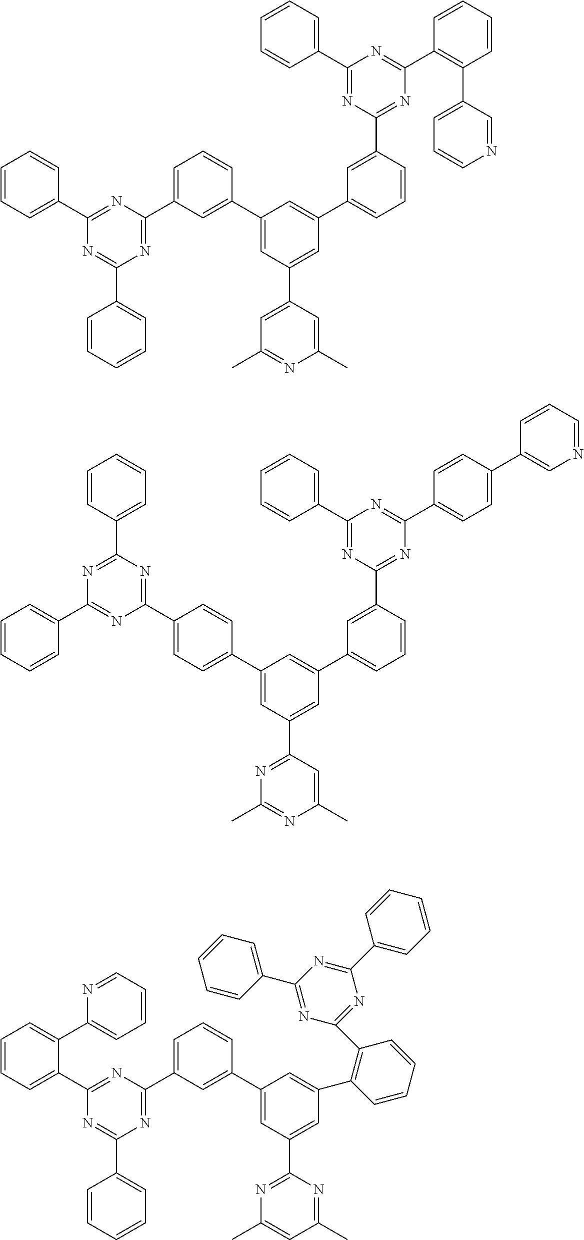 [SMILES: Cc1cc(-c2cc(-c3cccc(-c4nc(-c5ccccc5)nc(-c5ccccc5)n4)c3)cc(-c3cccc(-c4nc(-c5ccccc5)nc(-c5ccccc5-c5cccnc5)n4)c3)c2)cc(C)n1 ; Cc1cc(-c2cc(-c3ccc(-c4nc(-c5ccccc5)nc(-c5ccccc5)n4)cc3)cc(-c3cccc(-c4nc(-c5ccccc5)nc(-c5ccc(-c6cccnc6)cc5)n4)c3)c2)nc(C)n1 ; Cc1cc(C)nc(-c2cc(-c3cccc(-c4nc(-c5ccccc5)nc(-c5ccccc5-c5ccccn5)n4)c3)cc(-c3ccccc3-c3nc(-c4ccccc4)nc(-c4ccccc4)n3)c2)n1]

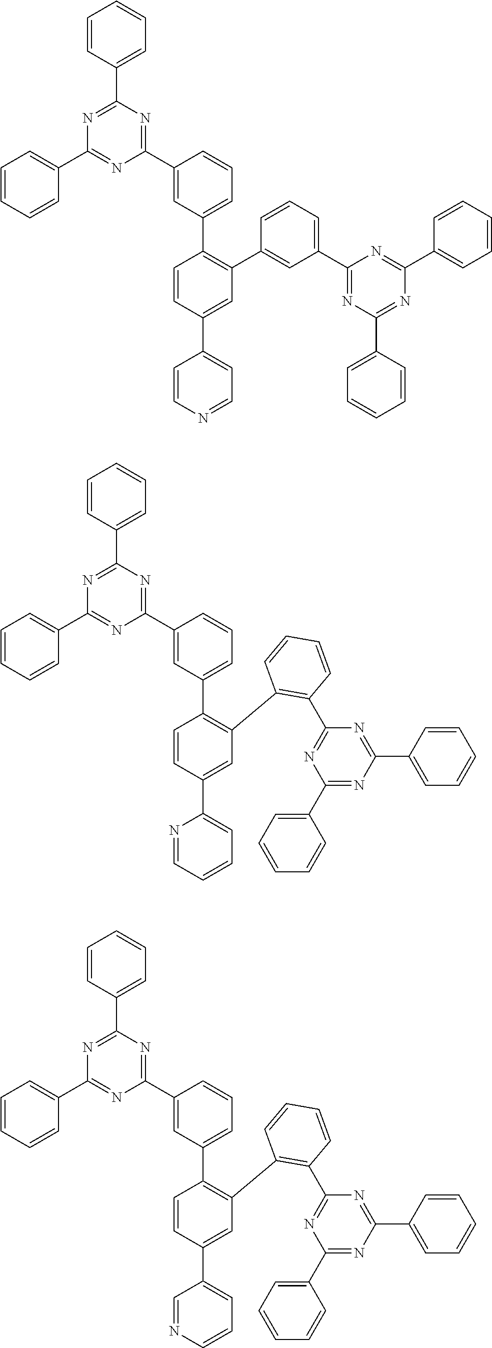 [SMILES: c1ccc(-c2nc(-c3ccccc3)nc(-c3cccc(-c4ccc(-c5ccncc5)cc4-c4cccc(-c5nc(-c6ccccc6)nc(-c6ccccc6)n5)c4)c3)n2)cc1 ; c1ccc(-c2nc(-c3ccccc3)nc(-c3cccc(-c4ccc(-c5ccccn5)cc4-c4ccccc4-c4nc(-c5ccccc5)nc(-c5ccccc5)n4)c3)n2)cc1 ; c1ccc(-c2nc(-c3ccccc3)nc(-c3cccc(-c4ccc(-c5cccnc5)cc4-c4ccccc4-c4nc(-c5ccccc5)nc(-c5ccccc5)n4)c3)n2)cc1]

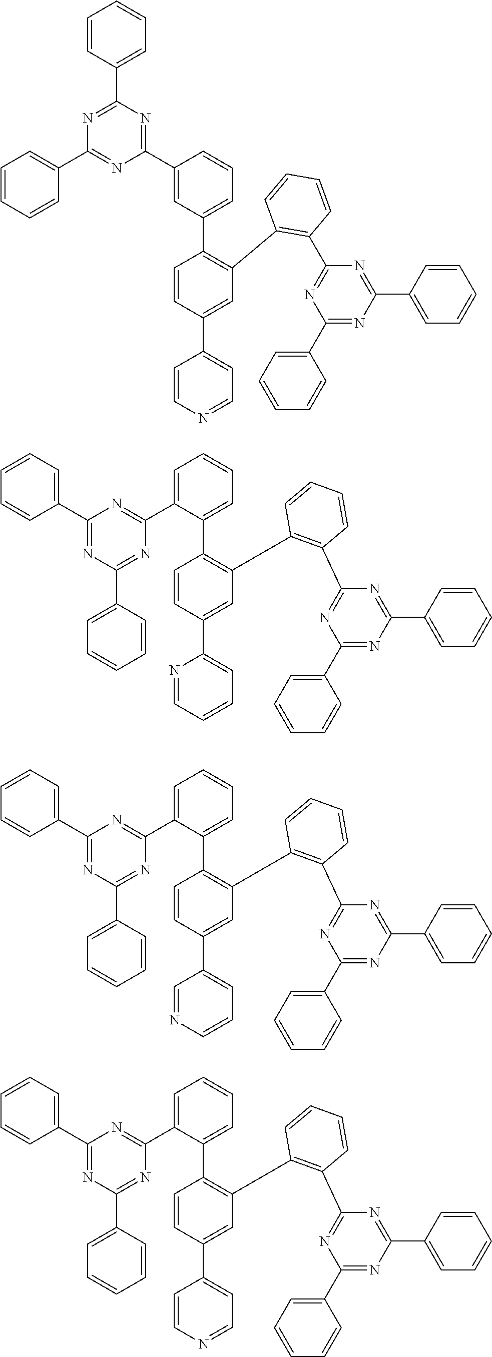

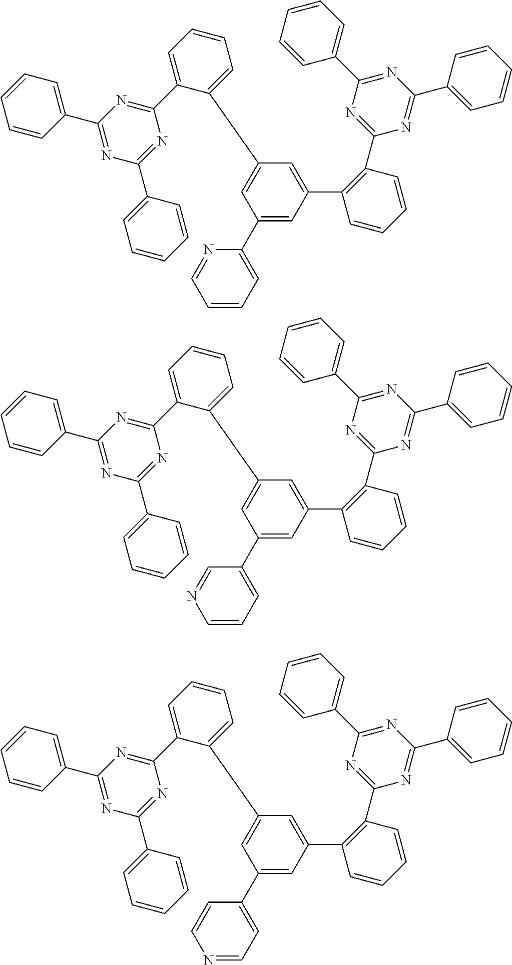

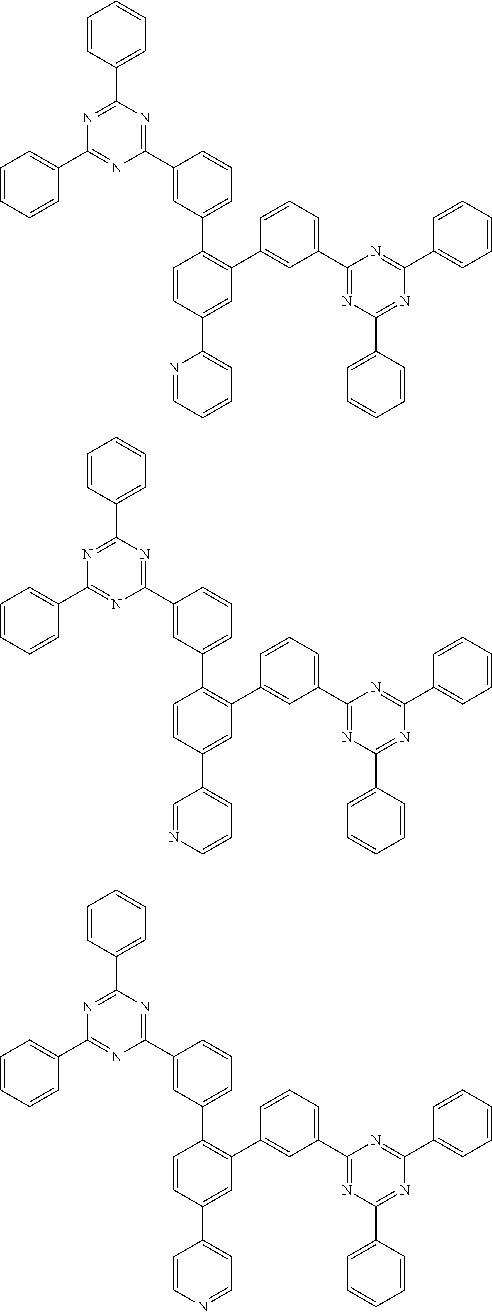 [SMILES: c1ccc(-c2nc(-c3ccccc3)nc(-c3cccc(-c4ccc(-c5ccccn5)cc4-c4cccc(-c5nc(-c6ccccc6)nc(-c6ccccc6)n5)c4)c3)n2)cc1 ; c1ccc(-c2nc(-c3ccccc3)nc(-c3cccc(-c4ccc(-c5cccnc5)cc4-c4cccc(-c5nc(-c6ccccc6)nc(-c6ccccc6)n5)c4)c3)n2)cc1 ; c1ccc(-c2nc(-c3ccccc3)nc(-c3cccc(-c4ccc(-c5ccncc5)cc4-c4cccc(-c5nc(-c6ccccc6)nc(-c6ccccc6)n5)c4)c3)n2)cc1]

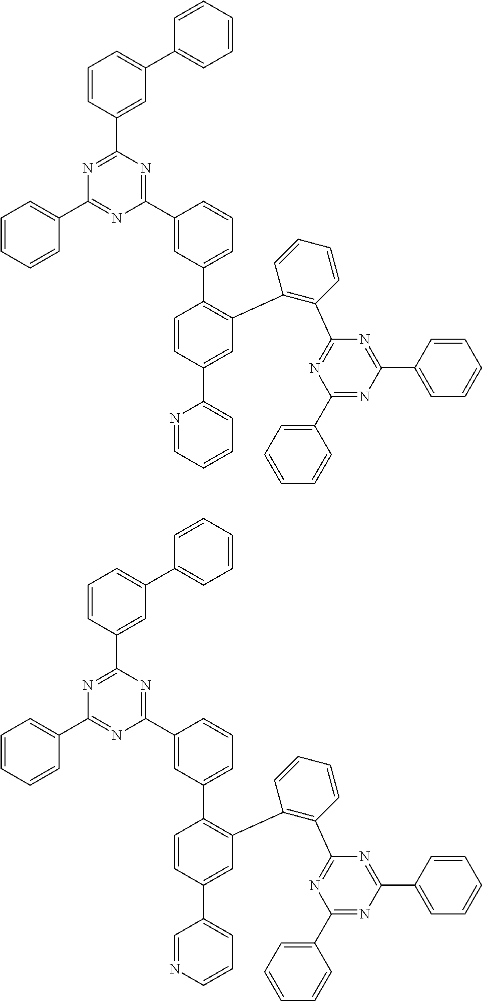 [SMILES: c1ccc(-c2cccc(-c3nc(-c4ccccc4)nc(-c4cccc(-c5ccc(-c6ccccn6)cc5-c5ccccc5-c5nc(-c6ccccc6)nc(-c6ccccc6)n5)c4)n3)c2)cc1 ; c1ccc(-c2cccc(-c3nc(-c4ccccc4)nc(-c4cccc(-c5ccc(-c6cccnc6)cc5-c5ccccc5-c5nc(-c6ccccc6)nc(-c6ccccc6)n5)c4)n3)c2)cc1]

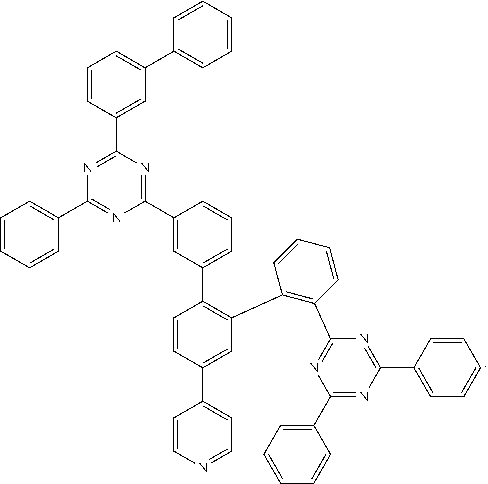 [SMILES: c1ccc(-c2cccc(-c3nc(-c4ccccc4)nc(-c4cccc(-c5ccc(-c6ccncc6)cc5-c5ccccc5-c5nc(-c6ccccc6)nc(-c6ccccc6)n5)c4)n3)c2)cc1]

An exemplary embodiment of the present specification provides an organic light emitting device including the compound represented by Chemical Formula 1.

When one member is disposed “on” another member in the present specification, this includes not only a case where the one member is brought into contact with another member, but also a case where still another member is present between the two members.

When one part “includes” one constituent element in the present specification, unless otherwise specifically described, this does not mean that another constituent element is excluded, but means that another constituent element may be further included.

In the present specification, the ‘layer’ has a meaning compatible with a ‘film’ usually used in the art, and means a coating covering a target region. The size of the ‘layer’ is not limited, and the sizes of the respective ‘layers’ may be the same as or different from one another. According to an exemplary embodiment, the size of the ‘layer’ may be the same as that of the entire device, may correspond to the size of a specific functional region, and may also be as small as a single sub-pixel.

In the present specification, when a specific A material is included in a B layer, this means both i) the fact that one or more A materials are included in one B layer and ii) the fact that the B layer is composed of one or more layers, and the A material is included in one or more layers of the multi-layered B layer.

In the present specification, when a specific A material is included in a C layer or a D layer, this means all of i) the fact that the A material is included in one or more layers of the C layer having one or more layers, ii) the fact that the A material is included in one or more layers of the D layer having one or more layers, and iii) the fact that the A material is included in each of the C layer having one or more layers and the D layer having one or more layers.

The present specification provides an organic light emitting device including: a first electrode; a second electrode; and an organic material layer having one or more layers provided between the first electrode and the second electrode, in which one or more layers of the organic material layer include the compound represented by Chemical Formula 1.

The organic material layer of the organic light emitting device of the present specification may also have a single-layered structure, but may have a multi-layered structure in which two or more organic material layers are stacked. For example, the organic material layer may have a structure including a hole injection layer, a hole transport layer, a light emitting layer, an electron transport layer, an electron injection layer, an electron blocking layer, a hole blocking layer, and the like. However, the structure of the organic light emitting device is not limited thereto, and may include a fewer number of organic layers.

According to an exemplary embodiment of the present specification, the organic material layer includes an electron injection layer, an electron transport layer, or an electron injection and transport layer, and the electron injection layer, the electron transport layer, or the electron injection and transport layer includes the compound.

According to an exemplary embodiment of the present specification, the organic material layer includes a hole blocking layer, and the hole blocking layer includes the compound.

According to an exemplary embodiment of the present specification, the organic material layer includes a light emitting layer.

According to an exemplary embodiment of the present specification, the organic material layer includes a hole injection layer, a hole transport layer, or a hole injection and transport layer.

According to an exemplary embodiment of the present specification, the organic material layer includes an electron blocking layer.

According to an exemplary embodiment of the present specification, the organic material layer includes a hole blocking layer.

According to an exemplary embodiment of the present specification, the organic light emitting device further includes one or two or more layers selected from the group consisting of a hole injection layer, a hole transport layer, a hole injection and transport layer, a light emitting layer, an electron transport layer, an electron injection layer, an electron injection and transport layer, a hole blocking layer, and an electron blocking layer.

According to an exemplary embodiment of the present specification, the organic light emitting device includes: a first electrode; a second electrode provided to face the first electrode; a light emitting layer provided between the first electrode and the second electrode; and an organic material layer having two or more layers provided between the light emitting layer and the first electrode, or between the light emitting layer and the second electrode.

According to an exemplary embodiment of the present specification, as the organic material layer having two or more layers, two or more may be selected from the group consisting of a hole injection layer, a hole transport layer, a hole injection and transport layer, a light emitting layer, an electron transport layer, an electron injection layer, an electron injection and transport layer, a hole blocking layer, and an electron blocking layer.

According to an exemplary embodiment of the present specification, a hole transport layer having two or more layers is included between the light emitting layer and the first electrode. The hole transport layer having two or more layers may include materials which are the same as or different from each other.

According to an exemplary embodiment of the present specification, the first electrode is an anode or a cathode.

According to an exemplary embodiment of the present specification, the second electrode is a cathode or an anode.

According to an exemplary embodiment of the present specification, the organic light emitting device may be a normal type organic light emitting device in which an anode, an organic material layer having one or more layers, and a cathode are sequentially stacked on a substrate.

According to an exemplary embodiment of the present specification, the organic light emitting device may be an inverted type organic light emitting device in which a cathode, an organic material layer having one or more layers, and an anode are sequentially stacked on a substrate.

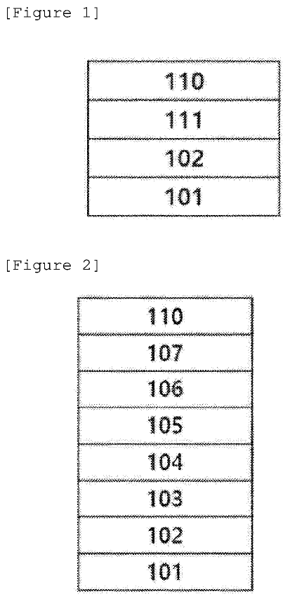

For example, the structure of the organic light emitting device according to an exemplary embodiment of the present specification is exemplified in FIGS. 1 and 2. FIGS. 1 and 2 exemplify an organic light emitting device, and the organic light emitting device is not limited thereto.

FIG. 1 exemplifies a structure of an organic light emitting device in which a first electrode 102, an organic material layer 111, and a second electrode 110 are sequentially stacked on a substrate 101. The compound represented by Chemical Formula 1 is included in the organic material layer.

FIG. 2 exemplifies a structure of an organic light emitting device in which a first electrode 102, a hole injection layer 103, a first hole transport layer 104, a second hole transport layer 105, a light emitting layer 106, an electron injection and transport layer 107, and a second electrode 110 are sequentially stacked on a substrate 101. The compound represented by Chemical Formula 1 is included in the electron injection and transport layer.

The organic light emitting device of the present specification may be manufactured by the materials and methods known in the art, except that an electron injection layer, an electron transport layer, an electron injection and transport layer, or a hole blocking layer includes the compound, that is, the compound represented by Chemical Formula 1.

When the organic light emitting device includes a plurality of organic material layers, the organic material layers may be formed of the same material or different materials.

For example, the organic light emitting device of the present specification may be manufactured by sequentially stacking a first electrode, an organic material layer, and a second electrode on a substrate. In this case, the organic light emitting device may be manufactured by depositing a metal or a metal oxide having conductivity, or an alloy thereof on a substrate to form an anode, forming an organic material layer including a hole injection layer, a hole transport layer, a light emitting layer, and an electron transport layer thereon, and then depositing a material, which may be used as a cathode, thereon, by using a physical vapor deposition (PVD) method such as sputtering or e-beam evaporation. In addition to the method described above, an organic light emitting device may be manufactured by sequentially depositing a cathode material, an organic material layer, and an anode material on a substrate.

Further, the compound represented by Chemical Formula 1 may be formed as an organic material layer by not only a vacuum deposition method, but also a solution application method when an organic light emitting device is manufactured. Here, the solution application method means spin coating, dip coating, doctor blading, inkjet printing, screen printing, a spray method, roll coating, and the like, but is not limited thereto.

In addition to the method described above, an organic light emitting device may be made by sequentially depositing a cathode material, an organic material layer, and an anode material on a substrate. However, the manufacturing method is not limited thereto.

As the anode material, materials having large work function are normally preferred so that hole injection to an organic material layer is smooth. Examples thereof include: a metal, such as vanadium, chromium, copper, zinc, and gold, or an alloy thereof; a metal oxide, such as zinc oxide, indium oxide, indium tin oxide (ITO), and indium zinc oxide (IZO); a combination of a metal and an oxide, such as Zno:Al or SnO₂:Sb; a conductive polymer, such as poly(3-methylthiophene), poly[3,4-(ethylene-1,2-dioxy)thiophene] (PEDOT), polypyrrole, and polyaniline; and the like, but are not limited thereto.

As the cathode material, materials having a low work function are usually preferred so as to facilitate the injection of electrons into an organic material layer. Examples thereof include: a metal, such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

The light emitting layer may include a host material and a dopant material. Examples of the host material include fused aromatic ring derivatives, or hetero ring-containing compounds, and the like. Specific examples of the fused aromatic ring derivative include anthracene derivatives, pyrene derivatives, naphthalene derivatives, pentacene derivatives, phenanthrene compounds, fluoranthene compounds, and the like, and specific examples of the hetero ring-containing compound include dibenzofuran derivatives, ladder-type furan compounds, pyrimidine derivatives, and the like, but the examples are not limited thereto.

According to an exemplary embodiment of the present specification, the host includes a compound represented by the following Chemical Formula H-1, but is not limited thereto.

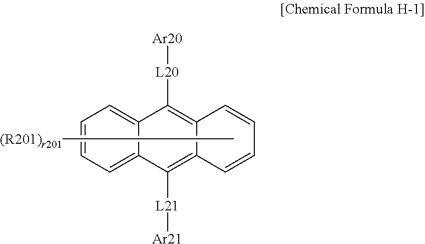

In Chemical Formula H-1,
L20 and L21 are the same as or different from each other, and are each independently a direct bond; a substituted or unsubstituted arylene group; or a substituted or unsubstituted divalent heterocyclic group,
Ar20 and Ar21 are the same as or different from each other, and are each independently hydrogen; deuterium; a substituted or unsubstituted aryl group; or a substituted or unsubstituted heterocyclic group,
R201 is hydrogen; deuterium; a halogen group; a substituted or unsubstituted alkyl group; a substituted or unsubstituted cycloalkyl group; a substituted or unsubstituted aryl group; or a substituted or unsubstituted heterocyclic group, and
r201 is an integer from 1 to 8, and when r201 is 2 or higher, two or more R201's are the same as or different from each other.

In an exemplary embodiment of the present specification, L20 and L21 are the same as or different from each other, and are each independently a direct bond; a monocyclic or polycyclic arylene group having 6 to 30 carbon atoms; or a monocyclic or polycyclic divalent heterocyclic group having 2 to 30 carbon atoms.

In an exemplary embodiment of the present specification, L20 and L21 are the same as or different from each other, and are each independently a direct bond; a phenylene group which is unsubstituted or substituted with deuterium; a biphenylylene group which is unsubstituted or substituted with deuterium; a naphthylene group which is unsubstituted or substituted with deuterium; a divalent dibenzofuran group; or a divalent dibenzothiophene group.

In an exemplary embodiment of the present specification, Ar20 and Ar21 are the same as or different from each other, and are each independently a substituted or unsubstituted monocyclic or polycyclic aryl group having 6 to 30 carbon atoms; or a substituted or unsubstituted monocyclic or polycyclic heterocyclic group having 2 to 30 carbon atoms.

In an exemplary embodiment of the present specification, Ar20 and Ar21 are the same as or different from each other, and are each independently a substituted or unsubstituted monocyclic to tetracyclic aryl group having 6 to 20 carbon atoms; or a substituted or unsubstituted monocyclic to tetracyclic heterocyclic group having 6 to 20 carbon atoms.

In an exemplary embodiment of the present specification, Ar20 and Ar21 are the same as or different from each other, and are each independently a phenyl group which is unsubstituted or substituted with deuterium or a monocyclic or polycyclic aryl group having 6 to 20 carbon atoms; a biphenyl group which is unsubstituted or substituted with deuterium or a monocyclic or polycyclic aryl group having 6 to 20 carbon atoms; a naphthyl group which is unsubstituted or substituted with a monocyclic or polycyclic aryl group having 6 to 20 carbon atoms; a thiophene group which is unsubstituted or substituted with a monocyclic or polycyclic aryl group having 6 to 30 carbon atoms; a dibenzofuran group which is unsubstituted or substituted with a monocyclic or polycyclic aryl group having 6 to 20 carbon atoms; a naphthobenzofuran group which is unsubstituted or substituted with a monocyclic or polycyclic aryl group having 6 to 20 carbon atoms; a dibenzothiophene group which is unsubstituted or substituted with a monocyclic or polycyclic aryl group having 6 to 20 carbon atoms; or a naphthobenzothiophene group which is unsubstituted or substituted with a monocyclic or polycyclic aryl group having 6 to 20 carbon atoms.

In an exemplary embodiment of the present specification, Ar20 and Ar21 are the same as or different from each other, and are each independently a phenyl group which is unsubstituted or substituted with deuterium; a biphenyl group which is unsubstituted or substituted with deuterium; a terphenyl group; a naphthyl group which is unsubstituted or substituted with deuterium; a thiophene group which is unsubstituted or substituted with a phenyl group; a phenanthrene group; a dibenzofuran group; a naphthobenzofuran group; a dibenzothiophene group; or a naphthobenzothiophene group.

In an exemplary embodiment of the present specification, Ar20 is a substituted or unsubstituted heterocyclic group, and Ar21 is a substituted or unsubstituted aryl group.

According to an exemplary embodiment of the present specification, R201 is hydrogen; or a phenyl group.

According to an exemplary embodiment of the present specification, Chemical Formula H-1 is represented by the following compound.

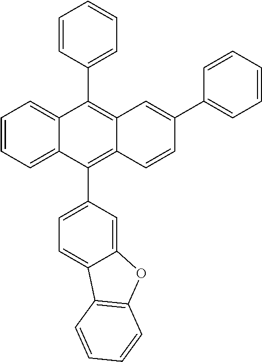 [SMILES: c1ccc(-c2ccc3c(-c4ccc5c(c4)oc4ccccc45)c4ccccc4c(-c4ccccc4)c3c2)cc1]

Examples of the dopant material include an aromatic amine derivative, a styrylamine compound, a boron complex, a fluoranthene compound, a metal complex, and the like. Specifically, the aromatic amine derivative is a fused aromatic ring derivative having a substituted or unsubstituted arylamine group, and examples thereof include pyrene, anthracene, chrysene, periflanthene, and the like having an arylamine group. Further, the styrylamine compound is a compound in which a substituted or unsubstituted arylamine is substituted with at least one arylvinyl group, and one or two or more substituents selected from the group consisting of an aryl group, a silyl group, an alkyl group, a cycloalkyl group, and an arylamine group are substituted or unsubstituted. Specific examples thereof include styrylamine, styryldiamine, styryltriamine, styryltetramine, and the like, but are not limited thereto. Further, examples of the metal complex include an iridium complex, a platinum complex, and the like, but are not limited thereto.

According to an exemplary embodiment of the present specification, the dopant includes a compound represented by the following Chemical Formula D-1, but is not limited thereto.

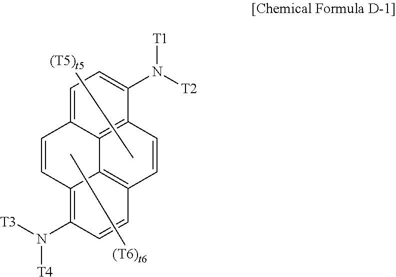

In Chemical Formula D-1,
T1 to T6 are the same as or different from each other, and are each independently hydrogen; a substituted or unsubstituted alkyl group; a substituted or unsubstituted alkylsilyl group; a substituted or unsubstituted aryl group; or a substituted or unsubstituted heteroaryl group,
t5 and t6 are each an integer from 1 to 4,
when t5 is 2 or higher, two or more T5's are the same as or different from each other, and
when t6 is 2 or higher, two or more T6's are the same as or different from each other.

According to an exemplary embodiment of the present specification, T1 to T6 are the same as or different from each other, and are each independently hydrogen; a substituted or unsubstituted straight-chained or branched alkyl group having 1 to 30 carbon atoms; a substituted or unsubstituted straight-chained or branched alkylsilyl group having 1 to 30 carbon atoms; a substituted or unsubstituted monocyclic or polycyclic aryl group having 6 to 30 carbon atoms; or a substituted or unsubstituted monocyclic or polycyclic heteroaryl group having 2 to 30 carbon atoms.

According to an exemplary embodiment of the present specification, T1 to T6 are the same as or different from each other, and are each independently hydrogen; a straight-chained or branched alkyl group having 1 to 30 carbon atoms; a straight-chained or branched alkylsilyl group having 1 to 30 carbon atoms; a monocyclic or polycyclic aryl group having 6 to 30 carbon atoms, which is unsubstituted or substituted with deuterium, a cyano group, or a straight-chained or branched alkyl group having 1 to 30 carbon atoms; or a monocyclic or polycyclic heteroaryl group having 2 to 30 carbon atoms.

According to an exemplary embodiment of the present specification, T1 to T6 are the same as or different from each other, and are each independently hydrogen; an isopropyl group; a trimethylsilyl group; a phenyl group substituted with deuterium; a phenyl group substituted with a cyano group; or a phenyl group substituted with a methyl group.

According to an exemplary embodiment of the present specification, Chemical Formula D-1 is represented by the following compound.

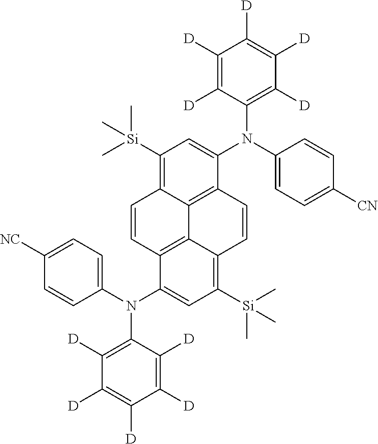 [SMILES: [2H]c1c([2H])c([2H])c(N(c2ccc(C#N)cc2)c2cc([Si](C)(C)C)c3ccc4c(N(c5ccc(C#N)cc5)c5c([2H])c([2H])c([2H])c([2H])c5[2H])cc([Si](C)(C)C)c5ccc2c3c45)c([2H])c1[2H]]

The hole injection layer is a layer which accepts holes from an electrode. It is preferred that the hole injection material has an ability to transport holes, and has an effect of accepting holes from an anode and an excellent hole injection effect for a light emitting layer or a light emitting material. Further, the hole injection material is preferably a material which is excellent in ability to prevent excitons produced from a light emitting layer from moving to an electron injection layer or an electron injection material. In addition, the hole injection material is preferably a material which is excellent in ability to form a thin film. In addition, the highest occupied molecular orbital (HOMO) of the hole injection material is preferably a value between the work function of the anode material and the HOMO of the neighboring organic material layer. Specific examples of the hole injection material include: metal porphyrin, oligothiophene, and arylamine-based organic materials; hexanitrile hexaazatriphenylene-based organic materials; quinacridone-based organic materials; perylene-based organic materials; polythiophene-based conductive polymers such as anthraquinone and polyaniline; and the like, but are not limited thereto.

According to an exemplary embodiment of the present specification, the hole injection layer includes a compound represented by the following Chemical Formula HI-1, but is not limited thereto.

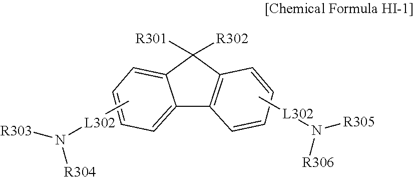

In Chemical Formula HI-1,
R301 to R306 are the same as or different from each other, and are each independently hydrogen; deuterium; a cyano group; a substituted or unsubstituted alkyl group; a substituted or unsubstituted amine group; a substituted or unsubstituted aryl group; or a substituted or unsubstituted heterocyclic group, and
L301 and L302 are the same as or different from each other, and are each independently a direct bond; a substituted or unsubstituted arylene group; or a substituted or unsubstituted divalent heterocyclic group.

According to an exemplary embodiment of the present specification, R301 and R302 are the same as or different from each other, and are each independently a straight-chained or branched alkyl group having 1 to 30 carbon atoms.

According to an exemplary embodiment of the present specification, R301 and R302 are a methyl group.

According to an exemplary embodiment of the present specification, L301 and L302 are a direct bond.

According to an exemplary embodiment of the present specification, R303 to R306 are the same as or different from each other, and are each independently a substituted or unsubstituted aryl group; or a substituted or unsubstituted heterocyclic group.

According to an exemplary embodiment of the present specification, R303 to R306 are the same as or different from each other, and are each independently a monocyclic or polycyclic aryl group having 6 to 30 carbon atoms; or a monocyclic or polycyclic heterocyclic group having 2 to 30 carbon atoms, which is unsubstituted or substituted with a monocyclic or polycyclic aryl group having 6 to 30 carbon atoms.

According to an exemplary embodiment of the present specification, R303 to R306 are the same as or different from each other, and are each independently a phenyl group; or a carbazole group unsubstituted or substituted with a phenyl group.

According to an exemplary embodiment of the present specification, Chemical Formula HI-1 is represented by the following compound.

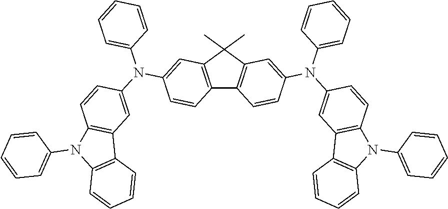 [SMILES: CC1(C)c2cc(N(c3ccccc3)c3ccc4c(c3)c3ccccc3n4-c3ccccc3)ccc2-c2ccc(N(c3ccccc3)c3ccc4c(c3)c3ccccc3n4-c3ccccc3)cc21]

The hole transport layer is a layer which accepts holes from a hole injection layer and transports the holes to a light emitting layer. A hole transport material is preferably a material having high hole mobility which may accept holes from an anode or a hole injection layer and transfer the holes to a light emitting layer. Specific examples thereof include arylamine-based organic materials, conductive polymers, block copolymers having both conjugated portions and non-conjugated portions, and the like, but are not limited thereto.

According to an exemplary embodiment of the present specification, the hole transport layer includes a compound represented by the following Chemical Formula HT-1, but is not limited thereto.

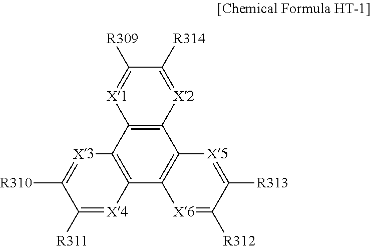

In Chemical Formula HT-1, at least one of X′1 to X′6 is N, and the others are CH, and
R309 to R314 are the same as or different from each other, and are each independently hydrogen; deuterium; a cyano group; a substituted or unsubstituted alkyl group; a substituted or unsubstituted amine group; a substituted or unsubstituted aryl group; or a substituted or unsubstituted heteroaryl group, or are bonded to an adjacent group to form a substituted or unsubstituted ring.

According to an exemplary embodiment of the present specification, X′1 to X′6 are N.

According to an exemplary embodiment of the present specification, R309 to R314 are a cyano group.

According to an exemplary embodiment of the present specification, Chemical Formula HT-1 is represented by the following compound.

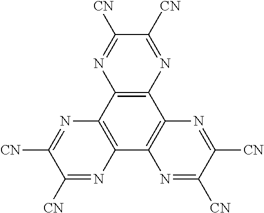

According to an exemplary embodiment of the present specification, the hole transport layer includes a compound represented by the following Chemical Formula HT-2, but is not limited thereto.

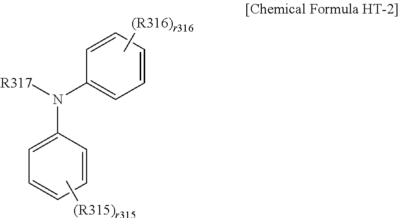

In Chemical Formula HT-2,
R315 to R317 are the same as or different from each other, and are each independently any one selected from the group consisting of hydrogen; deuterium; a substituted or unsubstituted alkyl group; a substituted or unsubstituted aryl group; a substituted or unsubstituted heteroaryl group; and a combination thereof, or are bonded to an adjacent group to form a substituted or unsubstituted ring,
r315 is an integer from 1 to 5, and when r315 is 2 or higher, two or more R315's are the same as or different from each other, and
r316 is an integer from 1 to 5, and when r316 is 2 or higher, two or more R316's are the same as or different from each other.

According to an exemplary embodiment of the present specification, R317 is any one selected from the group consisting of a substituted or unsubstituted aryl group; a substituted or unsubstituted heteroaryl group; and a combination thereof.

According to an exemplary embodiment of the present specification, R317 is any one selected from the group consisting of a carbazole group; a phenyl group; a biphenyl group; and a combination thereof.

According to an exemplary embodiment of the present specification, R315 and R316 are the same as or different from each other, and are each independently a substituted or unsubstituted aryl group, or are bonded to an adjacent group to form an aromatic hydrocarbon ring substituted with an alkyl group.

According to an exemplary embodiment of the present specification, R315 and R316 are the same as or different from each other, and are each independently a phenyl group, or are bonded to an adjacent group to form indene substituted with a methyl group.

According to an exemplary embodiment of the present specification, Chemical Formula HT-2 is represented by the following compound.

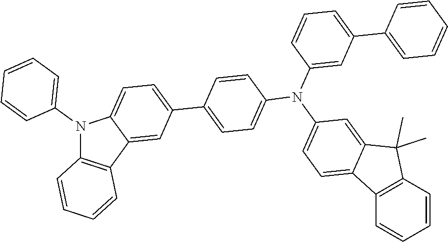 [SMILES: CC1(C)c2ccccc2-c2ccc(N(c3ccc(-c4ccc5c(c4)c4ccccc4n5-c4ccccc4)cc3)c3cccc(-c4ccccc4)c3)cc21]

The electron transport layer is a layer which accepts electrons from an electron injection layer and transports the electrons to a light emitting layer. When the organic light emitting device according to an exemplary embodiment of the present specification includes an additional electron transport layer other than the electron transport layer including Chemical Formula 1, an electron transport material is preferably a material having high electron mobility which may proficiently accept electrons from a cathode and transfer the electrons to a light emitting layer. Specific examples thereof include: Al complexes of 8-hydroxyquinoline; complexes including Alq₃; organic radical compounds; hydroxyflavone-metal complexes; and the like, but are not limited thereto. The electron transport layer may be used with any desired cathode material, as used according to the related art. In particular, an appropriate cathode material is a typical material which has a low work function, followed by an aluminum layer or a silver layer. Specific examples thereof include cesium, barium, calcium, ytterbium, and samarium, in each case followed by an aluminum layer or a silver layer.

The electron injection layer is a layer which accepts electrons from an electrode. When the organic light emitting device according to an exemplary embodiment of the present specification includes an additional electron injection layer other than the electron injection layer including Chemical Formula 1, it is preferred that an electron injection material is excellent in the ability to transport electrons, and has an effect of accepting electrons from a second electrode and an excellent effect of injecting electrons into a light emitting layer or light emitting material. Further, the electron injection material is preferably a material which prevents excitons produced from a light emitting layer from moving to a hole injection layer and is excellent in ability to form a thin film. Specific examples thereof include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, metal complex compounds, nitrogen-containing 5-membered ring derivatives, and the like, but are not limited thereto.

Examples of the metal complex compounds include 8-hydroxyquinolinato lithium, bis(8-hydroxyquinolinato) zinc, bis(8-hydroxyquinolinato) copper, bis(8-hydroxyquinolinato) manganese, tris(8-hydroxyquinolinato) aluminum, tris(2-methyl-8-hydroxyquinolinato) aluminum, tris(8-hydroxyquinolinato) gallium, bis(10-hydroxybenzo[h]quinolinato) beryllium, bis(10-hydroxybenzo[h]quinolinato) zinc, bis(2-methyl-8-quinolinato) chlorogallium, bis(2-methyl-8-quinolinato) (o-cresolato) gallium, bis(2-methyl-8-quinolinato) (1-naphtholato) aluminum, bis(2-methyl-8-quinolinato) (2-naphtholato) gallium, and the like, but are not limited thereto.

According to an exemplary embodiment of the present specification, the electron injection and transport layer is a layer that transports electrons to the light emitting layer. As the electron injection and transport layer material, the compound represented by Chemical Formula 1 may be used, and when an additional electron injection and transport layer other than the electron injection and electron transport layer including Chemical Formula 1 is included, the materials exemplified for the electron transport layer and the electron injection layer may be used, but are not limited thereto.

According to an exemplary embodiment of the present specification, the organic material layer includes an electron injection layer, an electron transport layer, or an electron injection and transport layer, and the electron injection layer, electron transport layer, or electron injection and transport layer includes the compound. The electron injection layer, electron transport layer, or electron injection and transport layer including the compound further includes a metal or a metal complex compound.

According to an exemplary embodiment of the present specification, the organic material layer includes an electron injection and transport layer, and the electron injection and transport layer includes the compound. The electron injection and transport layer including the compound further includes a metal or a metal complex compound.

According to an exemplary embodiment of the present specification, the organic material layer includes an electron injection and transport layer, and the electron injection and transport layer includes the compound. The electron injection and transport layer including the compound further includes a metal complex compound.

The materials as described above may be used as the metal or the metal complex compound.

According to an exemplary embodiment of the present specification, the compound: the metal or the metal complex is included at a weight ratio of 1:99 to 99:1, specifically 10:90 to 90 to 10, and more specifically 50:50.

The electron blocking layer is a layer which may improve the service life and efficiency of a device by preventing electrons injected from an electron injection layer from passing through a light emitting layer and entering a hole injection layer. As the electron blocking layer, the publicly-known material can be used without limitation, and the electron blocking layer may be formed between a light emitting layer and a hole injection layer, or between a light emitting layer and a layer which simultaneously injects and transports holes.

The hole blocking layer is a layer which blocks holes from reaching a cathode, and may be generally formed under the same conditions as those of the electron injection layer. When the organic light emitting device according to an exemplary embodiment of the present specification includes an additional hole blocking layer other than the hole blocking layer including Chemical Formula 1, specifically, an oxadiazole derivative or a triazole derivative, a phenanthroline derivative, an aluminum complex, and the like are used, but are not limited thereto.

The organic light emitting device according to the present specification may be a top-emission type, a bottom-emission type or a dual-emission type depending on the materials used.

The organic light emitting device according to the present specification may be included and used in various electronic devices. For example, the electronic device may be a display panel, a touch panel, a solar module, a lighting device, and the like, and is not limited thereto.

### MODE FOR INVENTION

Hereinafter, the present specification will be described in detail with reference to Examples, Comparative Examples, and the like for specifically describing the present specification. However, the Examples and the Comparative Examples according to the present specification may be modified in various forms, and it is not interpreted that the scope of the present specification is limited to the Examples and the Comparative Examples described below in detail. The Examples and the Comparative Examples of the present specification are provided to more completely explain the present specification to a person with ordinary skill in the art.

### Preparation Example 1. Preparation of Compound 1

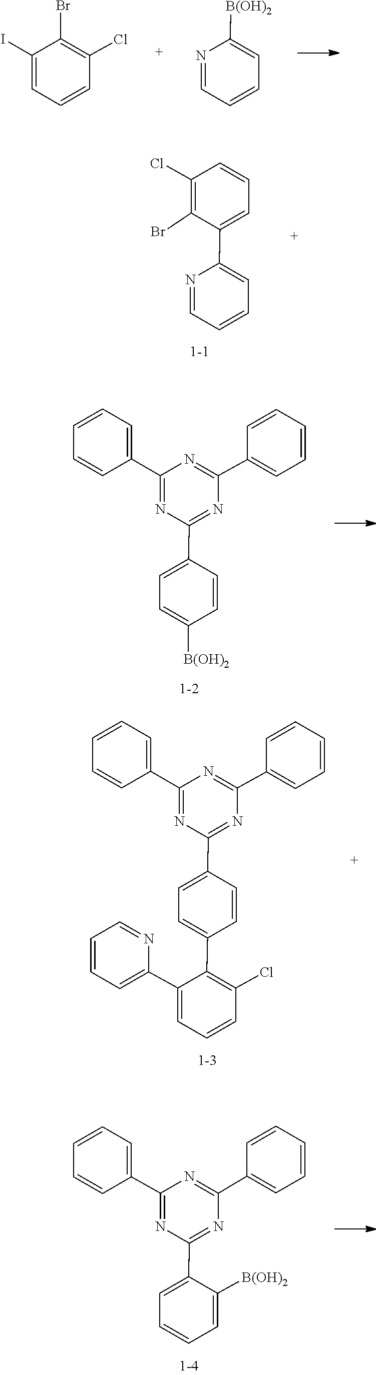 [SMILES: Clc1cccc(I)c1Br ; OB(O)c1ccccn1 ; Clc1cccc(-c2ccccn2)c1Br ; OB(O)c1ccc(-c2nc(-c3ccccc3)nc(-c3ccccc3)n2)cc1 ; Clc1cccc(-c2ccccn2)c1-c1ccc(-c2nc(-c3ccccc3)nc(-c3ccccc3)n2)cc1 ; OB(O)c1ccccc1-c1nc(-c2ccccc2)nc(-c2ccccc2)n1]

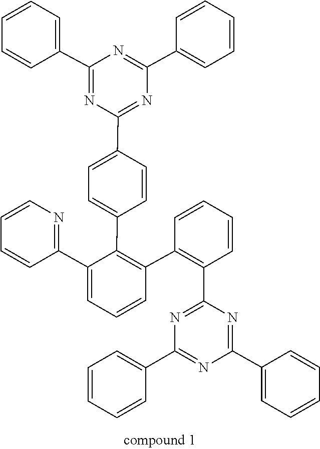 [SMILES: c1ccc(-c2nc(-c3ccccc3)nc(-c3ccc(-c4c(-c5ccccn5)cccc4-c4ccccc4-c4nc(-c5ccccc5)nc(-c5ccccc5)n4)cc3)n2)cc1]

1) Preparation of Compound 1-1 2-Bromo-1-chloro-3-iodobenzene (9.52 g, 30 mmol) and the compound pyridin-2-ylboronic acid (4.06 g, 33 mmol) were introduced into tetrahydrofuran (300 mL). After 2 M K₂CO₃ (200 mL) and tetrakis(triphenylphosphine) palladium (0) (0.3 g) were added thereto, the resulting mixture was stirred and refluxed for 5 hours. After the mixture was cooled to room temperature, a solid produced by filtering the mixture was recrystallized twice with toluene, thereby preparing Compound 1-1.

### 2) Preparation of Compound 1-3

Compound 1-1 (8.06 g, 30 mmol) and Compound 1-2 (11.66 g, 33 mmol) were introduced into tetrahydrofuran (300 mL). After 2 M K₂CO₃ (200 mL), palladium acetate (0.14 g) and a 2-dicyclohexylphosphino-2′, 6′-dimethoxybiphenyl (s-Phos) ligand (0.50 g) were added thereto, the resulting mixture was stirred and refluxed for 5 hours. After the mixture was cooled to room temperature, a solid produced by filtering the mixture was recrystallized twice with toluene, thereby preparing Compound 1-3.

### 3) Preparation of Compound 1

Compound 1-3 (14.91 g, 30 mmol) and Compound 1-4 (11.66 g, 33 mmol) were introduced into tetrahydrofuran (300 ml). After 2 M K₂CO₃ (200 ml), palladium acetate (0.14 g) and a 2-dicyclohexylphosphino-2′, 6′-dimethoxybiphenyl (s-Phos) ligand (0.50 g) were added thereto, the resulting mixture was stirred and refluxed for 5 hours. After the mixture was cooled to room temperature, a solid produced by filtering the mixture was recrystallized twice with toluene, thereby preparing Compound 1 (20.09 g, yield 87%, MS: [M+H]+=770).

### Preparation Example 2. Preparation of Compound 2

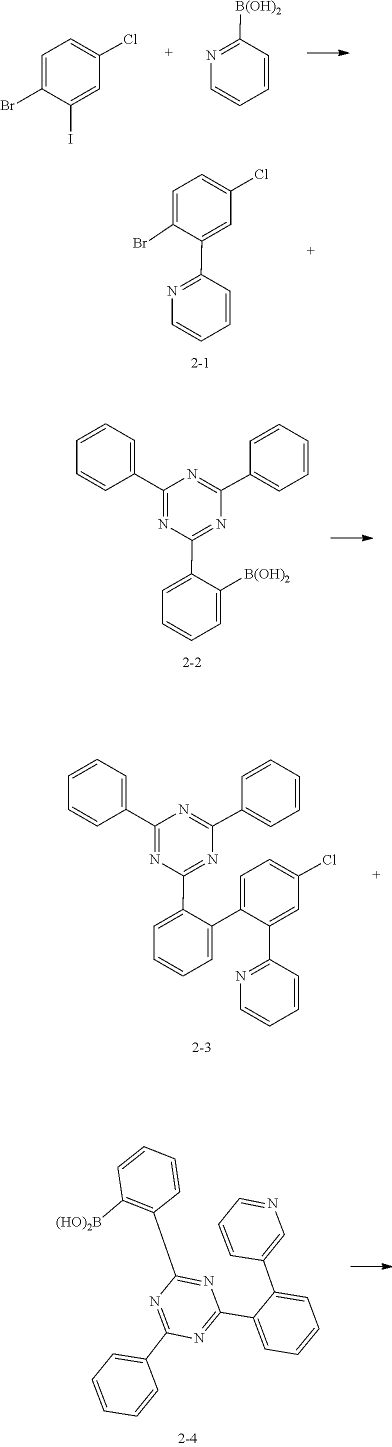 [SMILES: Clc1ccc(Br)c(I)c1 ; OB(O)c1ccccn1 ; Clc1ccc(Br)c(-c2ccccn2)c1 ; OB(O)c1ccccc1-c1nc(-c2ccccc2)nc(-c2ccccc2)n1 ; Clc1ccc(-c2ccccc2-c2nc(-c3ccccc3)nc(-c3ccccc3)n2)c(-c2ccccn2)c1 ; OB(O)c1ccccc1-c1nc(-c2ccccc2)nc(-c2ccccc2-c2cccnc2)n1]

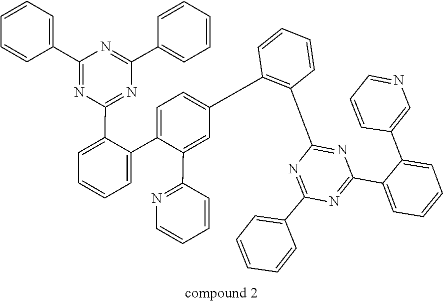 [SMILES: c1ccc(-c2nc(-c3ccccc3-c3cccnc3)nc(-c3ccccc3-c3ccc(-c4ccccc4-c4nc(-c5ccccc5)nc(-c5ccccc5)n4)c(-c4ccccn4)c3)n2)cc1]

Compound 2 was prepared in the same manner as in the preparation method in Preparation Example 1, except that each starting material was used as in the reaction scheme.

MS: [M+H]⁺=847

### Preparation Example 3. Preparation of Compound 3

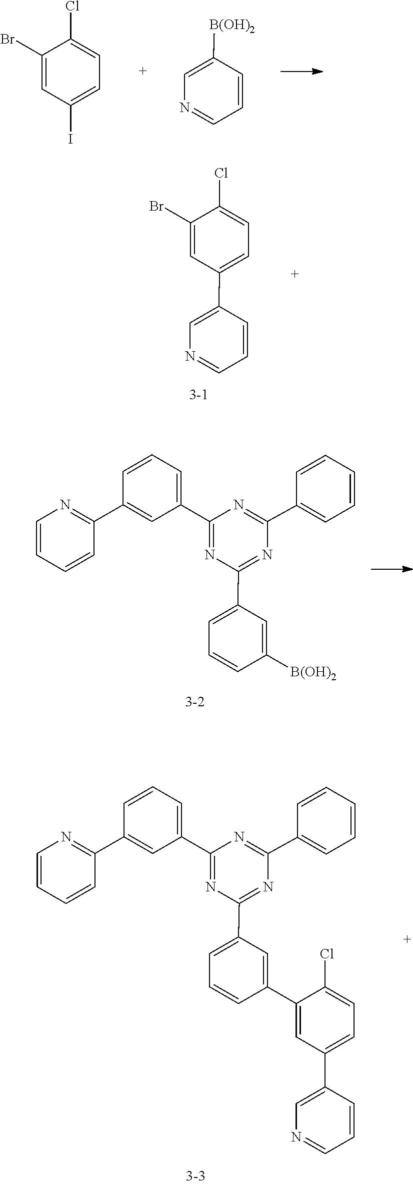 [SMILES: Clc1ccc(I)cc1Br ; OB(O)c1cccnc1 ; Clc1ccc(-c2cccnc2)cc1Br ; OB(O)c1cccc(-c2nc(-c3ccccc3)nc(-c3cccc(-c4ccccn4)c3)n2)c1 ; Clc1ccc(-c2cccnc2)cc1-c1cccc(-c2nc(-c3ccccc3)nc(-c3cccc(-c4ccccn4)c3)n2)c1]

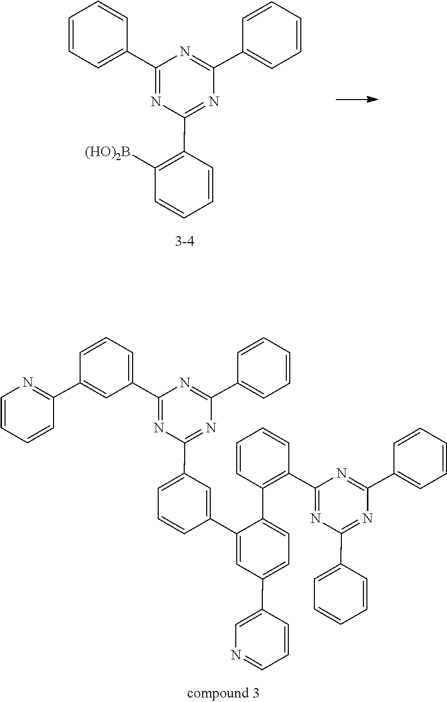 [SMILES: OB(O)c1ccccc1-c1nc(-c2ccccc2)nc(-c2ccccc2)n1 ; c1ccc(-c2nc(-c3cccc(-c4ccccn4)c3)nc(-c3cccc(-c4cc(-c5cccnc5)ccc4-c4ccccc4-c4nc(-c5ccccc5)nc(-c5ccccc5)n4)c3)n2)cc1]

Compound 3 was prepared in the same manner as in the preparation method in Preparation Example 1, except that each starting material was used as in the reaction scheme.

MS: [M+H]⁺=847

### Preparation Example 4. Preparation of Compound 4

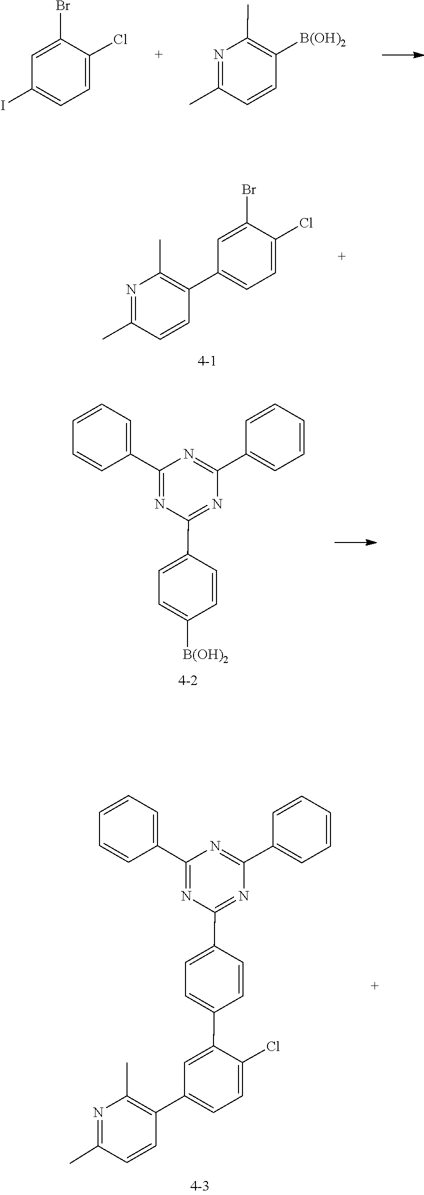 [SMILES: Clc1ccc(I)cc1Br ; Cc1ccc(B(O)O)c(C)n1 ; Cc1ccc(-c2ccc(Cl)c(Br)c2)c(C)n1 ; OB(O)c1ccc(-c2nc(-c3ccccc3)nc(-c3ccccc3)n2)cc1 ; Cc1ccc(-c2ccc(Cl)c(-c3ccc(-c4nc(-c5ccccc5)nc(-c5ccccc5)n4)cc3)c2)c(C)n1]

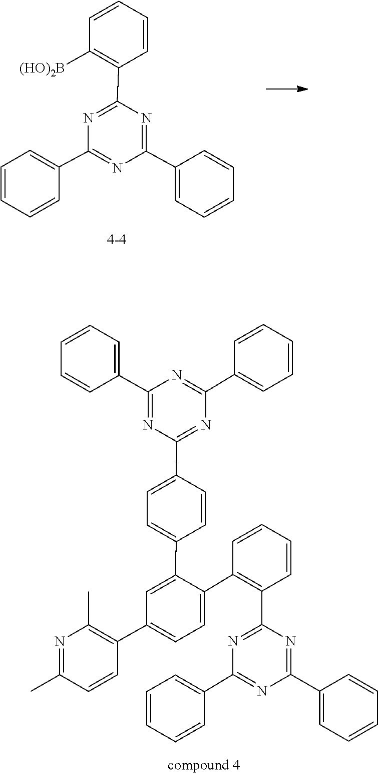 [SMILES: OB(O)c1ccccc1-c1nc(-c2ccccc2)nc(-c2ccccc2)n1 ; Cc1ccc(-c2ccc(-c3ccccc3-c3nc(-c4ccccc4)nc(-c4ccccc4)n3)c(-c3ccc(-c4nc(-c5ccccc5)nc(-c5ccccc5)n4)cc3)c2)c(C)n1]

Compound 4 was prepared in the same manner as in the preparation method in Preparation Example 1, except that each starting material was used as in the reaction scheme.

MS: [M+H]⁺=798

### Preparation Example 5. Preparation of Compound 5

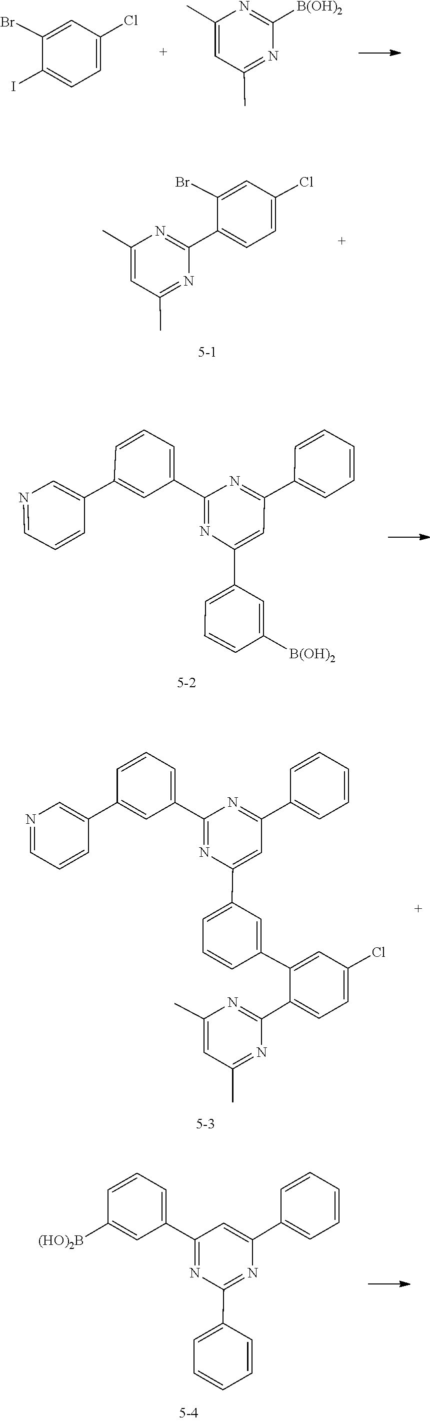 [SMILES: Clc1ccc(I)c(Br)c1 ; Cc1cc(C)nc(B(O)O)n1 ; Cc1cc(C)nc(-c2ccc(Cl)cc2Br)n1 ; OB(O)c1cccc(-c2cc(-c3ccccc3)nc(-c3cccc(-c4cccnc4)c3)n2)c1 ; Cc1cc(C)nc(-c2ccc(Cl)cc2-c2cccc(-c3cc(-c4ccccc4)nc(-c4cccc(-c5cccnc5)c4)n3)c2)n1 ; OB(O)c1cccc(-c2cc(-c3ccccc3)nc(-c3ccccc3)n2)c1]

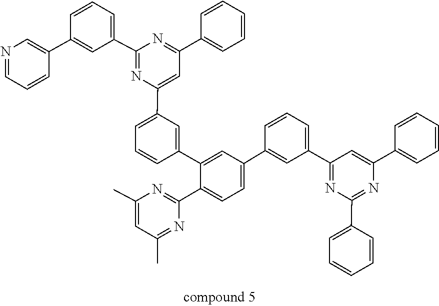 [SMILES: Cc1cc(C)nc(-c2ccc(-c3cccc(-c4cc(-c5ccccc5)nc(-c5ccccc5)n4)c3)cc2-c2cccc(-c3cc(-c4ccccc4)nc(-c4cccc(-c5cccnc5)c4)n3)c2)n1]

Compound 5 was prepared in the same manner as in the preparation method in Preparation Example 1, except that each starting material was used as in the reaction scheme.

MS: [M+H]⁺=874

### Preparation Example 6. Preparation of Compound 6

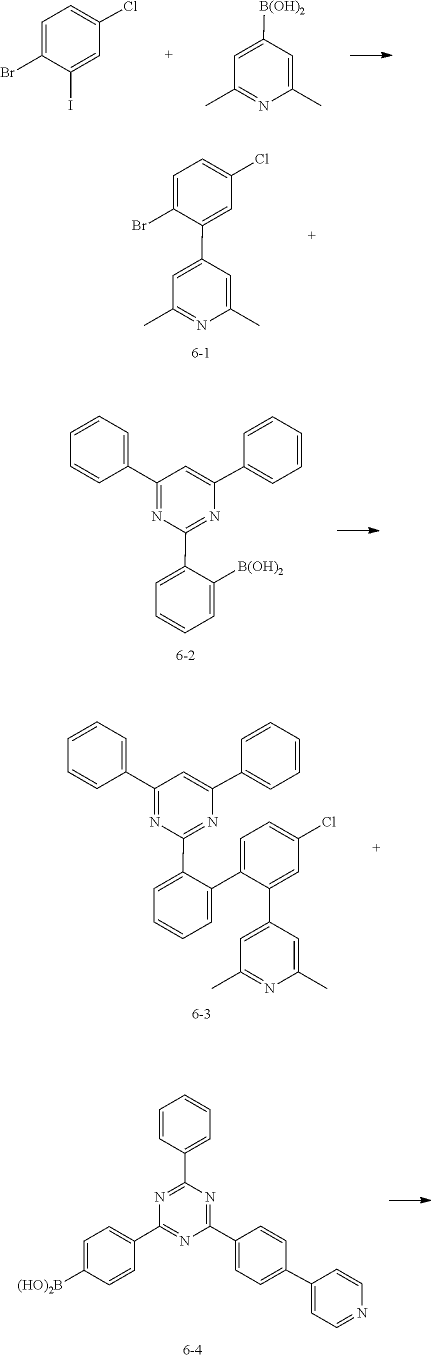

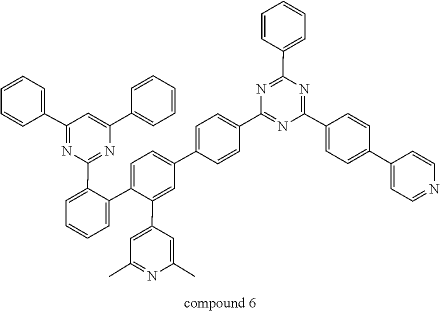 [SMILES: Cc1cc(-c2cc(-c3ccc(-c4nc(-c5ccccc5)nc(-c5ccc(-c6ccncc6)cc5)n4)cc3)ccc2-c2ccccc2-c2nc(-c3ccccc3)cc(-c3ccccc3)n2)cc(C)n1]

Compound 6 was prepared in the same manner as in the preparation method in Preparation Example 1, except that each starting material was used as in the reaction scheme.

MS: [M+H]⁺=874

### Preparation Example 7. Preparation of Compound 7

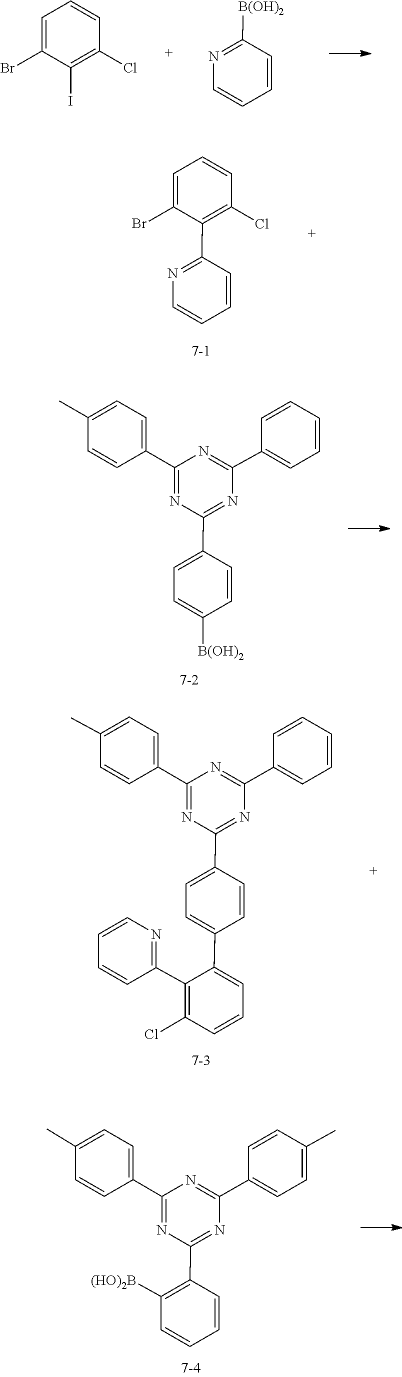

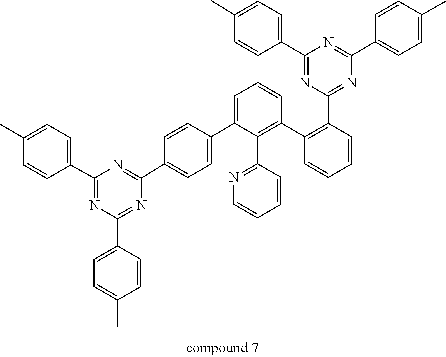

Compound 7 was prepared in the same manner as in the preparation method in Preparation Example 1, except that each starting material was used as in the reaction scheme.

MS: [M+H]⁺=826

### Preparation Example 8. Preparation of Compound 8

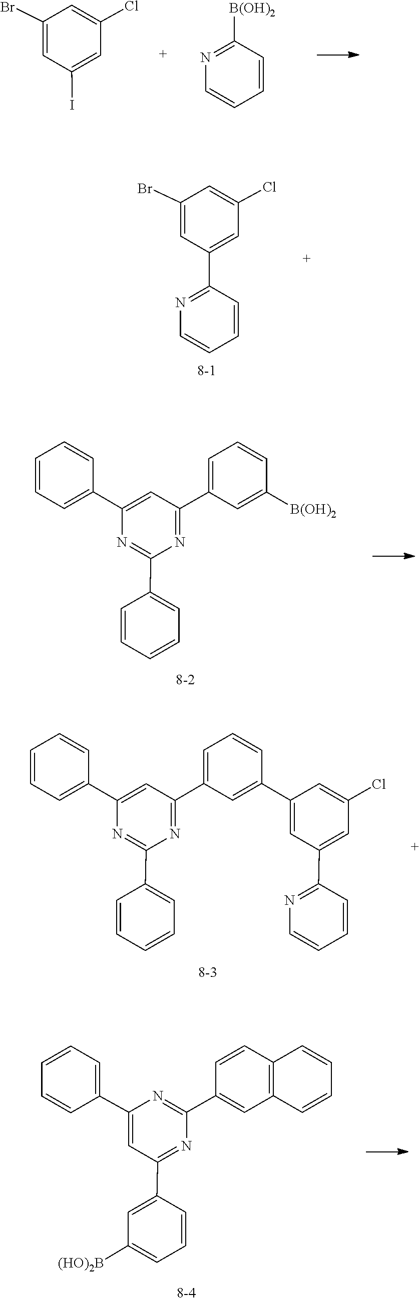

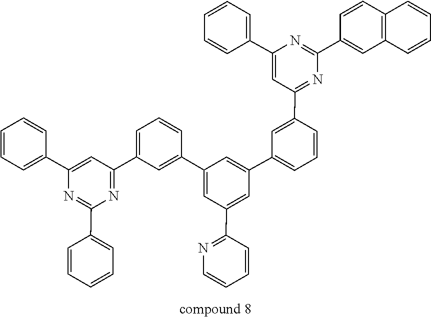 [SMILES: c1ccc(-c2cc(-c3cccc(-c4cc(-c5cccc(-c6cc(-c7ccccc7)nc(-c7ccc8ccccc8c7)n6)c5)cc(-c5ccccn5)c4)c3)nc(-c3ccccc3)n2)cc1]

Compound 8 was prepared in the same manner as in the preparation method in Preparation Example 1, except that each starting material was used as in the reaction scheme.

MS: [M+H]⁺=818

### Preparation Example 9. Preparation of Compound 9

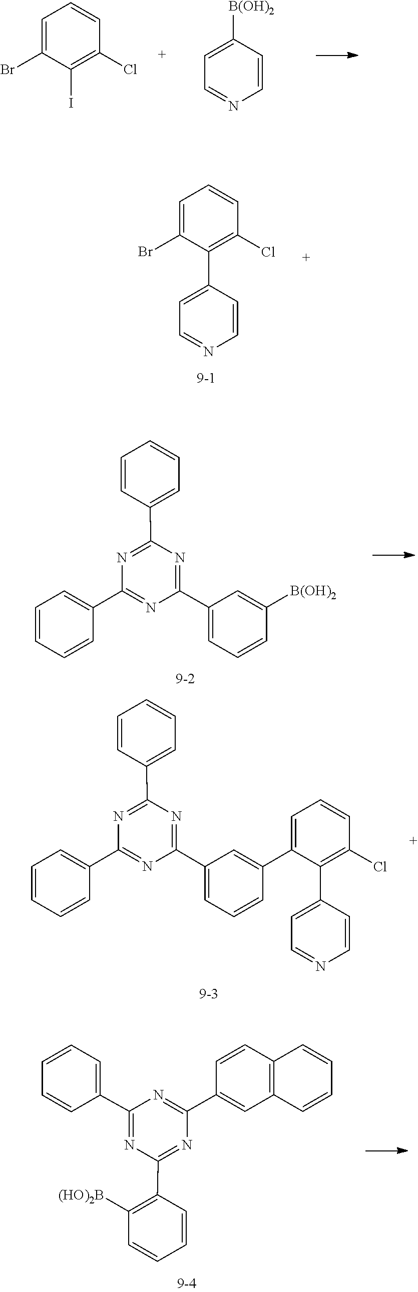

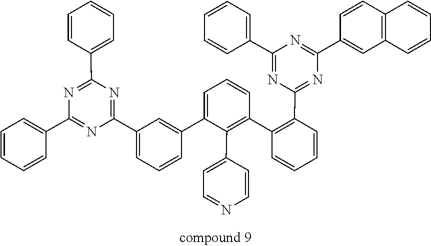 [SMILES: c1ccc(-c2nc(-c3ccccc3)nc(-c3cccc(-c4cccc(-c5ccccc5-c5nc(-c6ccccc6)nc(-c6ccc7ccccc7c6)n5)c4-c4ccncc4)c3)n2)cc1]

Compound 9 was prepared in the same manner as in the preparation method in Preparation Example 1, except that each starting material was used as in the reaction scheme.

MS: [M+H]⁺=820

### Preparation Example 10. Preparation of Compound 10

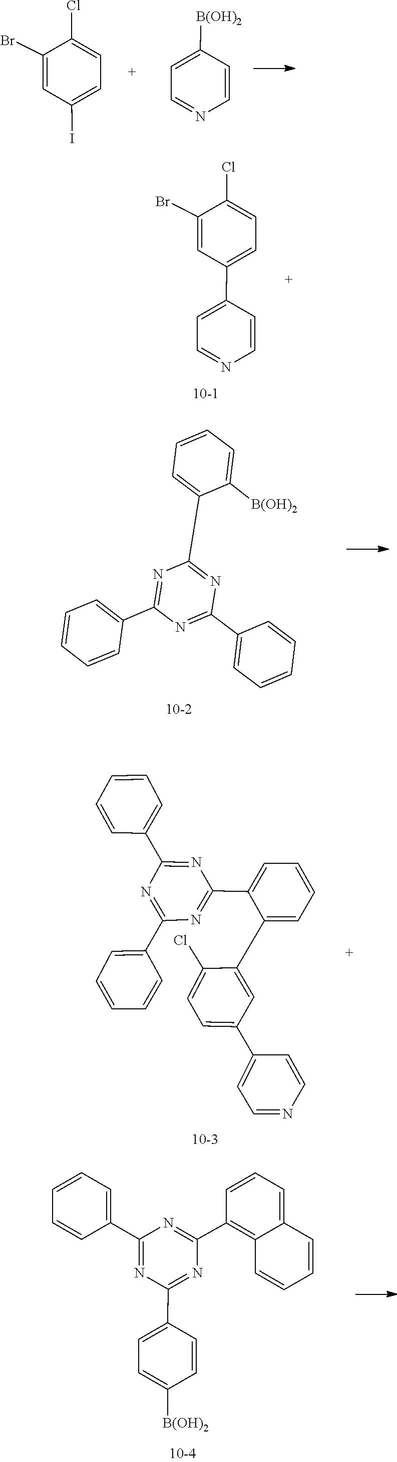 [SMILES: Clc1ccc(I)cc1Br ; OB(O)c1ccncc1 ; Clc1ccc(-c2ccncc2)cc1Br ; OB(O)c1ccccc1-c1nc(-c2ccccc2)nc(-c2ccccc2)n1 ; Clc1ccc(-c2ccncc2)cc1-c1ccccc1-c1nc(-c2ccccc2)nc(-c2ccccc2)n1 ; OB(O)c1ccc(-c2nc(-c3ccccc3)nc(-c3cccc4ccccc34)n2)cc1]

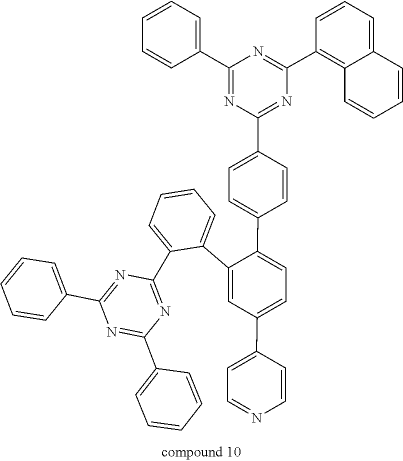 [SMILES: c1ccc(-c2nc(-c3ccccc3)nc(-c3ccccc3-c3cc(-c4ccncc4)ccc3-c3ccc(-c4nc(-c5ccccc5)nc(-c5cccc6ccccc56)n4)cc3)n2)cc1]

Compound 10 was prepared in the same manner as in the preparation method in Preparation Example 1, except that each starting material was used as in the reaction scheme.

MS: [M+H]⁺=820

### Preparation Example 11. Preparation of Compound 11

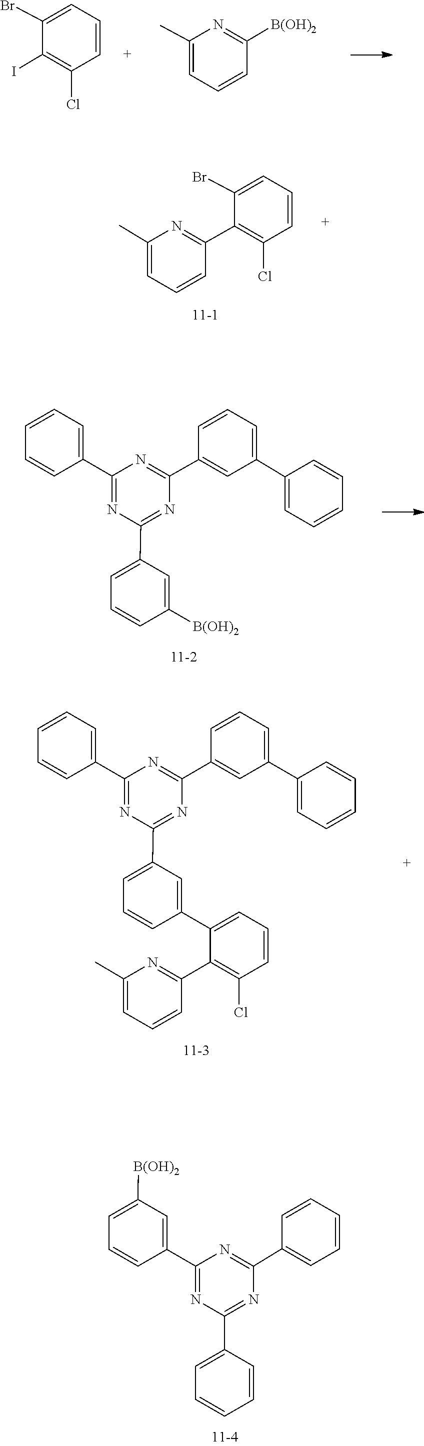

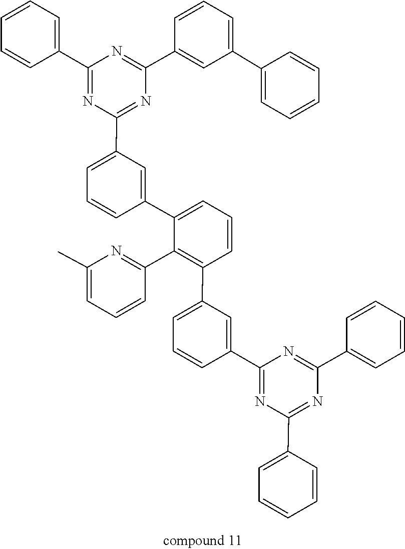 [SMILES: Cc1cccc(-c2c(-c3cccc(-c4nc(-c5ccccc5)nc(-c5ccccc5)n4)c3)cccc2-c2cccc(-c3nc(-c4ccccc4)nc(-c4cccc(-c5ccccc5)c4)n3)c2)n1]

Compound 11 was prepared in the same manner as in the preparation method in Preparation Example 1, except that each starting material was used as in the reaction scheme.

MS: [M+H]⁺=860

### Preparation Example 12. Preparation of Compound 12

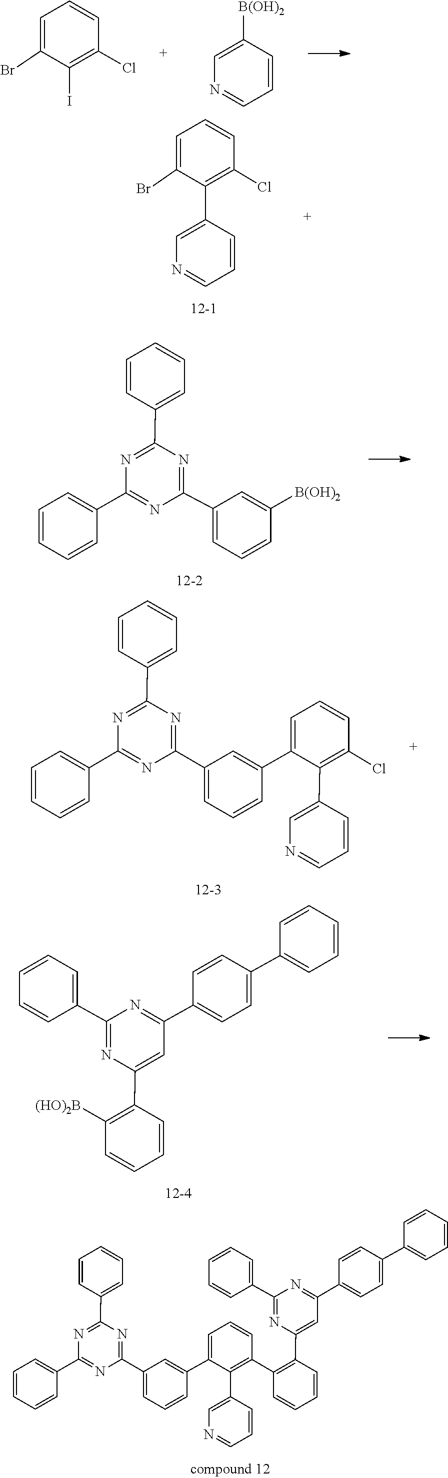

Compound 12 was prepared in the same manner as in the preparation method in Preparation Example 1, except that each starting material was used as in the reaction scheme.

MS: [M+H]⁺=845

### Preparation Example 13. Preparation of Compound 13

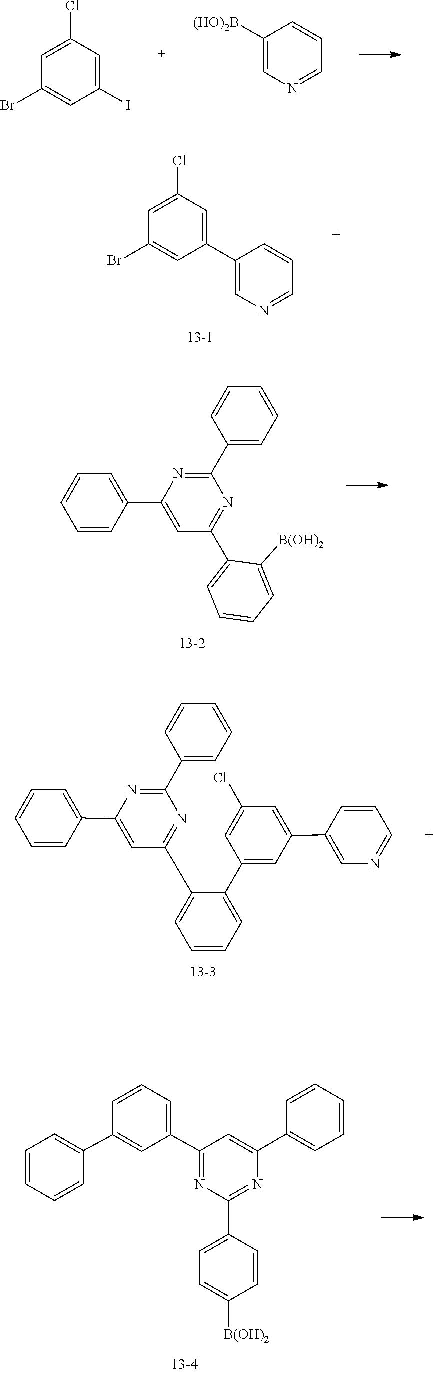 [SMILES: Clc1cc(Br)cc(I)c1 ; OB(O)c1cccnc1 ; Clc1cc(Br)cc(-c2cccnc2)c1 ; OB(O)c1ccccc1-c1cc(-c2ccccc2)nc(-c2ccccc2)n1 ; Clc1cc(-c2cccnc2)cc(-c2ccccc2-c2cc(-c3ccccc3)nc(-c3ccccc3)n2)c1 ; OB(O)c1ccc(-c2nc(-c3ccccc3)cc(-c3cccc(-c4ccccc4)c3)n2)cc1]

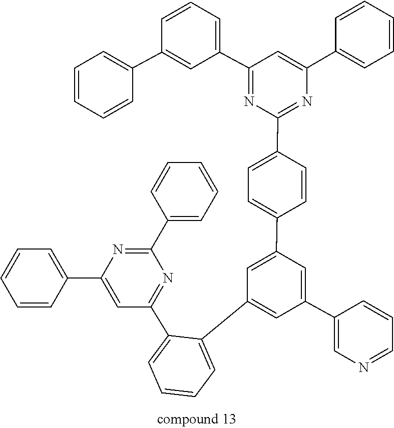 [SMILES: c1ccc(-c2cccc(-c3cc(-c4ccccc4)nc(-c4ccc(-c5cc(-c6cccnc6)cc(-c6ccccc6-c6cc(-c7ccccc7)nc(-c7ccccc7)n6)c5)cc4)n3)c2)cc1]

Compound 13 was prepared in the same manner as in the preparation method in Preparation Example 1, except that each starting material was used as in the reaction scheme.

MS: [M+H]⁺=844

### Preparation Example 14. Preparation of Compound 14

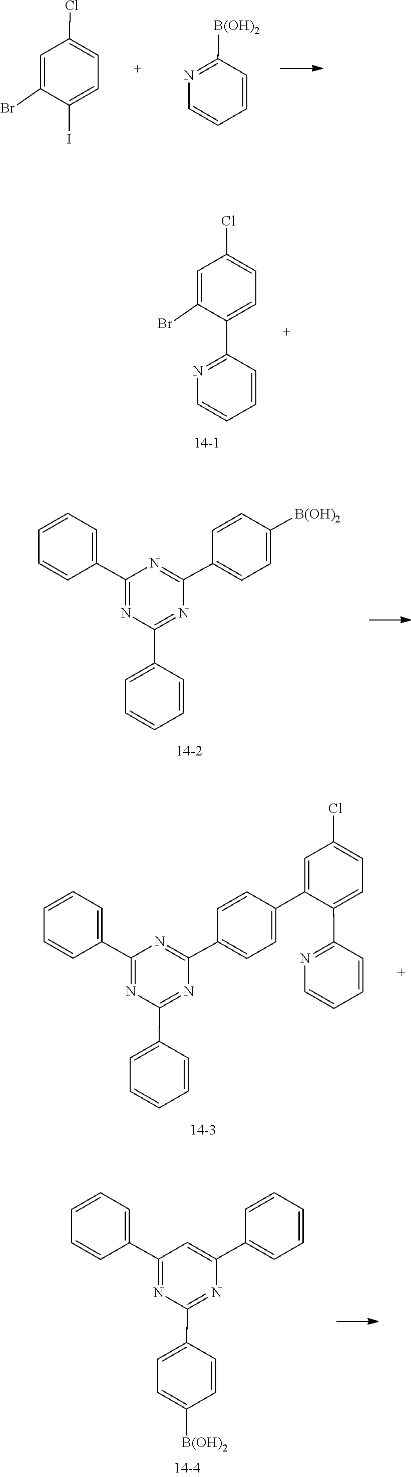 [SMILES: Clc1ccc(I)c(Br)c1 ; OB(O)c1ccccn1 ; Clc1ccc(-c2ccccn2)c(Br)c1 ; OB(O)c1ccc(-c2nc(-c3ccccc3)nc(-c3ccccc3)n2)cc1 ; Clc1ccc(-c2ccccn2)c(-c2ccc(-c3nc(-c4ccccc4)nc(-c4ccccc4)n3)cc2)c1 ; OB(O)c1ccc(-c2nc(-c3ccccc3)cc(-c3ccccc3)n2)cc1]

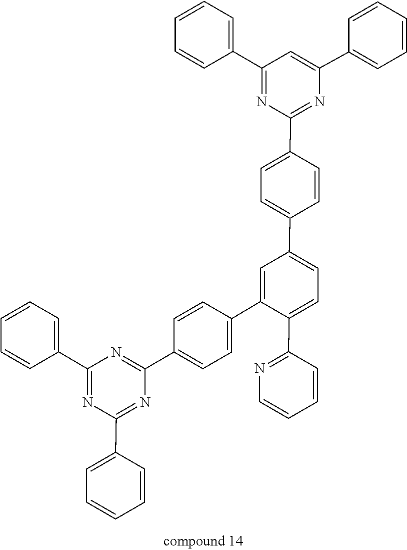 [SMILES: c1ccc(-c2cc(-c3ccccc3)nc(-c3ccc(-c4ccc(-c5ccccn5)c(-c5ccc(-c6nc(-c7ccccc7)nc(-c7ccccc7)n6)cc5)c4)cc3)n2)cc1]

Compound 14 was prepared in the same manner as in the preparation method in Preparation Example 1, except that each starting material was used as in the reaction scheme.

MS: [M+H]⁺=769

### Preparation Example 15. Preparation of Compound 15

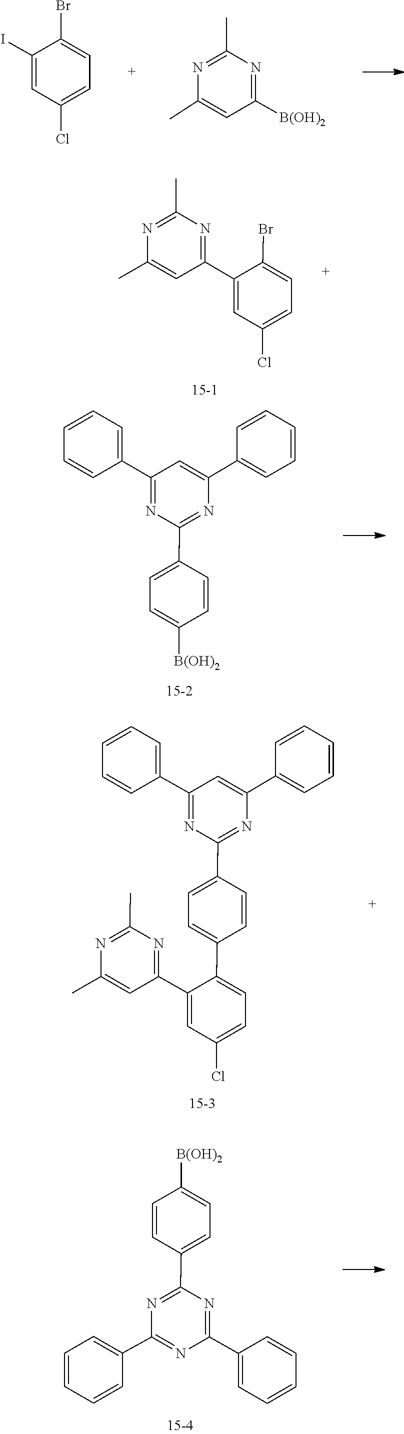 [SMILES: Clc1ccc(Br)c(I)c1 ; Cc1cc(B(O)O)nc(C)n1 ; Cc1cc(-c2cc(Cl)ccc2Br)nc(C)n1 ; OB(O)c1ccc(-c2nc(-c3ccccc3)cc(-c3ccccc3)n2)cc1 ; Cc1cc(-c2cc(Cl)ccc2-c2ccc(-c3nc(-c4ccccc4)cc(-c4ccccc4)n3)cc2)nc(C)n1 ; OB(O)c1ccc(-c2nc(-c3ccccc3)nc(-c3ccccc3)n2)cc1]

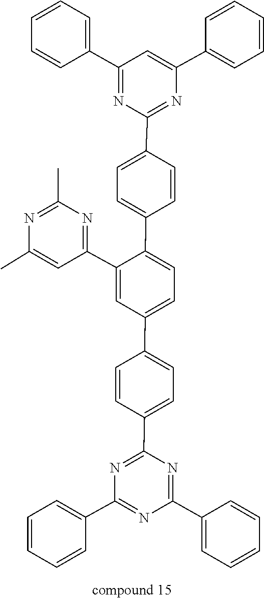 [SMILES: Cc1cc(-c2cc(-c3ccc(-c4nc(-c5ccccc5)nc(-c5ccccc5)n4)cc3)ccc2-c2ccc(-c3nc(-c4ccccc4)cc(-c4ccccc4)n3)cc2)nc(C)n1]

Compound 15 was prepared in the same manner as in the preparation method in Preparation Example 1, except that each starting material was used as in the reaction scheme.

### Preparation Example 16. Preparation of Compound 16

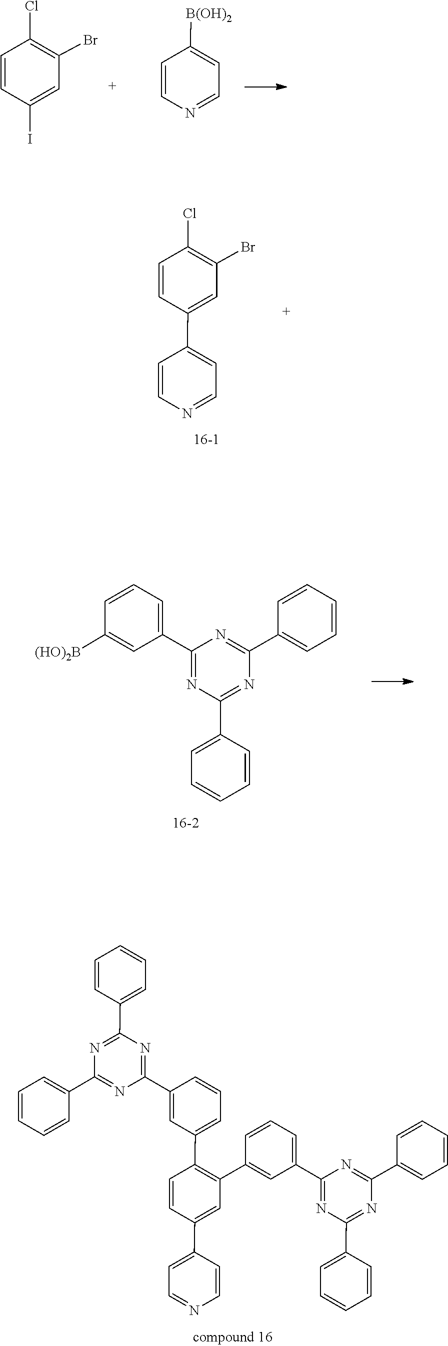 [SMILES: Clc1ccc(I)cc1Br ; OB(O)c1ccncc1 ; Clc1ccc(-c2ccncc2)cc1Br ; OB(O)c1cccc(-c2nc(-c3ccccc3)nc(-c3ccccc3)n2)c1 ; c1ccc(-c2nc(-c3ccccc3)nc(-c3cccc(-c4ccc(-c5ccncc5)cc4-c4cccc(-c5nc(-c6ccccc6)nc(-c6ccccc6)n5)c4)c3)n2)cc1]

Compound 16 was prepared in the same manner as in the preparation method in Preparation Example 1, except that each starting material was used as in the reaction scheme.

### Preparation Example 17. Preparation of Compound 17

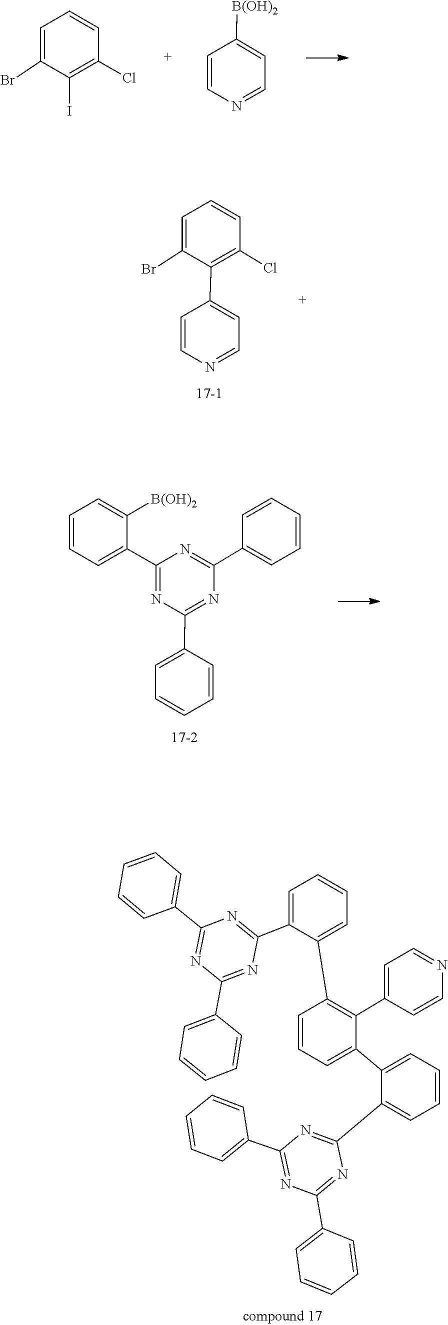 [SMILES: Clc1cccc(Br)c1I ; OB(O)c1ccncc1 ; Clc1cccc(Br)c1-c1ccncc1 ; OB(O)c1ccccc1-c1nc(-c2ccccc2)nc(-c2ccccc2)n1 ; c1ccc(-c2nc(-c3ccccc3)nc(-c3ccccc3-c3cccc(-c4ccccc4-c4nc(-c5ccccc5)nc(-c5ccccc5)n4)c3-c3ccncc3)n2)cc1]

Compound 17 was prepared in the same manner as in the preparation method in Preparation Example 1, except that each starting material was used as in the reaction scheme.

### Preparation Example 18. Preparation of Compound 18

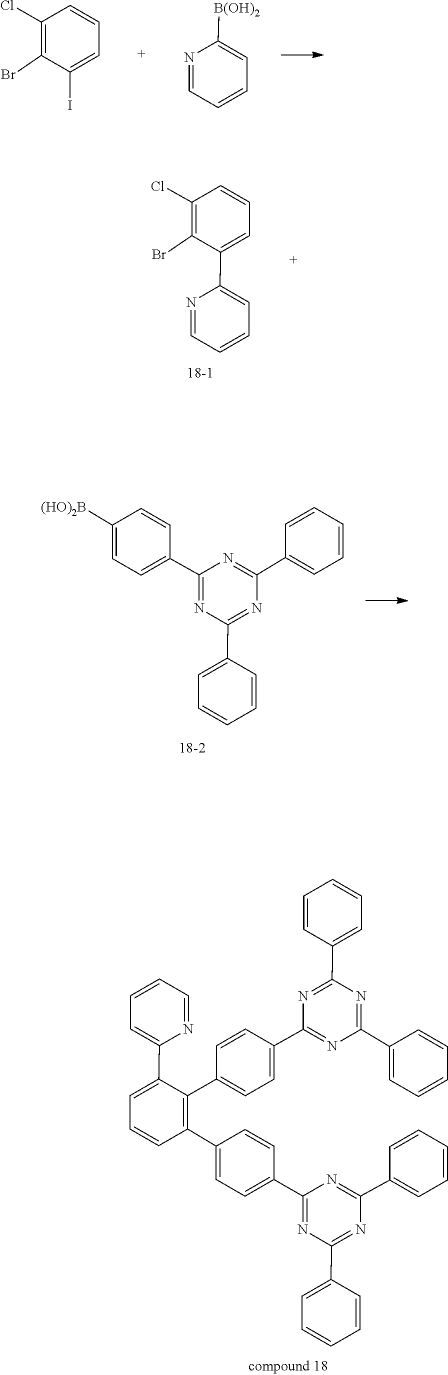 [SMILES: Clc1cccc(I)c1Br ; OB(O)c1ccccn1 ; Clc1cccc(-c2ccccn2)c1Br ; OB(O)c1ccc(-c2nc(-c3ccccc3)nc(-c3ccccc3)n2)cc1 ; c1ccc(-c2nc(-c3ccccc3)nc(-c3ccc(-c4cccc(-c5ccccn5)c4-c4ccc(-c5nc(-c6ccccc6)nc(-c6ccccc6)n5)cc4)cc3)n2)cc1]

Compound 18 was prepared in the same manner as in the preparation method in Preparation Example 1, except that each starting material was used as in the reaction scheme.

MS: [M+H]⁺=798

### Preparation Example 19. Preparation of Compound 19

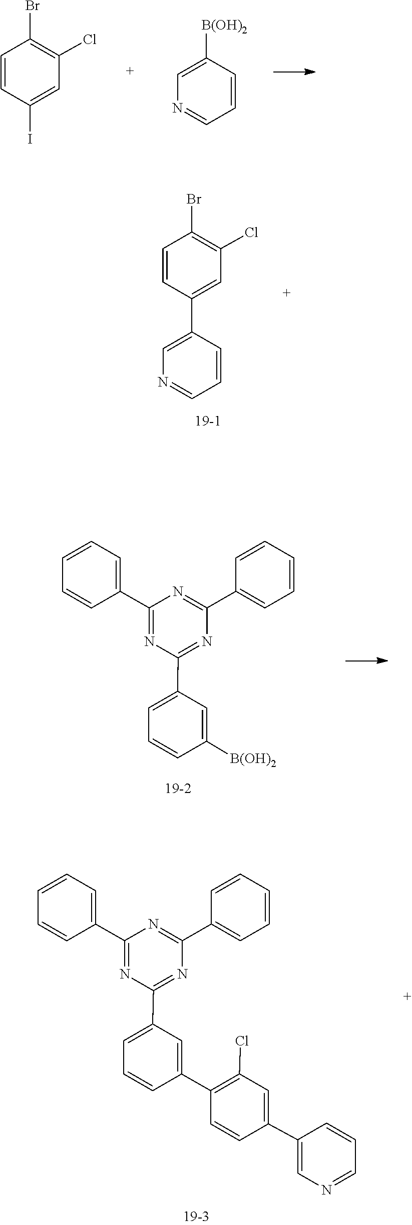 [SMILES: Clc1cc(I)ccc1Br ; OB(O)c1cccnc1 ; Clc1cc(-c2cccnc2)ccc1Br ; OB(O)c1cccc(-c2nc(-c3ccccc3)nc(-c3ccccc3)n2)c1 ; Clc1cc(-c2cccnc2)ccc1-c1cccc(-c2nc(-c3ccccc3)nc(-c3ccccc3)n2)c1]

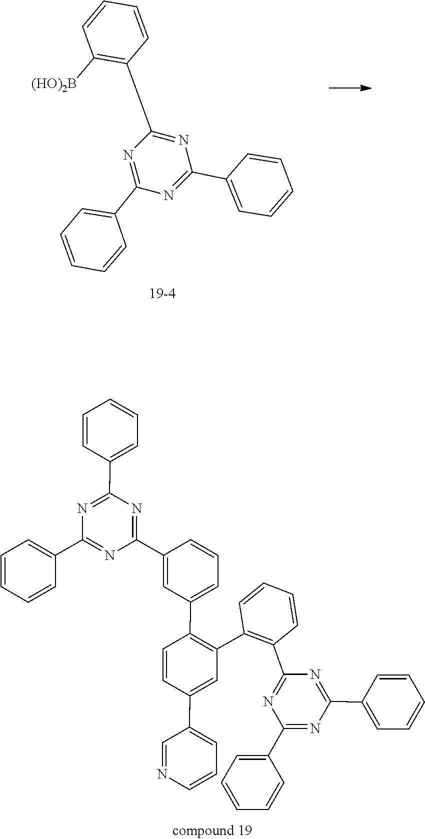 [SMILES: OB(O)c1ccccc1-c1nc(-c2ccccc2)nc(-c2ccccc2)n1 ; c1ccc(-c2nc(-c3ccccc3)nc(-c3cccc(-c4ccc(-c5cccnc5)cc4-c4ccccc4-c4nc(-c5ccccc5)nc(-c5ccccc5)n4)c3)n2)cc1]

Compound 19 was prepared in the same manner as in the preparation method in Preparation Example 1, except that each starting material was used as in the reaction scheme.

### Preparation Example 20. Preparation of Compound 20

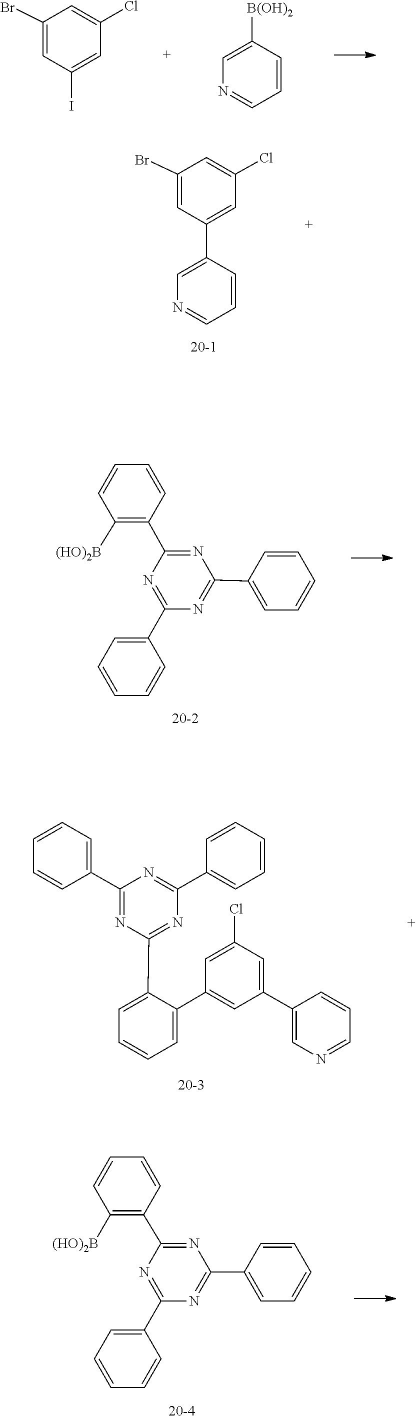 [SMILES: Clc1cc(Br)cc(I)c1 ; OB(O)c1cccnc1 ; Clc1cc(Br)cc(-c2cccnc2)c1 ; OB(O)c1ccccc1-c1nc(-c2ccccc2)nc(-c2ccccc2)n1 ; Clc1cc(-c2cccnc2)cc(-c2ccccc2-c2nc(-c3ccccc3)nc(-c3ccccc3)n2)c1 ; OB(O)c1ccccc1-c1nc(-c2ccccc2)nc(-c2ccccc2)n1]

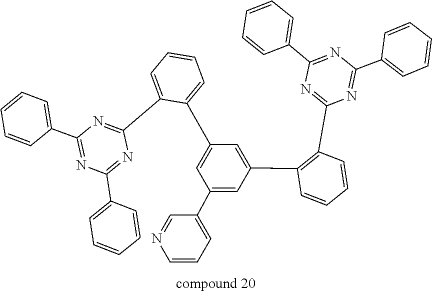 [SMILES: c1ccc(-c2nc(-c3ccccc3)nc(-c3ccccc3-c3cc(-c4cccnc4)cc(-c4ccccc4-c4nc(-c5ccccc5)nc(-c5ccccc5)n4)c3)n2)cc1]

Compound 20 was prepared in the same manner as in the preparation method in Preparation Example 1, except that each starting material was used as in the reaction scheme.

### Preparation Example 21. Preparation of Compound 21

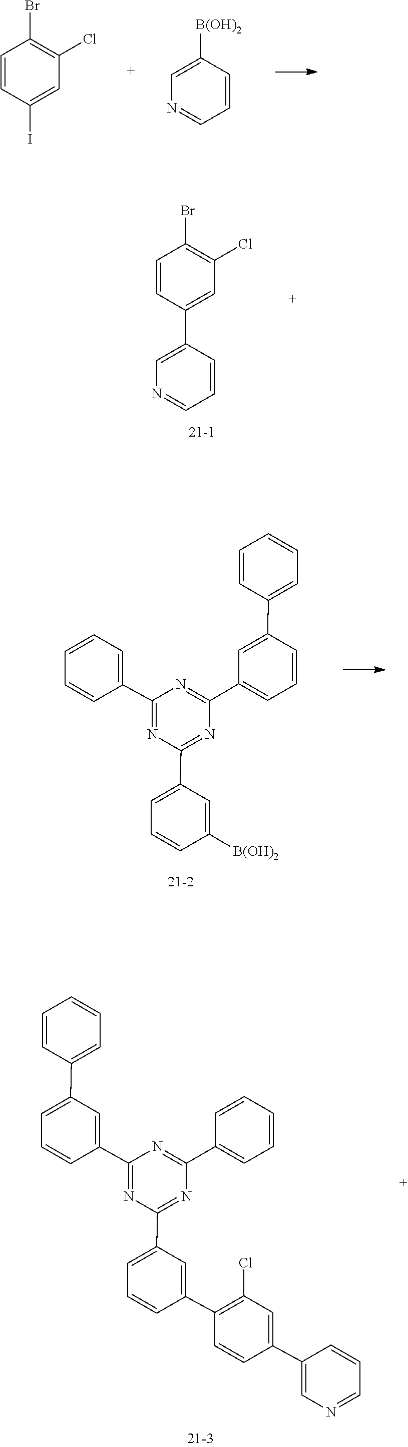 [SMILES: Clc1cc(I)ccc1Br ; OB(O)c1cccnc1 ; Clc1cc(-c2cccnc2)ccc1Br ; OB(O)c1cccc(-c2nc(-c3ccccc3)nc(-c3cccc(-c4ccccc4)c3)n2)c1 ; Clc1cc(-c2cccnc2)ccc1-c1cccc(-c2nc(-c3ccccc3)nc(-c3cccc(-c4ccccc4)c3)n2)c1]

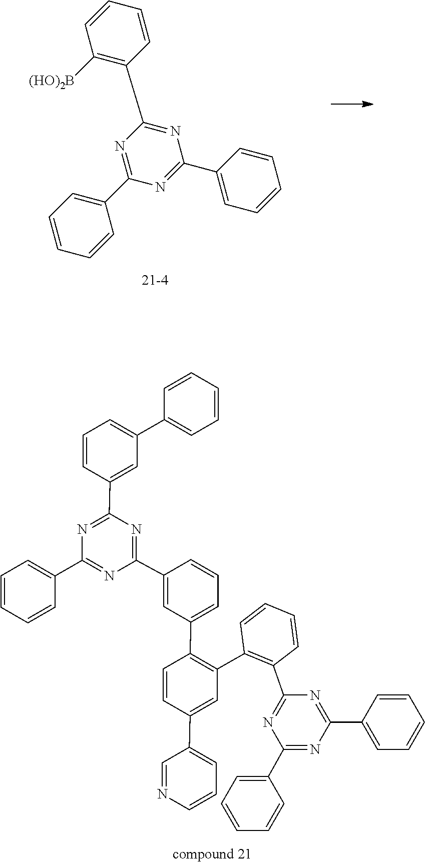 [SMILES: OB(O)c1ccccc1-c1nc(-c2ccccc2)nc(-c2ccccc2)n1 ; c1ccc(-c2cccc(-c3nc(-c4ccccc4)nc(-c4cccc(-c5ccc(-c6cccnc6)cc5-c5ccccc5-c5nc(-c6ccccc6)nc(-c6ccccc6)n5)c4)n3)c2)cc1]

Compound 21 was prepared in the same manner as in the preparation method in Preparation Example 1, except that each starting material was used as in the reaction scheme.

MS: [M+H]⁺=846

### EXAMPLES

### Example 1-1

A glass substrate thinly coated with indium tin oxide (ITO) to have a thickness of 1000 Å was put into distilled water in which a detergent was dissolved, and ultrasonically washed. In this case, a product manufactured by the Fischer Co., was used as the detergent, and distilled water twice filtered using a filter manufactured by Millipore Co., was used as the distilled water. After the ITO was washed for 30 minutes, ultrasonic washing was repeated twice by using distilled water for 10 minutes. After the washing using distilled water was completed, ultrasonic washing was conducted by using isopropyl alcohol, acetone, and methanol solvents, and the resulting product was dried and then transported to a plasma washing machine. Furthermore, the substrate was cleaned by using oxygen plasma for 5 minutes, and then was transported to a vacuum deposition machine.

The following Compound HI-A was thermally vacuum deposited to have a thickness of 600 Å on the transparent ITO electrode, which was thus prepared, thereby forming a hole injection layer. The following compound HAT and the following compound HT-A were sequentially vacuum-deposited to have a thickness of 50 Å and 60 Å, respectively, on the hole injection layer, thereby forming a first hole transport layer and a second hole transport layer.

Subsequently, the following compound BH and Compound BD were vacuum-deposited at a weight ratio of 25:1 to have a film thickness of 200 Å on the second hole transport layer, thereby forming a light emitting layer.

Compound 1 previously prepared and the following compound LiQ were vacuum-deposited at a weight ratio of 1:1 on the light emitting layer, thereby forming an electron injection and transport layer having a thickness of 350 Å. Lithium fluoride (LiF) and aluminum were sequentially deposited to have a thickness of 10 Å and 1000 Å, respectively, on the electron injection and transport layer, thereby forming a negative electrode.

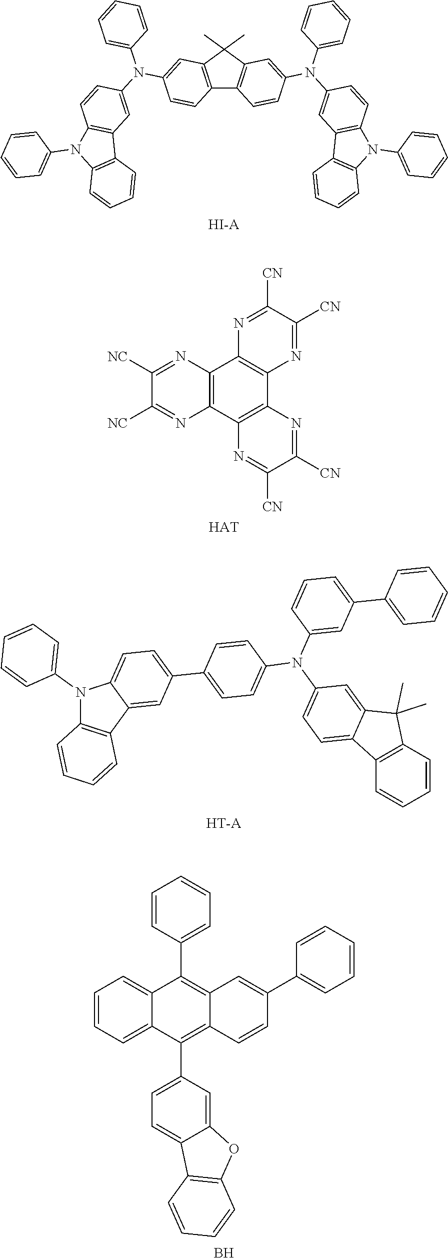

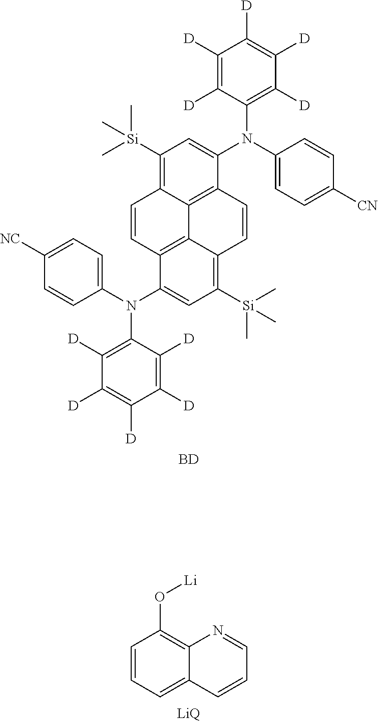

In the aforementioned procedure, the deposition rate of the organic material was maintained at 0.4 Å/sec to 0.9 Å/sec, the deposition rates of lithium fluoride and aluminum of the negative electrode were maintained at 0.3 Å/sec and at 2 Å/sec, respectively, and the degree of vacuum during the deposition was maintained at 1×10⁻⁷ to 5×10-5 torr, thereby manufacturing an organic light emitting device.

### Examples 1-2 to 1-21

Organic light emitting devices were manufactured in the same manner as in Example 1-1, except that Compounds 2 to 18 described in the following Table 1 were each used instead of Compound 1 in Example 1-1.

### Comparative Examples 1-1 to 1-7

Organic light emitting devices were manufactured in the same manner as in Example 1-1, except that Compounds ET-1 to ET-7 described in the following Table 1 were each used instead of Compound 1 in Example 1-1. The structures of Compounds ET-1 to ET-7 in the following Table 1 are as follows.

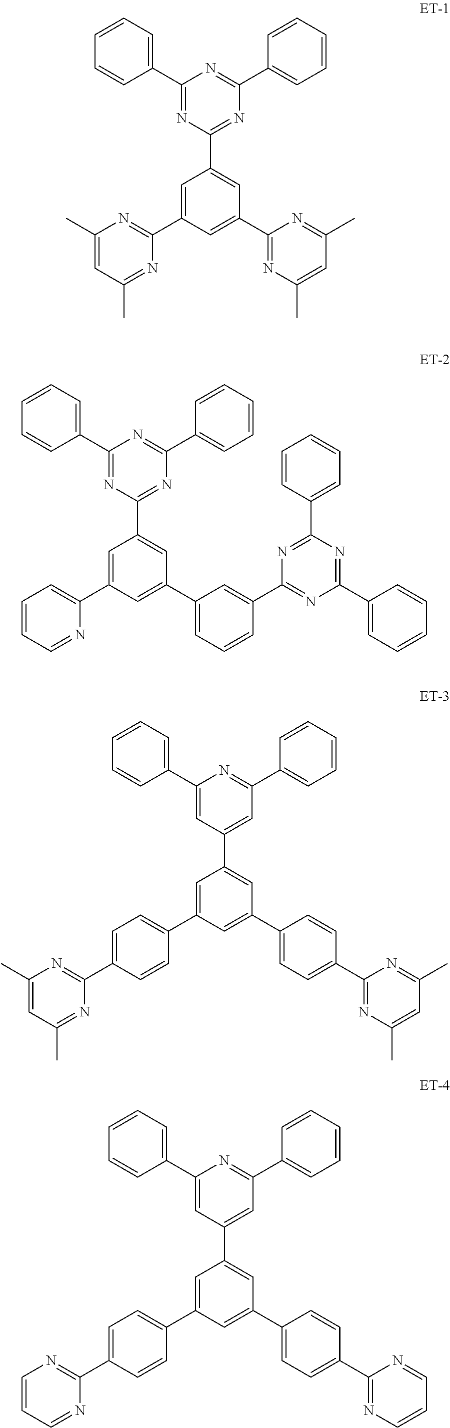

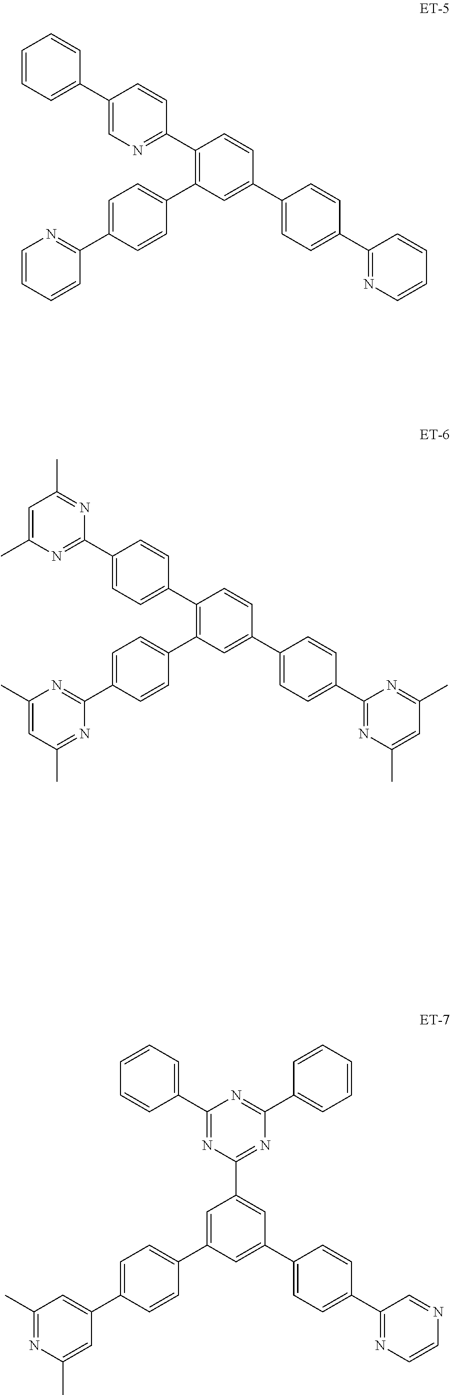

### Experimental Examples

For the organic light emitting devices manufactured in Examples 1-1 to 1-21 and Comparative Examples 1-1 to 1-7, the driving voltage and the light emitting efficiency were measured at a current density of 10 mA/cm², and a time (T90) for reaching a 90 value compared to the initial luminance was measured at a current density of 20 mA/cm². The results are shown in the following Table 1.

TABLE 1

| | | | | | Service |
|---|---|---|---|---|---|
| | | | Efficiency | Color | life (hr) |
| | | Voltage (V) | (cd/A) | coordinate | (T90 at |
| Classification | Compound | (@10 mA/cm²) | (@10 mA/cm²) | (x, y) | 20 mA/cm²) |
| Example 1-1 | 1 | 4.23 | 4.94 | (0.140, 0.092) | 181 |
| Example 1-2 | 2 | 4.19 | 5.01 | (0.140, 0.092) | 189 |
| Example 1-3 | 3 | 4.27 | 4.86 | (0.140, 0.092) | 235 |
| Example 1-4 | 4 | 4.23 | 4.97 | (0.141, 0.092) | 212 |
| Example 1-5 | 5 | 4.35 | 4.69 | (0.140, 0.093) | 220 |
| Example 1-6 | 6 | 4.29 | 4.76 | (0.140, 0.093) | 210 |
| Example 1-7 | 7 | 4.15 | 5.05 | (0.140, 0.093) | 198 |
| Example 1-8 | 8 | 4.27 | 4.90 | (0.140, 0.092) | 217 |
| Example 1-9 | 9 | 4.38 | 4.73 | (0.140, 0.092) | 201 |
| Example 1-10 | 10 | 4.43 | 4.70 | (0.140, 0.092) | 199 |
| Example 1-11 | 11 | 4.35 | 4.77 | (0.141, 0.092) | 200 |
| Example 1-12 | 12 | 4.42 | 4.65 | (0.141, 0.091) | 201 |
| Example 1-13 | 13 | 4.43 | 4.66 | (0.140, 0.094) | 200 |
| Example 1-14 | 14 | 4.51 | 4.58 | (0.140, 0.093) | 198 |
| Example 1-15 | 15 | 4.35 | 4.73 | (0.140, 0.092) | 188 |
| Example 1-16 | 16 | 4.44 | 4.77 | (0.140, 0.092) | 180 |
| Example 1-17 | 17 | 4.42 | 4.68 | (0.140, 0.092) | 170 |
| Example 1-18 | 18 | 4.52 | 4.80 | (0.140, 0.093) | 210 |
| Example 1-19 | 19 | 4.21 | 4.85 | (0.140, 0.092) | 120 |
| Example 1-20 | 20 | 4.20 | 4.71 | (0.140, 0.092) | 115 |
| Example 1-21 | 21 | 4.18 | 4.77 | (0.140, 0.092) | 120 |
| Comparative | ET-1 | 4.32 | 2.68 | (0.140, 0.092) | 100 |
| Example 1-1 | | | | | |
| Comparative | ET-2 | 4.29 | 4.18 | (0.140, 0.093) | 35 |
| Example 1-2 | | | | | |
| Comparative | ET-3 | 4.34 | 4.02 | (0.140, 0.092) | 36 |
| Example 1-3 | | | | | |
| Comparative | ET-4 | 4.35 | 4.14 | (0.141, 0.092) | 50 |
| Example 1-4 | | | | | |
| Comparative | ET-5 | 4.34 | 4.08 | (0.140, 0.093) | 55 |
| Example 1-5 | | | | | |
| Comparative | ET-6 | 4.64 | 2.42 | (0.141, 0.092) | 110 |
| Example 1-6 | | | | | |
| Comparative | ET-7 | 4.70 | 2.38 | (0.140, 0.092) | 110 |
| Example 1-7 | | | | | |

As described in Table 1, the compound represented by Chemical Formula 1 according to the present specification may be used for the electron injection and transport layer of the organic light emitting device. Since Chemical Formula 1 according to an exemplary embodiment of the present specification is a compound which has a benzene core to which a monocyclic hetero ring including one N or two N's is bonded, inevitably includes an arylene group as a linking group on both sides of benzene, and includes a monocyclic or bicyclic heteroaryl group including two or three N's as a substituent, and may increase the polarity (dipole moment) of the molecule by having electron depletion structure, electron mobility is smoothly adjusted during the manufacture of an organic light emitting device including the compound represented by Chemical Formula 1, so that Examples 1-1 to 1-21, which are an organic light emitting device including the compound represented by Chemical Formula 1, have an effect of low voltage, high efficiency, and long service life.

Specifically, it can be seen that Examples 1-1 to 1-21 are better in terms of efficiency and service life than Comparative Examples 1-1, 1-2, and 1-7 including compounds in which at least one of L1 and L2, which are linking groups, is a direct bond on both sides of benzene of Chemical Formula 1 of the present specification.

Furthermore, it can be seen that Examples 1-1 to 1-21 are better in efficiency and service life than Comparative Examples 1-3 to 1-5 including compounds in which R3 of Chemical Formula 1 of the present specification is an aryl group.

It can be seen that Examples 1-1 to 1-21 are more effective in terms of low voltage, high efficiency, and long service life than Comparative Examples 1-6 and 1-7, in which a monocyclic hetero ring including benzene and Y1 to Y5 of Chemical Formula 1 of the present specification includes a linking group.

1. A compound of the following Chemical Formula 1: 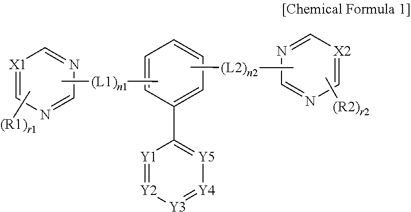
wherein, in Chemical Formula 1,
any one or two of Y1 to Y5 is or are N, and the others are CR3,
X1 is N or CR′1,
X2 is N or CR′2,
R′1 is hydrogen; deuterium; a substituted or unsubstituted alkyl group; a substituted or unsubstituted aryl group; a substituted or unsubstituted heteroaryl group; or a moiety bonded to L1, or is bonded to R1 to form a substituted or unsubstituted aromatic hydrocarbon ring,
R′2 is hydrogen; deuterium; a substituted or unsubstituted alkyl group; a substituted or unsubstituted aryl group; a substituted or unsubstituted heteroaryl group; or a moiety bonded to L2, or is boned to R2 to form a substituted or unsubstituted aromatic hydrocarbon ring,
R1 and R2 are the same as or different from each other, and are each independently a substituted or unsubstituted alkyl group; a substituted or unsubstituted aryl group; or a substituted or unsubstituted heteroaryl group, or are bonded to an adjacent group to form a substituted or unsubstituted aromatic hydrocarbon ring,
R3 is hydrogen; deuterium; or a substituted or unsubstituted alkyl group,
L1 and L2 are the same as or different from each other, and are each independently a substituted or unsubstituted arylene group,
n1 and n2 are each an integer from 1 to 5,
r1 and r2 are each an integer from 1 to 3,
when n1 is 2 or higher, two or more L1's are the same as or different from each other,
when n2 is 2 or higher, two or more L2's are the same as or different from each other,
when r1 is 2 or higher, two or more R1's are the same as or different from each other, and
when r2 is 2 or higher, two or more R2's are the same as or different from each other.

2. The compound of claim 1, wherein Chemical Formula 1 is any one of the following Chemical Formulae 1-1 to 1-7: 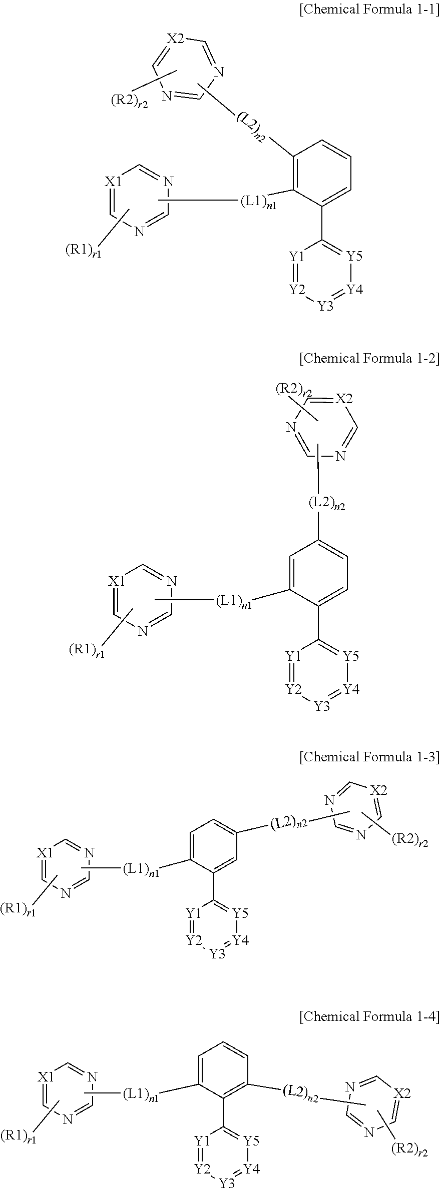 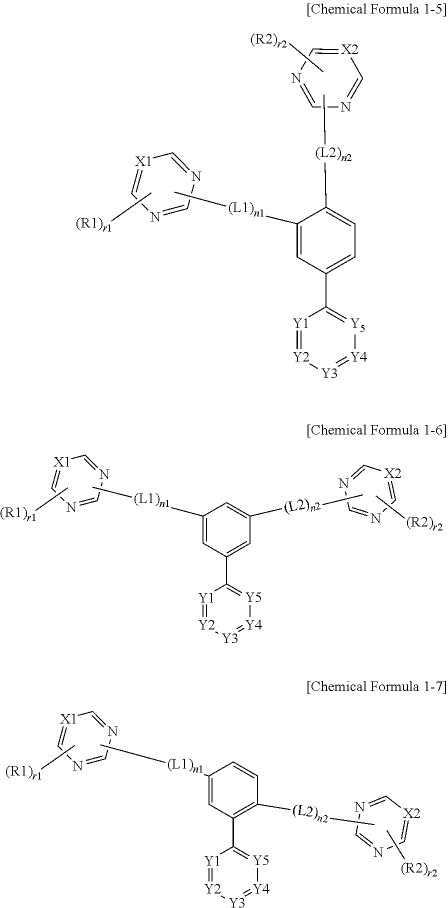
in Chemical Formulae 1-1 to 1-7,
the definitions of Y1 to Y5, X1, X2, R1, R2, r1, r2, L1, L2, n1 and n2 are the same as those defined in Chemical Formula 1.

3. The compound of claim 1, wherein Chemical Formula 1 is any one of the following Chemical Formulae 2 to 10: 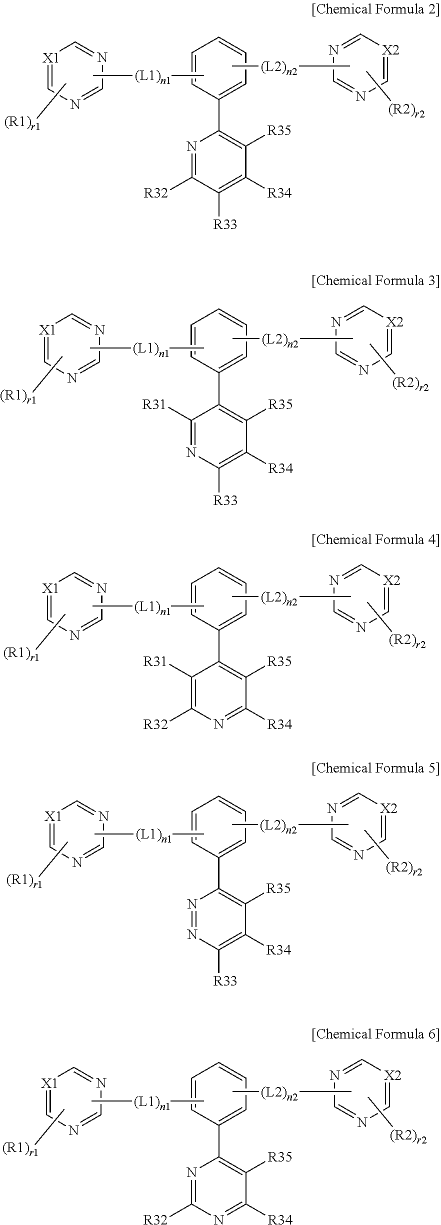 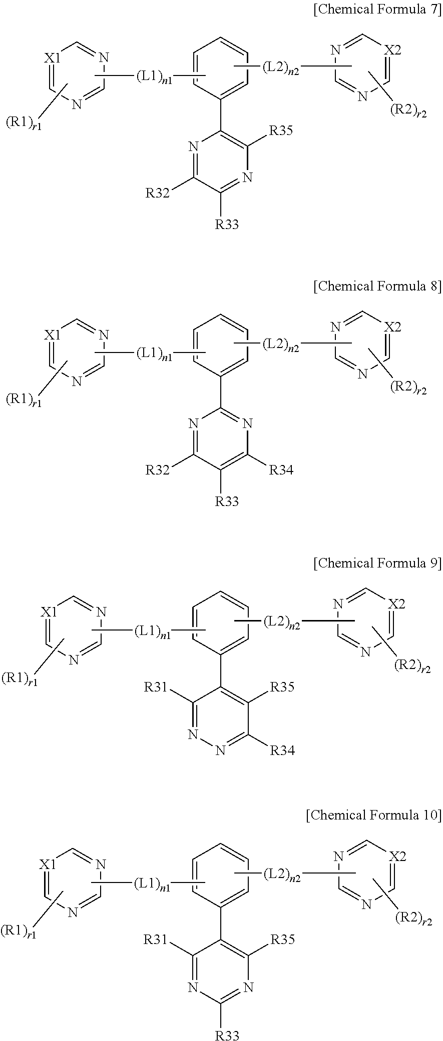
in Chemical Formulae 2 to 10,
the definitions of X1, X2, R1, R2, r1, r2, L1, L2, n1 and n2 are the same as those defined in Chemical Formula 1, and
R31 to R35 are the same as or different from each other, and are each independently hydrogen; deuterium; or a substituted or unsubstituted alkyl group.

4. The compound of claim 1, wherein 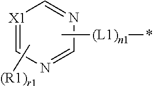
of Chemical Formula 1 is any one selected from the following structures: 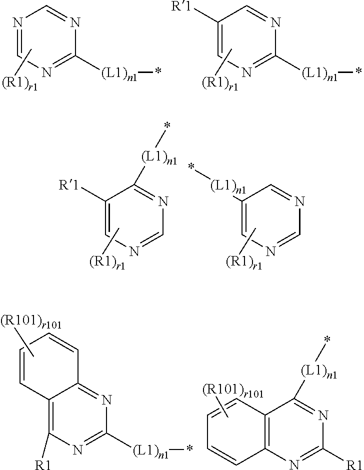
in the structures,
* is a moiety bonded to Chemical Formula 1,
the definitions of L1, n1, R1, r1 and R′1 are the same as those defined in Chemical Formula 1,
R101 is hydrogen; deuterium; a substituted or unsubstituted alkyl group; a substituted or unsubstituted aryl group; or a substituted or unsubstituted heteroaryl group,
r101 is an integer from 1 to 4, and
when r101 is 2 or higher, two or more R101's are the same as or different from each other.

5. The compound of claim 1, wherein 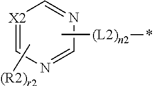
of Chemical Formula 1 is any one selected from the following structures: 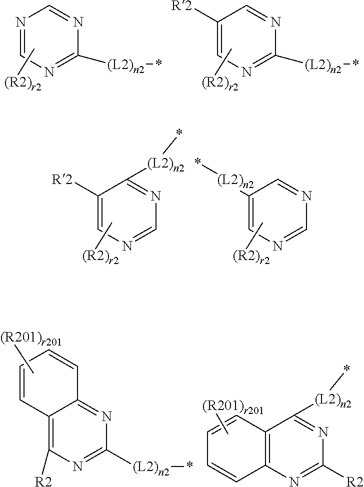
in the structures,
* is a moiety bonded to Chemical Formula 1,
the definitions of L2, n2, R2, r2 and R′2 are the same as those defined in Chemical Formula 1,
R201 is hydrogen; deuterium; a substituted or unsubstituted alkyl group; a substituted or unsubstituted aryl group; or a substituted or unsubstituted heteroaryl group,
R201 is an integer from 1 to 4, and
when r201 is 2 or higher, two or more R201's are the same as or different from each other.

6. The compound of claim 1, wherein 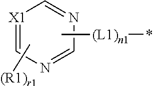
of Chemical Formula 1 are the same as each other:
* is a moiety bonded to Chemical Formula 1, and
the definitions of L2, n2, R2, r2 and R′2 are the same as those defined in Chemical Formula 1.

7. The compound of claim 1, wherein R1 and R2 are the same as or different from each other, and are each independently a straight-chained or branched alkyl group having 1 to 30 carbon atoms; a monocyclic or polycyclic aryl group having 6 to 30 carbon atoms, which is unsubstituted or substituted with a monocyclic or polycyclic heteroaryl group having 2 to 30 carbon atoms, which is unsubstituted or substituted with a straight-chained or branched alkyl group having 1 to 30 carbon atoms, a monocyclic or polycyclic cycloalkyl group having 3 to 30 carbon atoms, or a straight-chained or branched alkyl group having 1 to 30 carbon atoms; or a monocyclic or polycyclic heteroaryl group having 2 to 30 carbon atoms, which is unsubstituted or substituted with a straight-chained or branched alkyl group having 1 to 30 carbon atoms, or are bonded to an adjacent group to form a monocyclic or polycyclic aromatic hydrocarbon ring having 6 to 30 carbon atoms,
R′1 is hydrogen; deuterium; a straight-chained or branched alkyl group having 1 to 30 carbon atoms; a monocyclic or polycyclic aryl group having 6 to 30 carbon atoms; or a moiety bonded to L1, or is bonded to R1 to form a monocyclic or polycyclic aromatic hydrocarbon ring having 6 to 30 carbon atoms,
R′2 is hydrogen; deuterium; a straight-chained or branched alkyl group having 1 to 30 carbon atoms; a monocyclic or polycyclic aryl group having 6 to 30 carbon atoms; or a moiety bonded to L2, or is bonded to R2 to form a monocyclic or polycyclic aromatic hydrocarbon ring having 6 to 30 carbon atoms,
L1 and L2 are the same as or different from each other, and are each independently a monocyclic or polycyclic arylene group having 6 to 30 carbon atoms, and
R3 is hydrogen; deuterium; or a straight-chained or branched alkyl group having 1 to 30 carbon atoms.

8. The compound of claim 1, wherein Chemical Formula 1 is any one selected from the following compounds:

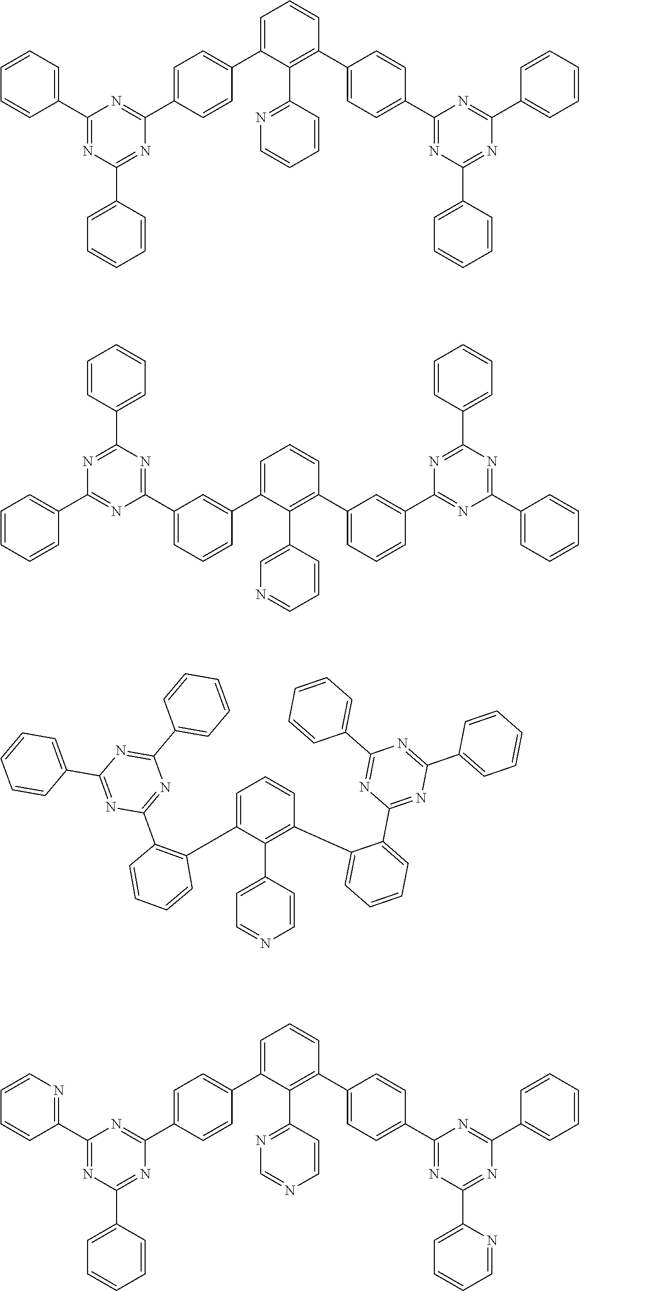 [SMILES: c1ccc(-c2nc(-c3ccccc3)nc(-c3ccc(-c4cccc(-c5ccc(-c6nc(-c7ccccc7)nc(-c7ccccc7)n6)cc5)c4-c4ccccn4)cc3)n2)cc1 ; c1ccc(-c2nc(-c3ccccc3)nc(-c3cccc(-c4cccc(-c5cccc(-c6nc(-c7ccccc7)nc(-c7ccccc7)n6)c5)c4-c4cccnc4)c3)n2)cc1 ; c1ccc(-c2nc(-c3ccccc3)nc(-c3ccccc3-c3cccc(-c4ccccc4-c4nc(-c5ccccc5)nc(-c5ccccc5)n4)c3-c3ccncc3)n2)cc1 ; c1ccc(-c2nc(-c3ccc(-c4cccc(-c5ccc(-c6nc(-c7ccccc7)nc(-c7ccccn7)n6)cc5)c4-c4ccncn4)cc3)nc(-c3ccccn3)n2)cc1]

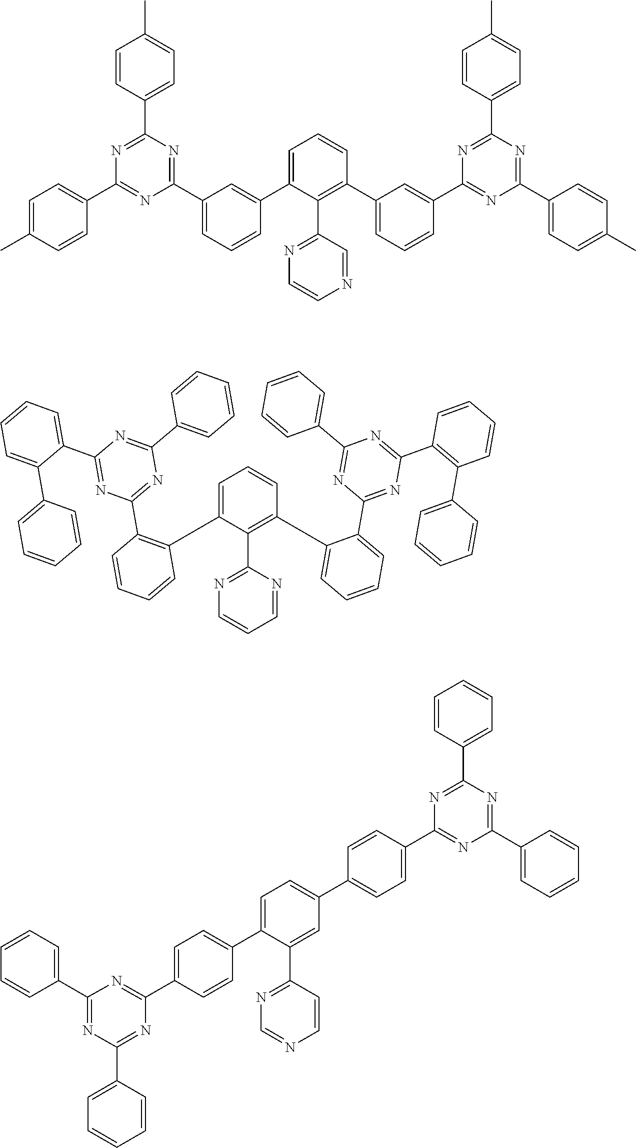

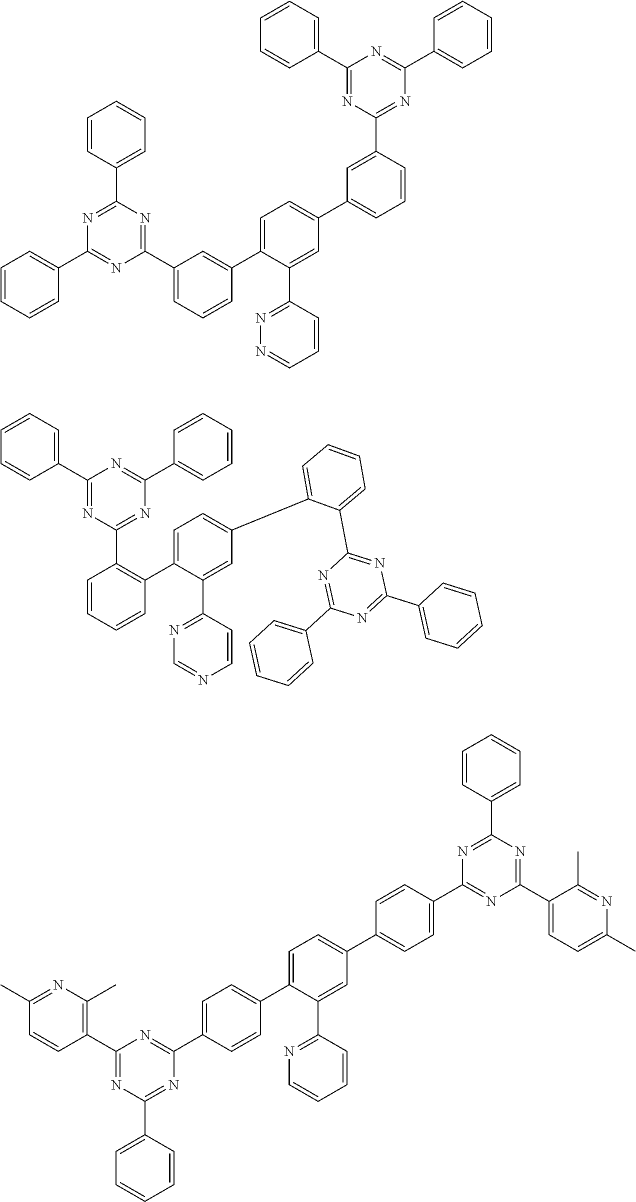

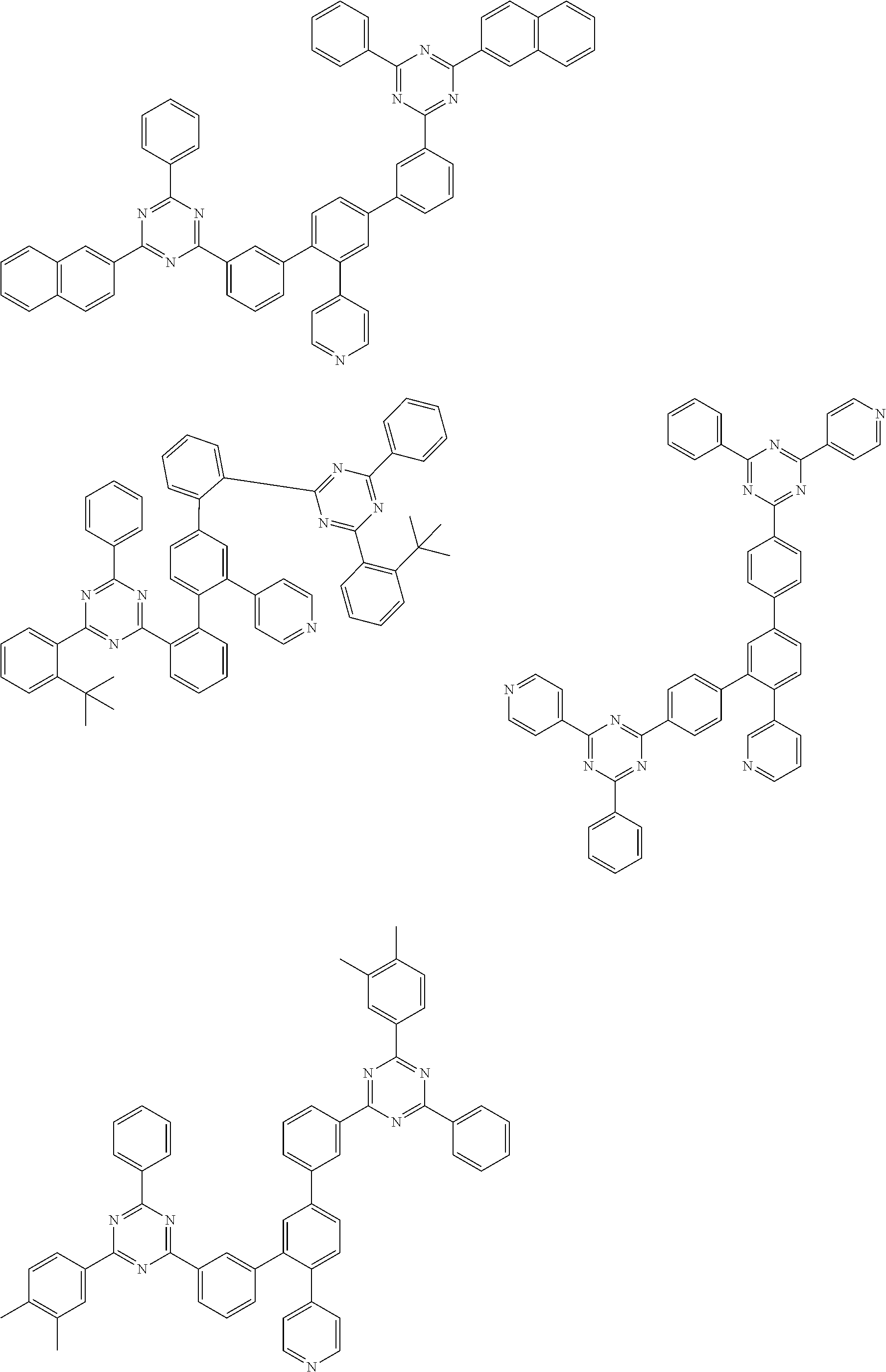 [SMILES: c1ccc(-c2nc(-c3cccc(-c4ccc(-c5cccc(-c6nc(-c7ccccc7)nc(-c7ccc8ccccc8c7)n6)c5)c(-c5ccncc5)c4)c3)nc(-c3ccc4ccccc4c3)n2)cc1 ; CC(C)(C)c1ccccc1-c1nc(-c2ccccc2)nc(-c2ccccc2-c2ccc(-c3ccccc3-c3nc(-c4ccccc4)nc(-c4ccccc4C(C)(C)C)n3)c(-c3ccncc3)c2)n1 ; c1ccc(-c2nc(-c3ccncc3)nc(-c3ccc(-c4ccc(-c5cccnc5)c(-c5ccc(-c6nc(-c7ccccc7)nc(-c7ccncc7)n6)cc5)c4)cc3)n2)cc1 ; Cc1ccc(-c2nc(-c3ccccc3)nc(-c3cccc(-c4ccc(-c5ccncc5)c(-c5cccc(-c6nc(-c7ccccc7)nc(-c7ccc(C)c(C)c7)n6)c5)c4)c3)n2)cc1C]

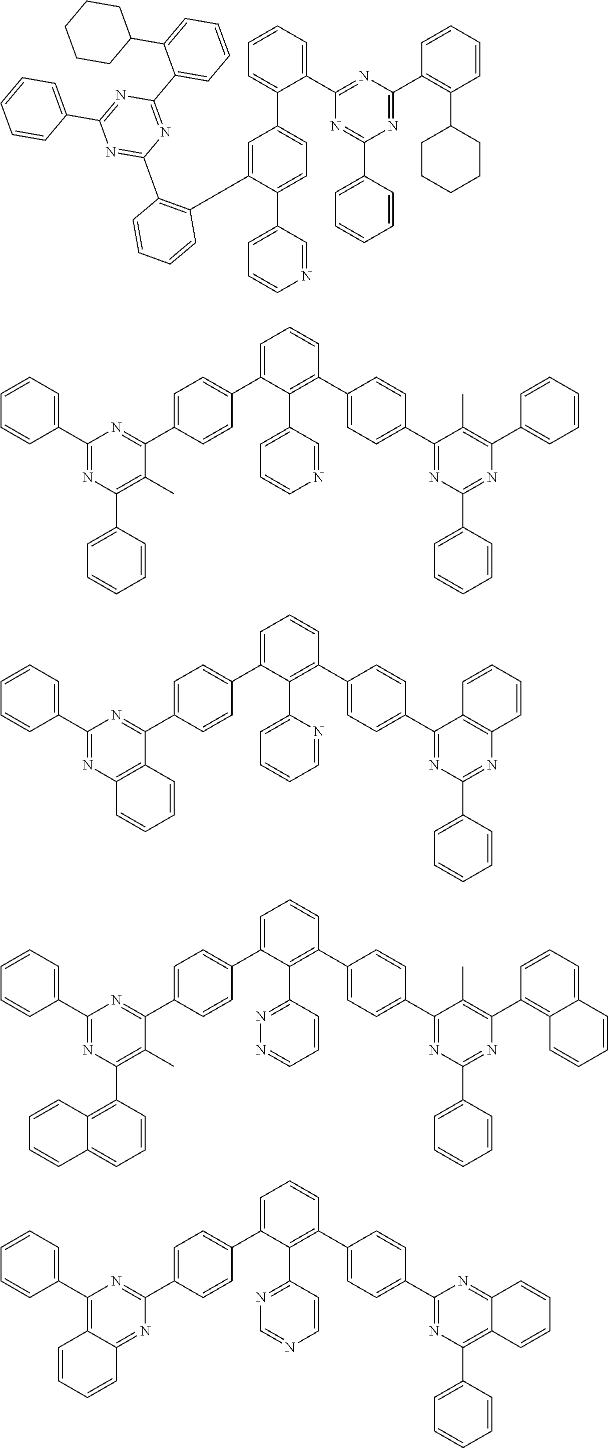

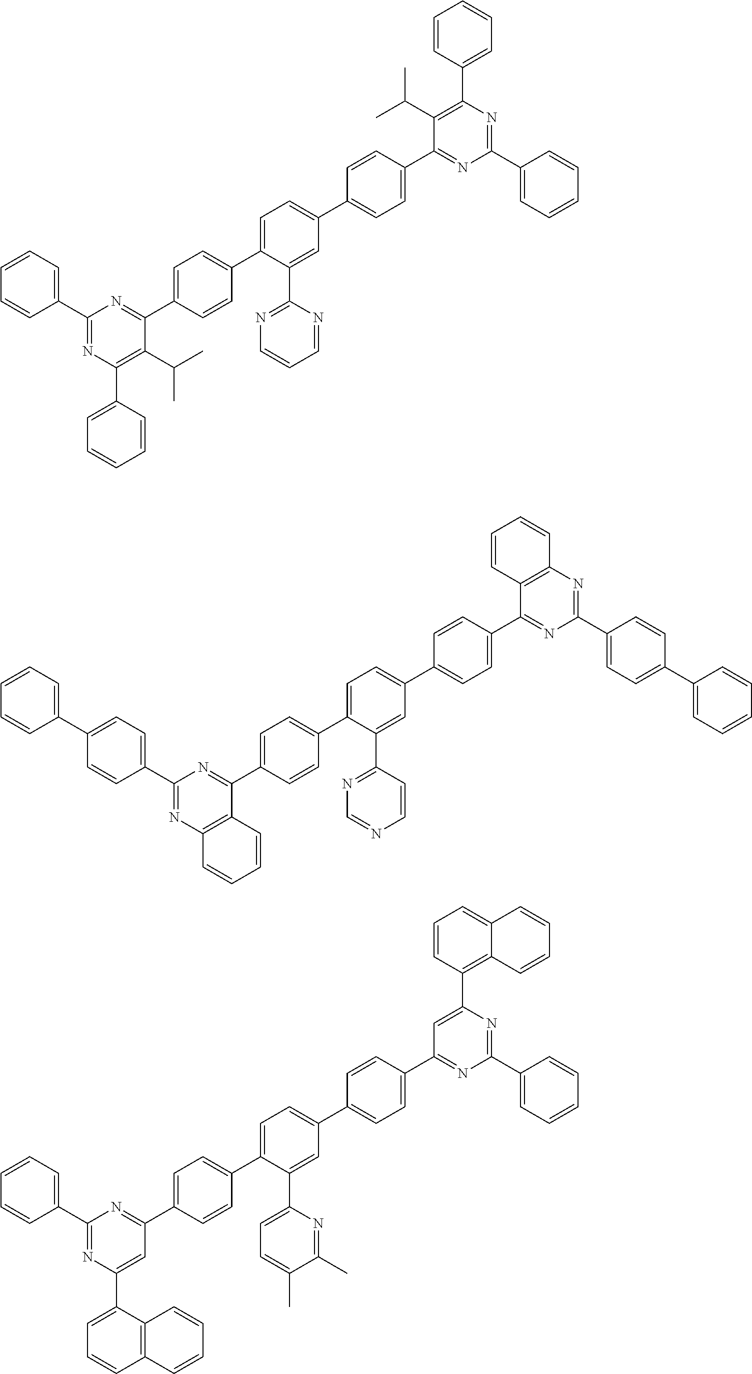 [SMILES: CC(C)c1c(-c2ccccc2)nc(-c2ccccc2)nc1-c1ccc(-c2ccc(-c3ccc(-c4nc(-c5ccccc5)nc(-c5ccccc5)c4C(C)C)cc3)c(-c3ncccn3)c2)cc1 ; c1ccc(-c2ccc(-c3nc(-c4ccc(-c5ccc(-c6ccc(-c7nc(-c8ccc(-c9ccccc9)cc8)nc8ccccc78)cc6)c(-c6ccncn6)c5)cc4)c4ccccc4n3)cc2)cc1 ; Cc1ccc(-c2cc(-c3ccc(-c4cc(-c5cccc6ccccc56)nc(-c5ccccc5)n4)cc3)ccc2-c2ccc(-c3cc(-c4cccc5ccccc45)nc(-c4ccccc4)n3)cc2)nc1C]

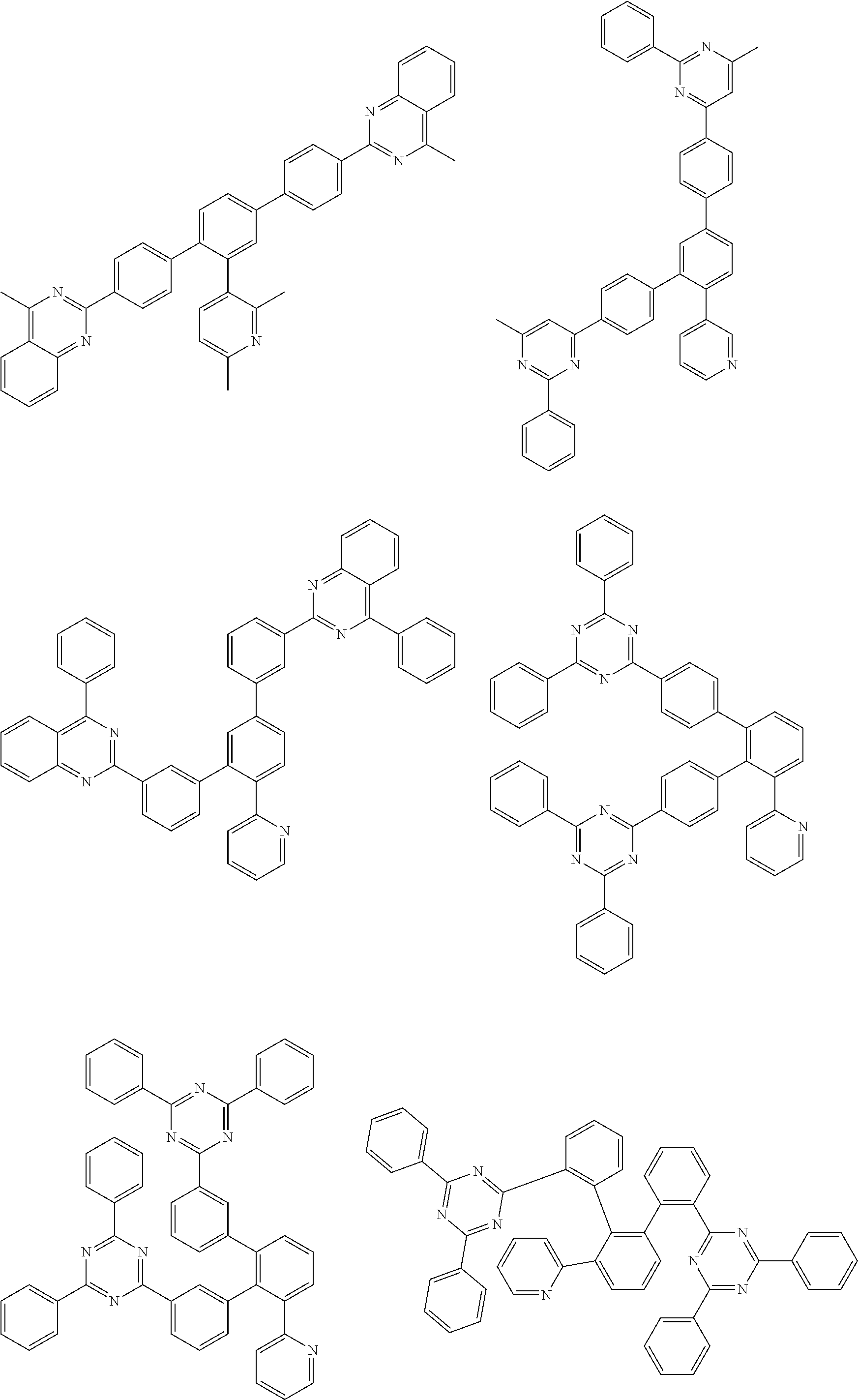

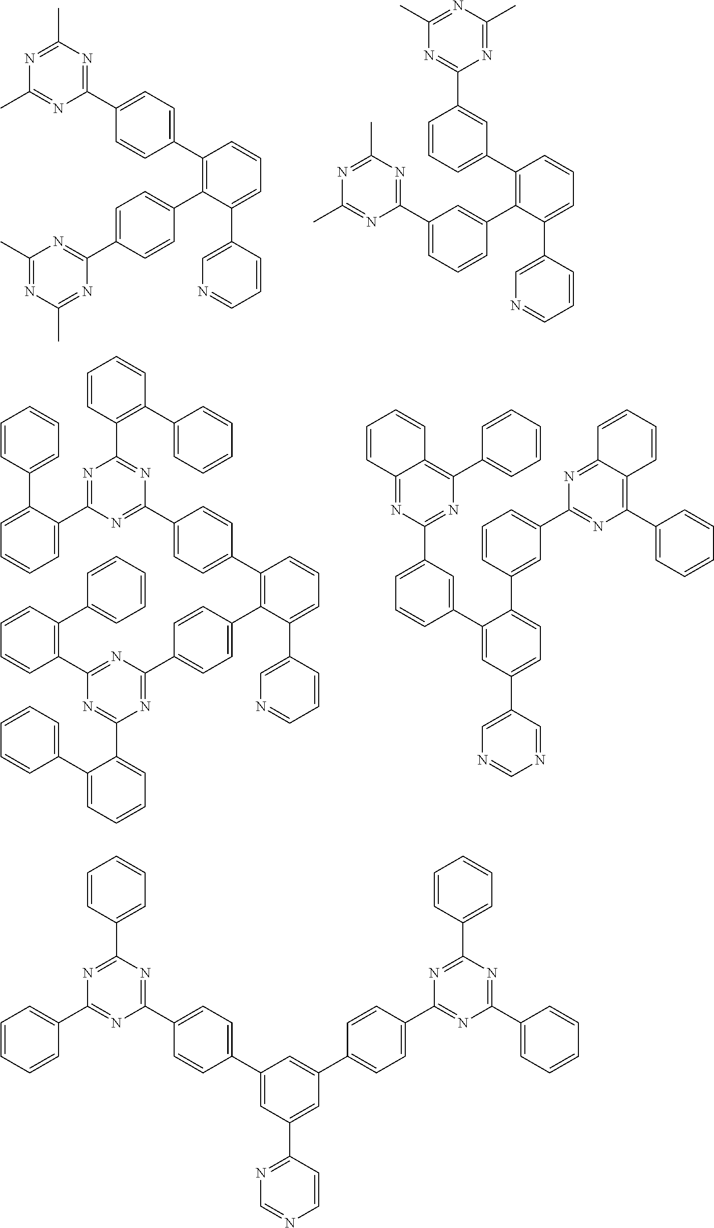

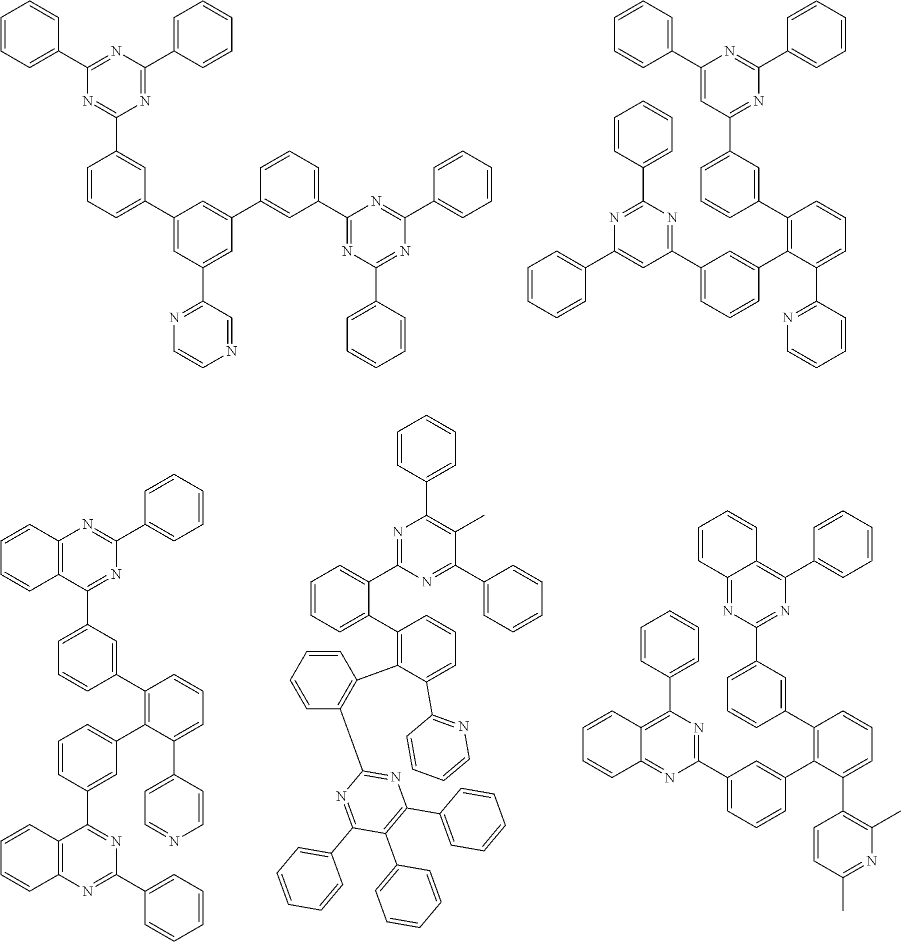

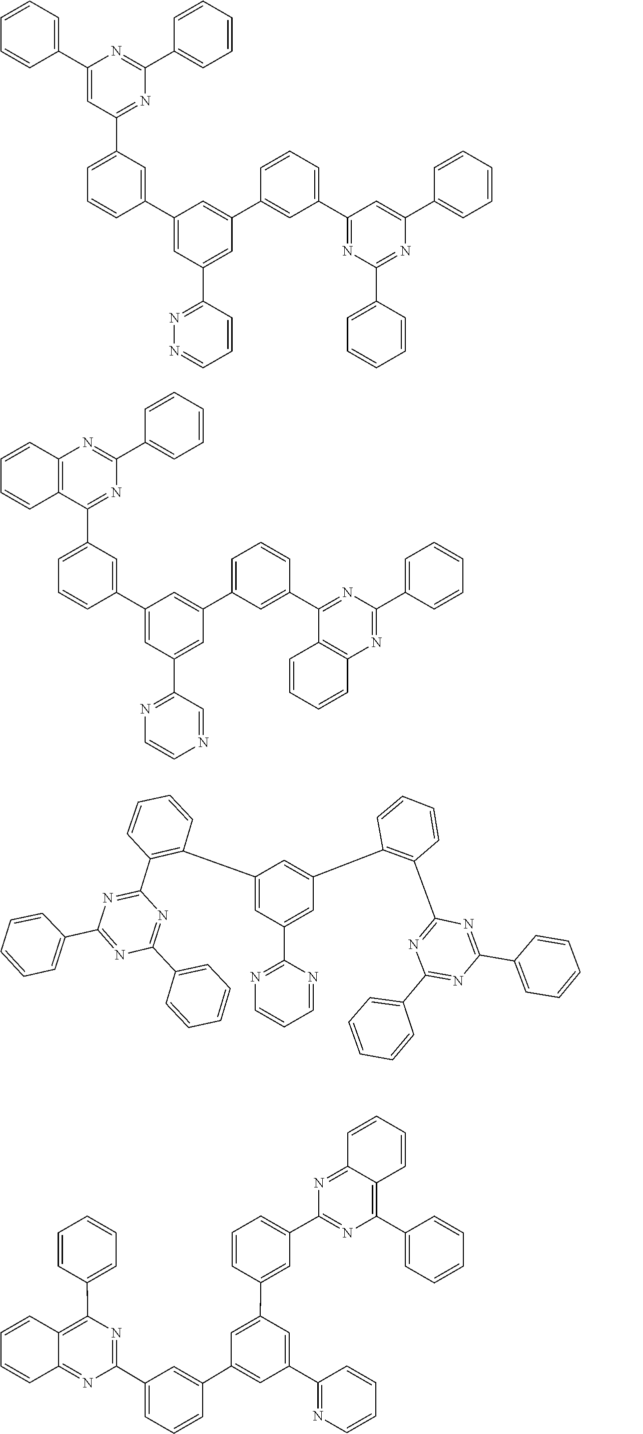

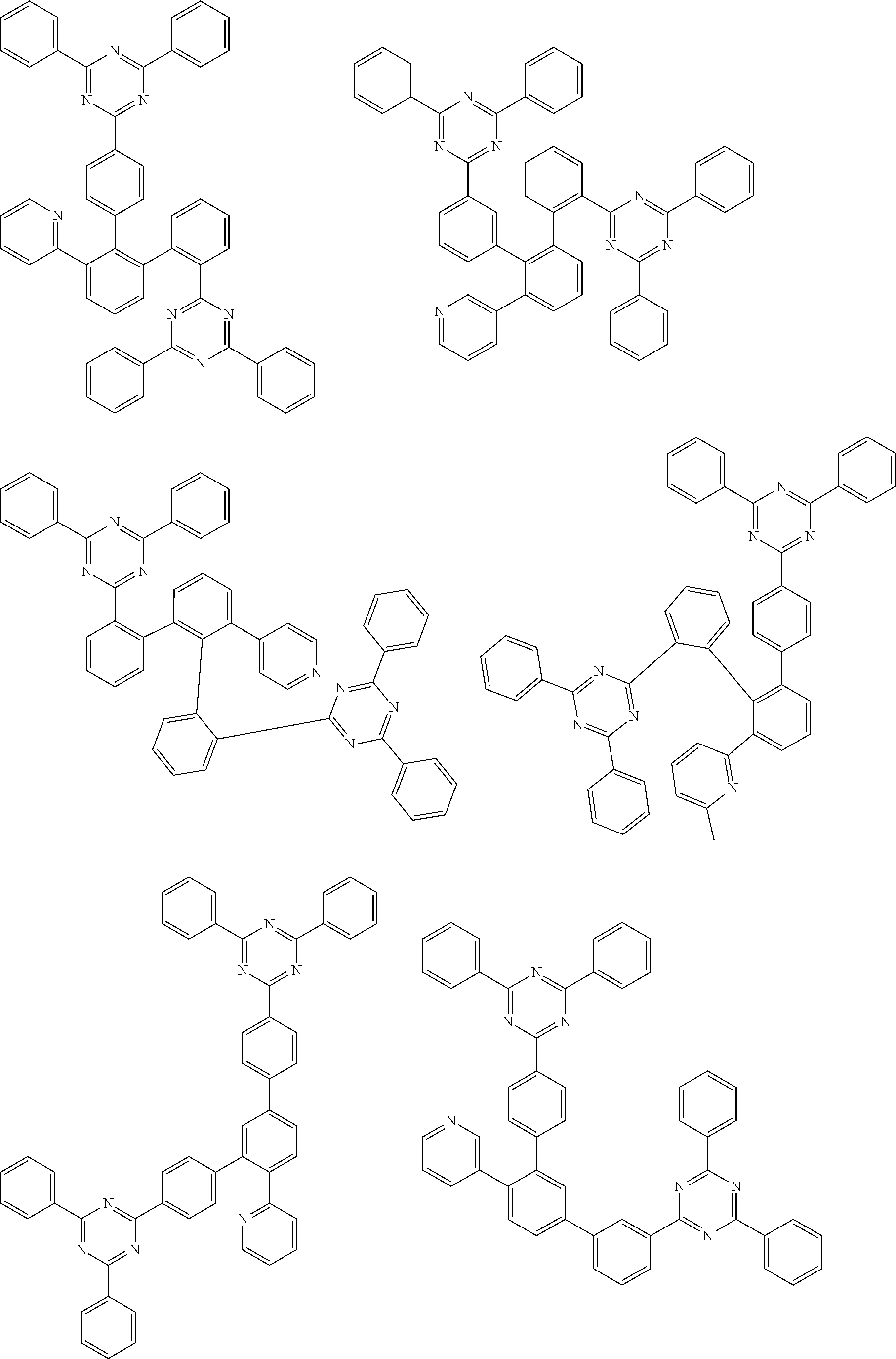

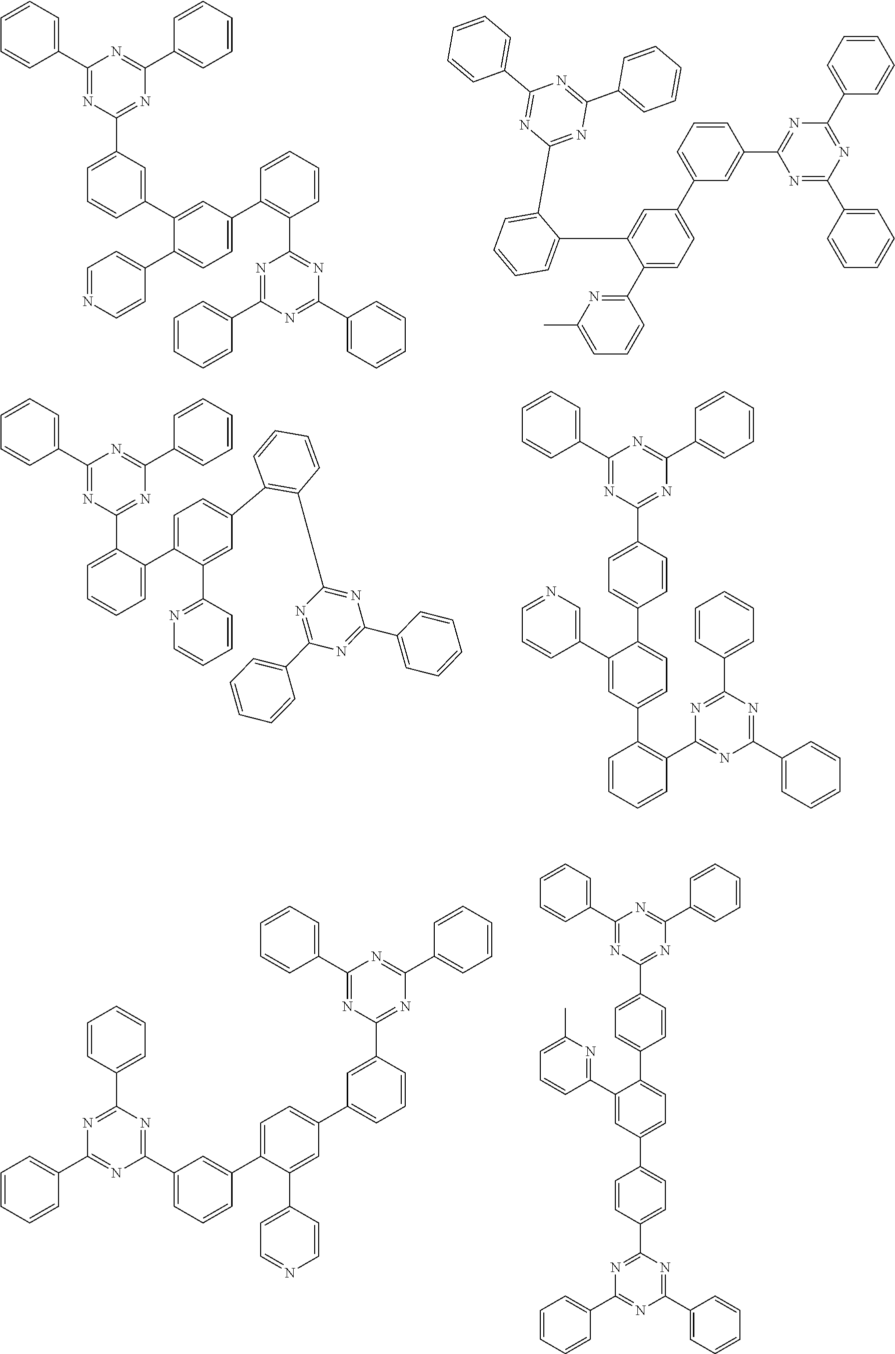

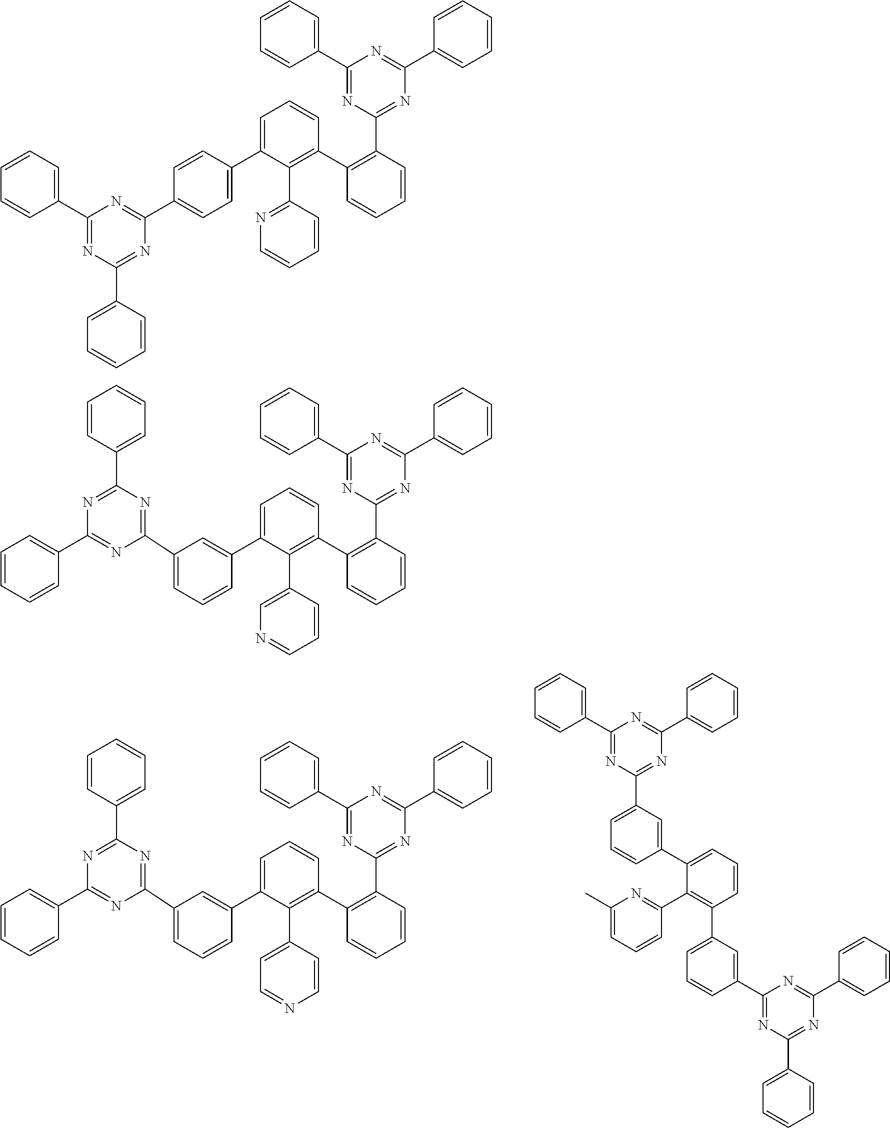

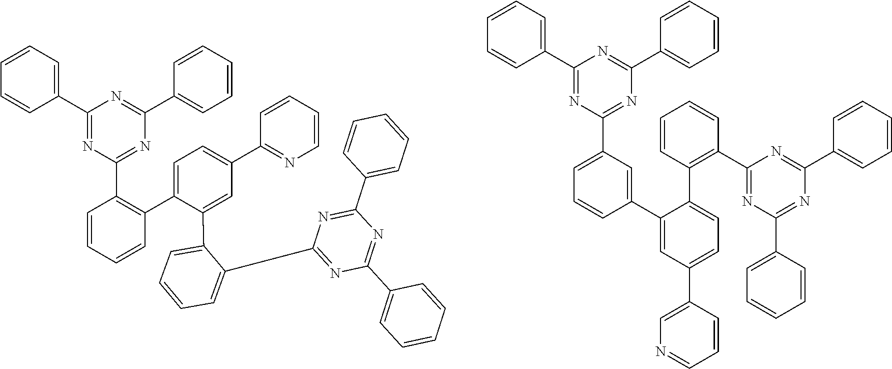 [SMILES: c1ccc(-c2nc(-c3ccccc3)nc(-c3ccccc3-c3ccc(-c4ccccn4)cc3-c3ccccc3-c3nc(-c4ccccc4)nc(-c4ccccc4)n3)n2)cc1 ; c1ccc(-c2nc(-c3ccccc3)nc(-c3cccc(-c4cc(-c5cccnc5)ccc4-c4ccccc4-c4nc(-c5ccccc5)nc(-c5ccccc5)n4)c3)n2)cc1]

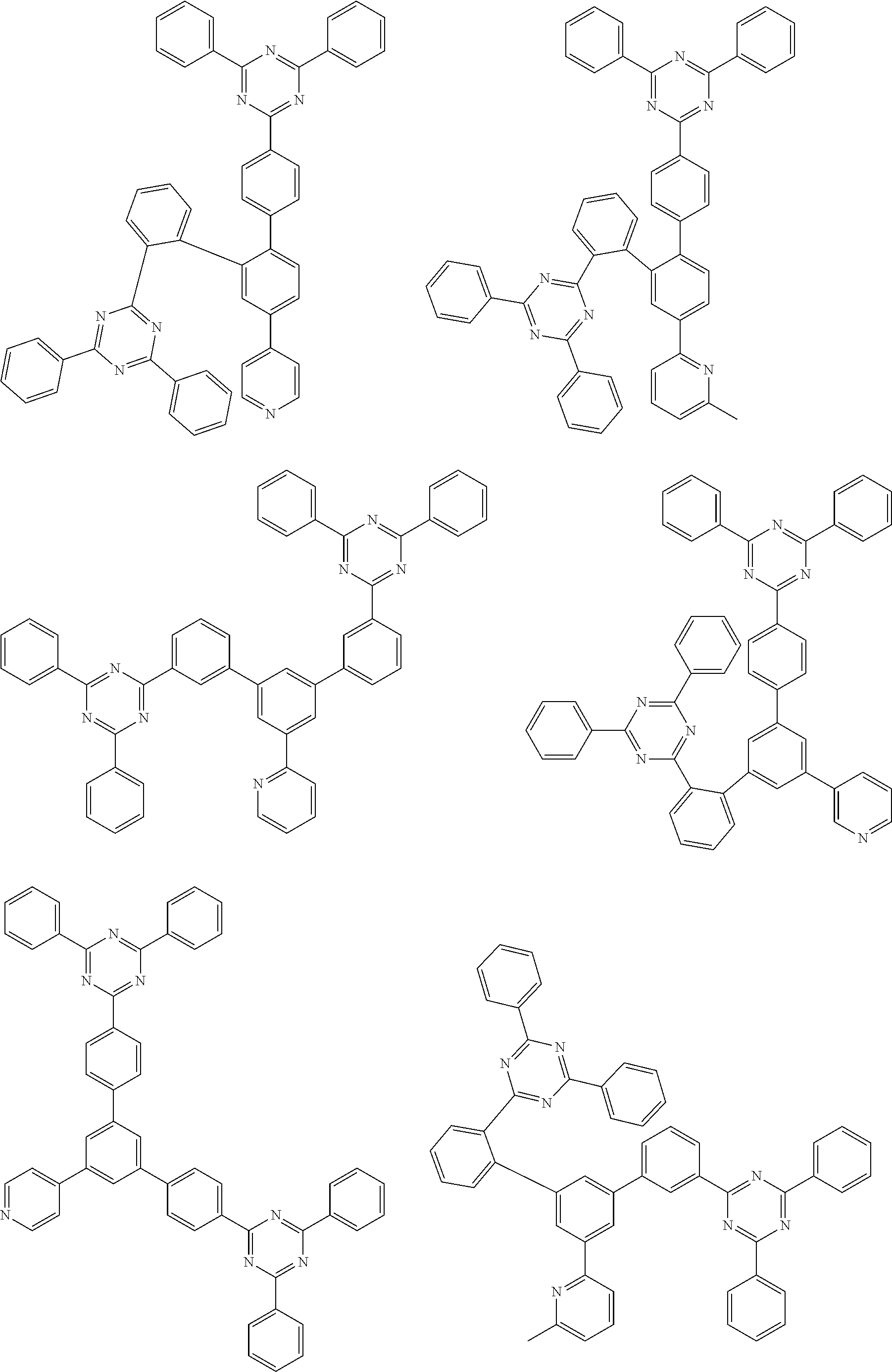

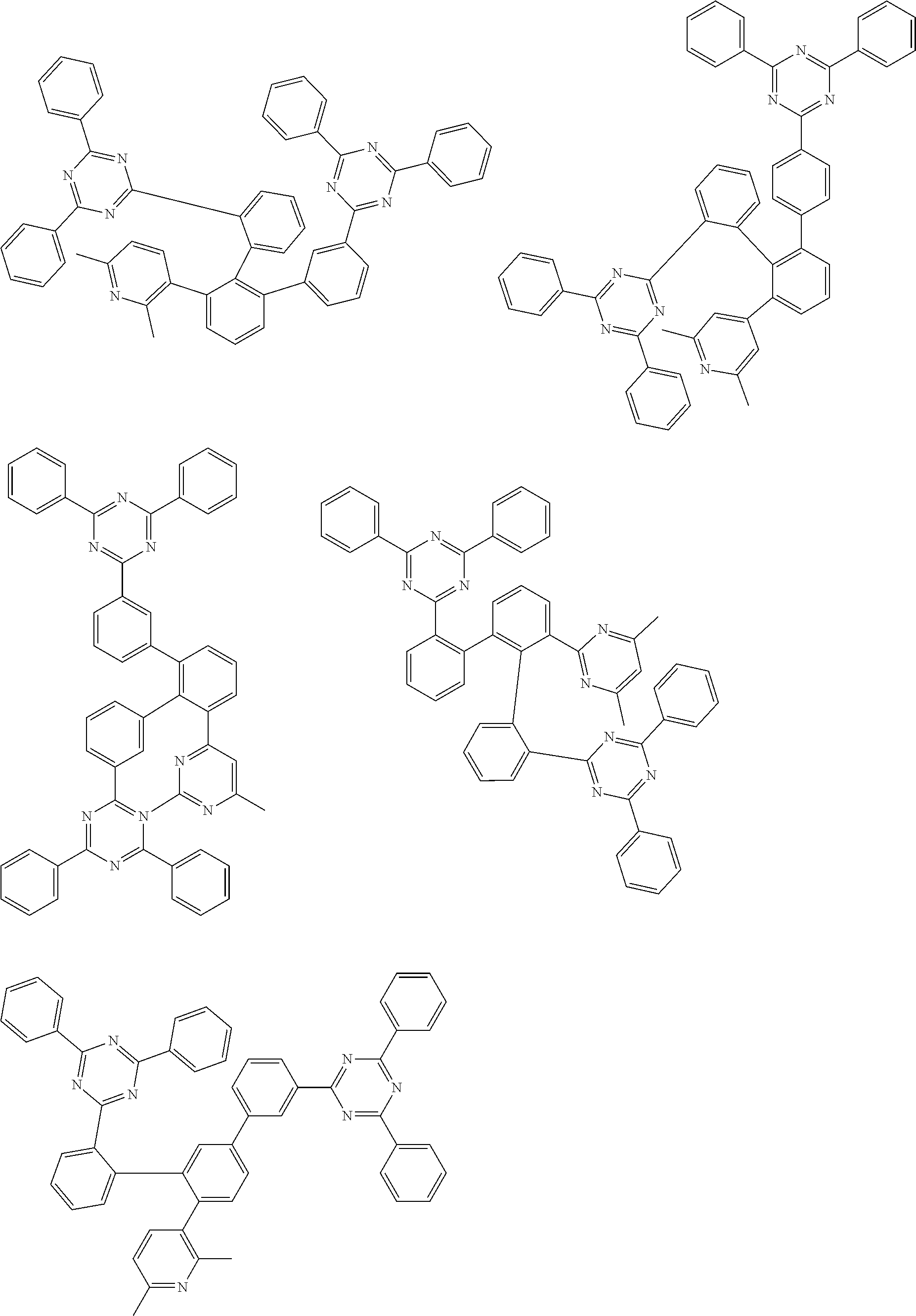

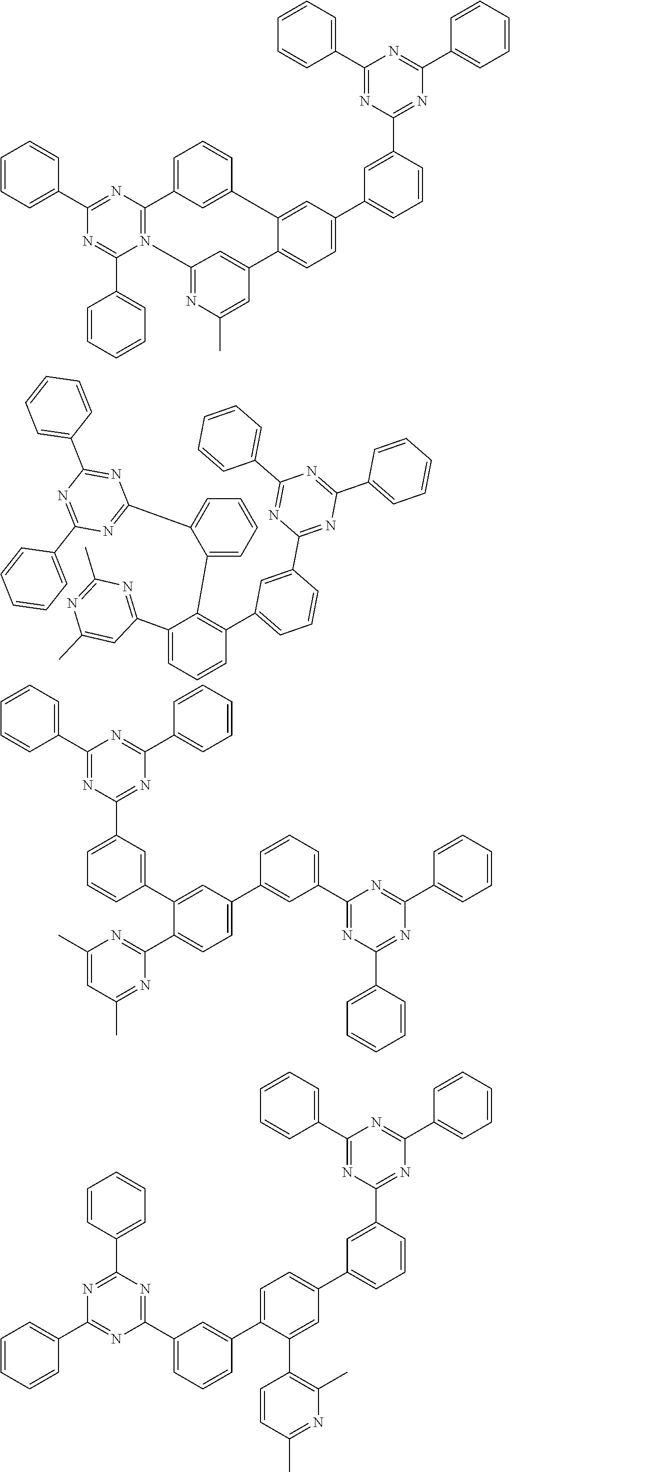

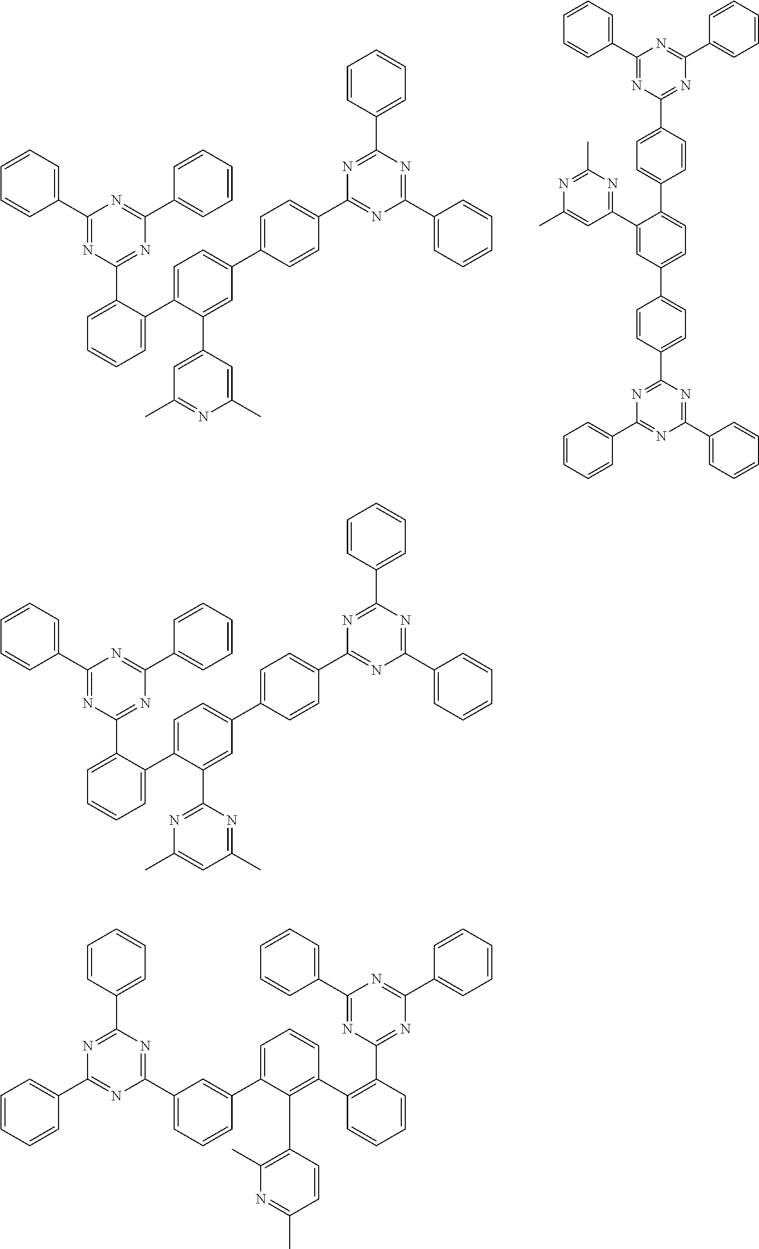 [SMILES: Cc1cc(-c2cc(-c3ccc(-c4nc(-c5ccccc5)nc(-c5ccccc5)n4)cc3)ccc2-c2ccccc2-c2nc(-c3ccccc3)nc(-c3ccccc3)n2)cc(C)n1 ; Cc1cc(-c2cc(-c3ccc(-c4nc(-c5ccccc5)nc(-c5ccccc5)n4)cc3)ccc2-c2ccc(-c3nc(-c4ccccc4)nc(-c4ccccc4)n3)cc2)nc(C)n1 ; Cc1cc(C)nc(-c2cc(-c3ccc(-c4nc(-c5ccccc5)nc(-c5ccccc5)n4)cc3)ccc2-c2ccccc2-c2nc(-c3ccccc3)nc(-c3ccccc3)n2)n1 ; Cc1ccc(-c2c(-c3cccc(-c4nc(-c5ccccc5)nc(-c5ccccc5)n4)c3)cccc2-c2ccccc2-c2nc(-c3ccccc3)nc(-c3ccccc3)n2)c(C)n1]

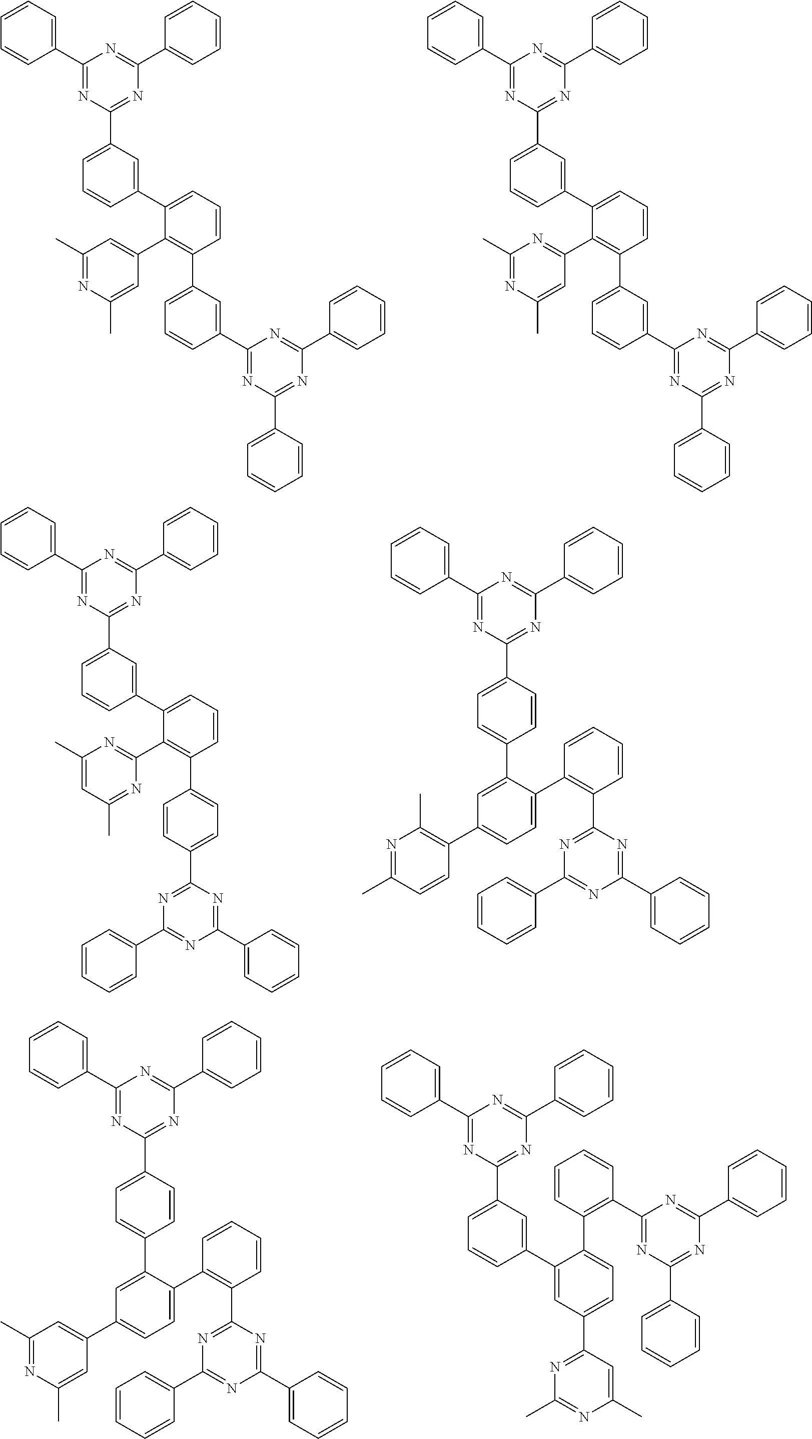

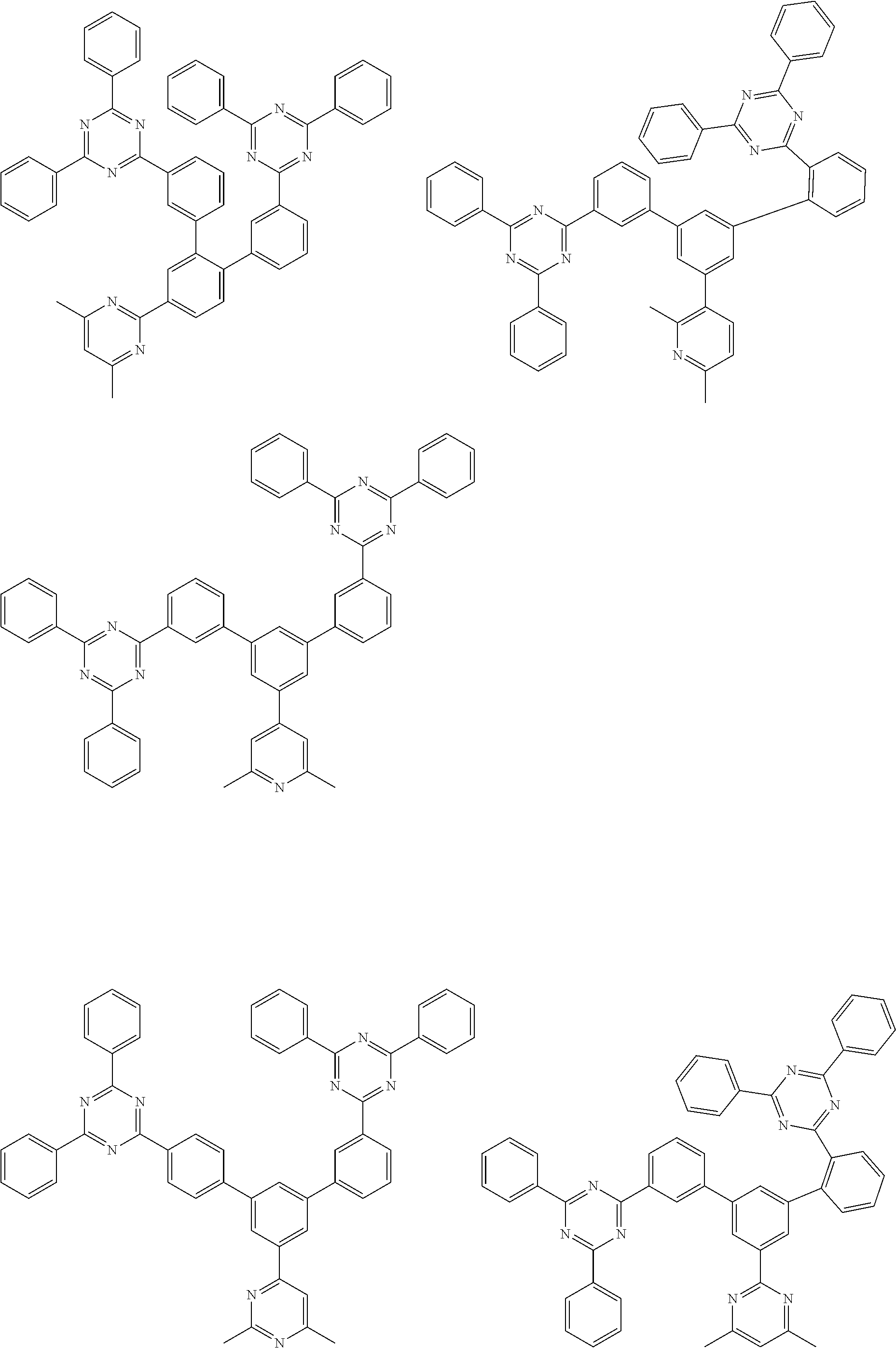

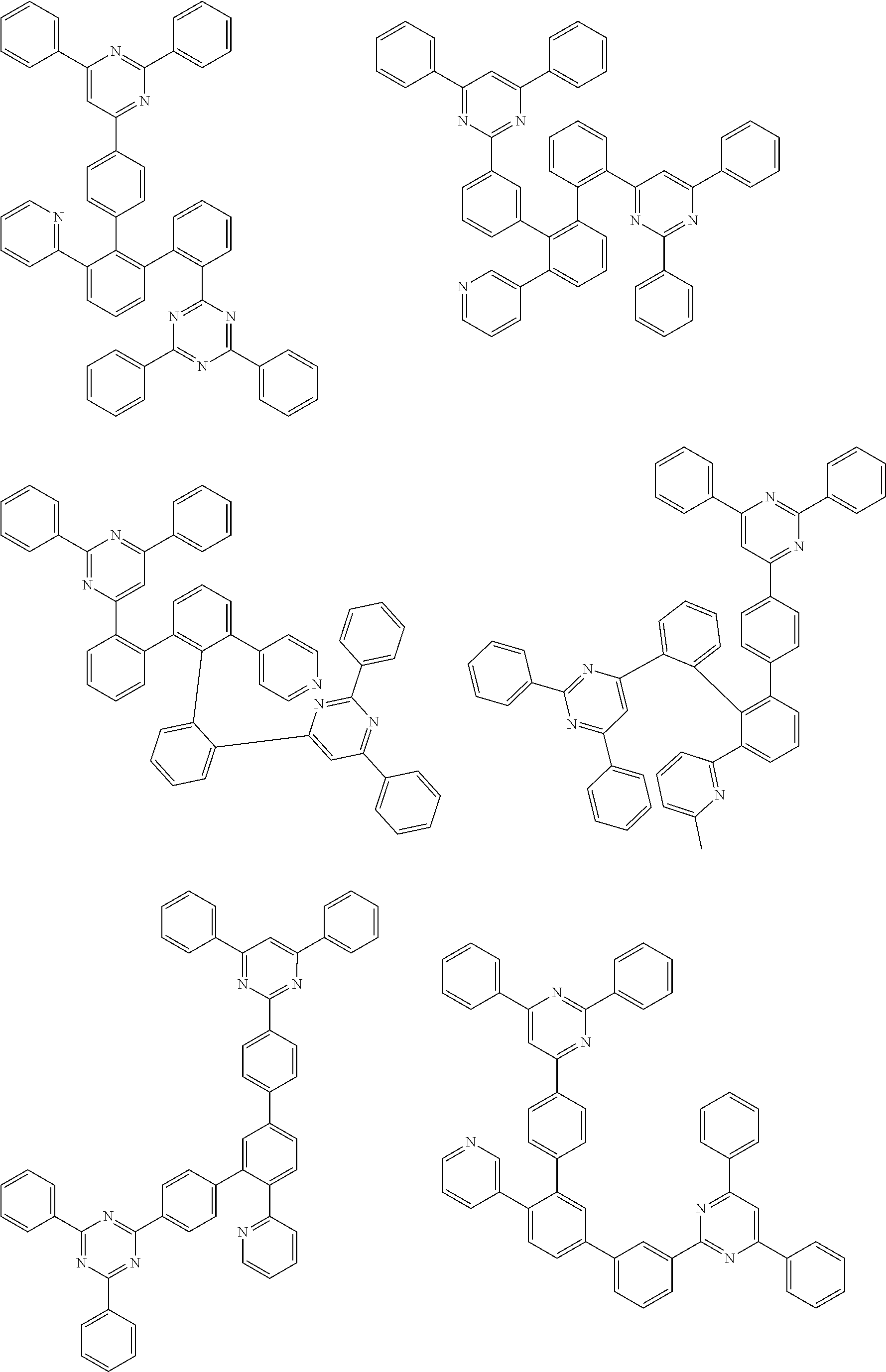

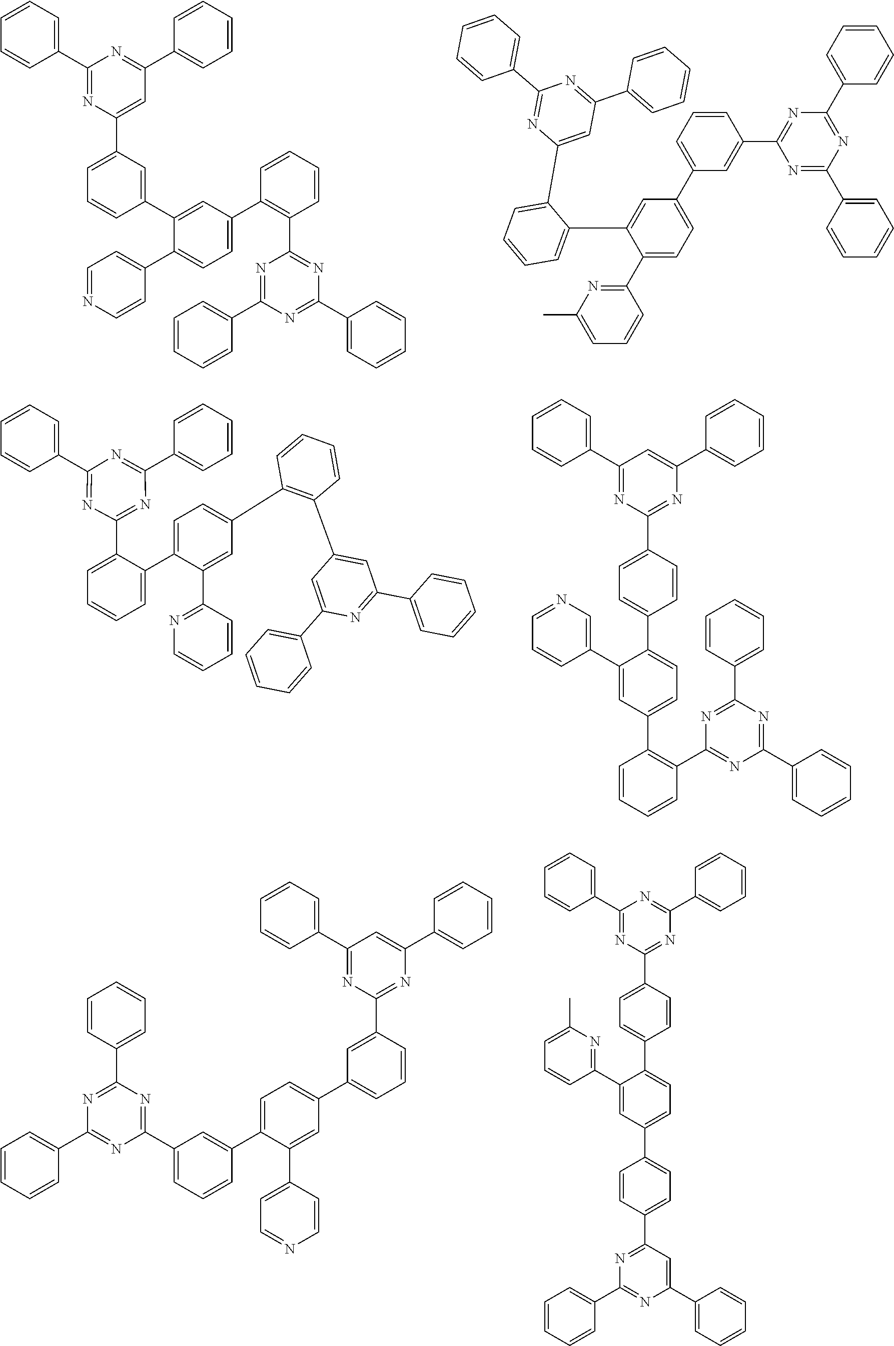

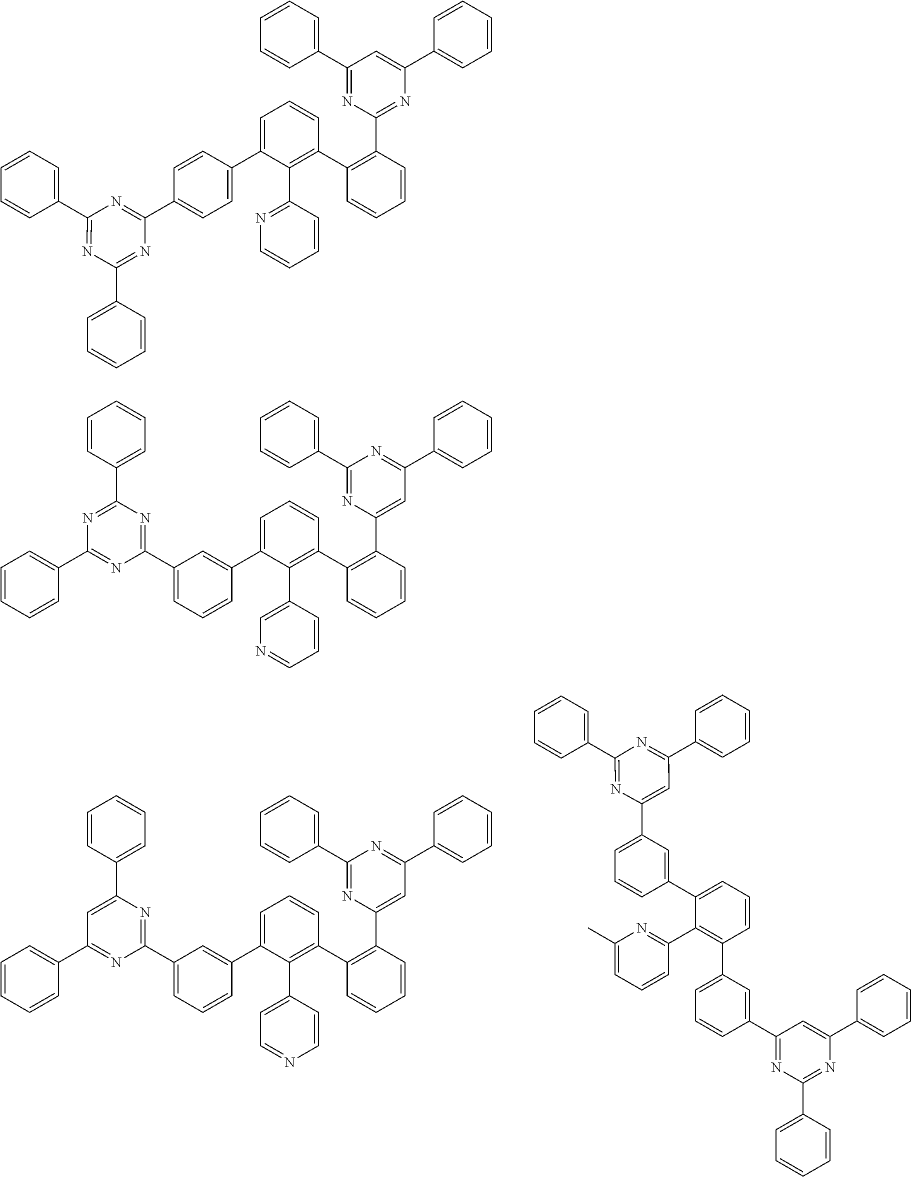

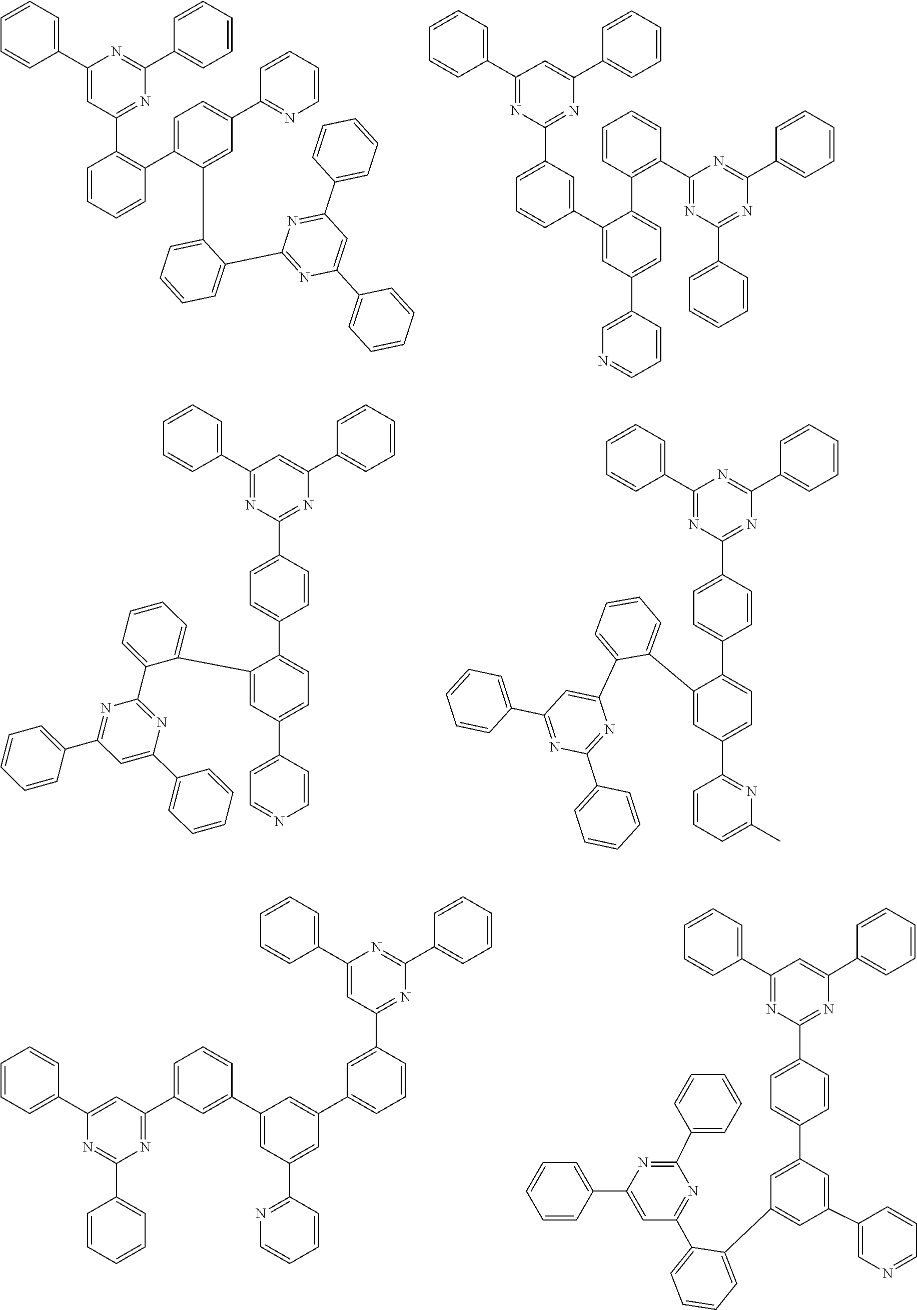

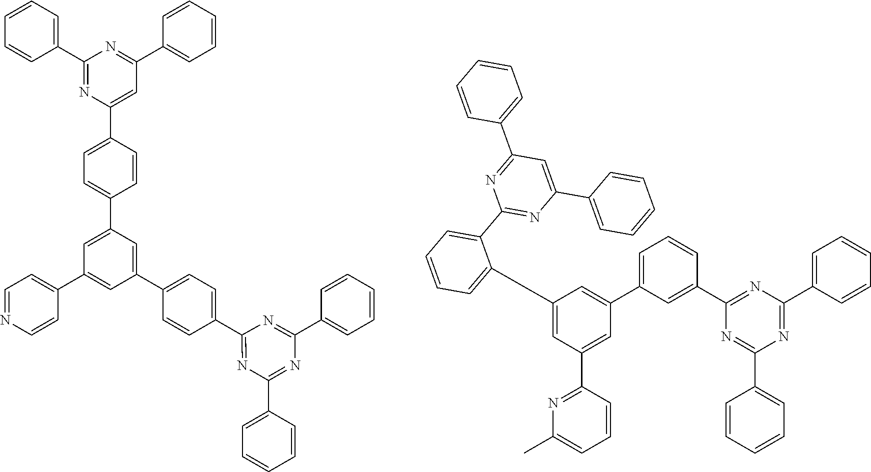 [SMILES: c1ccc(-c2cc(-c3ccc(-c4cc(-c5ccncc5)cc(-c5ccc(-c6nc(-c7ccccc7)nc(-c7ccccc7)n6)cc5)c4)cc3)nc(-c3ccccc3)n2)cc1 ; Cc1cccc(-c2cc(-c3cccc(-c4nc(-c5ccccc5)nc(-c5ccccc5)n4)c3)cc(-c3ccccc3-c3nc(-c4ccccc4)cc(-c4ccccc4)n3)c2)n1]

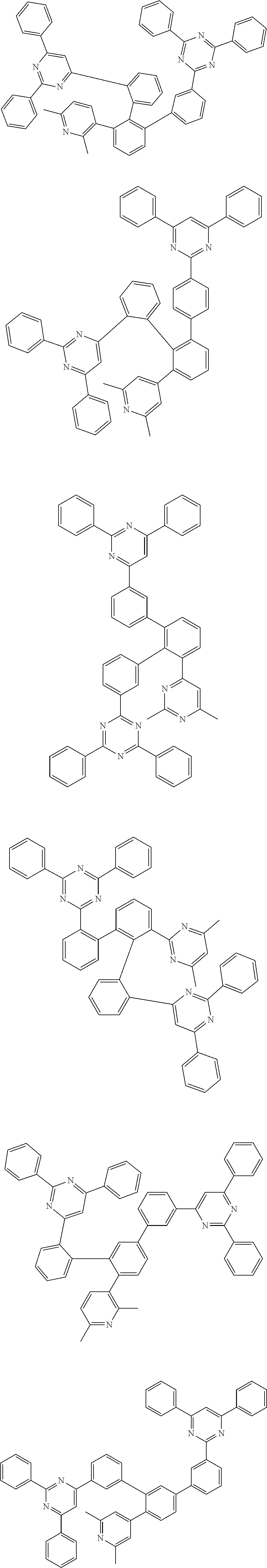

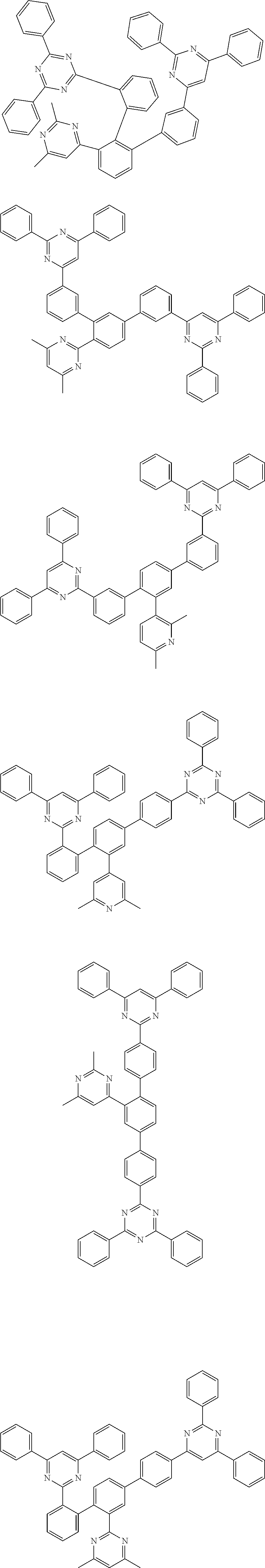

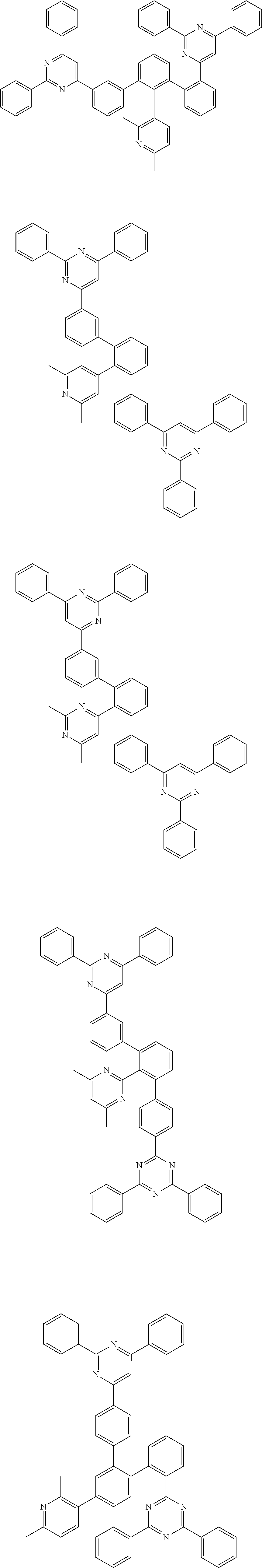

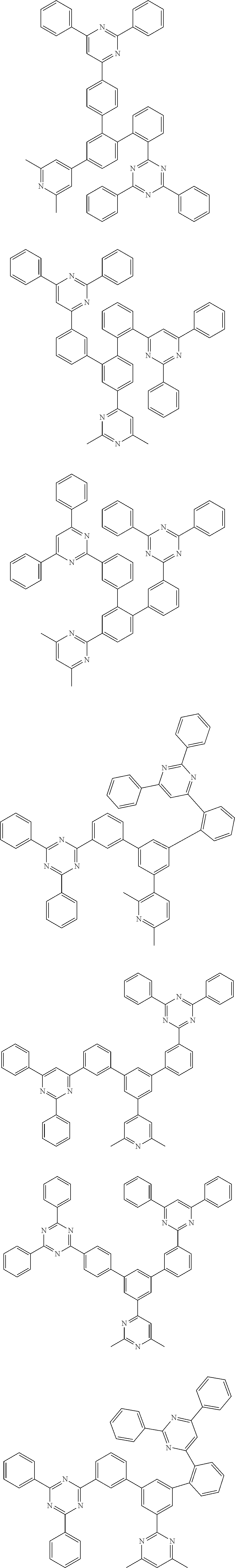

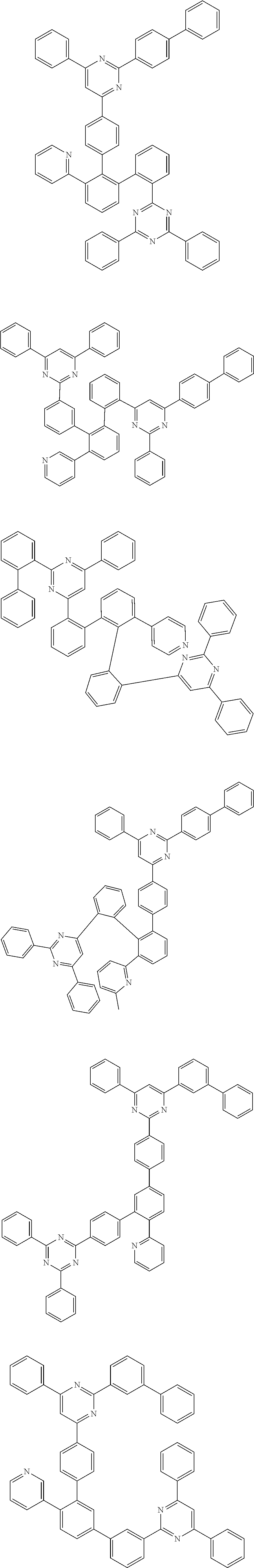

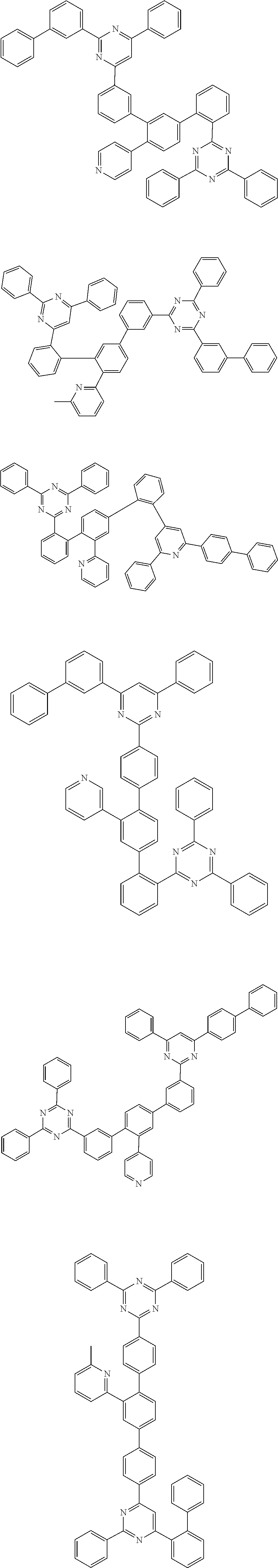

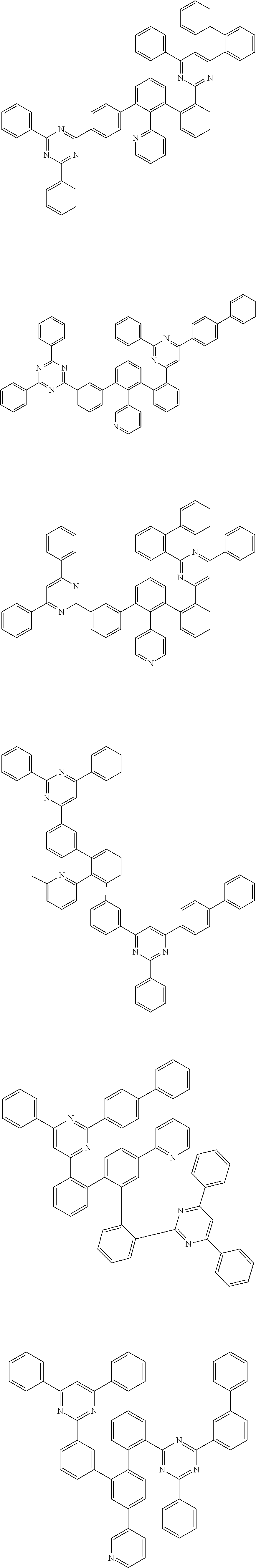

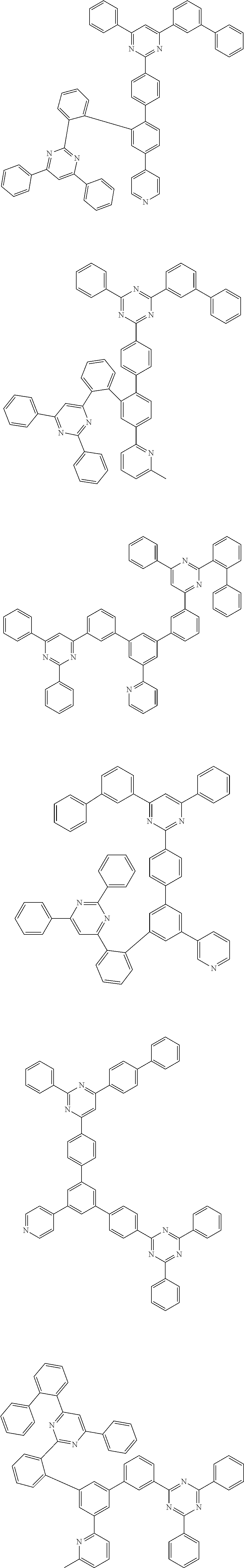

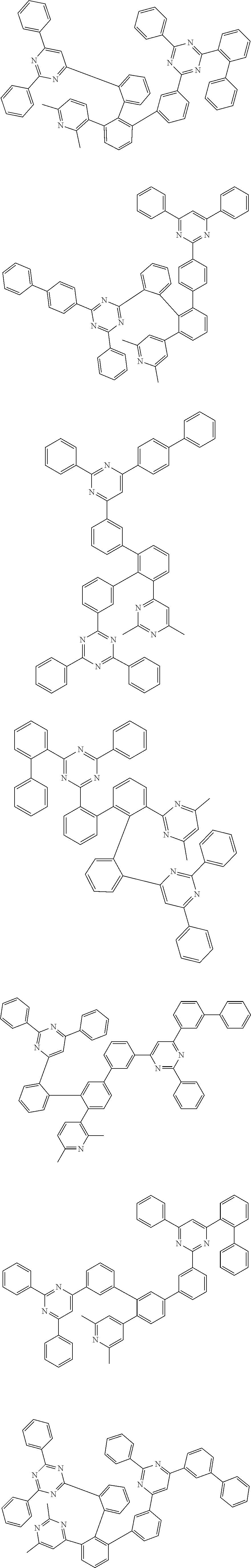

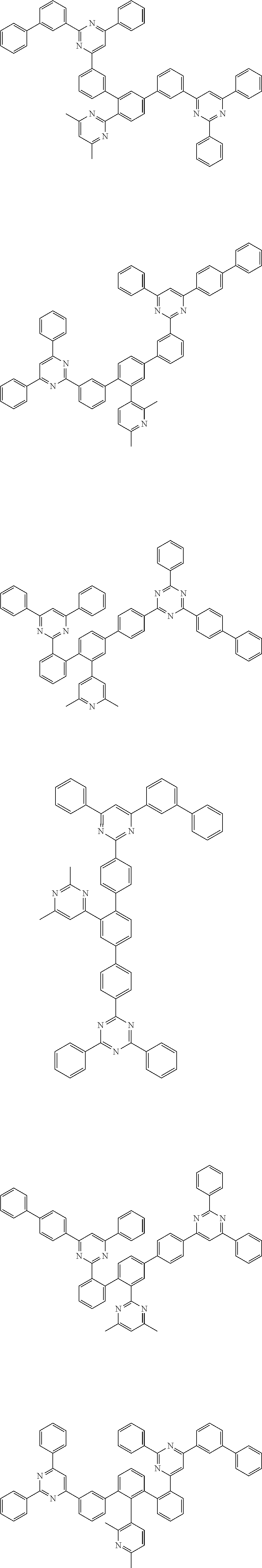

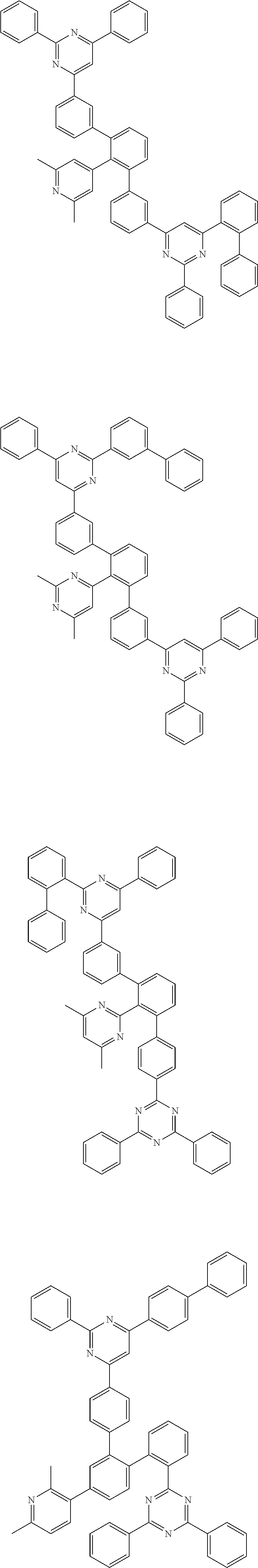 [SMILES: Cc1cc(-c2c(-c3cccc(-c4cc(-c5ccccc5)nc(-c5ccccc5)n4)c3)cccc2-c2cccc(-c3cc(-c4ccccc4-c4ccccc4)nc(-c4ccccc4)n3)c2)cc(C)n1 ; Cc1cc(-c2c(-c3cccc(-c4cc(-c5ccccc5)nc(-c5ccccc5)n4)c3)cccc2-c2cccc(-c3cc(-c4ccccc4)nc(-c4cccc(-c5ccccc5)c4)n3)c2)nc(C)n1 ; Cc1cc(C)nc(-c2c(-c3ccc(-c4nc(-c5ccccc5)nc(-c5ccccc5)n4)cc3)cccc2-c2cccc(-c3cc(-c4ccccc4)nc(-c4ccccc4-c4ccccc4)n3)c2)n1 ; Cc1ccc(-c2ccc(-c3ccccc3-c3nc(-c4ccccc4)nc(-c4ccccc4)n3)c(-c3ccc(-c4cc(-c5ccc(-c6ccccc6)cc5)nc(-c5ccccc5)n4)cc3)c2)c(C)n1]

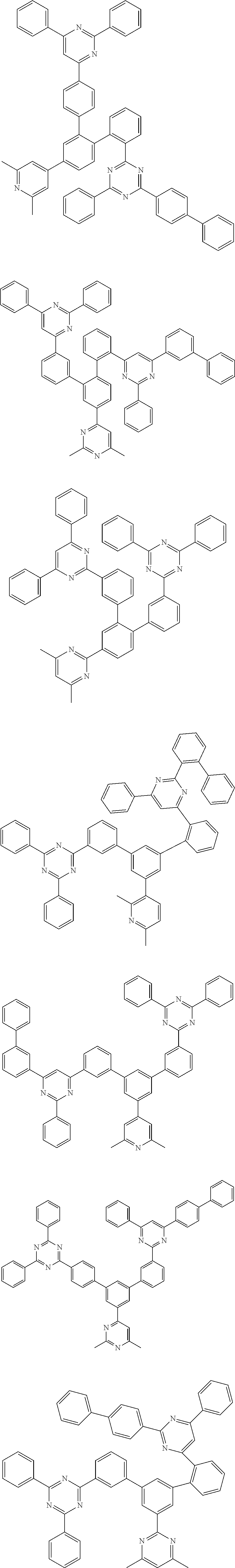

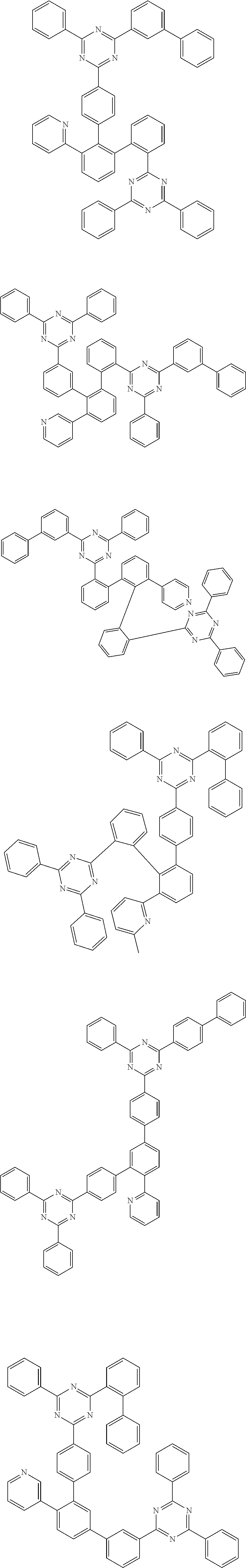

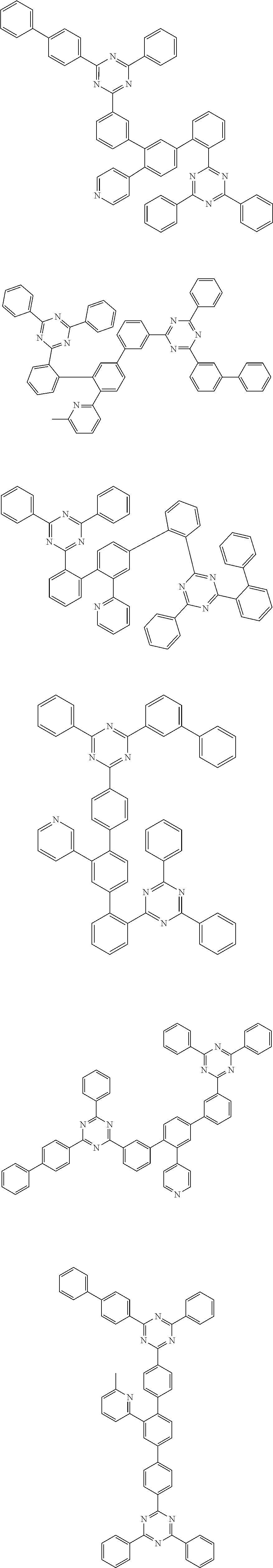

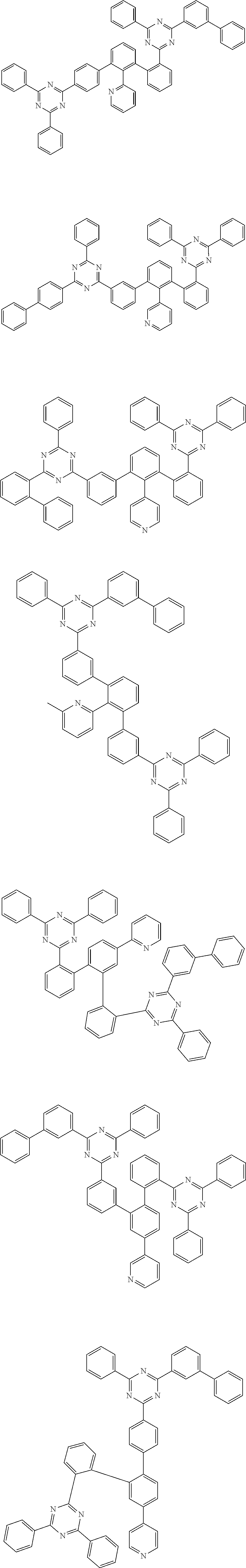

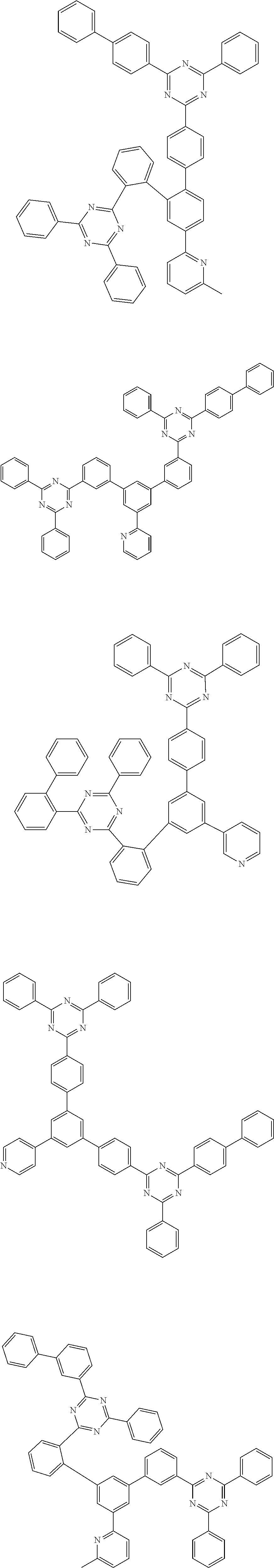

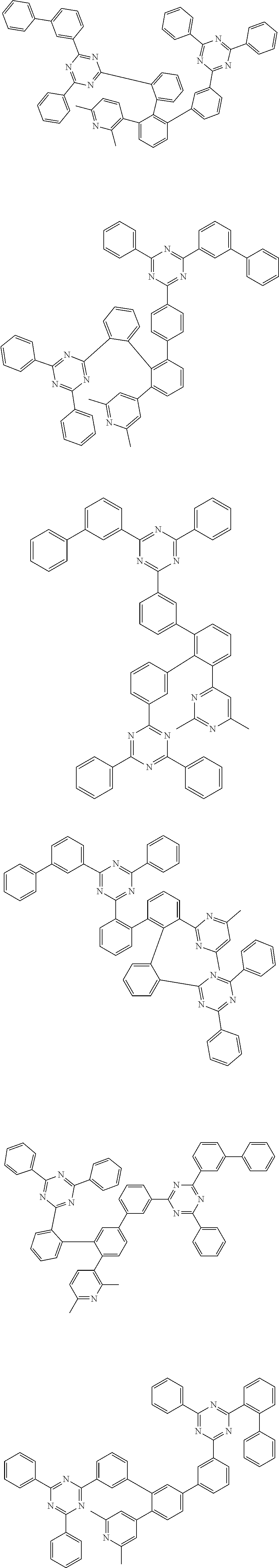

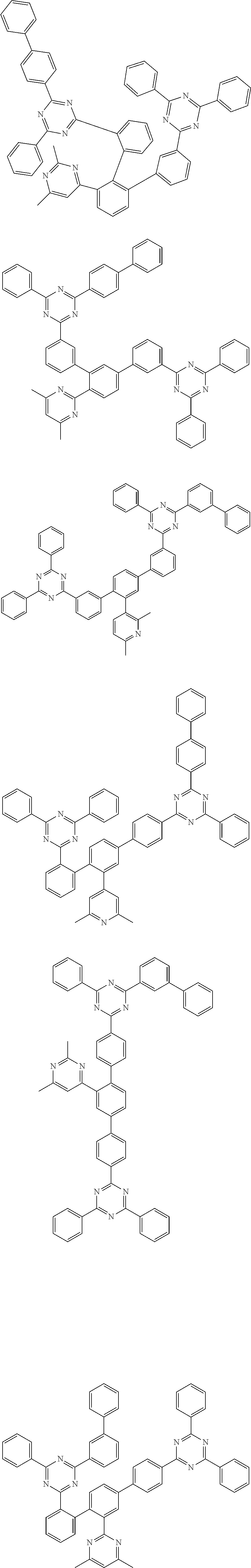

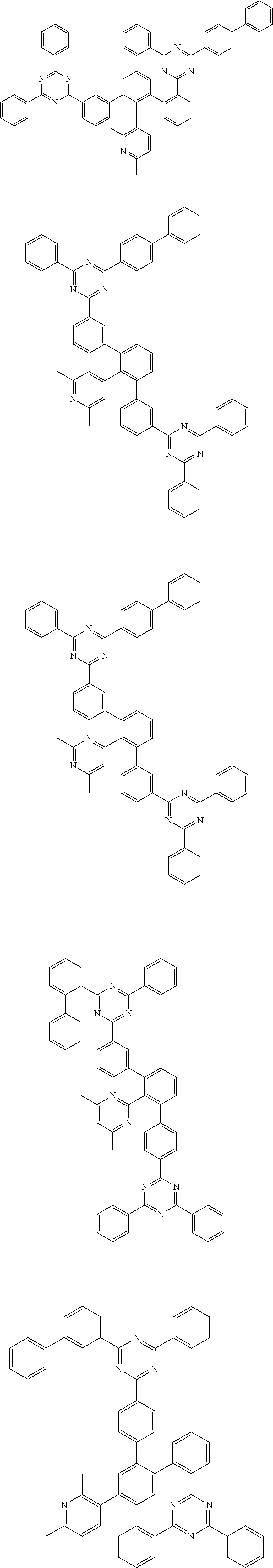

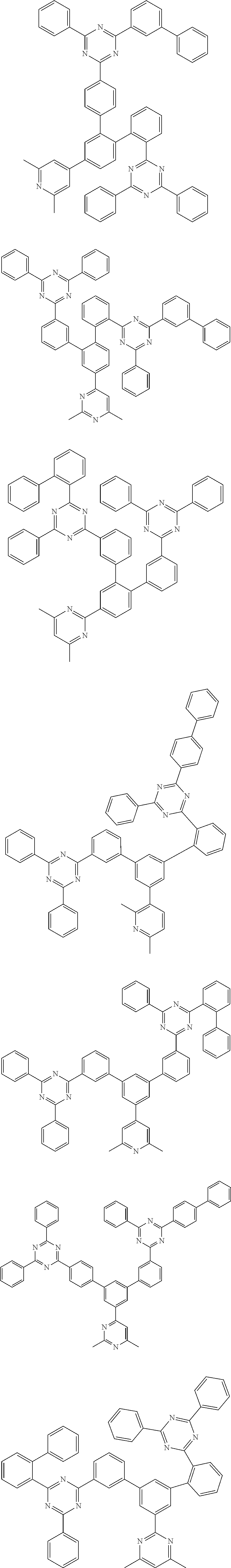

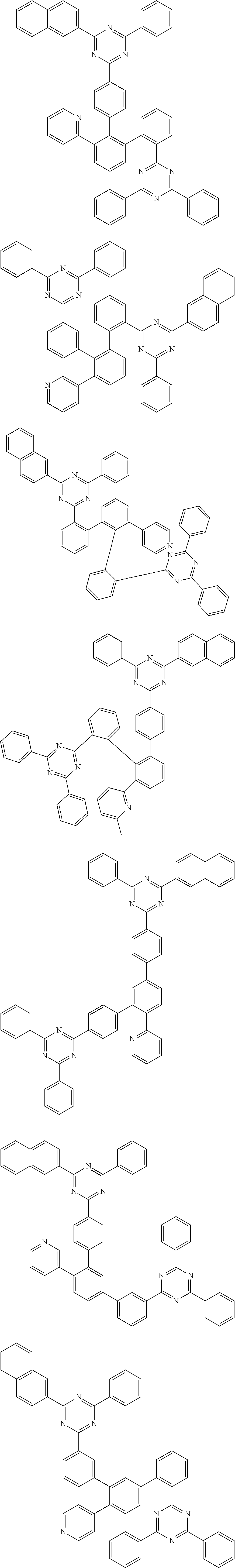

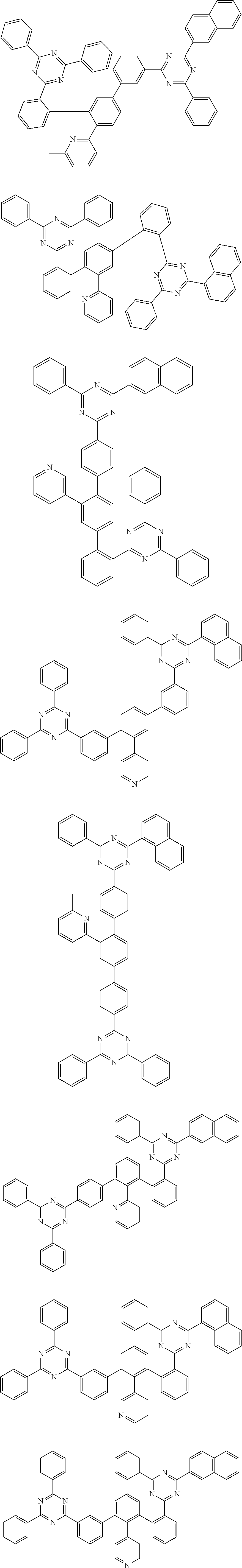

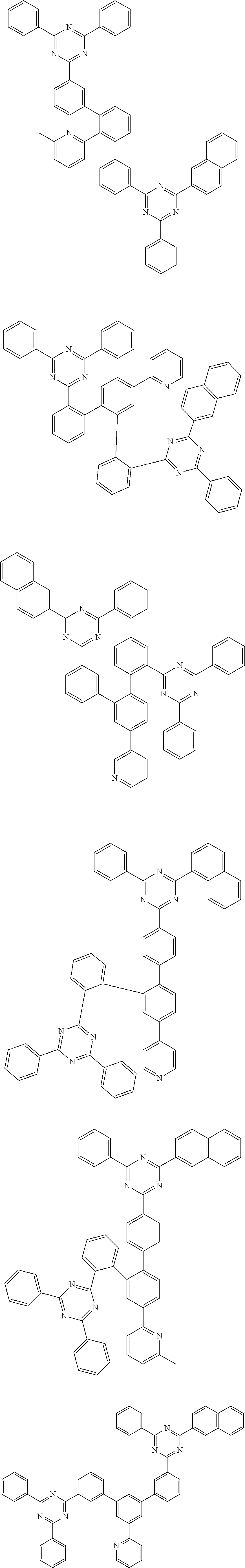

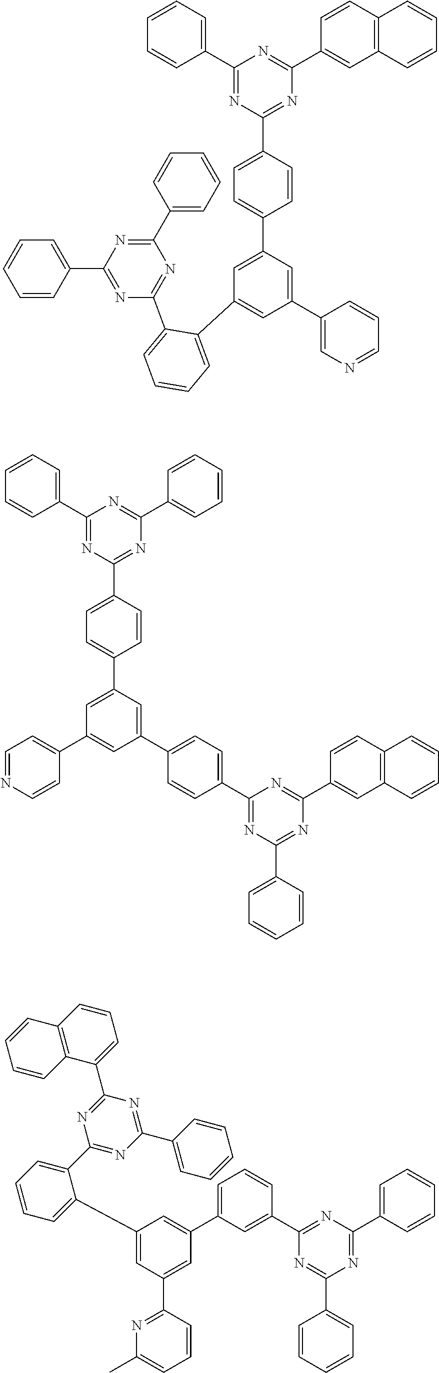 [SMILES: c1ccc(-c2nc(-c3ccc(-c4cc(-c5cccnc5)cc(-c5ccccc5-c5nc(-c6ccccc6)nc(-c6ccccc6)n5)c4)cc3)nc(-c3ccc4ccccc4c3)n2)cc1 ; c1ccc(-c2nc(-c3ccccc3)nc(-c3ccc(-c4cc(-c5ccncc5)cc(-c5ccc(-c6nc(-c7ccccc7)nc(-c7ccc8ccccc8c7)n6)cc5)c4)cc3)n2)cc1 ; Cc1cccc(-c2cc(-c3cccc(-c4nc(-c5ccccc5)nc(-c5ccccc5)n4)c3)cc(-c3ccccc3-c3nc(-c4ccccc4)nc(-c4cccc5ccccc45)n3)c2)n1]

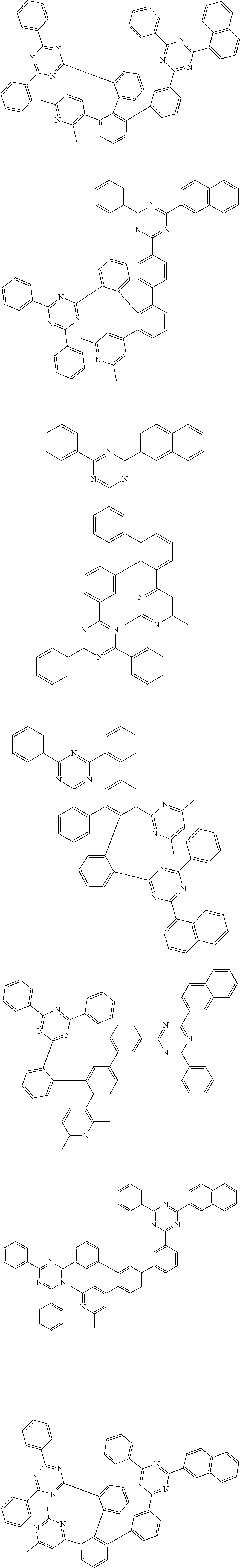

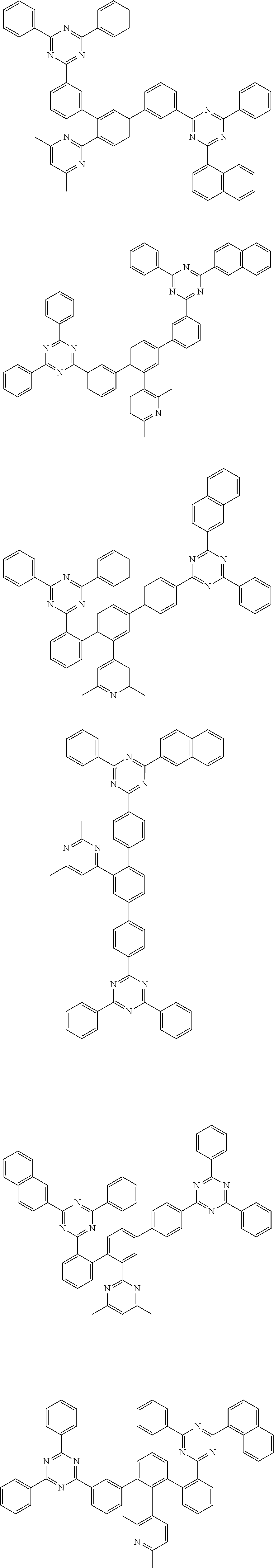

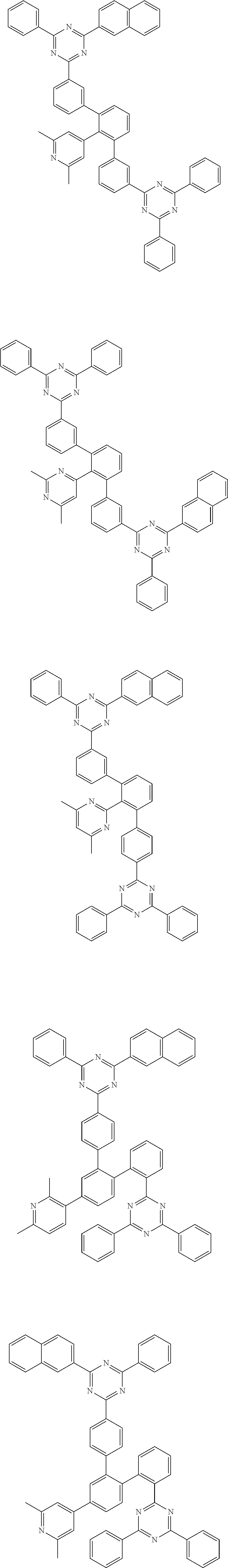

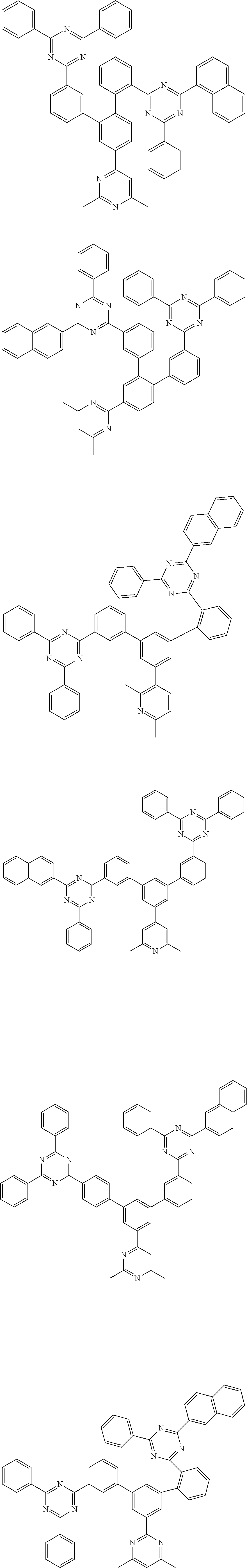

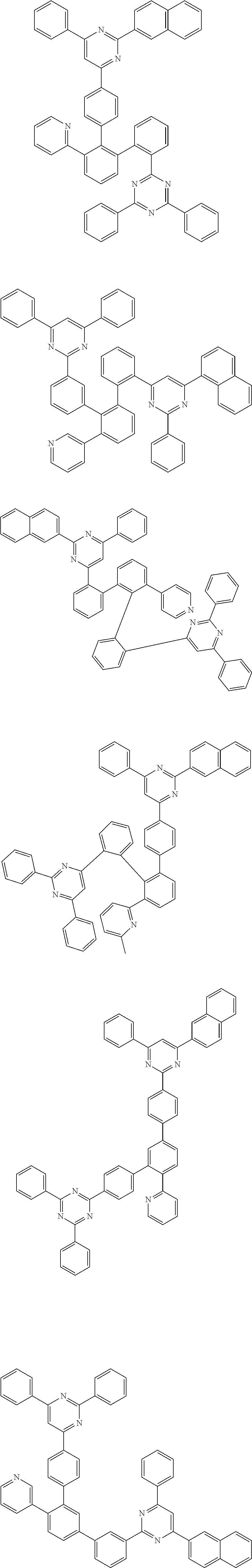

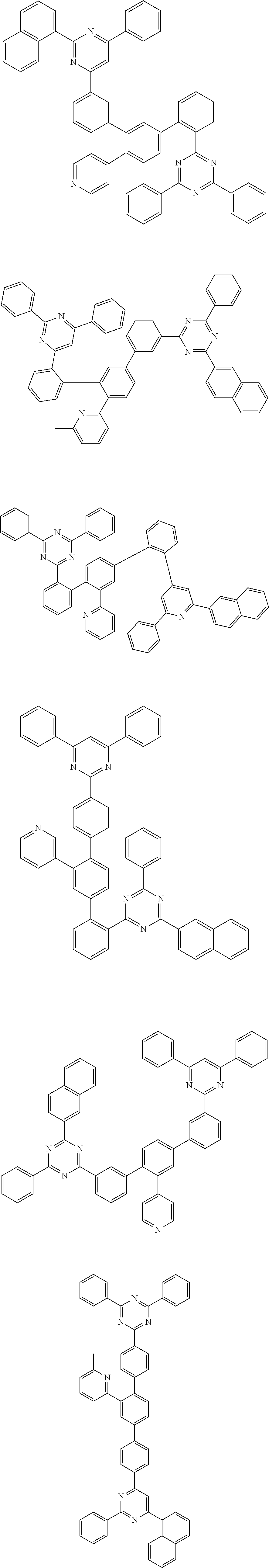

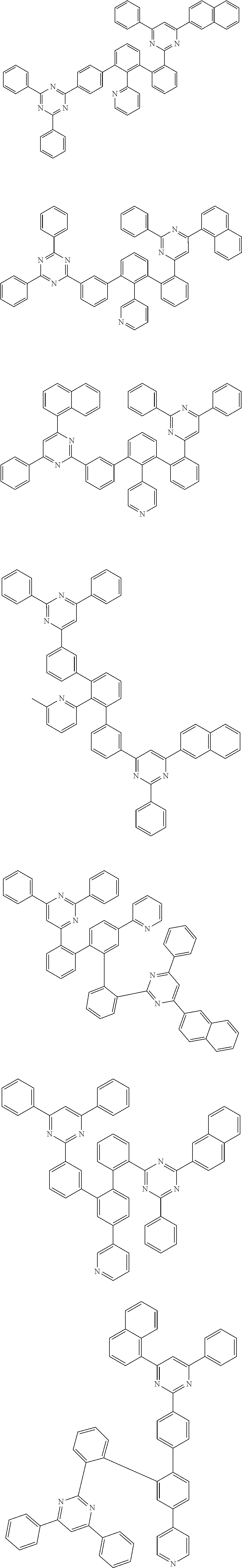

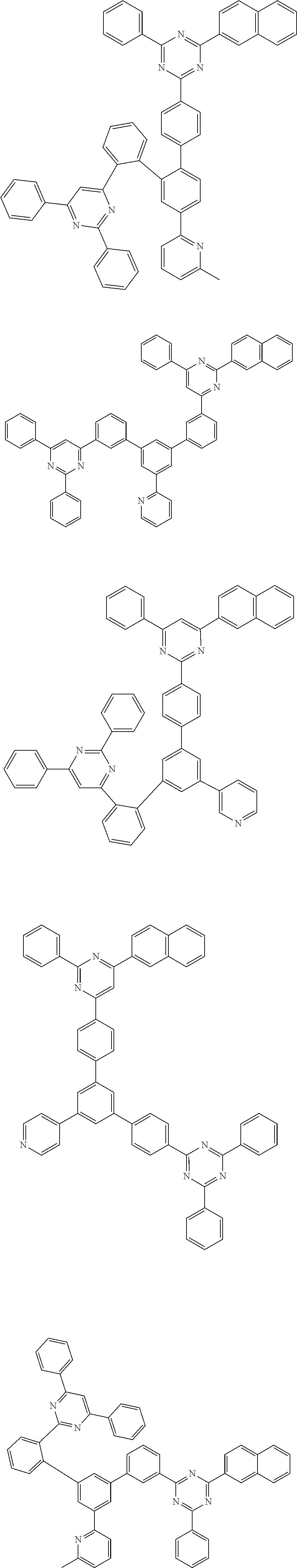

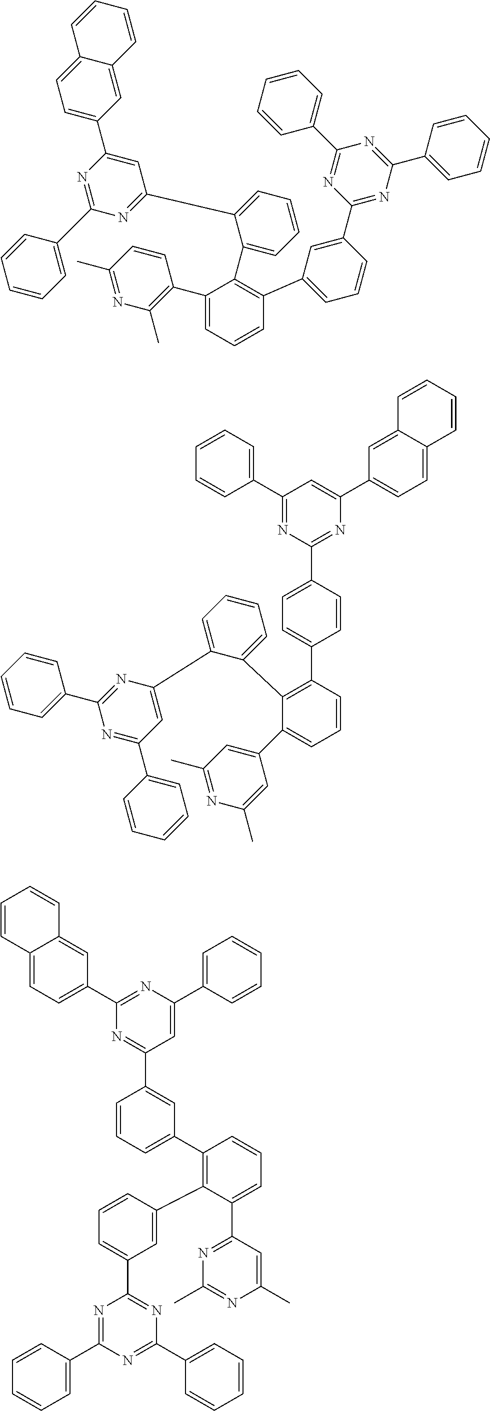 [SMILES: Cc1ccc(-c2cccc(-c3cccc(-c4nc(-c5ccccc5)nc(-c5ccccc5)n4)c3)c2-c2ccccc2-c2cc(-c3ccc4ccccc4c3)nc(-c3ccccc3)n2)c(C)n1 ; Cc1cc(-c2cccc(-c3ccc(-c4nc(-c5ccccc5)cc(-c5ccc6ccccc6c5)n4)cc3)c2-c2ccccc2-c2cc(-c3ccccc3)nc(-c3ccccc3)n2)cc(C)n1 ; Cc1cc(-c2cccc(-c3cccc(-c4cc(-c5ccccc5)nc(-c5ccc6ccccc6c5)n4)c3)c2-c2cccc(-c3nc(-c4ccccc4)nc(-c4ccccc4)n3)c2)nc(C)n1]

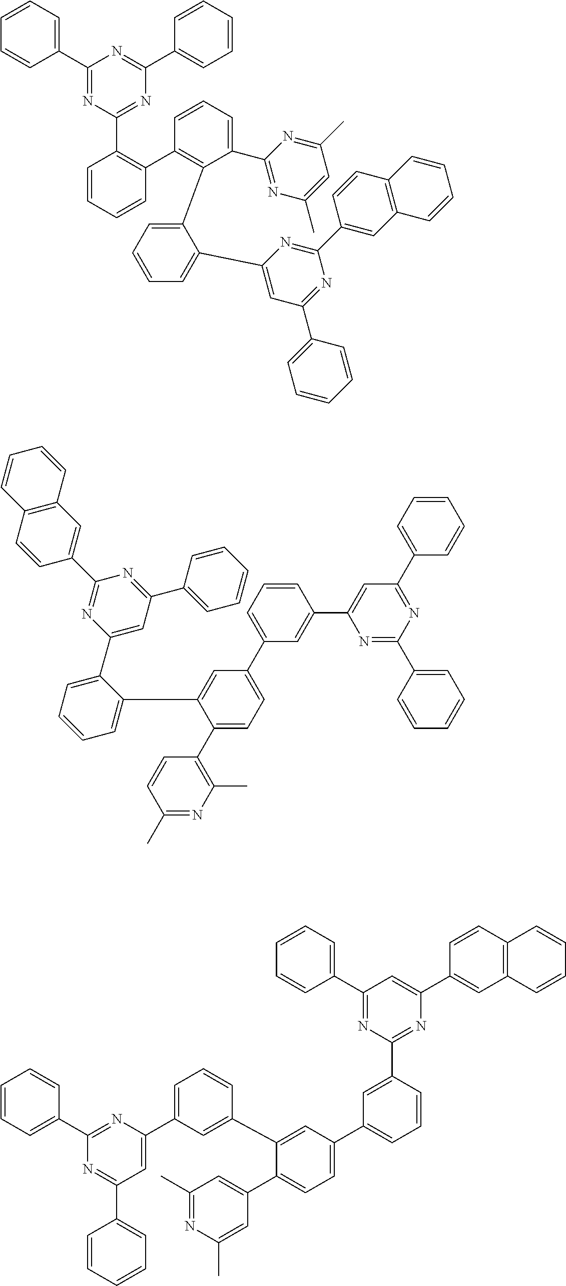 [SMILES: Cc1cc(C)nc(-c2cccc(-c3ccccc3-c3nc(-c4ccccc4)nc(-c4ccccc4)n3)c2-c2ccccc2-c2cc(-c3ccccc3)nc(-c3ccc4ccccc4c3)n2)n1 ; Cc1ccc(-c2ccc(-c3cccc(-c4cc(-c5ccccc5)nc(-c5ccccc5)n4)c3)cc2-c2ccccc2-c2cc(-c3ccccc3)nc(-c3ccc4ccccc4c3)n2)c(C)n1 ; Cc1cc(-c2ccc(-c3cccc(-c4nc(-c5ccccc5)cc(-c5ccc6ccccc6c5)n4)c3)cc2-c2cccc(-c3cc(-c4ccccc4)nc(-c4ccccc4)n3)c2)cc(C)n1]

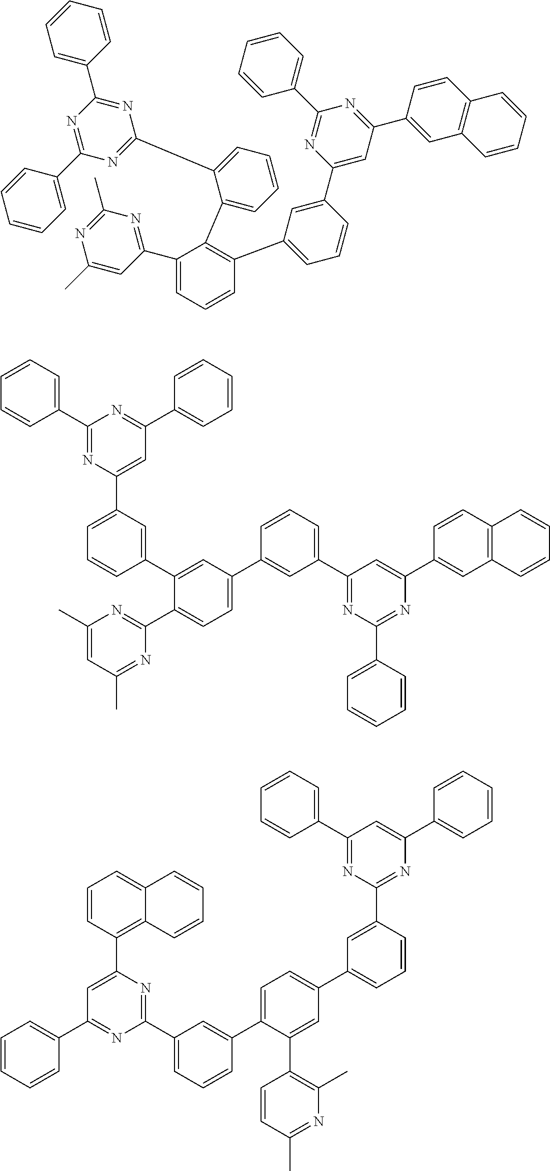 [SMILES: Cc1cc(-c2cccc(-c3cccc(-c4cc(-c5ccc6ccccc6c5)nc(-c5ccccc5)n4)c3)c2-c2ccccc2-c2nc(-c3ccccc3)nc(-c3ccccc3)n2)nc(C)n1 ; Cc1cc(C)nc(-c2ccc(-c3cccc(-c4cc(-c5ccc6ccccc6c5)nc(-c5ccccc5)n4)c3)cc2-c2cccc(-c3cc(-c4ccccc4)nc(-c4ccccc4)n3)c2)n1 ; Cc1ccc(-c2cc(-c3cccc(-c4nc(-c5ccccc5)cc(-c5ccccc5)n4)c3)ccc2-c2cccc(-c3nc(-c4ccccc4)cc(-c4cccc5ccccc45)n3)c2)c(C)n1]

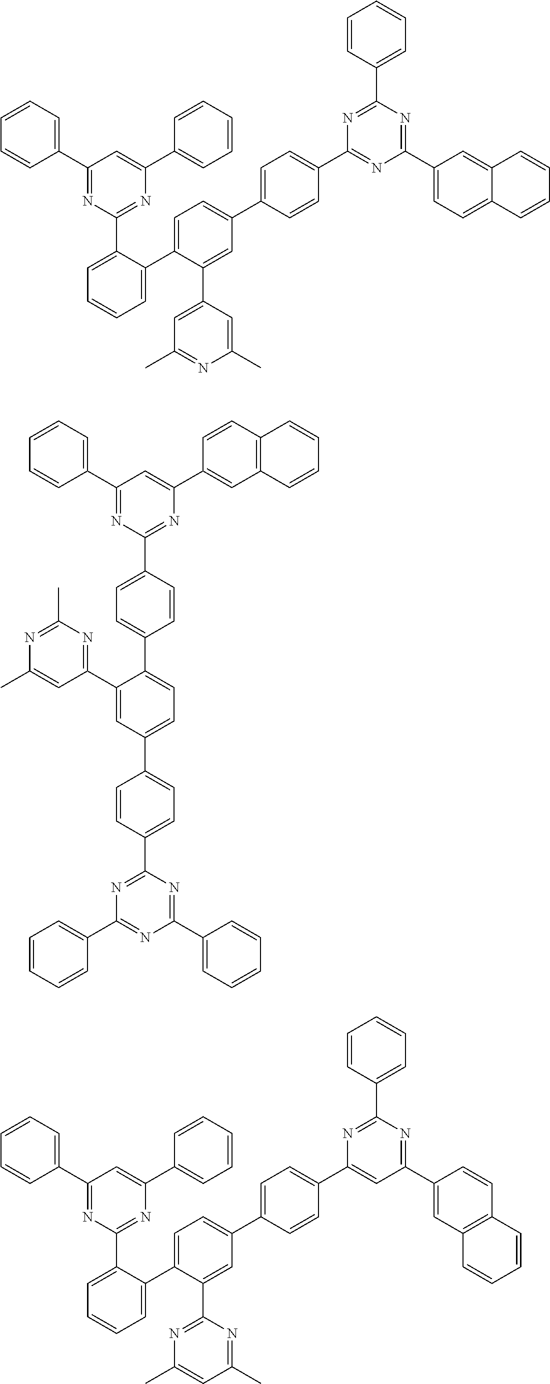 [SMILES: Cc1cc(-c2cc(-c3ccc(-c4nc(-c5ccccc5)nc(-c5ccc6ccccc6c5)n4)cc3)ccc2-c2ccccc2-c2nc(-c3ccccc3)cc(-c3ccccc3)n2)cc(C)n1 ; Cc1cc(-c2cc(-c3ccc(-c4nc(-c5ccccc5)nc(-c5ccccc5)n4)cc3)ccc2-c2ccc(-c3nc(-c4ccccc4)cc(-c4ccc5ccccc5c4)n3)cc2)nc(C)n1 ; Cc1cc(C)nc(-c2cc(-c3ccc(-c4cc(-c5ccc6ccccc6c5)nc(-c5ccccc5)n4)cc3)ccc2-c2ccccc2-c2nc(-c3ccccc3)cc(-c3ccccc3)n2)n1]

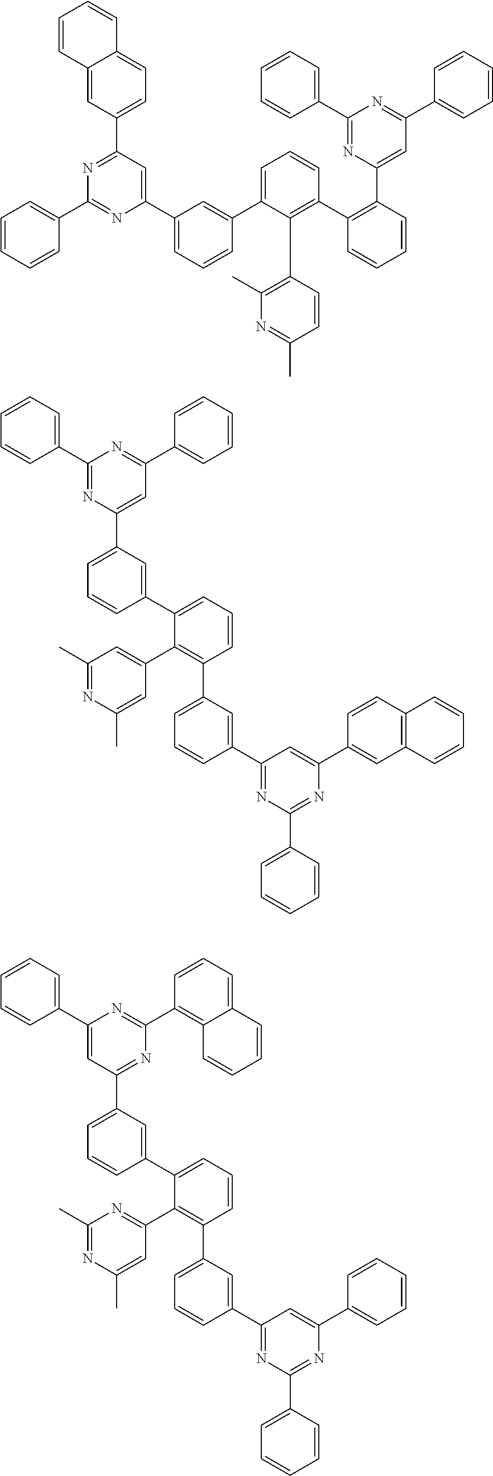 [SMILES: Cc1ccc(-c2c(-c3cccc(-c4cc(-c5ccc6ccccc6c5)nc(-c5ccccc5)n4)c3)cccc2-c2ccccc2-c2cc(-c3ccccc3)nc(-c3ccccc3)n2)c(C)n1 ; Cc1cc(-c2c(-c3cccc(-c4cc(-c5ccccc5)nc(-c5ccccc5)n4)c3)cccc2-c2cccc(-c3cc(-c4ccc5ccccc5c4)nc(-c4ccccc4)n3)c2)cc(C)n1 ; Cc1cc(-c2c(-c3cccc(-c4cc(-c5ccccc5)nc(-c5ccccc5)n4)c3)cccc2-c2cccc(-c3cc(-c4ccccc4)nc(-c4cccc5ccccc45)n3)c2)nc(C)n1]

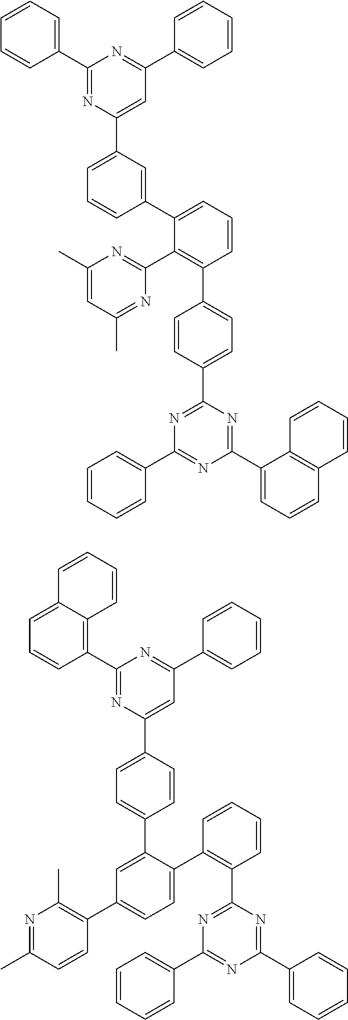

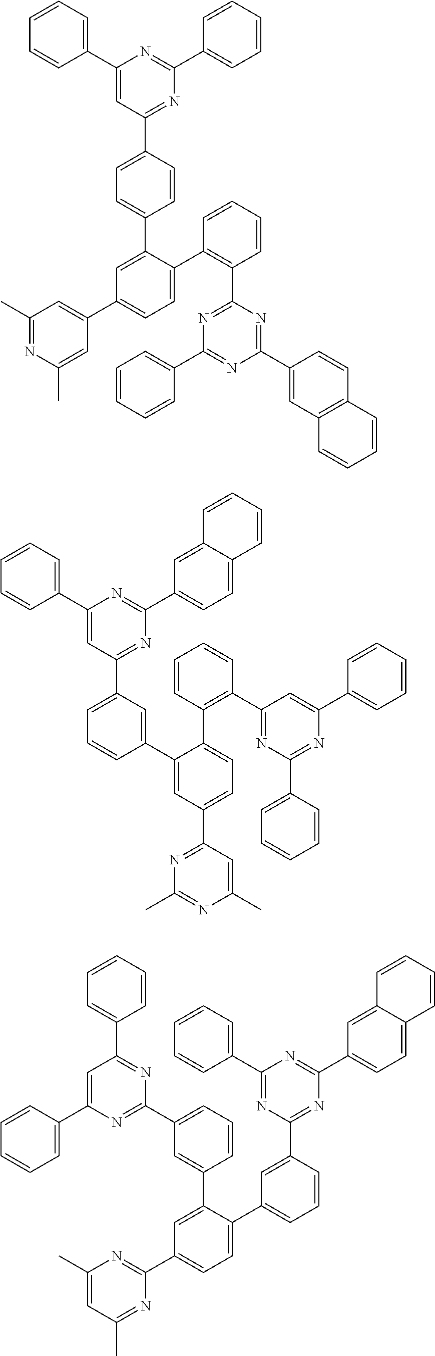 [SMILES: Cc1cc(-c2ccc(-c3ccccc3-c3nc(-c4ccccc4)nc(-c4ccc5ccccc5c4)n3)c(-c3ccc(-c4cc(-c5ccccc5)nc(-c5ccccc5)n4)cc3)c2)cc(C)n1 ; Cc1cc(-c2ccc(-c3ccccc3-c3cc(-c4ccccc4)nc(-c4ccccc4)n3)c(-c3cccc(-c4cc(-c5ccccc5)nc(-c5ccc6ccccc6c5)n4)c3)c2)nc(C)n1 ; Cc1cc(C)nc(-c2ccc(-c3cccc(-c4nc(-c5ccccc5)nc(-c5ccc6ccccc6c5)n4)c3)c(-c3cccc(-c4nc(-c5ccccc5)cc(-c5ccccc5)n4)c3)c2)n1]

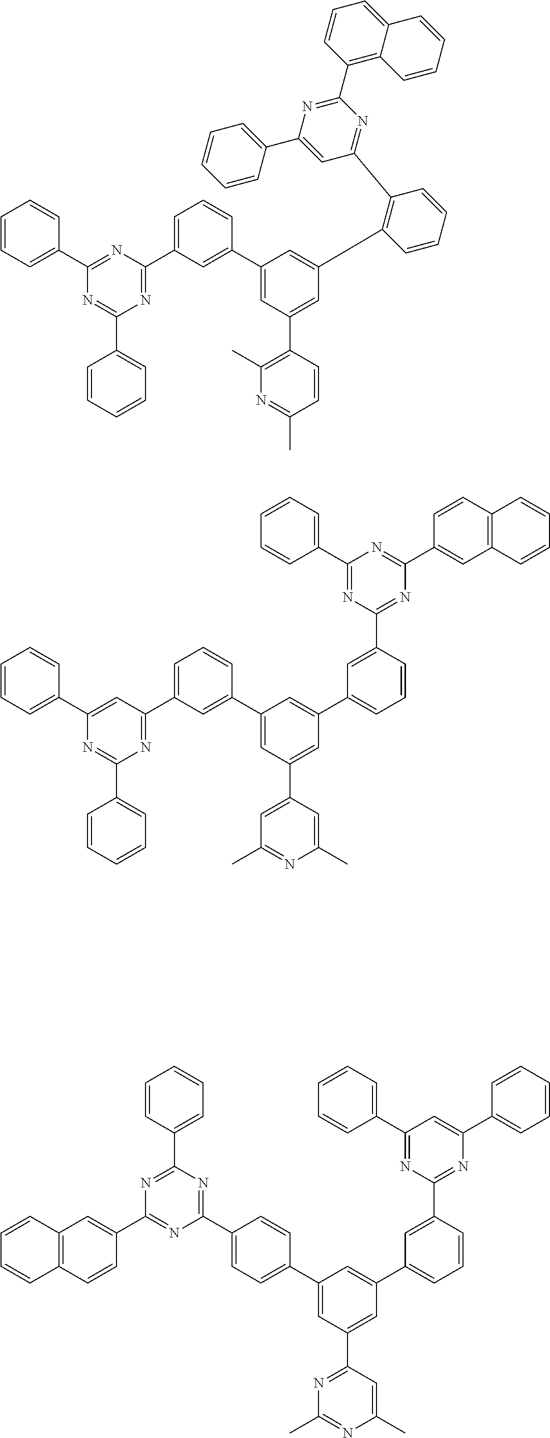 [SMILES: Cc1ccc(-c2cc(-c3cccc(-c4nc(-c5ccccc5)nc(-c5ccccc5)n4)c3)cc(-c3ccccc3-c3cc(-c4ccccc4)nc(-c4cccc5ccccc45)n3)c2)c(C)n1 ; Cc1cc(-c2cc(-c3cccc(-c4cc(-c5ccccc5)nc(-c5ccccc5)n4)c3)cc(-c3cccc(-c4nc(-c5ccccc5)nc(-c5ccc6ccccc6c5)n4)c3)c2)cc(C)n1 ; Cc1cc(-c2cc(-c3ccc(-c4nc(-c5ccccc5)nc(-c5ccc6ccccc6c5)n4)cc3)cc(-c3cccc(-c4nc(-c5ccccc5)cc(-c5ccccc5)n4)c3)c2)nc(C)n1]

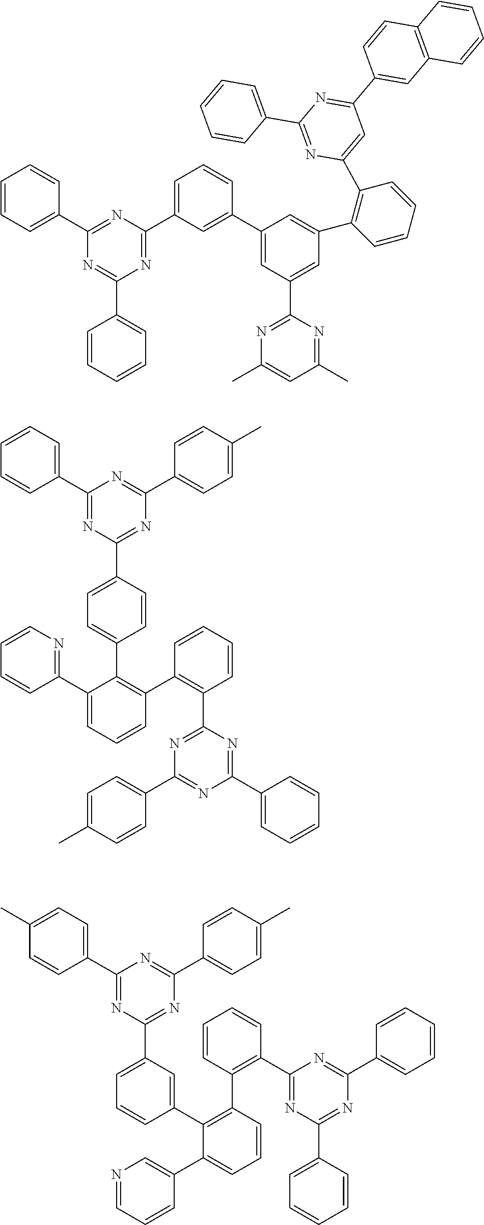 [SMILES: Cc1cc(C)nc(-c2cc(-c3cccc(-c4nc(-c5ccccc5)nc(-c5ccccc5)n4)c3)cc(-c3ccccc3-c3cc(-c4ccc5ccccc5c4)nc(-c4ccccc4)n3)c2)n1 ; Cc1ccc(-c2nc(-c3ccccc3)nc(-c3ccc(-c4c(-c5ccccn5)cccc4-c4ccccc4-c4nc(-c5ccccc5)nc(-c5ccc(C)cc5)n4)cc3)n2)cc1 ; Cc1ccc(-c2nc(-c3ccc(C)cc3)nc(-c3cccc(-c4c(-c5cccnc5)cccc4-c4ccccc4-c4nc(-c5ccccc5)nc(-c5ccccc5)n4)c3)n2)cc1]

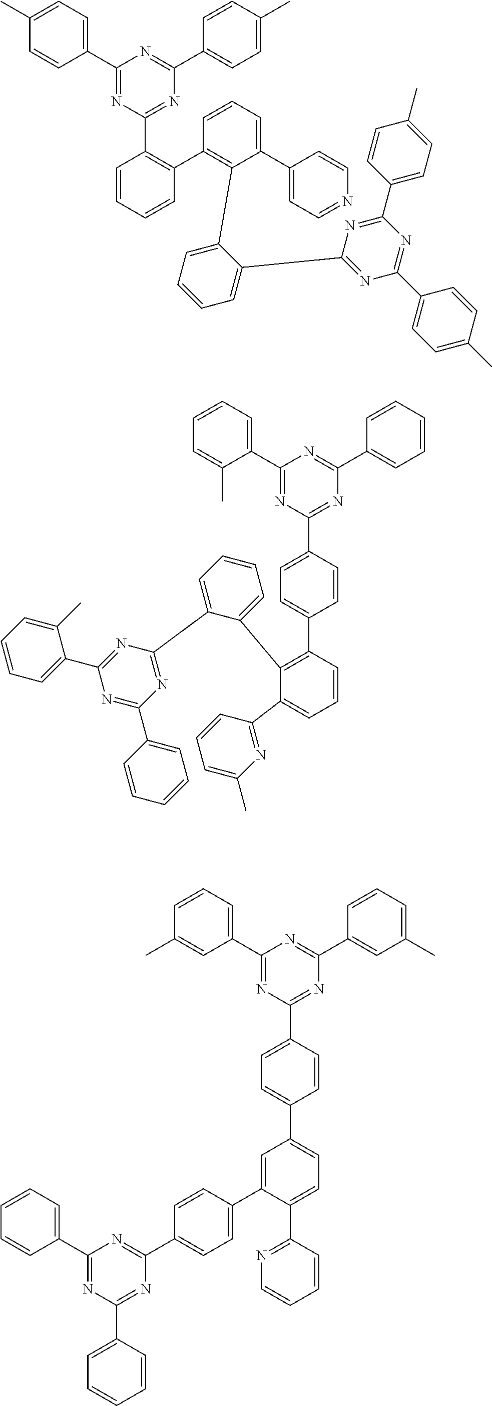

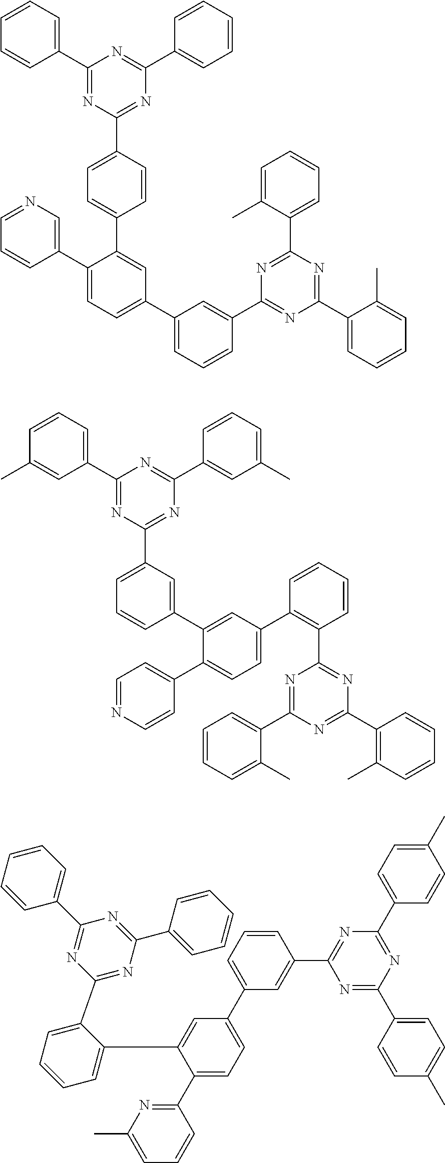 [SMILES: Cc1ccccc1-c1nc(-c2cccc(-c3ccc(-c4cccnc4)c(-c4ccc(-c5nc(-c6ccccc6)nc(-c6ccccc6)n5)cc4)c3)c2)nc(-c2ccccc2C)n1 ; Cc1cccc(-c2nc(-c3cccc(C)c3)nc(-c3cccc(-c4cc(-c5ccccc5-c5nc(-c6ccccc6C)nc(-c6ccccc6C)n5)ccc4-c4ccncc4)c3)n2)c1 ; Cc1ccc(-c2nc(-c3ccc(C)cc3)nc(-c3cccc(-c4ccc(-c5cccc(C)n5)c(-c5ccccc5-c5nc(-c6ccccc6)nc(-c6ccccc6)n5)c4)c3)n2)cc1]

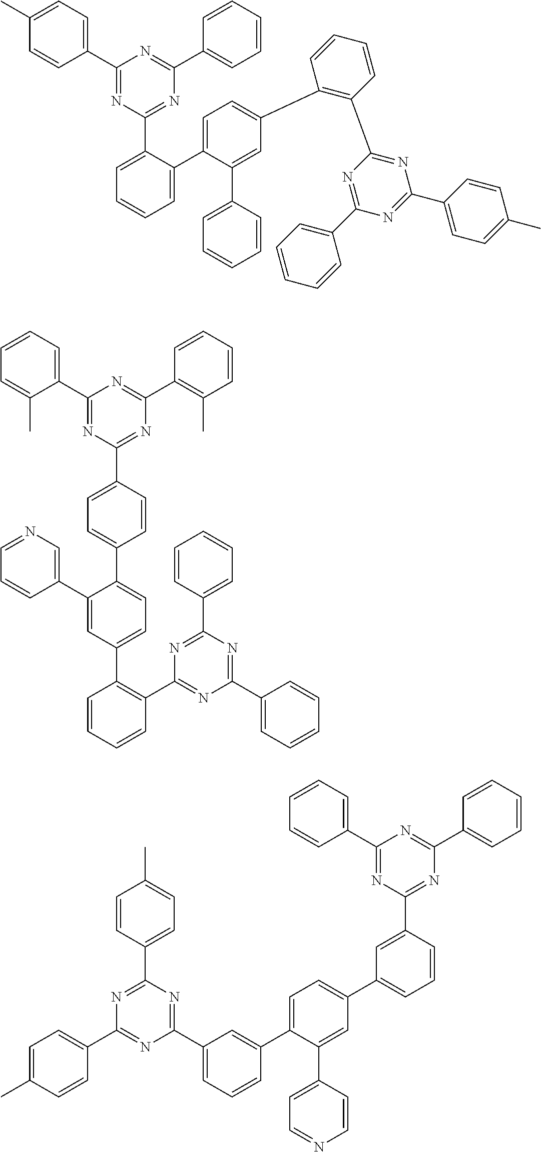

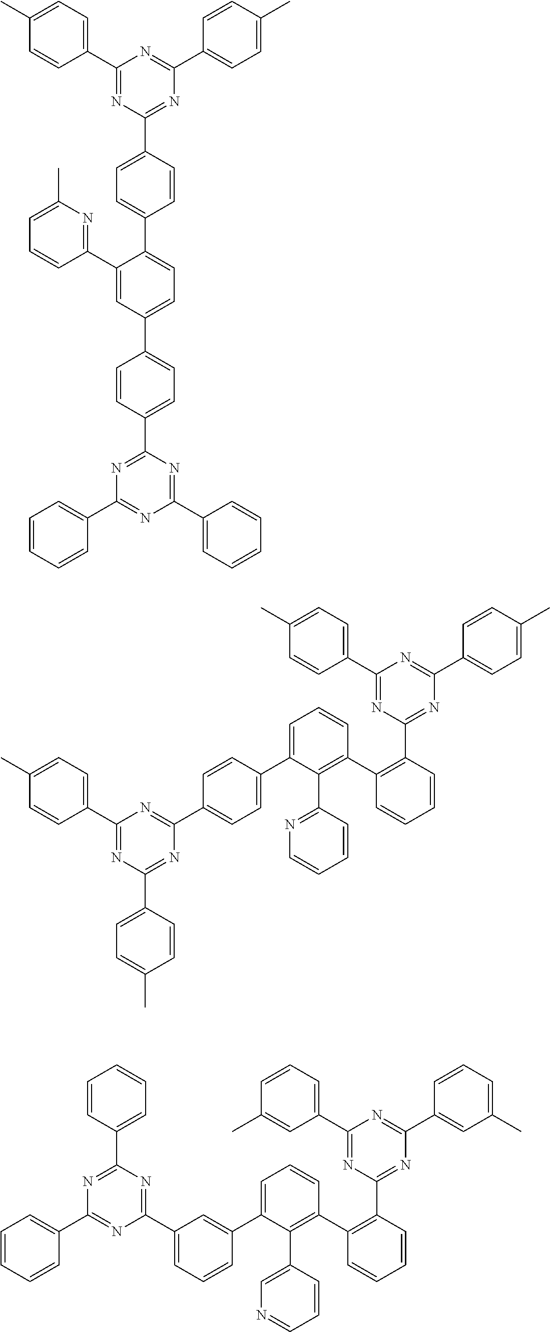

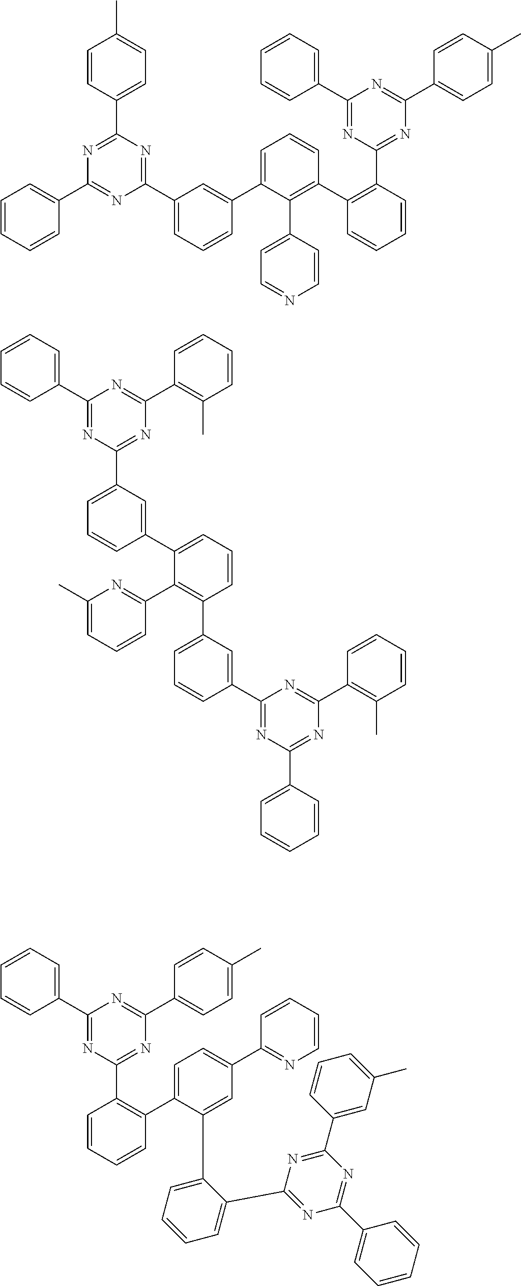 [SMILES: Cc1ccc(-c2nc(-c3ccccc3)nc(-c3cccc(-c4cccc(-c5ccccc5-c5nc(-c6ccccc6)nc(-c6ccc(C)cc6)n5)c4-c4ccncc4)c3)n2)cc1 ; Cc1cccc(-c2c(-c3cccc(-c4nc(-c5ccccc5)nc(-c5ccccc5C)n4)c3)cccc2-c2cccc(-c3nc(-c4ccccc4)nc(-c4ccccc4C)n3)c2)n1 ; Cc1ccc(-c2nc(-c3ccccc3)nc(-c3ccccc3-c3ccc(-c4ccccn4)cc3-c3ccccc3-c3nc(-c4ccccc4)nc(-c4cccc(C)c4)n3)n2)cc1]

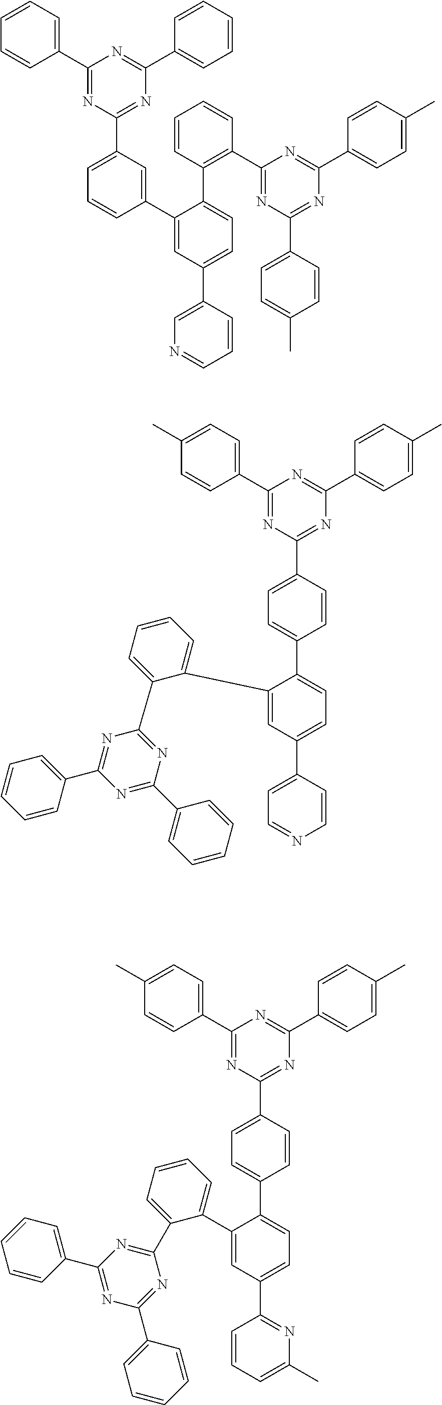 [SMILES: Cc1ccc(-c2nc(-c3ccc(C)cc3)nc(-c3ccccc3-c3ccc(-c4cccnc4)cc3-c3cccc(-c4nc(-c5ccccc5)nc(-c5ccccc5)n4)c3)n2)cc1 ; Cc1ccc(-c2nc(-c3ccc(C)cc3)nc(-c3ccc(-c4ccc(-c5ccncc5)cc4-c4ccccc4-c4nc(-c5ccccc5)nc(-c5ccccc5)n4)cc3)n2)cc1 ; Cc1ccc(-c2nc(-c3ccc(C)cc3)nc(-c3ccc(-c4ccc(-c5cccc(C)n5)cc4-c4ccccc4-c4nc(-c5ccccc5)nc(-c5ccccc5)n4)cc3)n2)cc1]

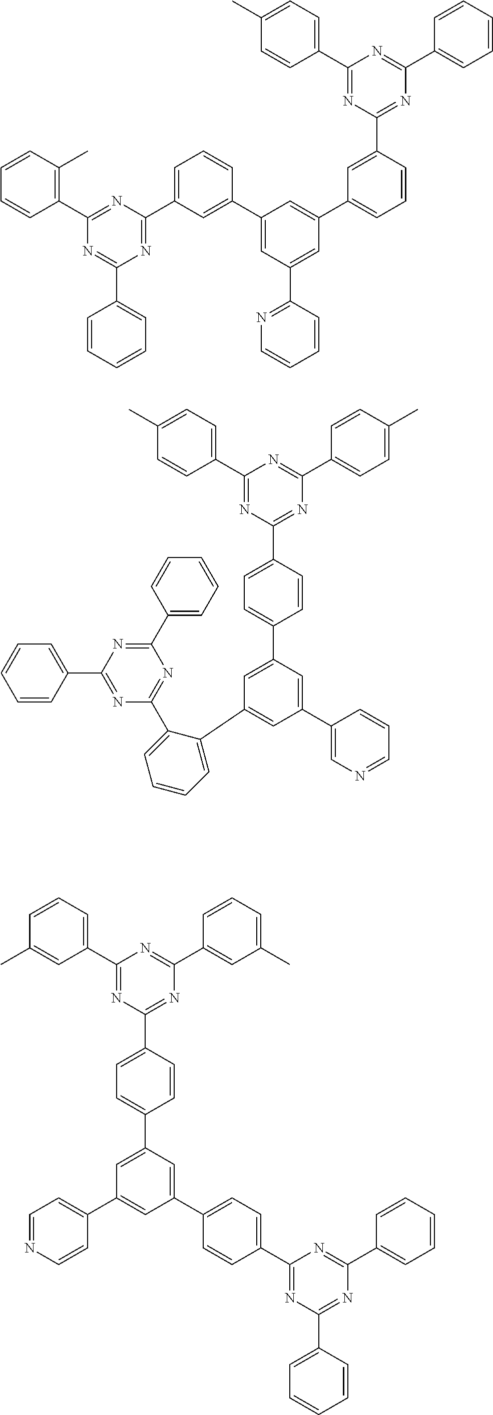

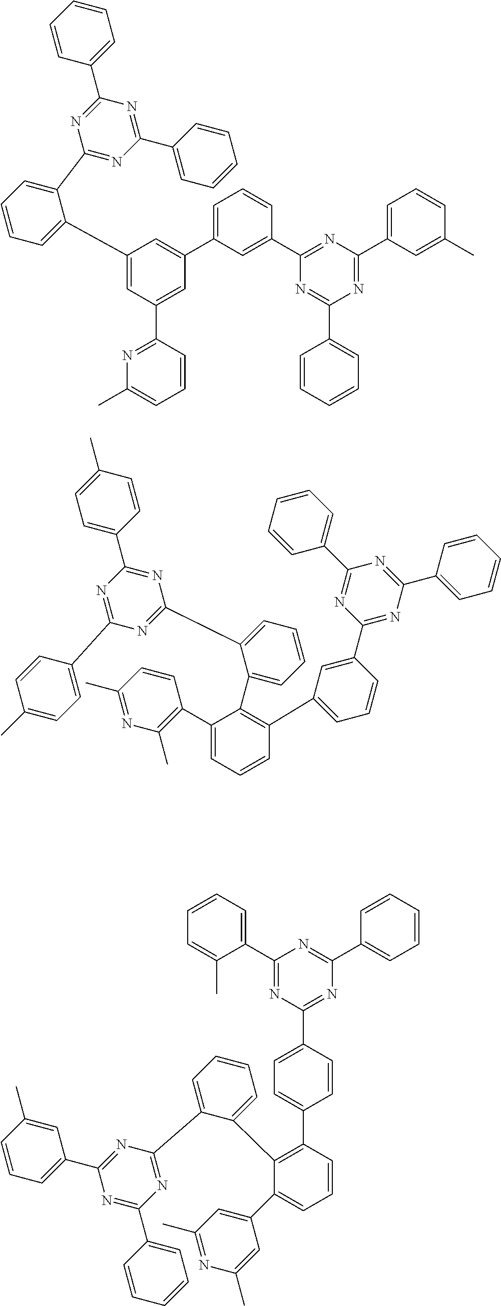

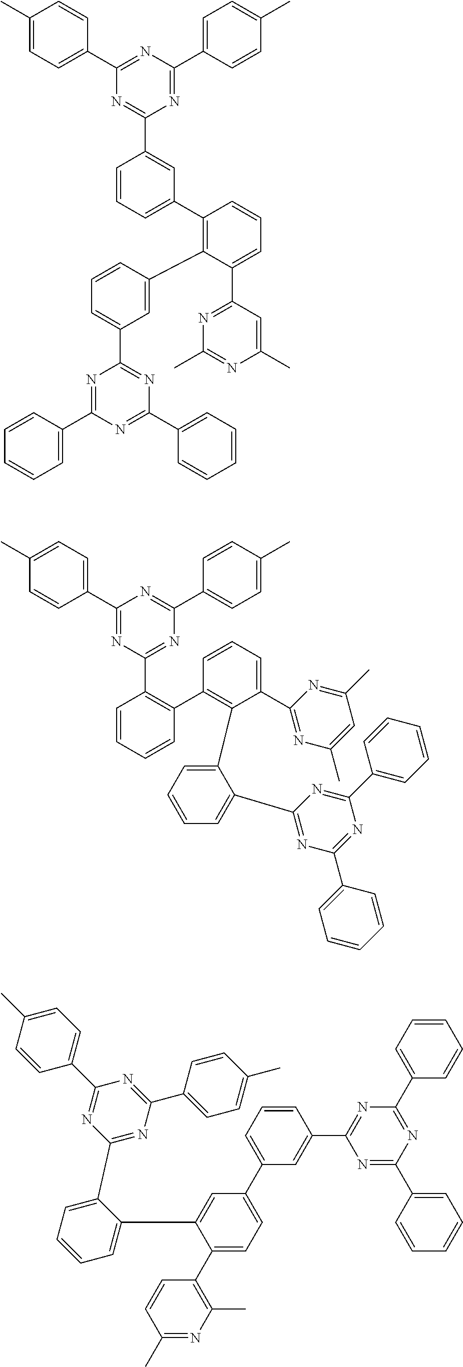

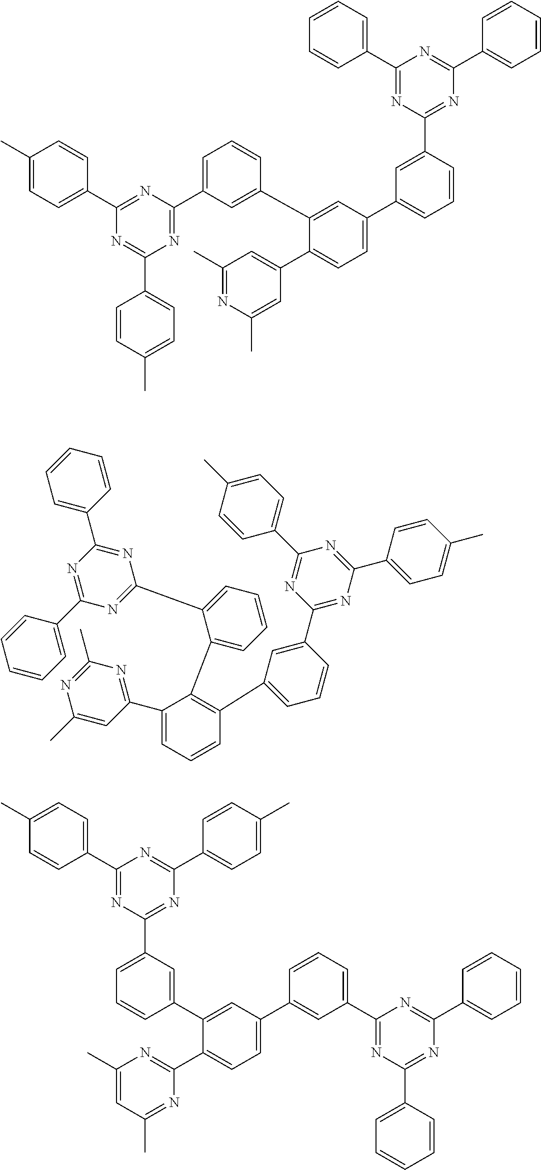

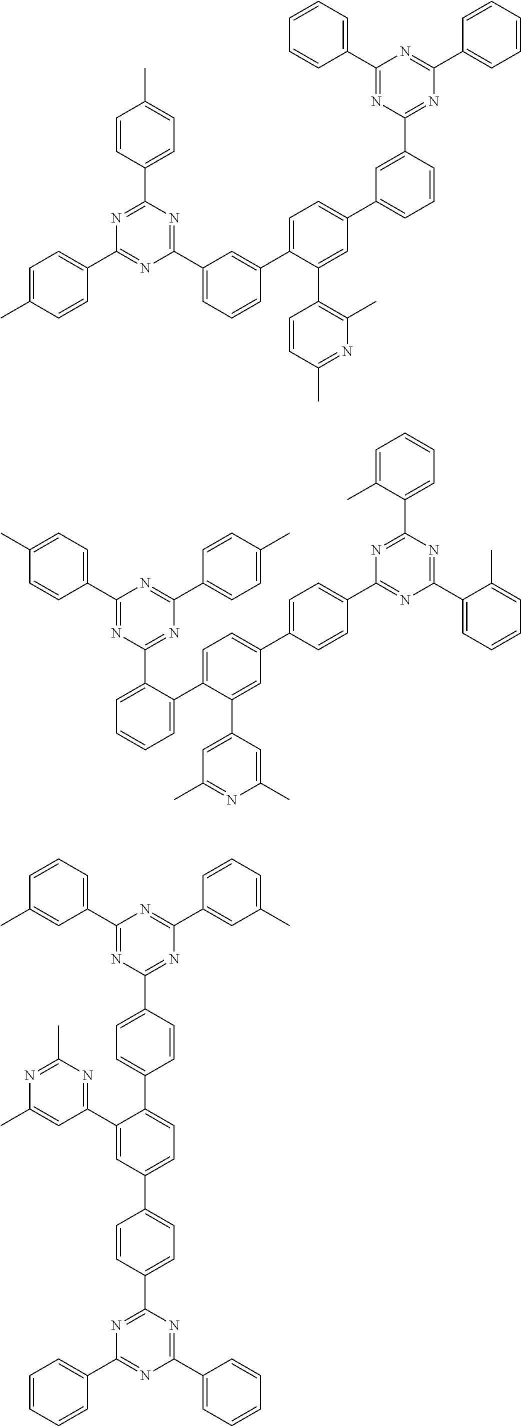

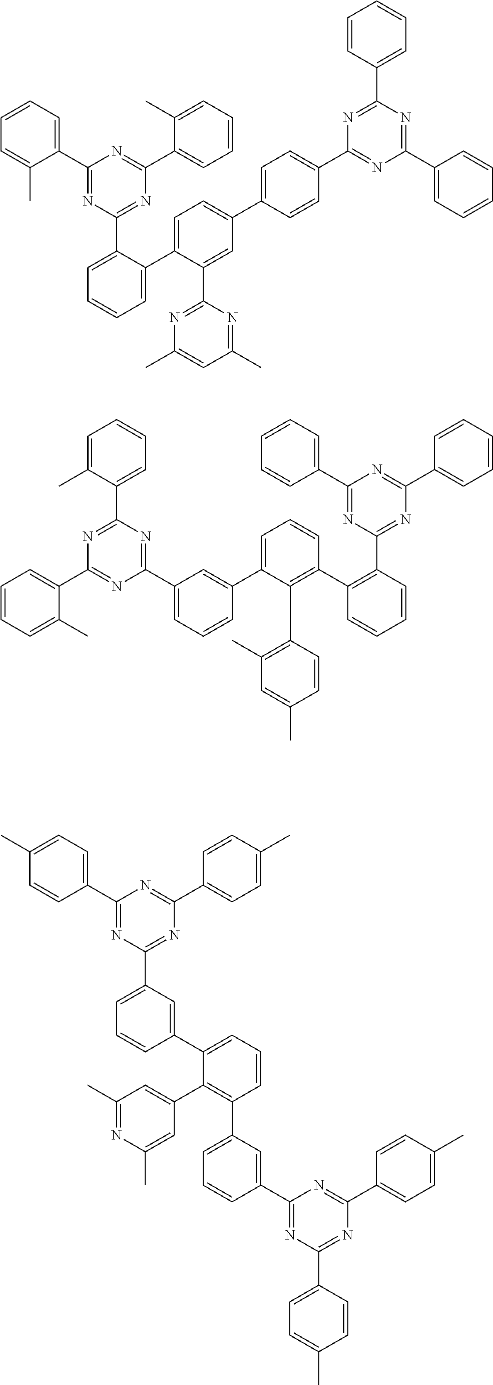

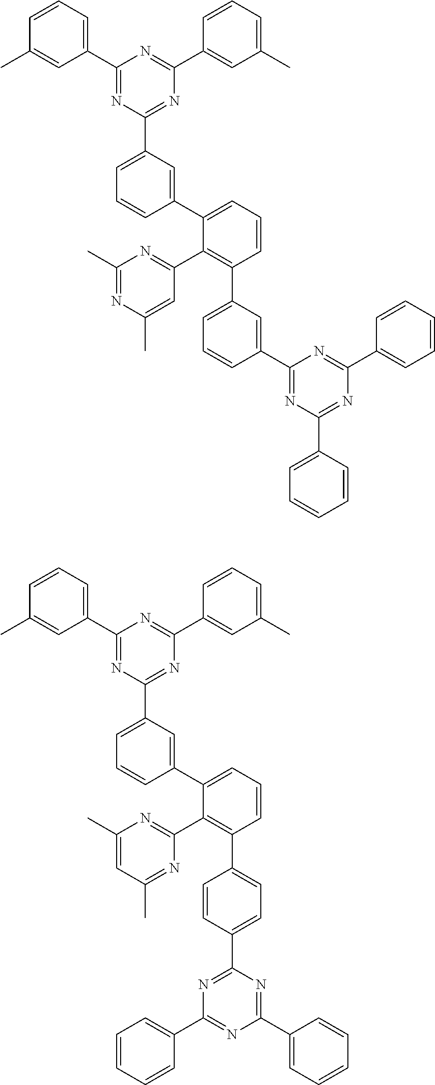 [SMILES: Cc1cccc(-c2nc(-c3cccc(C)c3)nc(-c3cccc(-c4cccc(-c5cccc(-c6nc(-c7ccccc7)nc(-c7ccccc7)n6)c5)c4-c4cc(C)nc(C)n4)c3)n2)c1 ; Cc1cccc(-c2nc(-c3cccc(C)c3)nc(-c3cccc(-c4cccc(-c5ccc(-c6nc(-c7ccccc7)nc(-c7ccccc7)n6)cc5)c4-c4nc(C)cc(C)n4)c3)n2)c1]

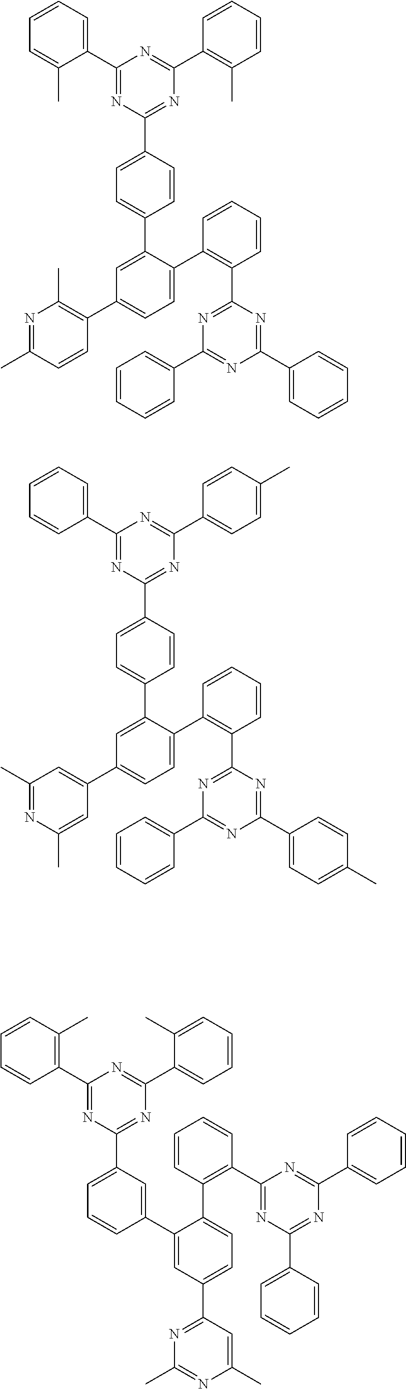

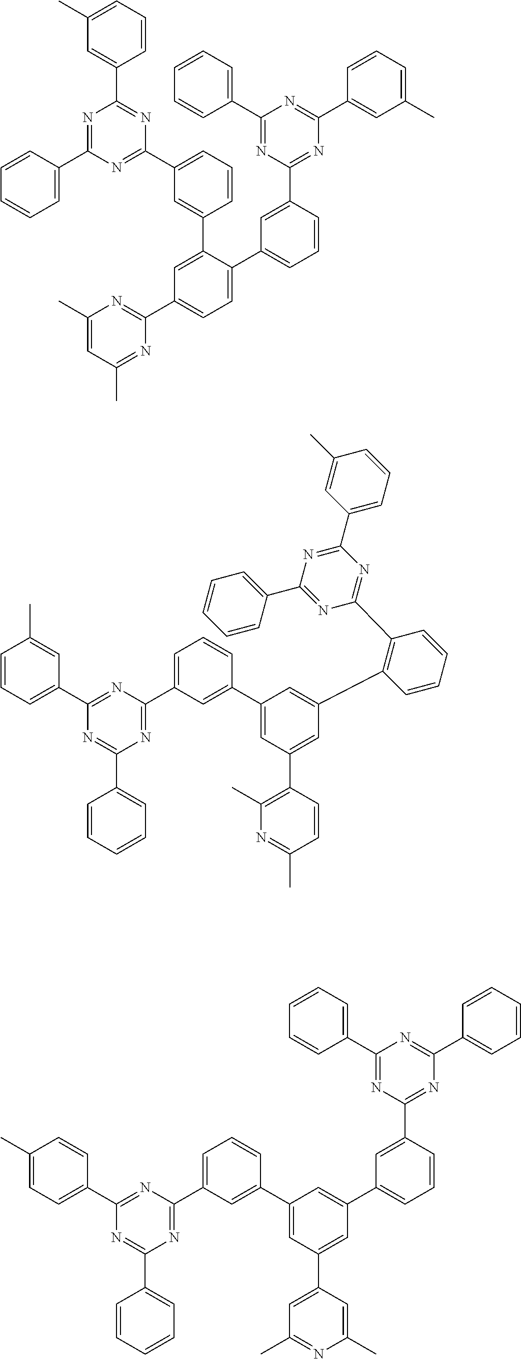

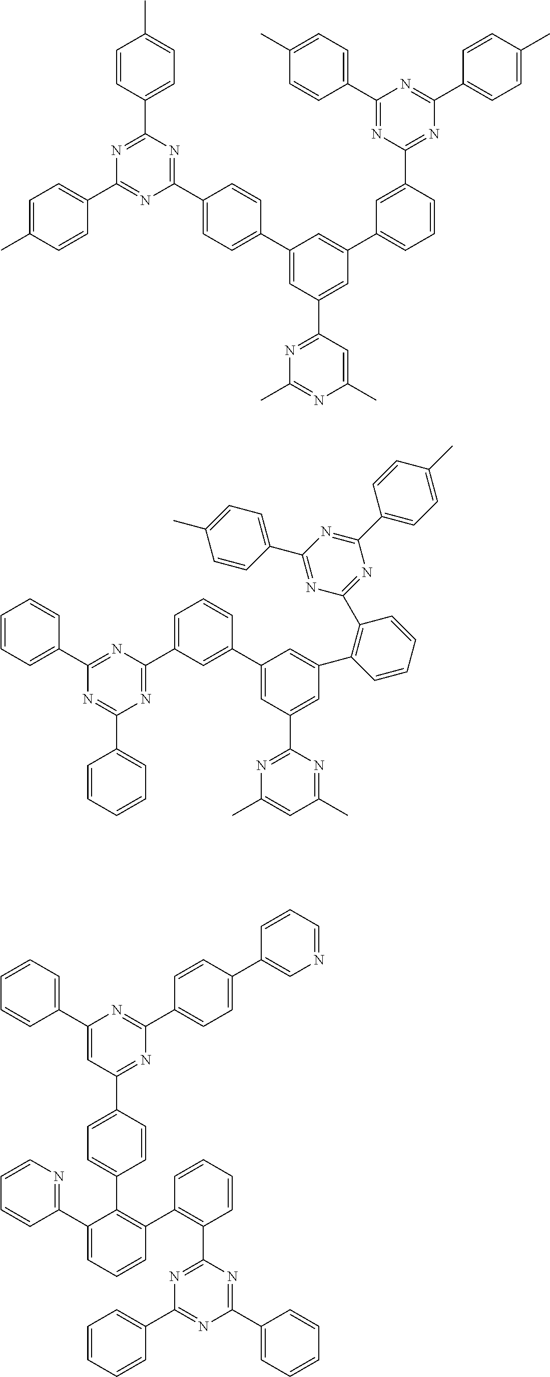

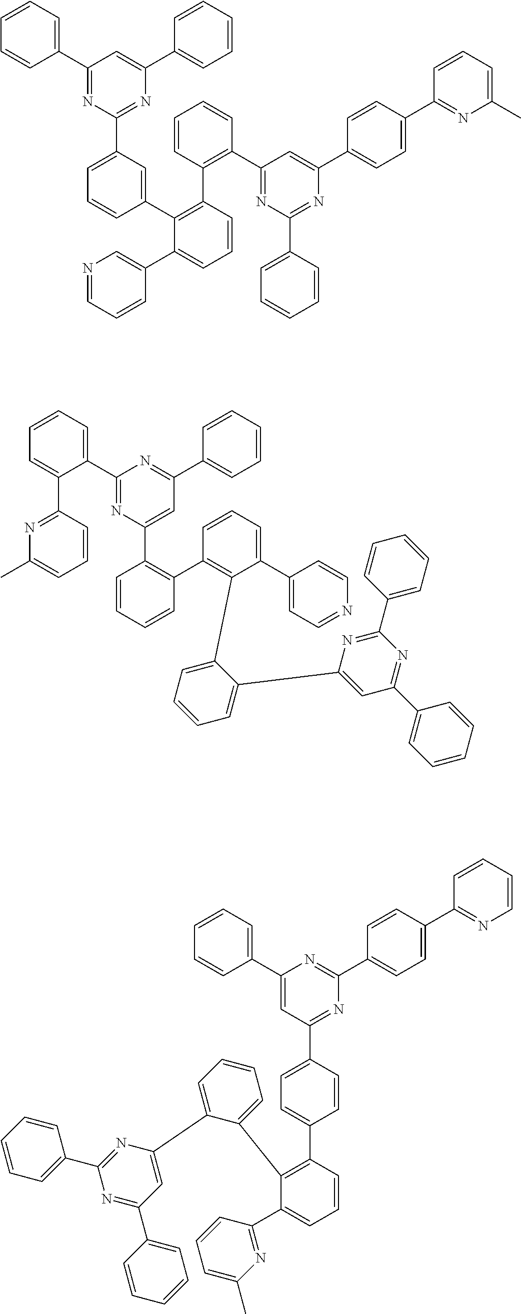

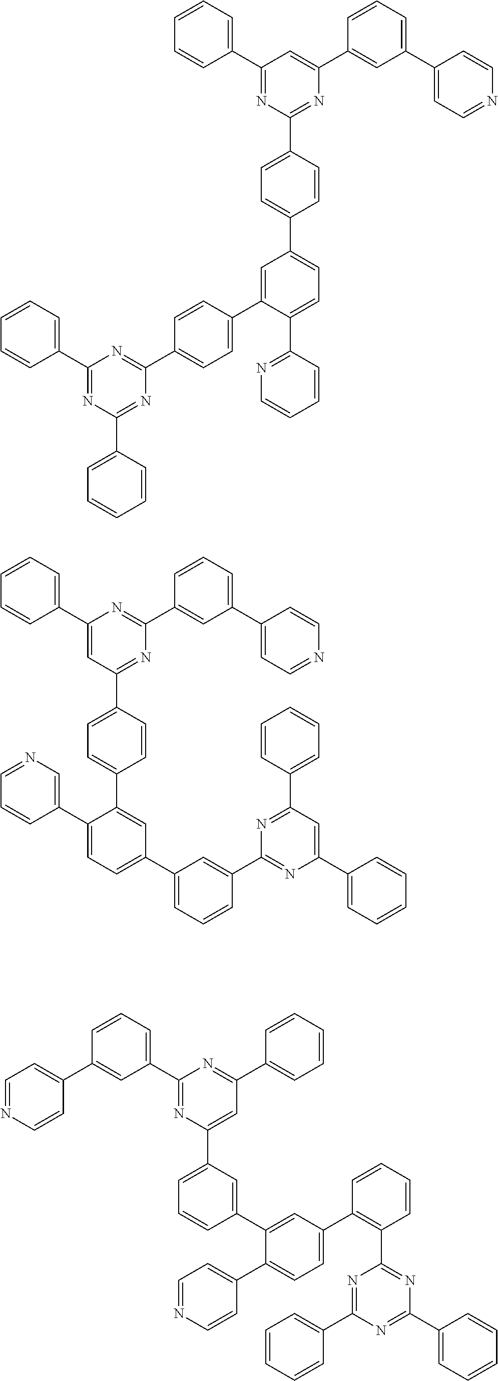 [SMILES: c1ccc(-c2cc(-c3cccc(-c4ccncc4)c3)nc(-c3ccc(-c4ccc(-c5ccccn5)c(-c5ccc(-c6nc(-c7ccccc7)nc(-c7ccccc7)n6)cc5)c4)cc3)n2)cc1 ; c1ccc(-c2cc(-c3ccc(-c4cc(-c5cccc(-c6nc(-c7ccccc7)cc(-c7ccccc7)n6)c5)ccc4-c4cccnc4)cc3)nc(-c3cccc(-c4ccncc4)c3)n2)cc1 ; c1ccc(-c2cc(-c3cccc(-c4cc(-c5ccccc5-c5nc(-c6ccccc6)nc(-c6ccccc6)n5)ccc4-c4ccncc4)c3)nc(-c3cccc(-c4ccncc4)c3)n2)cc1]

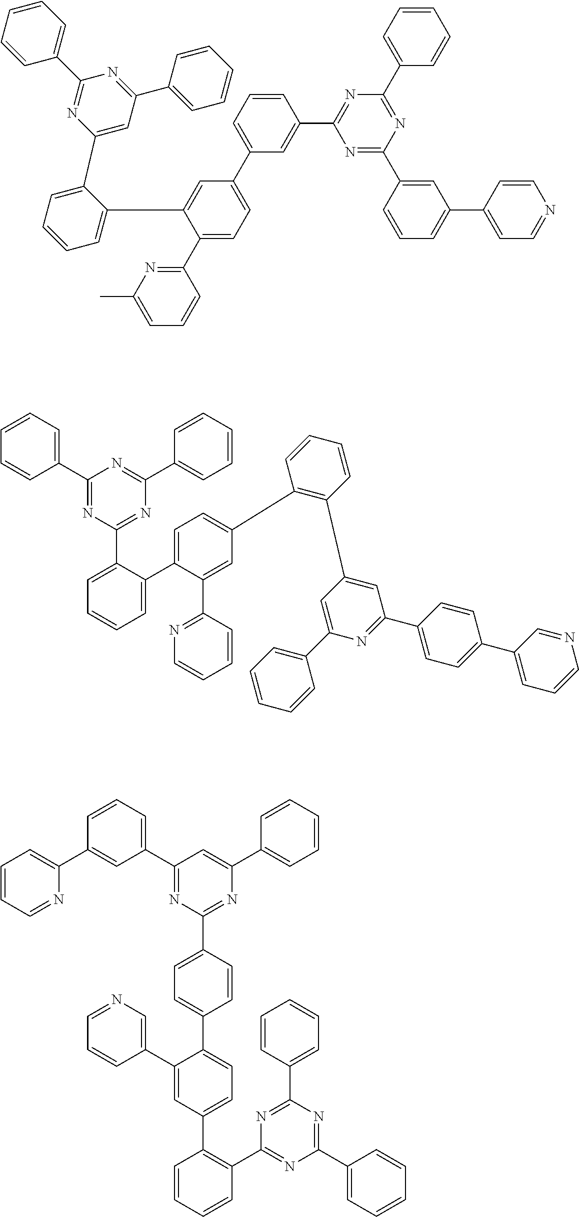 [SMILES: Cc1cccc(-c2ccc(-c3cccc(-c4nc(-c5ccccc5)nc(-c5cccc(-c6ccncc6)c5)n4)c3)cc2-c2ccccc2-c2cc(-c3ccccc3)nc(-c3ccccc3)n2)n1 ; c1ccc(-c2cc(-c3ccccc3-c3ccc(-c4ccccc4-c4nc(-c5ccccc5)nc(-c5ccccc5)n4)c(-c4ccccn4)c3)cc(-c3ccc(-c4cccnc4)cc3)n2)cc1 ; c1ccc(-c2cc(-c3cccc(-c4ccccn4)c3)nc(-c3ccc(-c4ccc(-c5ccccc5-c5nc(-c6ccccc6)nc(-c6ccccc6)n5)cc4-c4cccnc4)cc3)n2)cc1]

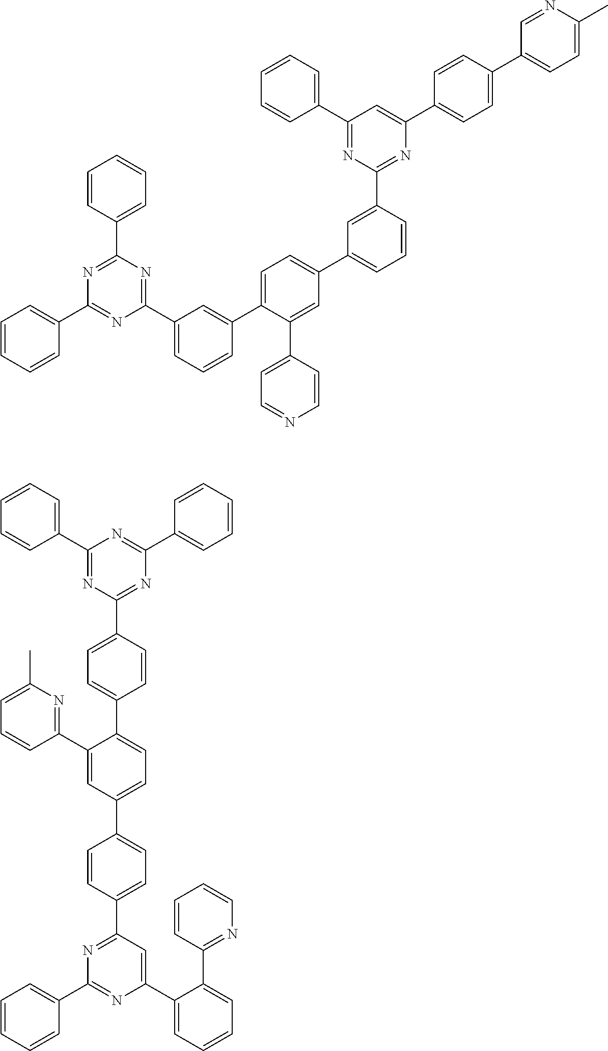 [SMILES: Cc1ccc(-c2ccc(-c3cc(-c4ccccc4)nc(-c4cccc(-c5ccc(-c6cccc(-c7nc(-c8ccccc8)nc(-c8ccccc8)n7)c6)c(-c6ccncc6)c5)c4)n3)cc2)cn1 ; Cc1cccc(-c2cc(-c3ccc(-c4cc(-c5ccccc5-c5ccccn5)nc(-c5ccccc5)n4)cc3)ccc2-c2ccc(-c3nc(-c4ccccc4)nc(-c4ccccc4)n3)cc2)n1]

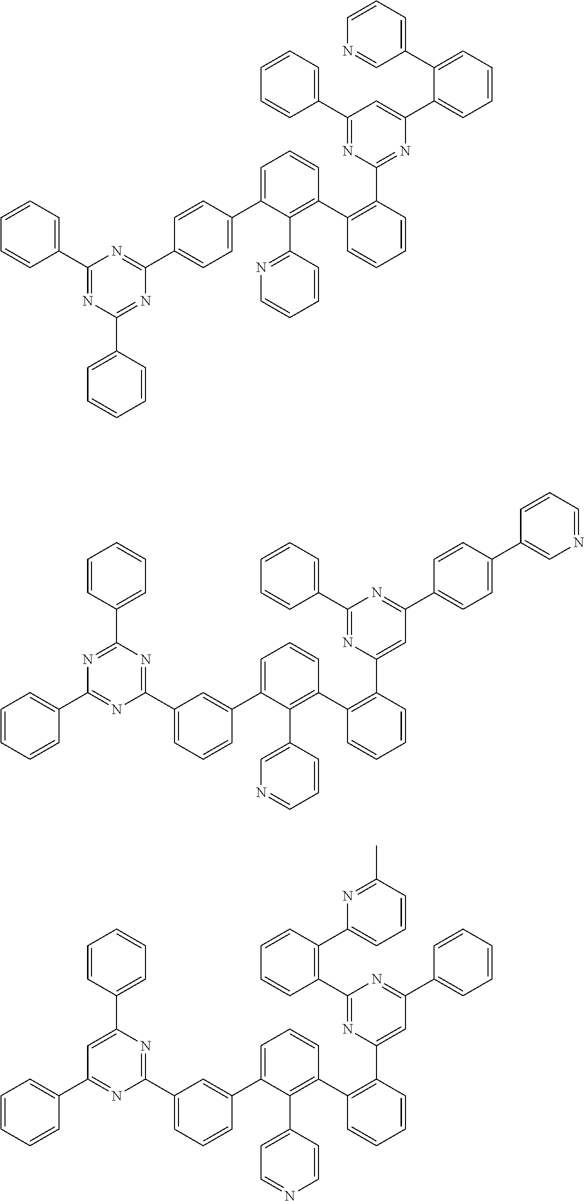 [SMILES: c1ccc(-c2cc(-c3ccccc3-c3cccnc3)nc(-c3ccccc3-c3cccc(-c4ccc(-c5nc(-c6ccccc6)nc(-c6ccccc6)n5)cc4)c3-c3ccccn3)n2)cc1 ; c1ccc(-c2nc(-c3ccc(-c4cccnc4)cc3)cc(-c3ccccc3-c3cccc(-c4cccc(-c5nc(-c6ccccc6)nc(-c6ccccc6)n5)c4)c3-c3cccnc3)n2)cc1 ; Cc1cccc(-c2ccccc2-c2nc(-c3ccccc3)cc(-c3ccccc3-c3cccc(-c4cccc(-c5nc(-c6ccccc6)cc(-c6ccccc6)n5)c4)c3-c3ccncc3)n2)n1]

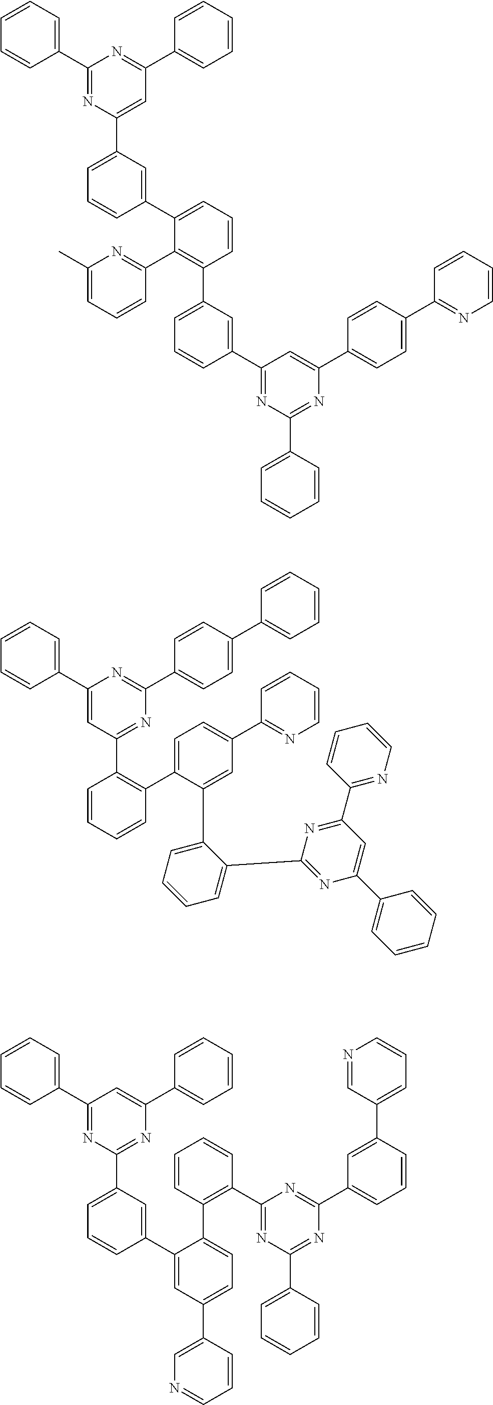 [SMILES: Cc1cccc(-c2c(-c3cccc(-c4cc(-c5ccccc5)nc(-c5ccccc5)n4)c3)cccc2-c2cccc(-c3cc(-c4ccc(-c5ccccn5)cc4)nc(-c4ccccc4)n3)c2)n1 ; c1ccc(-c2ccc(-c3nc(-c4ccccc4)cc(-c4ccccc4-c4ccc(-c5ccccn5)cc4-c4ccccc4-c4nc(-c5ccccc5)cc(-c5ccccn5)n4)n3)cc2)cc1 ; c1ccc(-c2cc(-c3ccccc3)nc(-c3cccc(-c4cc(-c5cccnc5)ccc4-c4ccccc4-c4nc(-c5ccccc5)nc(-c5cccc(-c6cccnc6)c5)n4)c3)n2)cc1]

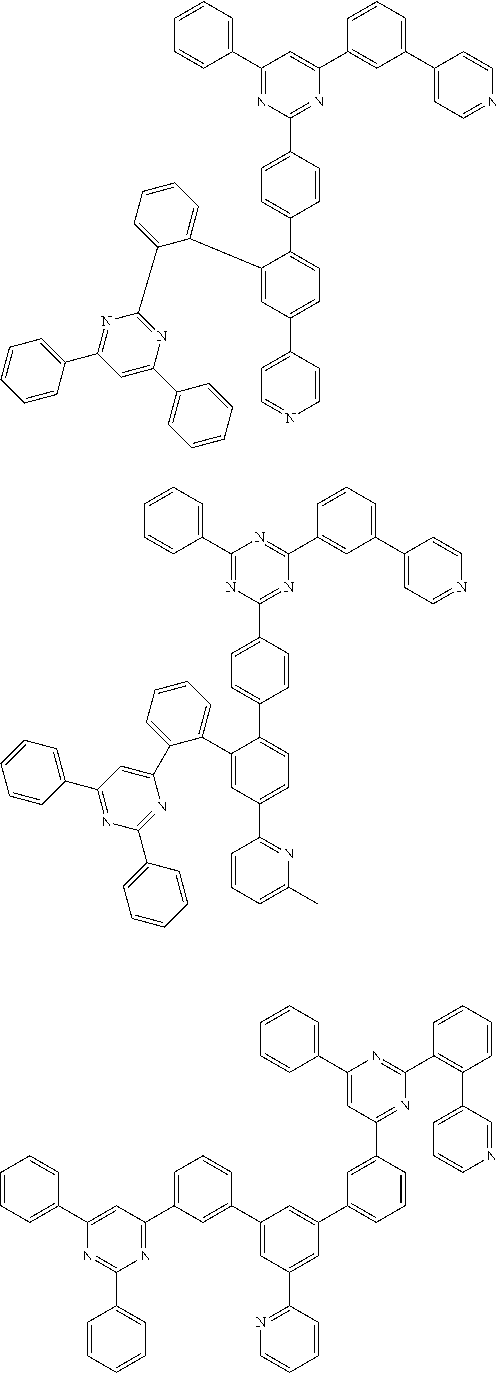

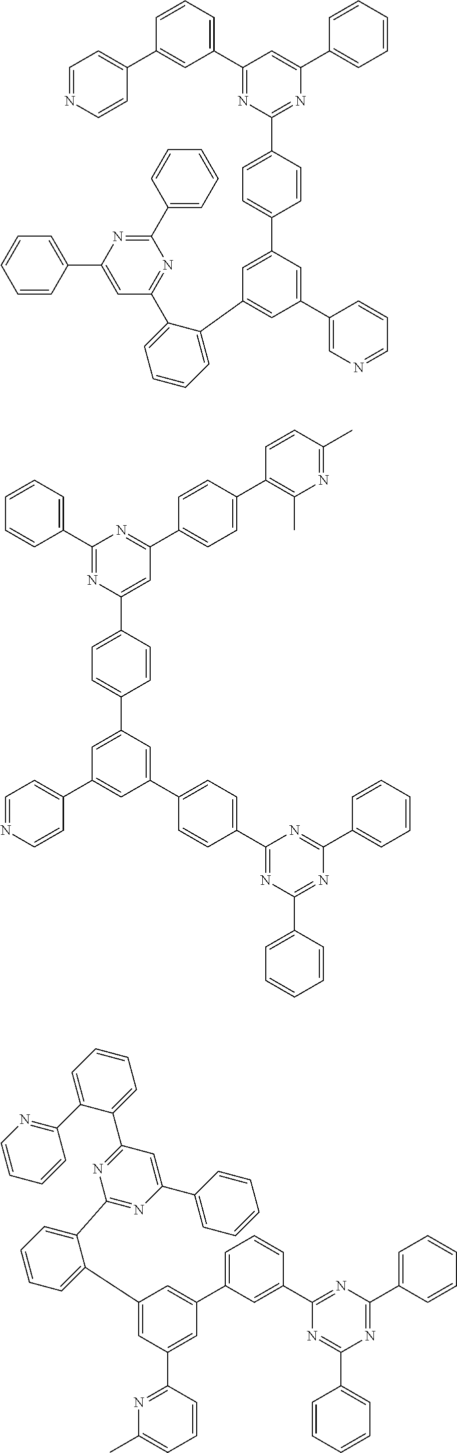 [SMILES: c1ccc(-c2cc(-c3cccc(-c4ccncc4)c3)nc(-c3ccc(-c4cc(-c5cccnc5)cc(-c5ccccc5-c5cc(-c6ccccc6)nc(-c6ccccc6)n5)c4)cc3)n2)cc1 ; Cc1ccc(-c2ccc(-c3cc(-c4ccc(-c5cc(-c6ccncc6)cc(-c6ccc(-c7nc(-c8ccccc8)nc(-c8ccccc8)n7)cc6)c5)cc4)nc(-c4ccccc4)n3)cc2)c(C)n1 ; Cc1cccc(-c2cc(-c3cccc(-c4nc(-c5ccccc5)nc(-c5ccccc5)n4)c3)cc(-c3ccccc3-c3nc(-c4ccccc4)cc(-c4ccccc4-c4ccccn4)n3)c2)n1]

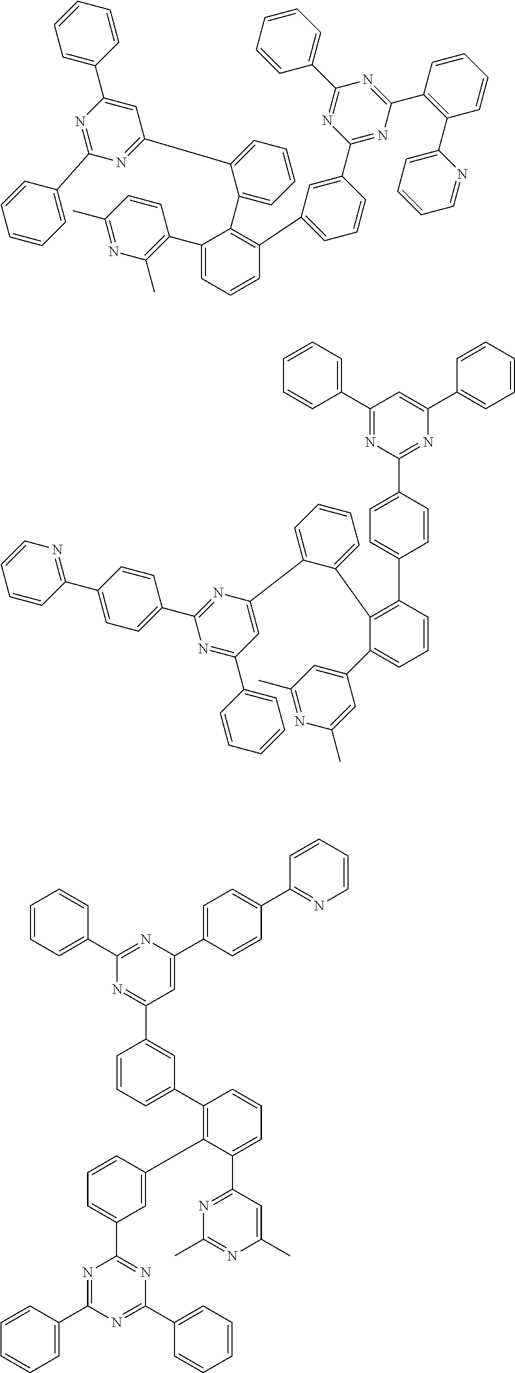 [SMILES: Cc1ccc(-c2cccc(-c3cccc(-c4nc(-c5ccccc5)nc(-c5ccccc5-c5ccccn5)n4)c3)c2-c2ccccc2-c2cc(-c3ccccc3)nc(-c3ccccc3)n2)c(C)n1 ; Cc1cc(-c2cccc(-c3ccc(-c4nc(-c5ccccc5)cc(-c5ccccc5)n4)cc3)c2-c2ccccc2-c2cc(-c3ccccc3)nc(-c3ccc(-c4ccccn4)cc3)n2)cc(C)n1 ; Cc1cc(-c2cccc(-c3cccc(-c4cc(-c5ccc(-c6ccccn6)cc5)nc(-c5ccccc5)n4)c3)c2-c2cccc(-c3nc(-c4ccccc4)nc(-c4ccccc4)n3)c2)nc(C)n1]

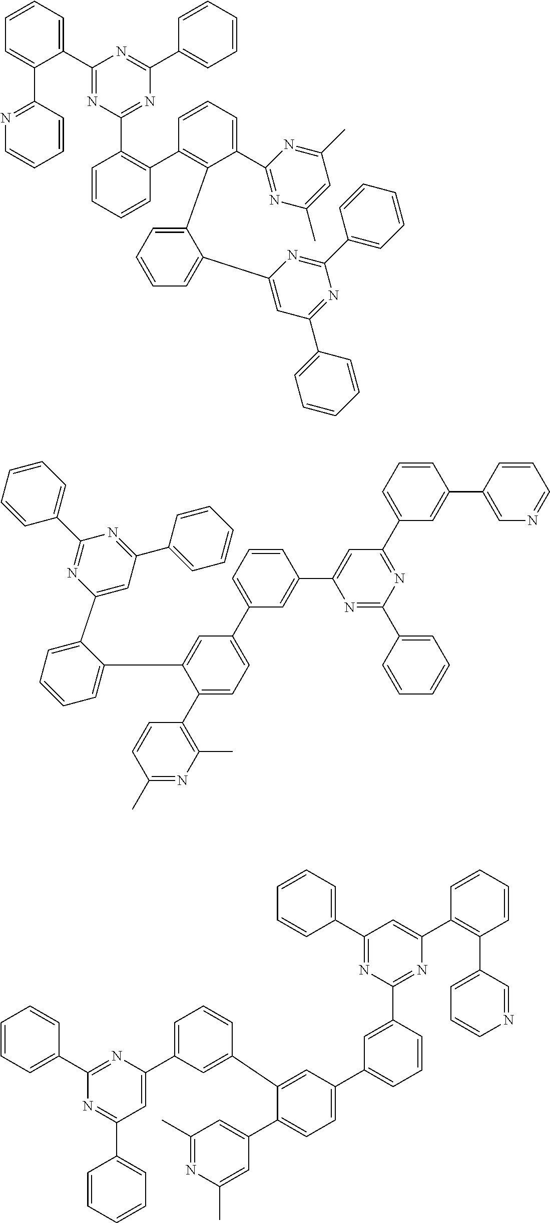 [SMILES: Cc1cc(C)nc(-c2cccc(-c3ccccc3-c3nc(-c4ccccc4)nc(-c4ccccc4-c4ccccn4)n3)c2-c2ccccc2-c2cc(-c3ccccc3)nc(-c3ccccc3)n2)n1 ; Cc1ccc(-c2ccc(-c3cccc(-c4cc(-c5cccc(-c6cccnc6)c5)nc(-c5ccccc5)n4)c3)cc2-c2ccccc2-c2cc(-c3ccccc3)nc(-c3ccccc3)n2)c(C)n1 ; Cc1cc(-c2ccc(-c3cccc(-c4nc(-c5ccccc5)cc(-c5ccccc5-c5cccnc5)n4)c3)cc2-c2cccc(-c3cc(-c4ccccc4)nc(-c4ccccc4)n3)c2)cc(C)n1]

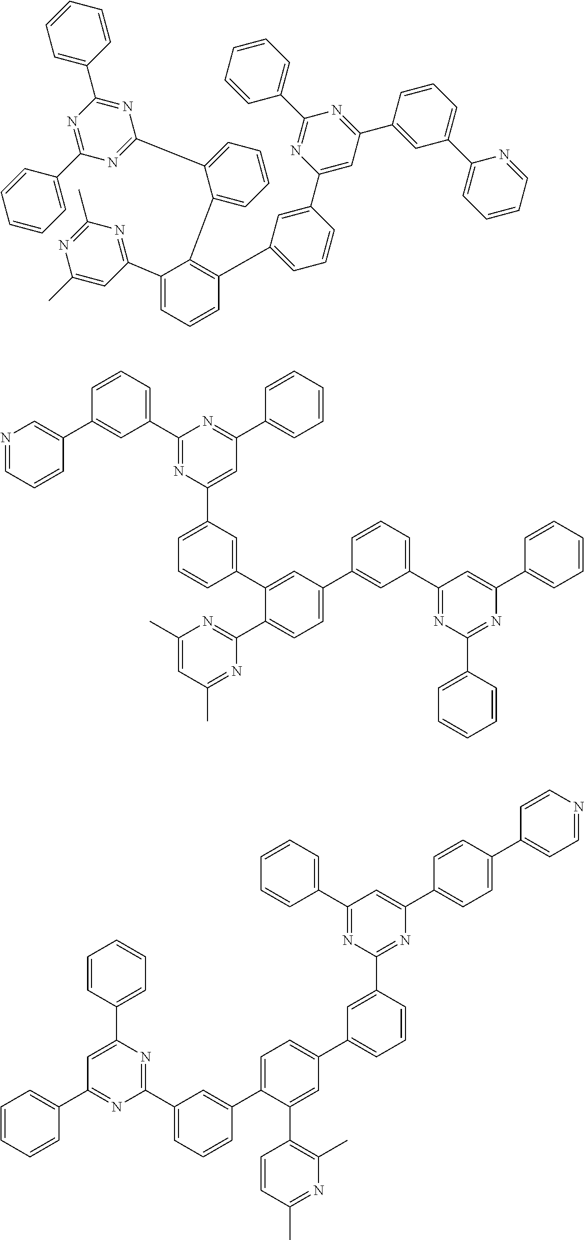 [SMILES: Cc1cc(-c2cccc(-c3cccc(-c4cc(-c5cccc(-c6ccccn6)c5)nc(-c5ccccc5)n4)c3)c2-c2ccccc2-c2nc(-c3ccccc3)nc(-c3ccccc3)n2)nc(C)n1 ; Cc1cc(C)nc(-c2ccc(-c3cccc(-c4cc(-c5ccccc5)nc(-c5ccccc5)n4)c3)cc2-c2cccc(-c3cc(-c4ccccc4)nc(-c4cccc(-c5cccnc5)c4)n3)c2)n1 ; Cc1ccc(-c2cc(-c3cccc(-c4nc(-c5ccccc5)cc(-c5ccc(-c6ccncc6)cc5)n4)c3)ccc2-c2cccc(-c3nc(-c4ccccc4)cc(-c4ccccc4)n3)c2)c(C)n1]

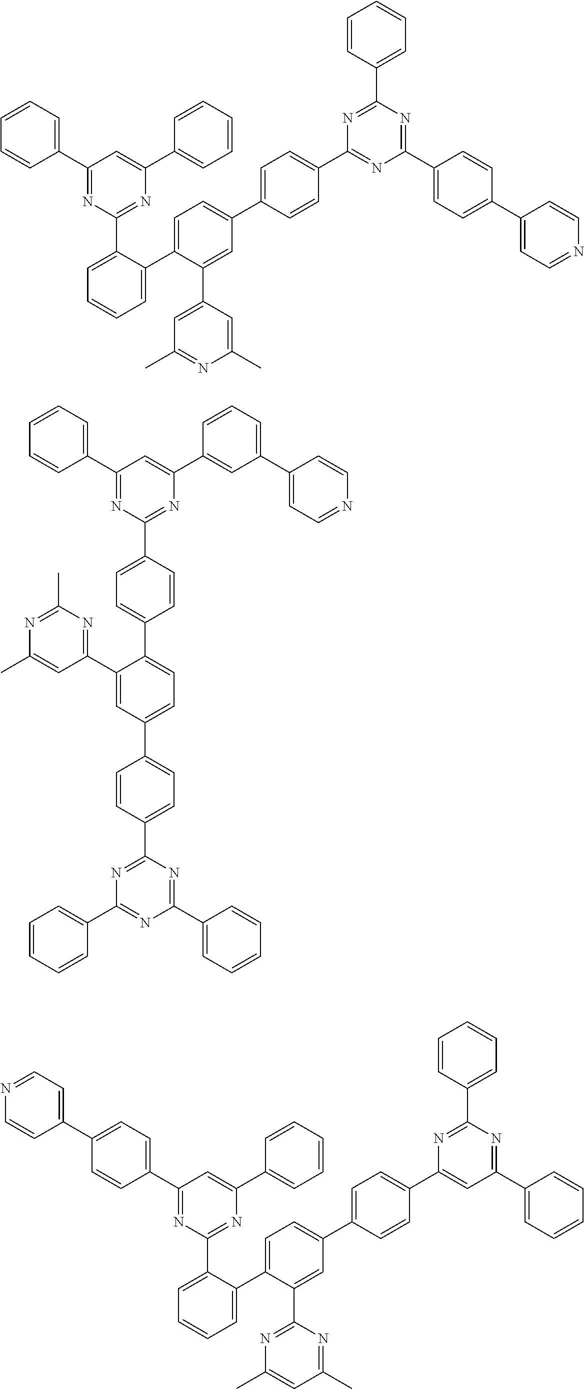 [SMILES: Cc1cc(-c2cc(-c3ccc(-c4nc(-c5ccccc5)nc(-c5ccc(-c6ccncc6)cc5)n4)cc3)ccc2-c2ccccc2-c2nc(-c3ccccc3)cc(-c3ccccc3)n2)cc(C)n1 ; Cc1cc(-c2cc(-c3ccc(-c4nc(-c5ccccc5)nc(-c5ccccc5)n4)cc3)ccc2-c2ccc(-c3nc(-c4ccccc4)cc(-c4cccc(-c5ccncc5)c4)n3)cc2)nc(C)n1 ; Cc1cc(C)nc(-c2cc(-c3ccc(-c4cc(-c5ccccc5)nc(-c5ccccc5)n4)cc3)ccc2-c2ccccc2-c2nc(-c3ccccc3)cc(-c3ccc(-c4ccncc4)cc3)n2)n1]

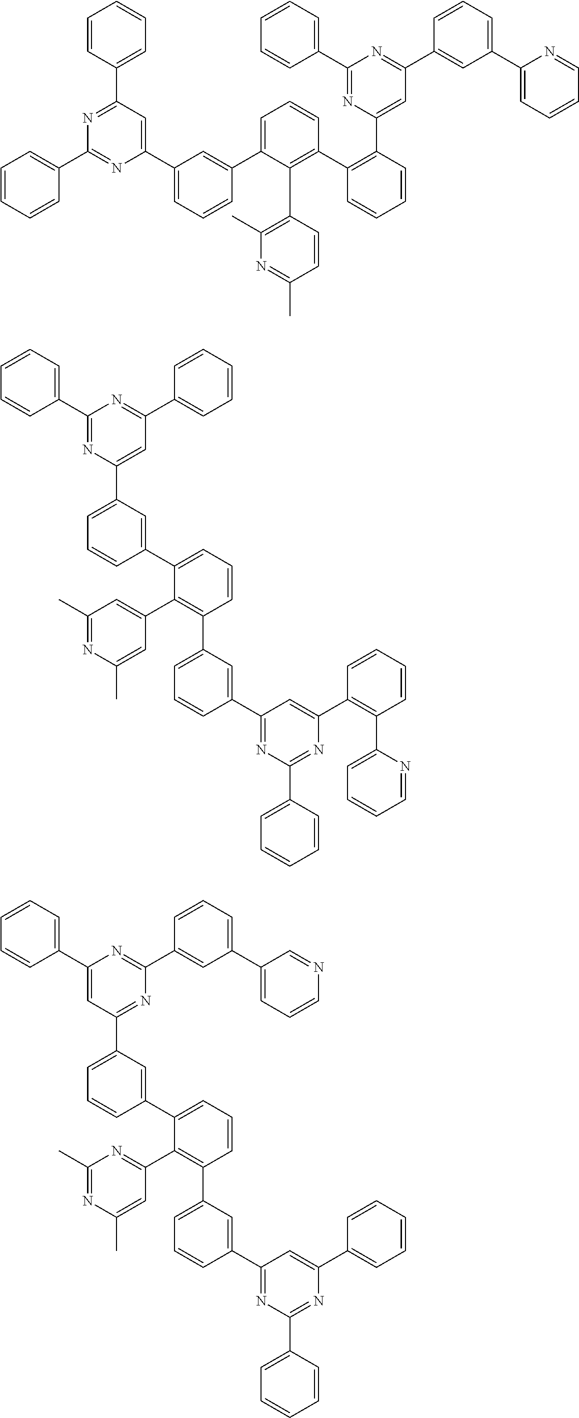 [SMILES: Cc1ccc(-c2c(-c3cccc(-c4cc(-c5ccccc5)nc(-c5ccccc5)n4)c3)cccc2-c2ccccc2-c2cc(-c3cccc(-c4ccccn4)c3)nc(-c3ccccc3)n2)c(C)n1 ; Cc1cc(-c2c(-c3cccc(-c4cc(-c5ccccc5)nc(-c5ccccc5)n4)c3)cccc2-c2cccc(-c3cc(-c4ccccc4-c4ccccn4)nc(-c4ccccc4)n3)c2)cc(C)n1 ; Cc1cc(-c2c(-c3cccc(-c4cc(-c5ccccc5)nc(-c5ccccc5)n4)c3)cccc2-c2cccc(-c3cc(-c4ccccc4)nc(-c4cccc(-c5cccnc5)c4)n3)c2)nc(C)n1]

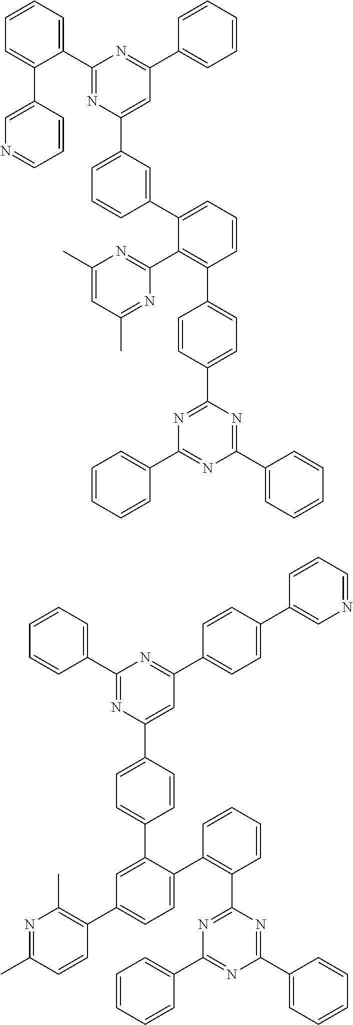

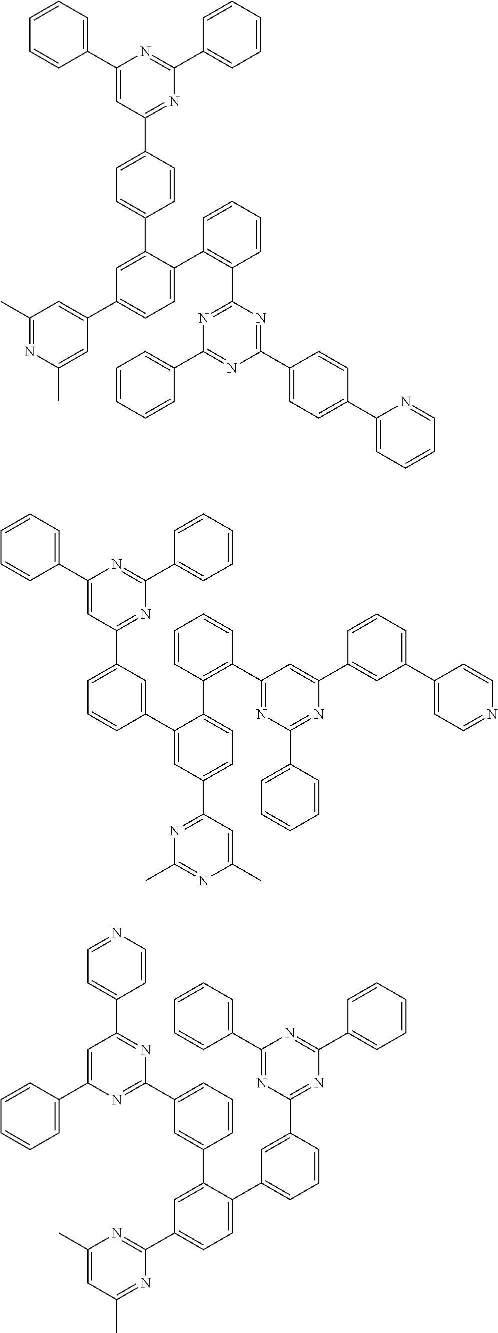 [SMILES: Cc1cc(-c2ccc(-c3ccccc3-c3nc(-c4ccccc4)nc(-c4ccc(-c5ccccn5)cc4)n3)c(-c3ccc(-c4cc(-c5ccccc5)nc(-c5ccccc5)n4)cc3)c2)cc(C)n1 ; Cc1cc(-c2ccc(-c3ccccc3-c3cc(-c4cccc(-c5ccncc5)c4)nc(-c4ccccc4)n3)c(-c3cccc(-c4cc(-c5ccccc5)nc(-c5ccccc5)n4)c3)c2)nc(C)n1 ; Cc1cc(C)nc(-c2ccc(-c3cccc(-c4nc(-c5ccccc5)nc(-c5ccccc5)n4)c3)c(-c3cccc(-c4nc(-c5ccccc5)cc(-c5ccncc5)n4)c3)c2)n1]

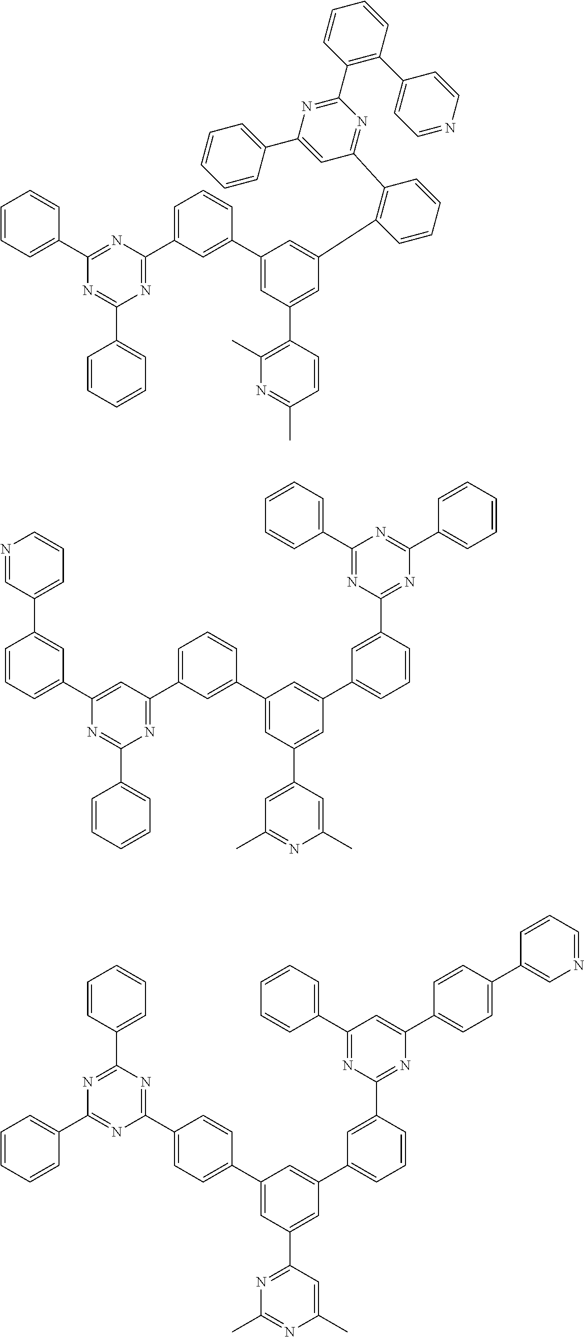

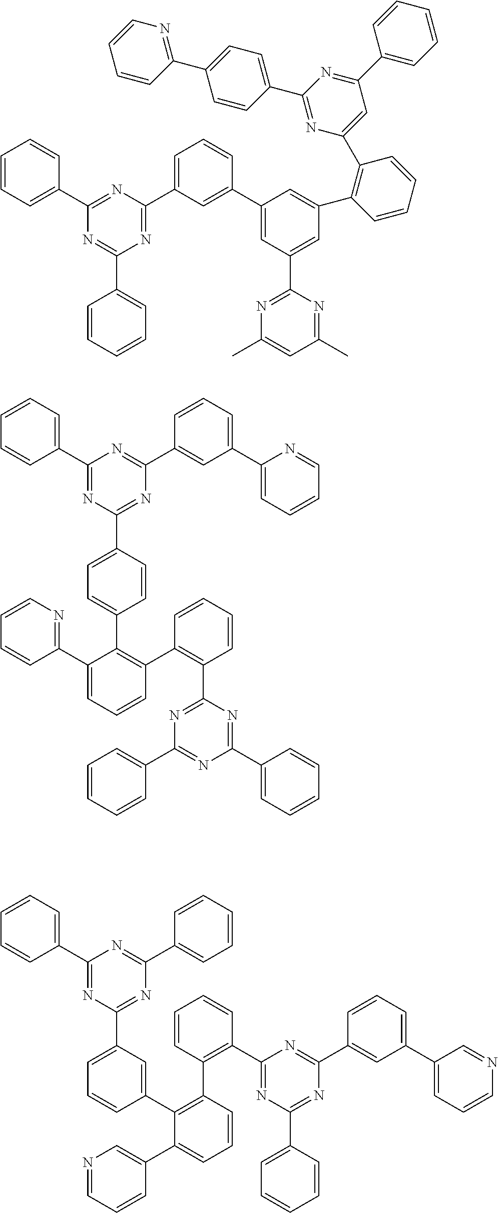 [SMILES: Cc1cc(C)nc(-c2cc(-c3cccc(-c4nc(-c5ccccc5)nc(-c5ccccc5)n4)c3)cc(-c3ccccc3-c3cc(-c4ccccc4)nc(-c4ccc(-c5ccccn5)cc4)n3)c2)n1 ; c1ccc(-c2nc(-c3ccc(-c4c(-c5ccccn5)cccc4-c4ccccc4-c4nc(-c5ccccc5)nc(-c5ccccc5)n4)cc3)nc(-c3cccc(-c4ccccn4)c3)n2)cc1 ; c1ccc(-c2nc(-c3ccccc3)nc(-c3cccc(-c4c(-c5cccnc5)cccc4-c4ccccc4-c4nc(-c5ccccc5)nc(-c5cccc(-c6cccnc6)c5)n4)c3)n2)cc1]

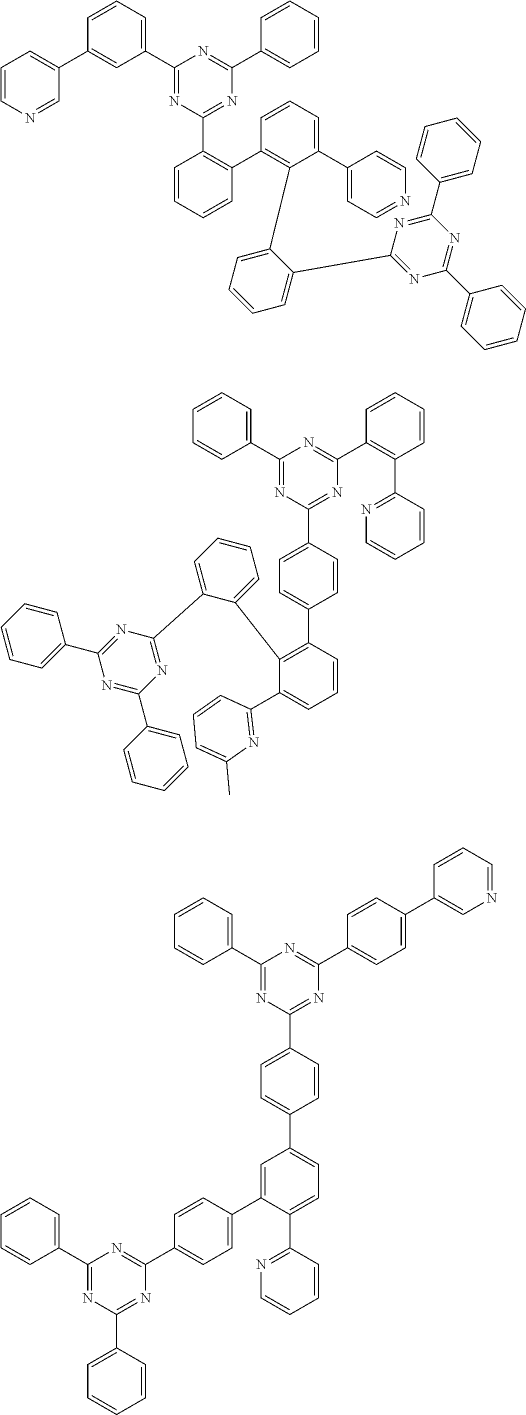 [SMILES: c1ccc(-c2nc(-c3cccc(-c4cccnc4)c3)nc(-c3ccccc3-c3cccc(-c4ccncc4)c3-c3ccccc3-c3nc(-c4ccccc4)nc(-c4ccccc4)n3)n2)cc1 ; Cc1cccc(-c2cccc(-c3ccc(-c4nc(-c5ccccc5)nc(-c5ccccc5-c5ccccn5)n4)cc3)c2-c2ccccc2-c2nc(-c3ccccc3)nc(-c3ccccc3)n2)n1 ; c1ccc(-c2nc(-c3ccc(-c4cccnc4)cc3)nc(-c3ccc(-c4ccc(-c5ccccn5)c(-c5ccc(-c6nc(-c7ccccc7)nc(-c7ccccc7)n6)cc5)c4)cc3)n2)cc1]

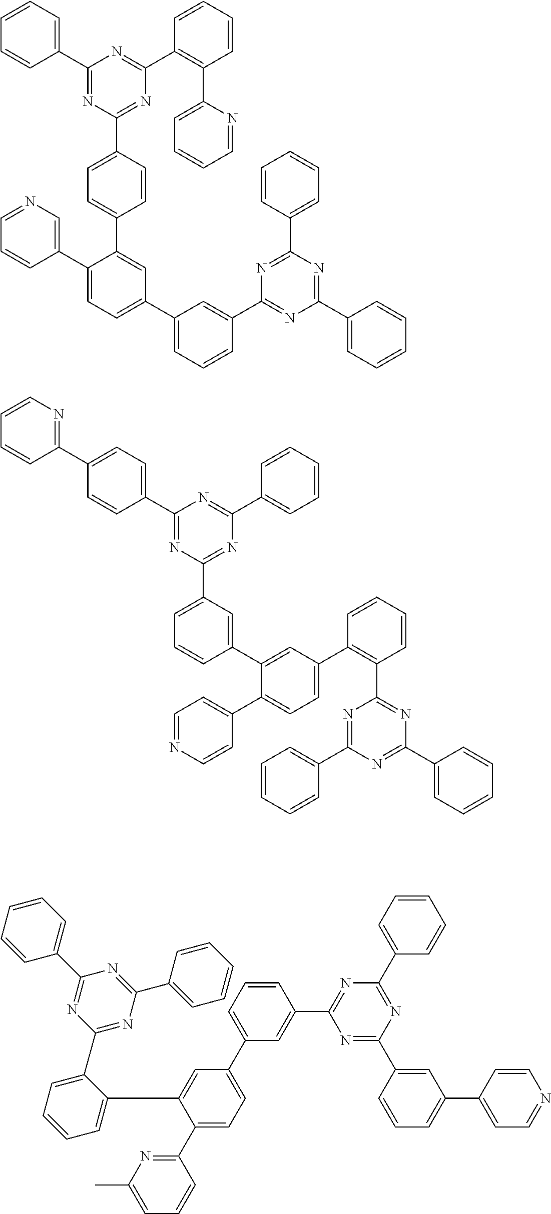 [SMILES: c1ccc(-c2nc(-c3ccccc3)nc(-c3cccc(-c4ccc(-c5cccnc5)c(-c5ccc(-c6nc(-c7ccccc7)nc(-c7ccccc7-c7ccccn7)n6)cc5)c4)c3)n2)cc1 ; c1ccc(-c2nc(-c3ccc(-c4ccccn4)cc3)nc(-c3cccc(-c4cc(-c5ccccc5-c5nc(-c6ccccc6)nc(-c6ccccc6)n5)ccc4-c4ccncc4)c3)n2)cc1 ; Cc1cccc(-c2ccc(-c3cccc(-c4nc(-c5ccccc5)nc(-c5cccc(-c6ccncc6)c5)n4)c3)cc2-c2ccccc2-c2nc(-c3ccccc3)nc(-c3ccccc3)n2)n1]

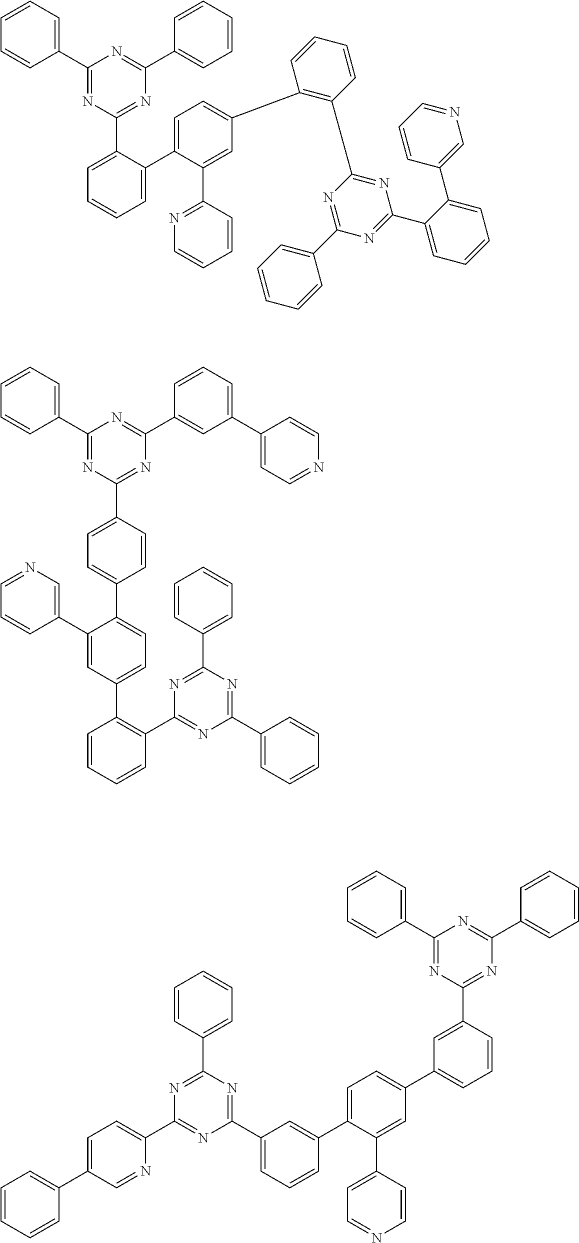 [SMILES: c1ccc(-c2nc(-c3ccccc3-c3cccnc3)nc(-c3ccccc3-c3ccc(-c4ccccc4-c4nc(-c5ccccc5)nc(-c5ccccc5)n4)c(-c4ccccn4)c3)n2)cc1 ; c1ccc(-c2nc(-c3ccc(-c4ccc(-c5ccccc5-c5nc(-c6ccccc6)nc(-c6ccccc6)n5)cc4-c4cccnc4)cc3)nc(-c3cccc(-c4ccncc4)c3)n2)cc1 ; c1ccc(-c2ccc(-c3nc(-c4ccccc4)nc(-c4cccc(-c5ccc(-c6cccc(-c7nc(-c8ccccc8)nc(-c8ccccc8)n7)c6)cc5-c5ccncc5)c4)n3)nc2)cc1]

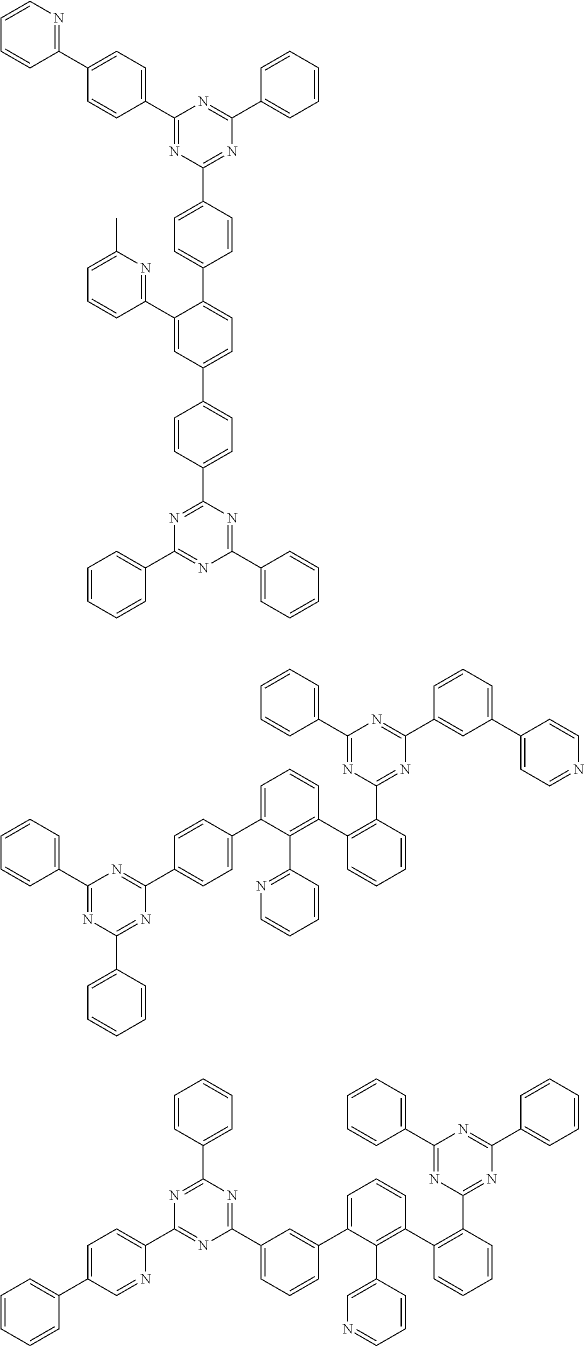 [SMILES: Cc1cccc(-c2cc(-c3ccc(-c4nc(-c5ccccc5)nc(-c5ccccc5)n4)cc3)ccc2-c2ccc(-c3nc(-c4ccccc4)nc(-c4ccc(-c5ccccn5)cc4)n3)cc2)n1 ; c1ccc(-c2nc(-c3ccccc3)nc(-c3ccc(-c4cccc(-c5ccccc5-c5nc(-c6ccccc6)nc(-c6cccc(-c7ccncc7)c6)n5)c4-c4ccccn4)cc3)n2)cc1 ; c1ccc(-c2ccc(-c3nc(-c4ccccc4)nc(-c4cccc(-c5cccc(-c6ccccc6-c6nc(-c7ccccc7)nc(-c7ccccc7)n6)c5-c5cccnc5)c4)n3)nc2)cc1]

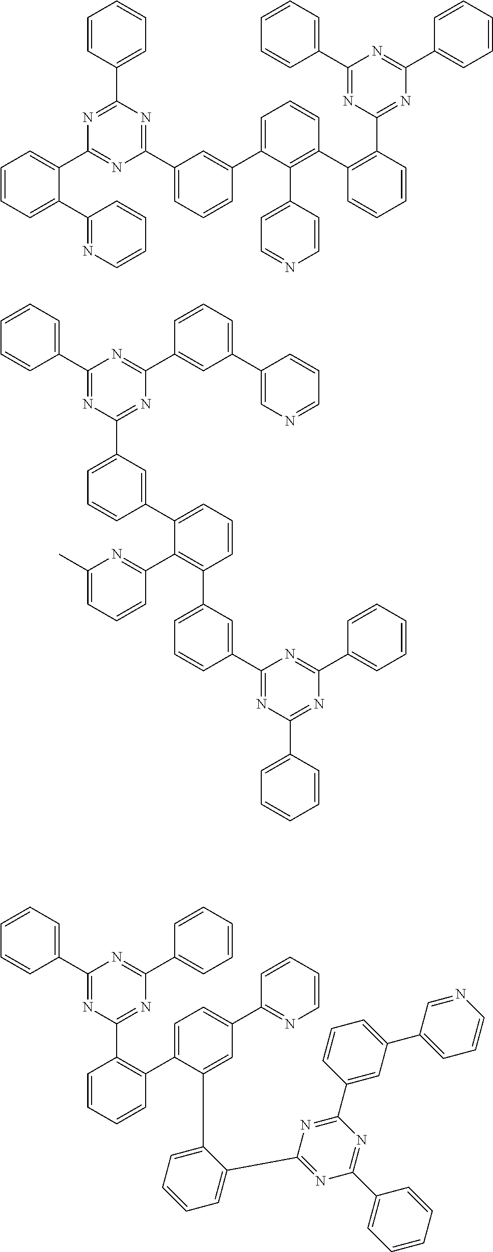 [SMILES: c1ccc(-c2nc(-c3cccc(-c4cccc(-c5ccccc5-c5nc(-c6ccccc6)nc(-c6ccccc6)n5)c4-c4ccncc4)c3)nc(-c3ccccc3-c3ccccn3)n2)cc1 ; Cc1cccc(-c2c(-c3cccc(-c4nc(-c5ccccc5)nc(-c5ccccc5)n4)c3)cccc2-c2cccc(-c3nc(-c4ccccc4)nc(-c4cccc(-c5cccnc5)c4)n3)c2)n1 ; c1ccc(-c2nc(-c3ccccc3)nc(-c3ccccc3-c3ccc(-c4ccccn4)cc3-c3ccccc3-c3nc(-c4ccccc4)nc(-c4cccc(-c5cccnc5)c4)n3)n2)cc1]

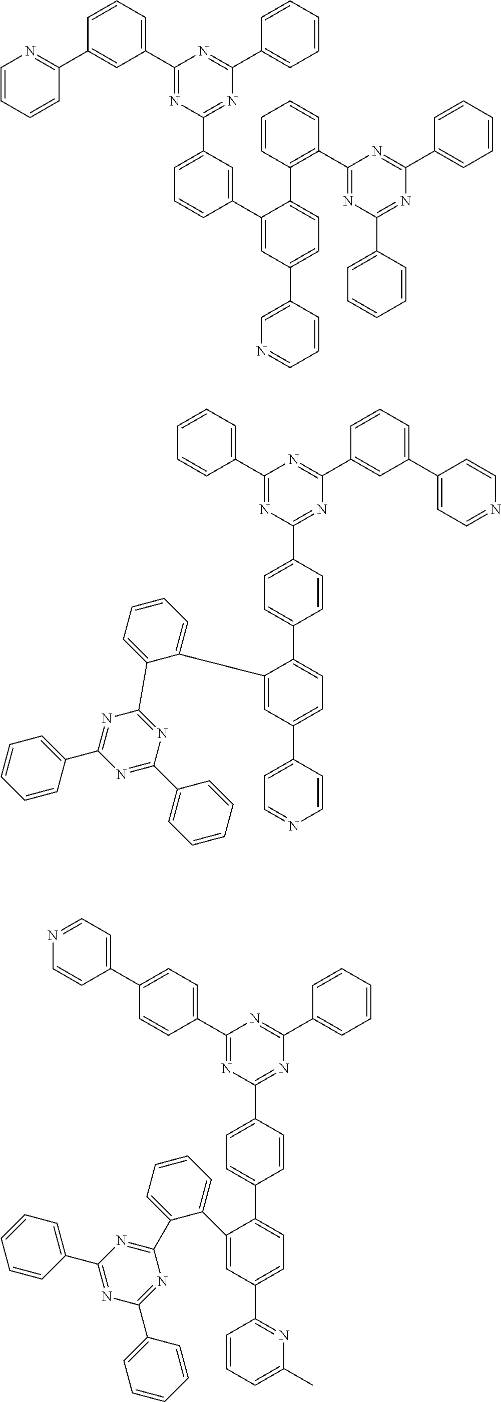 [SMILES: c1ccc(-c2nc(-c3cccc(-c4ccccn4)c3)nc(-c3cccc(-c4cc(-c5cccnc5)ccc4-c4ccccc4-c4nc(-c5ccccc5)nc(-c5ccccc5)n4)c3)n2)cc1 ; c1ccc(-c2nc(-c3ccc(-c4ccc(-c5ccncc5)cc4-c4ccccc4-c4nc(-c5ccccc5)nc(-c5ccccc5)n4)cc3)nc(-c3cccc(-c4ccncc4)c3)n2)cc1 ; Cc1cccc(-c2ccc(-c3ccc(-c4nc(-c5ccccc5)nc(-c5ccc(-c6ccncc6)cc5)n4)cc3)c(-c3ccccc3-c3nc(-c4ccccc4)nc(-c4ccccc4)n3)c2)n1]

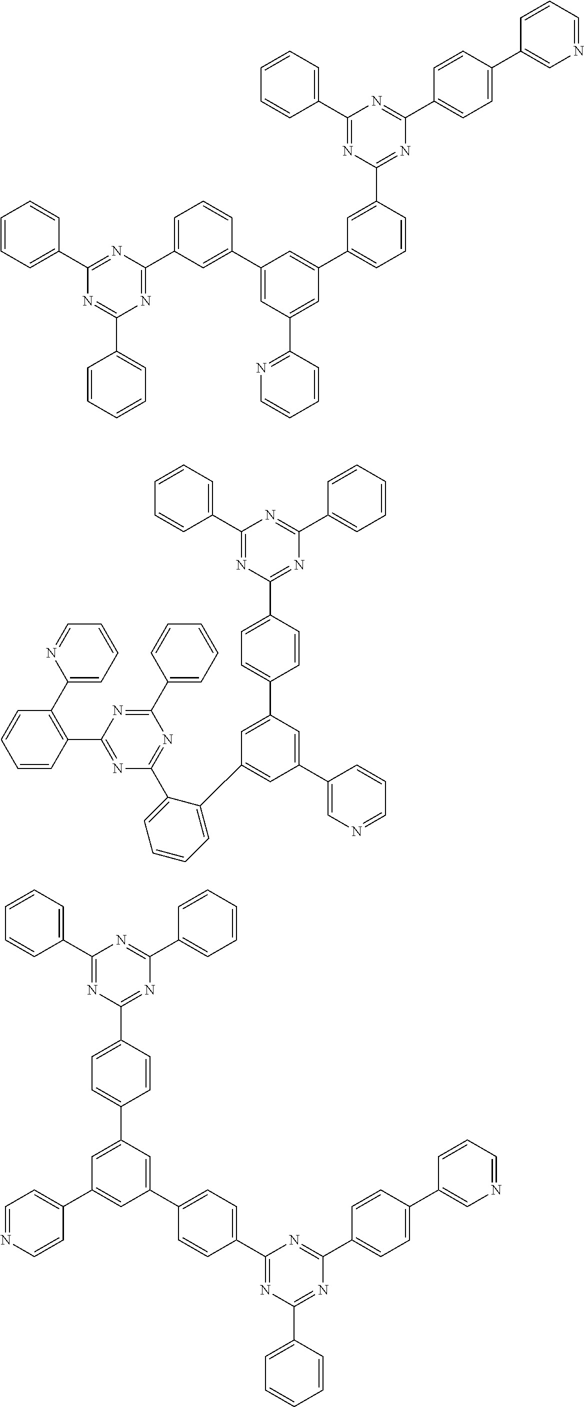

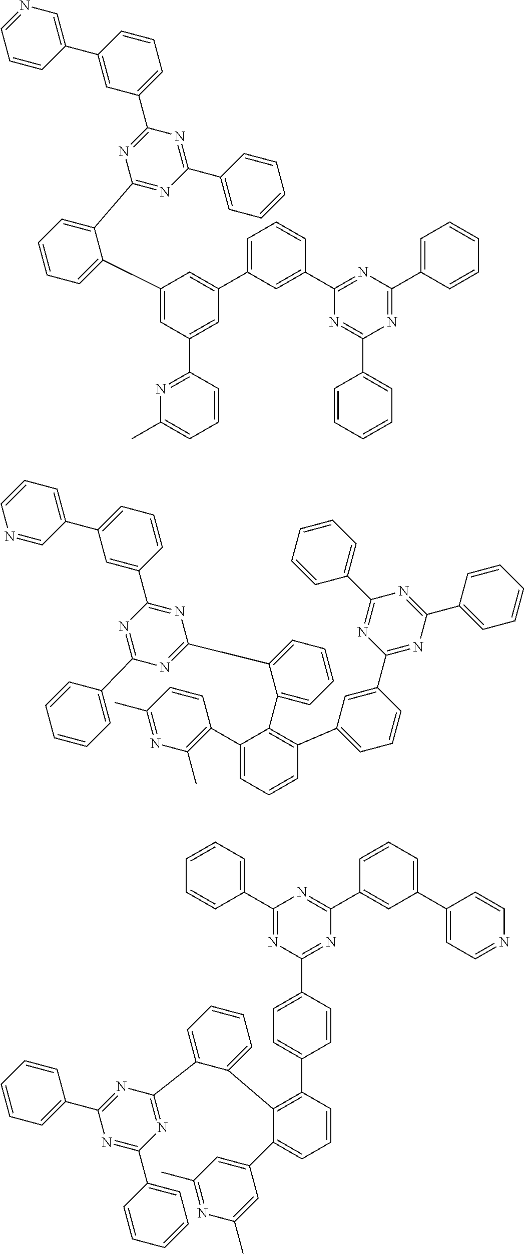 [SMILES: Cc1cccc(-c2cc(-c3cccc(-c4nc(-c5ccccc5)nc(-c5ccccc5)n4)c3)cc(-c3ccccc3-c3nc(-c4ccccc4)nc(-c4cccc(-c5cccnc5)c4)n3)c2)n1 ; Cc1ccc(-c2cccc(-c3cccc(-c4nc(-c5ccccc5)nc(-c5ccccc5)n4)c3)c2-c2ccccc2-c2nc(-c3ccccc3)nc(-c3cccc(-c4cccnc4)c3)n2)c(C)n1 ; Cc1cc(-c2cccc(-c3ccc(-c4nc(-c5ccccc5)nc(-c5cccc(-c6ccncc6)c5)n4)cc3)c2-c2ccccc2-c2nc(-c3ccccc3)nc(-c3ccccc3)n2)cc(C)n1]

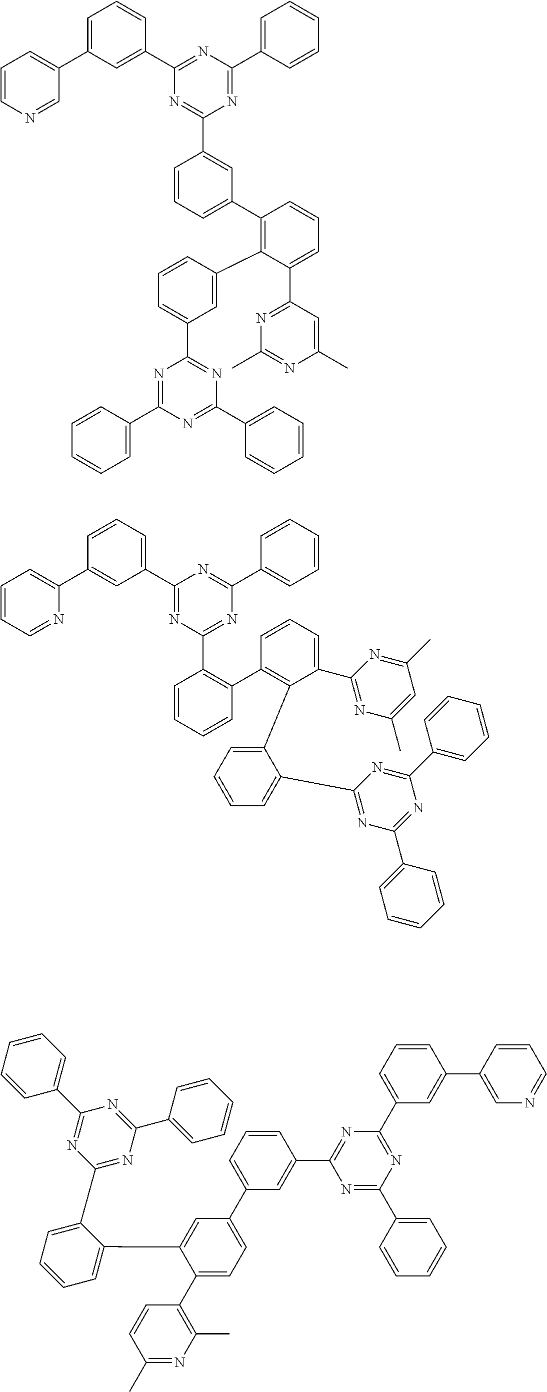 [SMILES: Cc1cc(-c2cccc(-c3cccc(-c4nc(-c5ccccc5)nc(-c5cccc(-c6cccnc6)c5)n4)c3)c2-c2cccc(-c3nc(-c4ccccc4)nc(-c4ccccc4)n3)c2)nc(C)n1 ; Cc1cc(C)nc(-c2cccc(-c3ccccc3-c3nc(-c4ccccc4)nc(-c4cccc(-c5ccccn5)c4)n3)c2-c2ccccc2-c2nc(-c3ccccc3)nc(-c3ccccc3)n2)n1 ; Cc1ccc(-c2ccc(-c3cccc(-c4nc(-c5ccccc5)nc(-c5cccc(-c6cccnc6)c5)n4)c3)cc2-c2ccccc2-c2nc(-c3ccccc3)nc(-c3ccccc3)n2)c(C)n1]

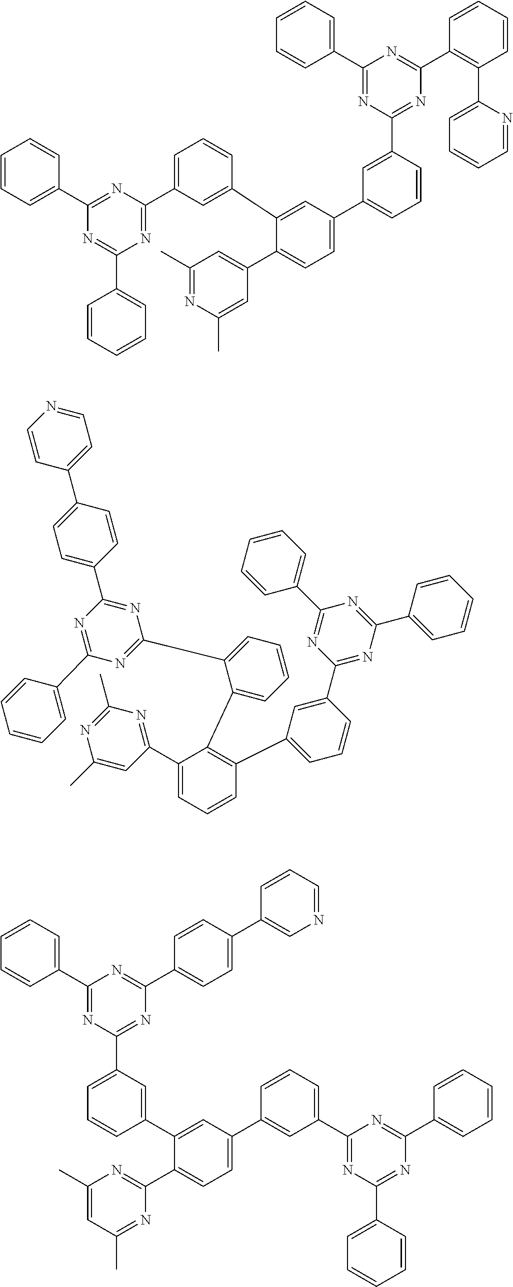 [SMILES: Cc1cc(-c2ccc(-c3cccc(-c4nc(-c5ccccc5)nc(-c5ccccc5-c5ccccn5)n4)c3)cc2-c2cccc(-c3nc(-c4ccccc4)nc(-c4ccccc4)n3)c2)cc(C)n1 ; Cc1cc(-c2cccc(-c3cccc(-c4nc(-c5ccccc5)nc(-c5ccccc5)n4)c3)c2-c2ccccc2-c2nc(-c3ccccc3)nc(-c3ccc(-c4ccncc4)cc3)n2)nc(C)n1 ; Cc1cc(C)nc(-c2ccc(-c3cccc(-c4nc(-c5ccccc5)nc(-c5ccccc5)n4)c3)cc2-c2cccc(-c3nc(-c4ccccc4)nc(-c4ccc(-c5cccnc5)cc4)n3)c2)n1]

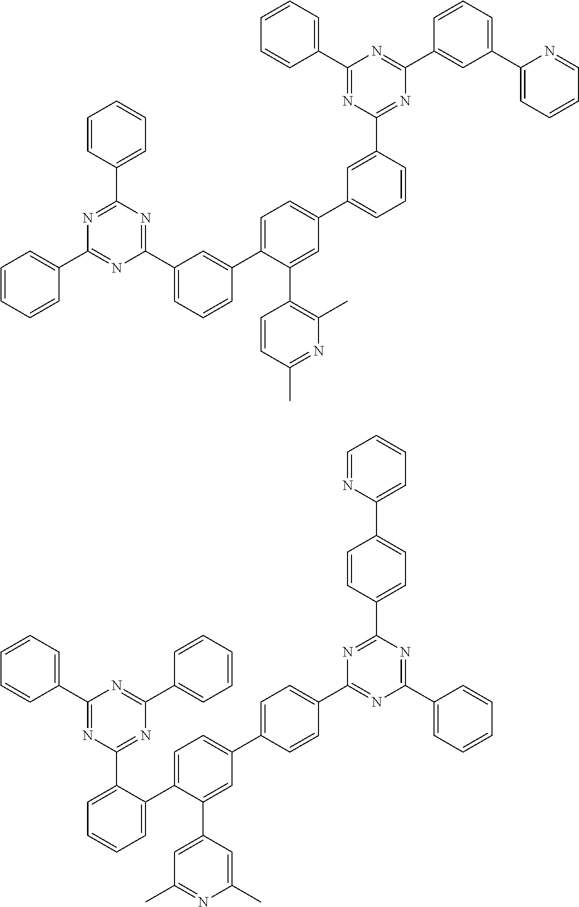 [SMILES: Cc1ccc(-c2cc(-c3cccc(-c4nc(-c5ccccc5)nc(-c5cccc(-c6ccccn6)c5)n4)c3)ccc2-c2cccc(-c3nc(-c4ccccc4)nc(-c4ccccc4)n3)c2)c(C)n1 ; Cc1cc(-c2cc(-c3ccc(-c4nc(-c5ccccc5)nc(-c5ccc(-c6ccccn6)cc5)n4)cc3)ccc2-c2ccccc2-c2nc(-c3ccccc3)nc(-c3ccccc3)n2)cc(C)n1]

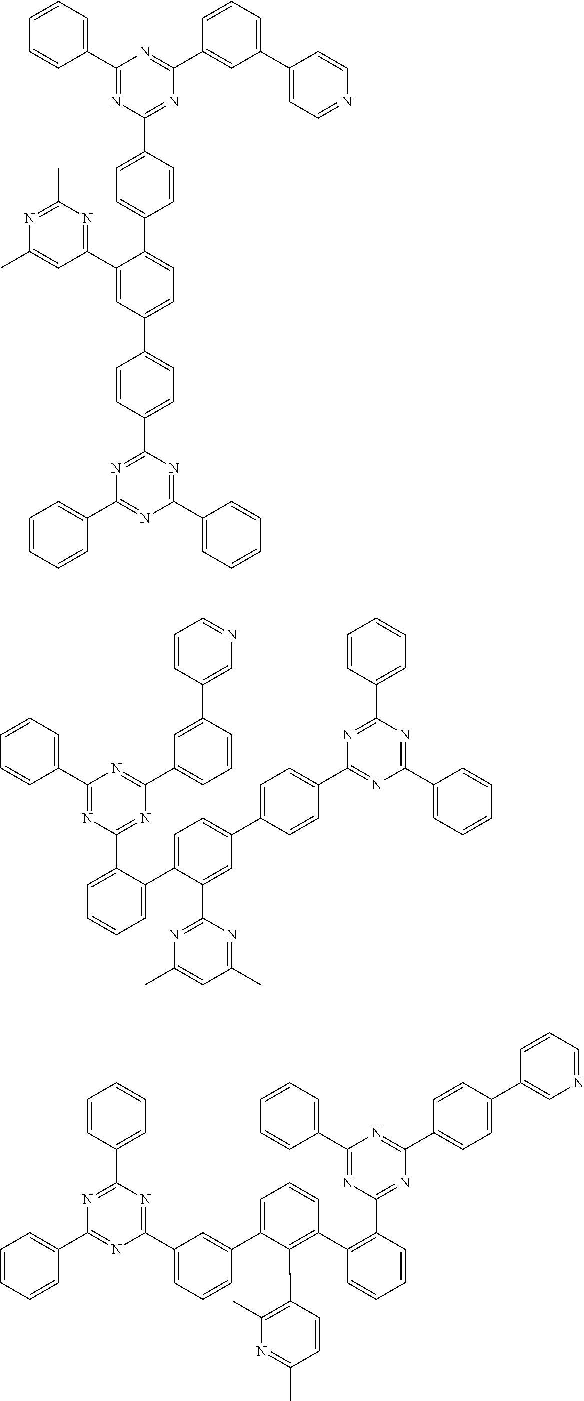 [SMILES: Cc1cc(-c2cc(-c3ccc(-c4nc(-c5ccccc5)nc(-c5ccccc5)n4)cc3)ccc2-c2ccc(-c3nc(-c4ccccc4)nc(-c4cccc(-c5ccncc5)c4)n3)cc2)nc(C)n1 ; Cc1cc(C)nc(-c2cc(-c3ccc(-c4nc(-c5ccccc5)nc(-c5ccccc5)n4)cc3)ccc2-c2ccccc2-c2nc(-c3ccccc3)nc(-c3cccc(-c4cccnc4)c3)n2)n1 ; Cc1ccc(-c2c(-c3cccc(-c4nc(-c5ccccc5)nc(-c5ccccc5)n4)c3)cccc2-c2ccccc2-c2nc(-c3ccccc3)nc(-c3ccc(-c4cccnc4)cc3)n2)c(C)n1]

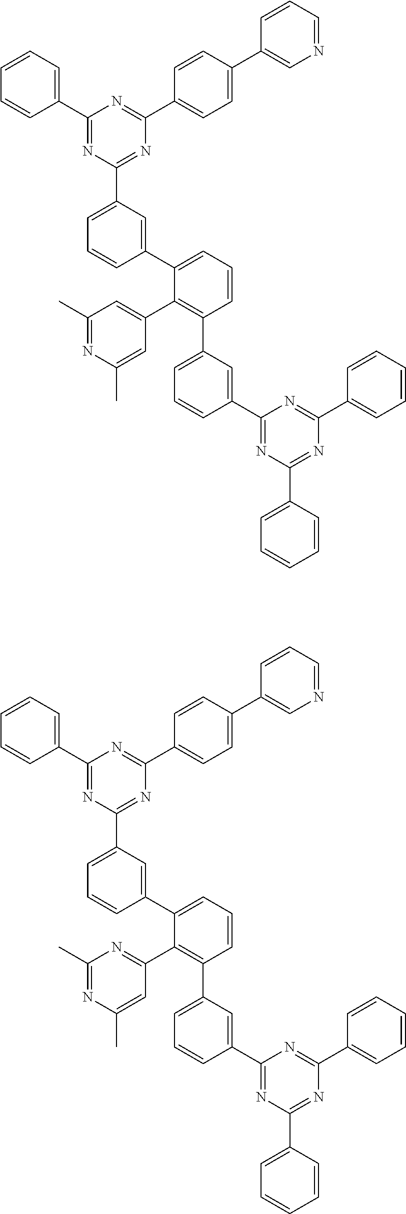 [SMILES: Cc1cc(-c2c(-c3cccc(-c4nc(-c5ccccc5)nc(-c5ccccc5)n4)c3)cccc2-c2cccc(-c3nc(-c4ccccc4)nc(-c4ccc(-c5cccnc5)cc4)n3)c2)cc(C)n1 ; Cc1cc(-c2c(-c3cccc(-c4nc(-c5ccccc5)nc(-c5ccccc5)n4)c3)cccc2-c2cccc(-c3nc(-c4ccccc4)nc(-c4ccc(-c5cccnc5)cc4)n3)c2)nc(C)n1]

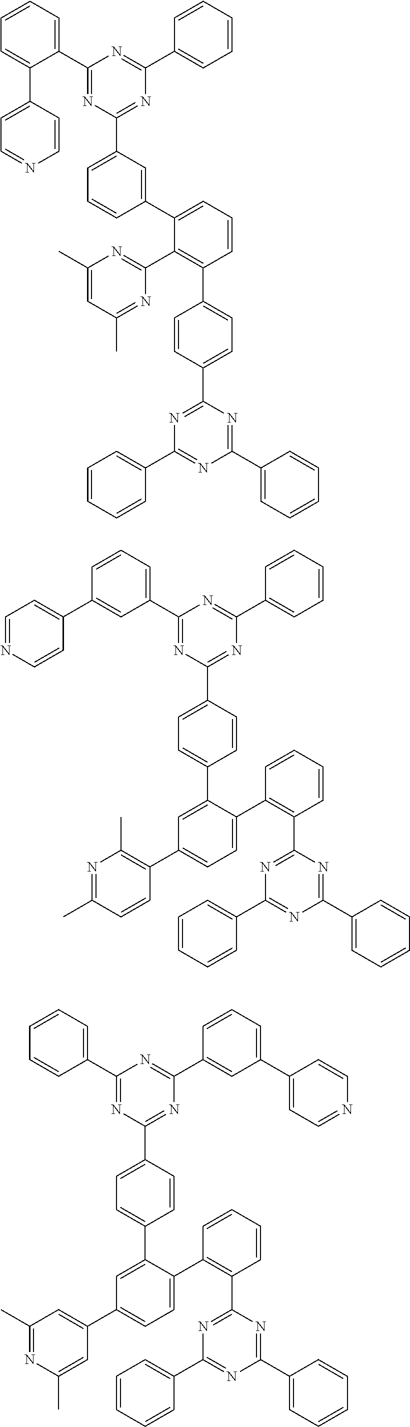

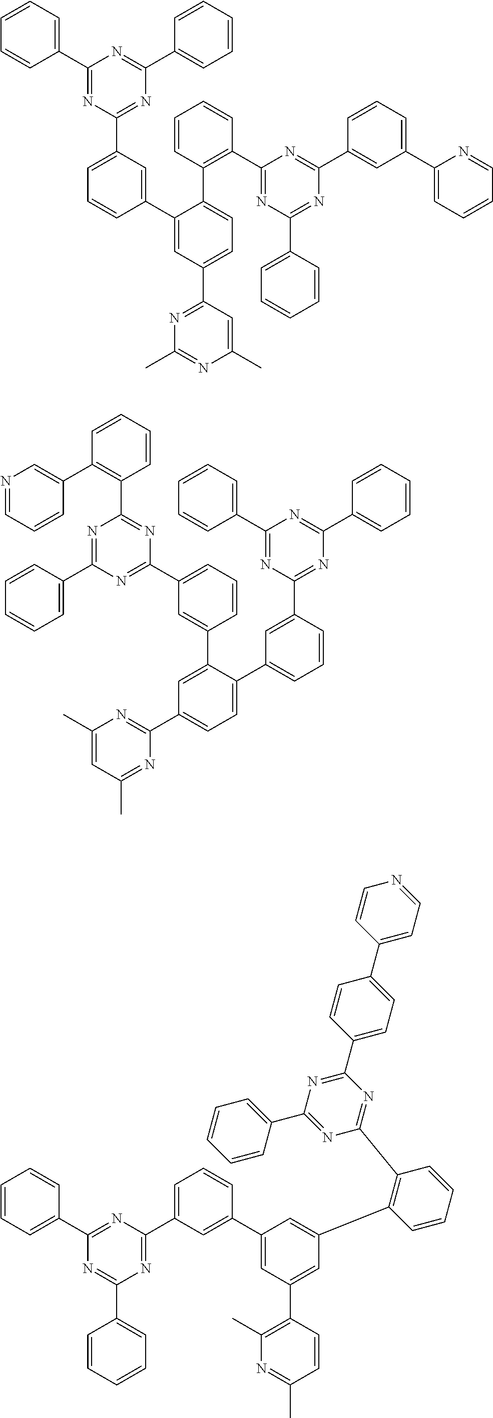 [SMILES: Cc1cc(-c2ccc(-c3ccccc3-c3nc(-c4ccccc4)nc(-c4cccc(-c5ccccn5)c4)n3)c(-c3cccc(-c4nc(-c5ccccc5)nc(-c5ccccc5)n4)c3)c2)nc(C)n1 ; Cc1cc(C)nc(-c2ccc(-c3cccc(-c4nc(-c5ccccc5)nc(-c5ccccc5)n4)c3)c(-c3cccc(-c4nc(-c5ccccc5)nc(-c5ccccc5-c5cccnc5)n4)c3)c2)n1 ; Cc1ccc(-c2cc(-c3cccc(-c4nc(-c5ccccc5)nc(-c5ccccc5)n4)c3)cc(-c3ccccc3-c3nc(-c4ccccc4)nc(-c4ccc(-c5ccncc5)cc4)n3)c2)c(C)n1]

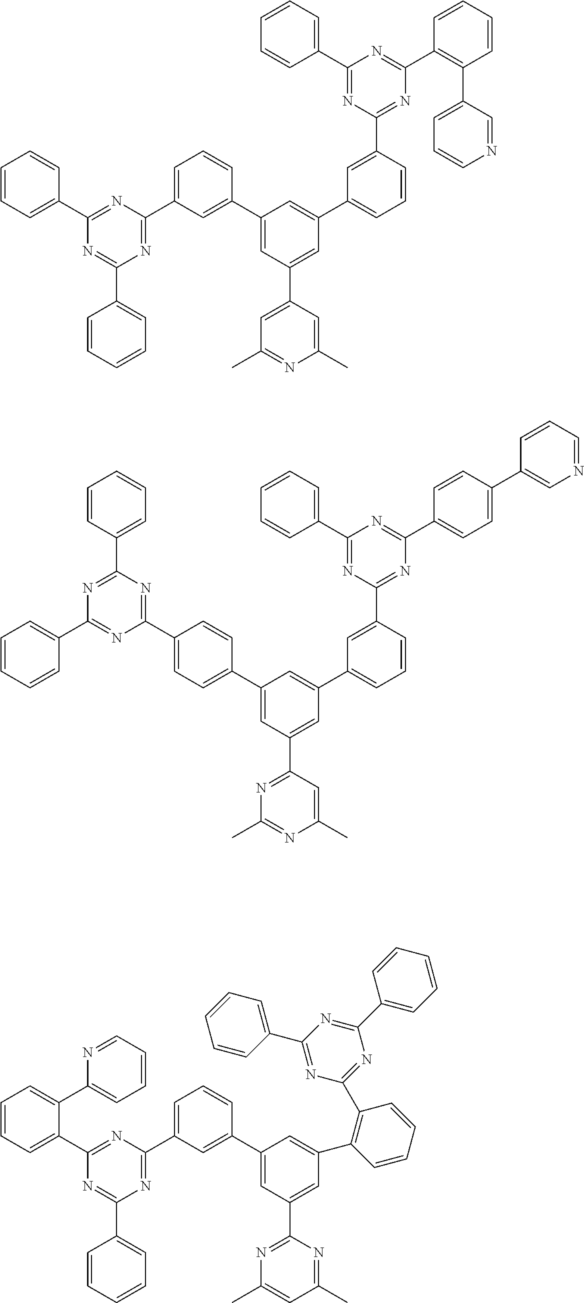 [SMILES: Cc1cc(-c2cc(-c3cccc(-c4nc(-c5ccccc5)nc(-c5ccccc5)n4)c3)cc(-c3cccc(-c4nc(-c5ccccc5)nc(-c5ccccc5-c5cccnc5)n4)c3)c2)cc(C)n1 ; Cc1cc(-c2cc(-c3ccc(-c4nc(-c5ccccc5)nc(-c5ccccc5)n4)cc3)cc(-c3cccc(-c4nc(-c5ccccc5)nc(-c5ccc(-c6cccnc6)cc5)n4)c3)c2)nc(C)n1 ; Cc1cc(C)nc(-c2cc(-c3cccc(-c4nc(-c5ccccc5)nc(-c5ccccc5-c5ccccn5)n4)c3)cc(-c3ccccc3-c3nc(-c4ccccc4)nc(-c4ccccc4)n3)c2)n1]

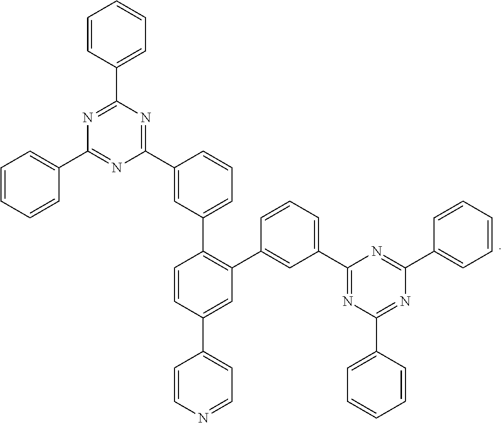 [SMILES: c1ccc(-c2nc(-c3ccccc3)nc(-c3cccc(-c4ccc(-c5ccncc5)cc4-c4cccc(-c5nc(-c6ccccc6)nc(-c6ccccc6)n5)c4)c3)n2)cc1]

9. An organic light emitting device comprising:
a first electrode;
a second electrode; and
an organic material layer having one or more layers provided between the first electrode and the second electrode,
wherein one or more layers of the organic material layer comprise the compound of claim 1.

10. The organic light emitting device of claim 9, wherein the organic material layer comprises an electron injection layer, an electron transport layer, or an electron injection and transport layer, and the electron injection layer, the electron transport layer, or the electron injection and transport layer comprises the compound.

11. The organic light emitting device of claim 9, wherein the organic material layer comprises a hole blocking layer, and the hole blocking layer comprises the compound.

12. The compound of claim 1, wherein Chemical Formula 1 is any one selected from the following compounds: 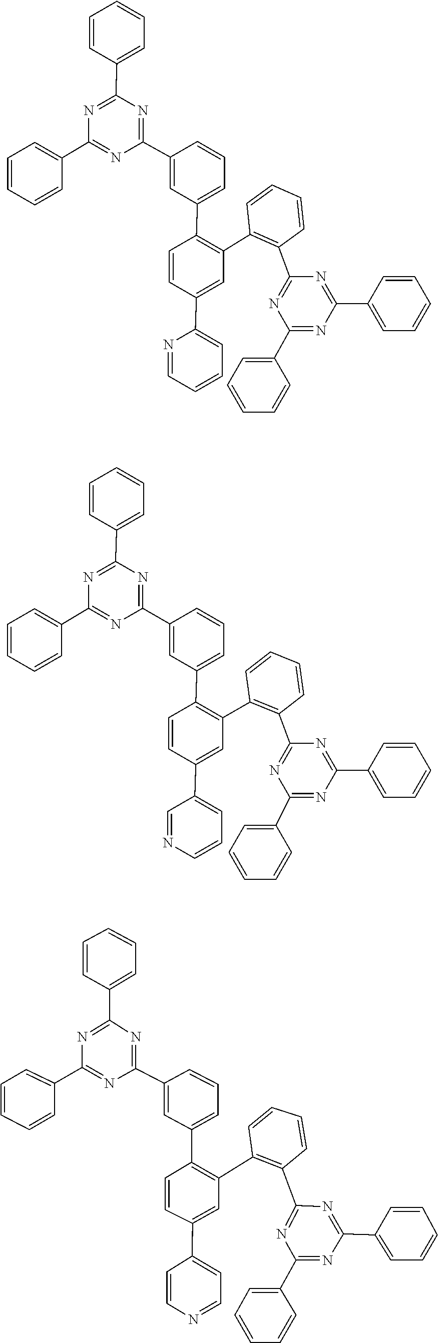 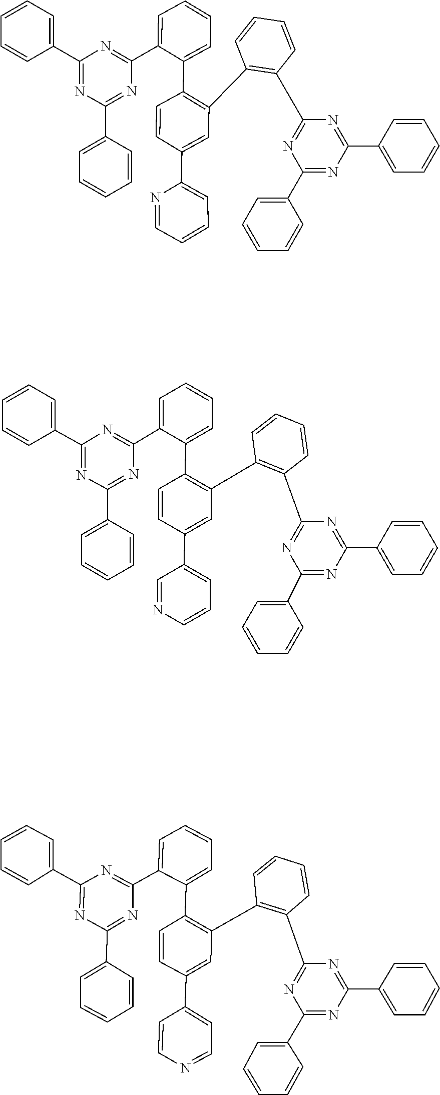 [SMILES: c1ccc(-c2nc(-c3ccccc3)nc(-c3ccccc3-c3ccc(-c4ccccn4)cc3-c3ccccc3-c3nc(-c4ccccc4)nc(-c4ccccc4)n3)n2)cc1 ; c1ccc(-c2nc(-c3ccccc3)nc(-c3ccccc3-c3ccc(-c4cccnc4)cc3-c3ccccc3-c3nc(-c4ccccc4)nc(-c4ccccc4)n3)n2)cc1 ; c1ccc(-c2nc(-c3ccccc3)nc(-c3ccccc3-c3ccc(-c4ccncc4)cc3-c3ccccc3-c3nc(-c4ccccc4)nc(-c4ccccc4)n3)n2)cc1] 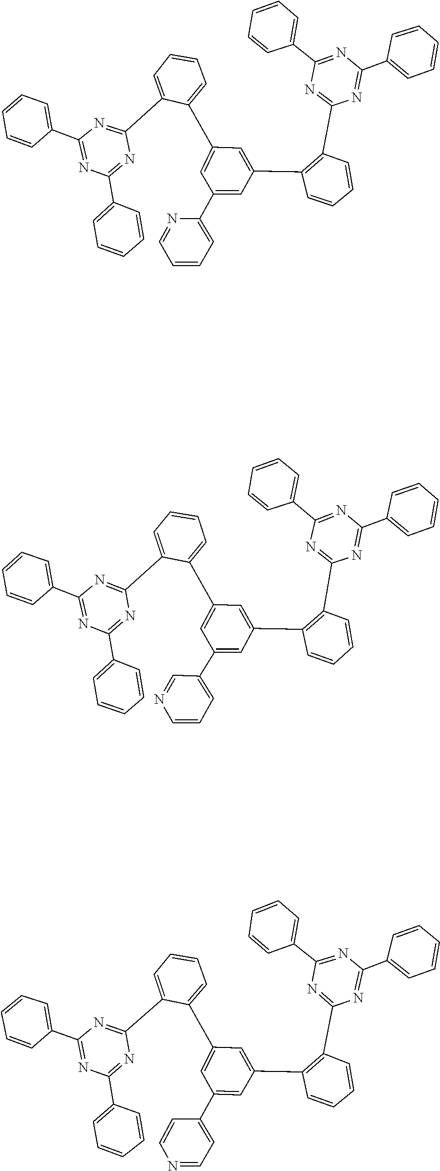 [SMILES: c1ccc(-c2nc(-c3ccccc3)nc(-c3ccccc3-c3cc(-c4ccccn4)cc(-c4ccccc4-c4nc(-c5ccccc5)nc(-c5ccccc5)n4)c3)n2)cc1 ; c1ccc(-c2nc(-c3ccccc3)nc(-c3ccccc3-c3cc(-c4cccnc4)cc(-c4ccccc4-c4nc(-c5ccccc5)nc(-c5ccccc5)n4)c3)n2)cc1 ; c1ccc(-c2nc(-c3ccccc3)nc(-c3ccccc3-c3cc(-c4ccncc4)cc(-c4ccccc4-c4nc(-c5ccccc5)nc(-c5ccccc5)n4)c3)n2)cc1] 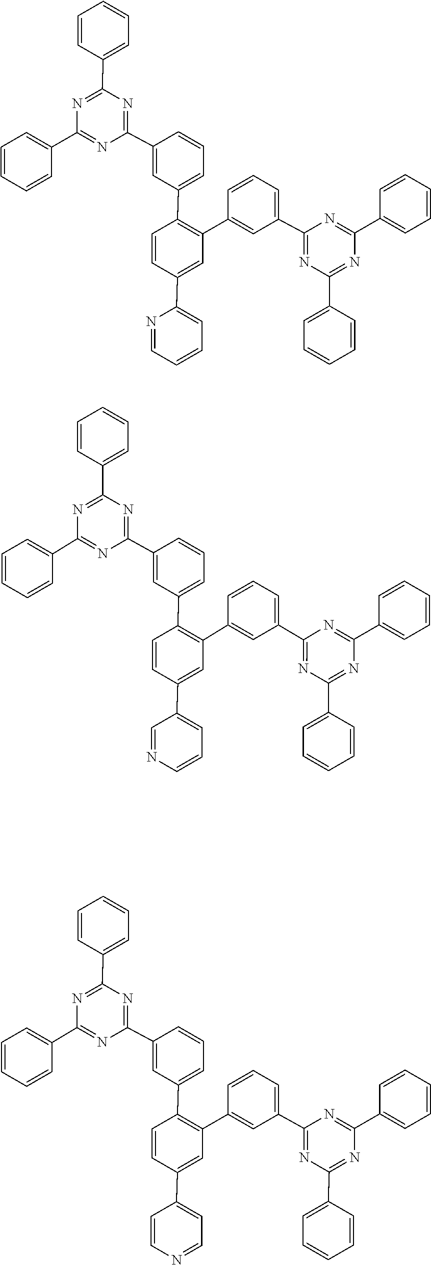 [SMILES: c1ccc(-c2nc(-c3ccccc3)nc(-c3cccc(-c4ccc(-c5ccccn5)cc4-c4cccc(-c5nc(-c6ccccc6)nc(-c6ccccc6)n5)c4)c3)n2)cc1 ; c1ccc(-c2nc(-c3ccccc3)nc(-c3cccc(-c4ccc(-c5cccnc5)cc4-c4cccc(-c5nc(-c6ccccc6)nc(-c6ccccc6)n5)c4)c3)n2)cc1 ; c1ccc(-c2nc(-c3ccccc3)nc(-c3cccc(-c4ccc(-c5ccncc5)cc4-c4cccc(-c5nc(-c6ccccc6)nc(-c6ccccc6)n5)c4)c3)n2)cc1] 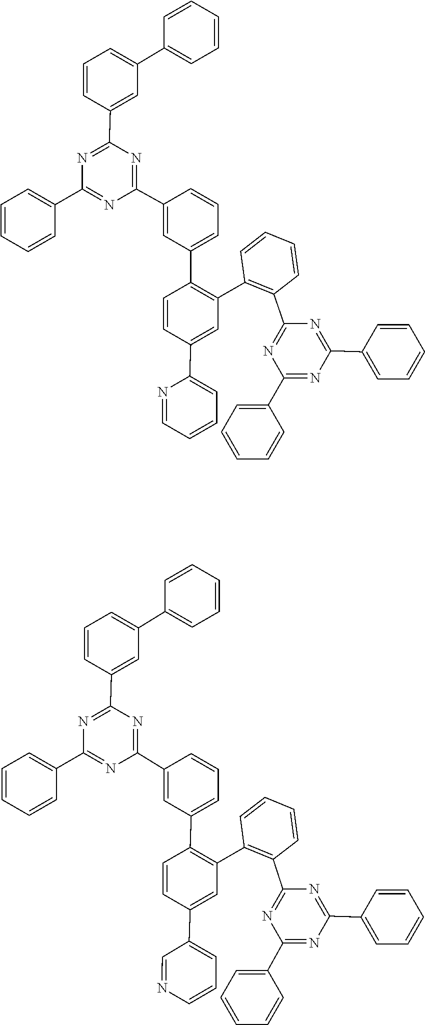 [SMILES: c1ccc(-c2cccc(-c3nc(-c4ccccc4)nc(-c4cccc(-c5ccc(-c6ccccn6)cc5-c5ccccc5-c5nc(-c6ccccc6)nc(-c6ccccc6)n5)c4)n3)c2)cc1 ; c1ccc(-c2cccc(-c3nc(-c4ccccc4)nc(-c4cccc(-c5ccc(-c6cccnc6)cc5-c5ccccc5-c5nc(-c6ccccc6)nc(-c6ccccc6)n5)c4)n3)c2)cc1] 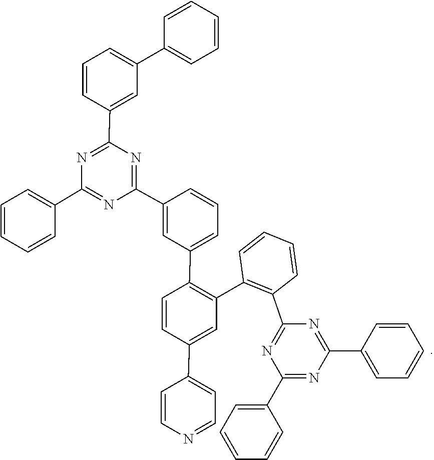 [SMILES: c1ccc(-c2cccc(-c3nc(-c4ccccc4)nc(-c4cccc(-c5ccc(-c6ccncc6)cc5-c5ccccc5-c5nc(-c6ccccc6)nc(-c6ccccc6)n5)c4)n3)c2)cc1]
